(19) United States
(12) Patent Application Publication
(10) Pub. No.: US 2013/0041136 A1
(43) Pub. Date: Feb. 14, 2013

(54) LIGANDS THAT HAVE BINDING SPECIFICITY FOR EGFR AND/OR VEGF AND METHODS OF USE THEREFOR

(71) Applicant: Domantis Limited, Middlesex (GB)

(72) Inventors: Roland BECKMANN, Cambridge (GB); Rudolf M T De Wildt, Stevenage (GB); Steve Holmes, Cambridge (GB); Olga Ignatovich, Cambridge (GB); Laurent S. Jespers, Cambridge (GB); Haiqun Liu, Cambridge (GB); Malgorzata Pupecka, Cambridge (GB); Armin Sepp, Cambridge (GB); Michael Steward, Stevenage (GB)

(73) Assignee: Domantis Limited, Middlesex (GB)

(21) Appl. No.: 13/644,414

(22) Filed: Oct. 26, 2012

Related U.S. Application Data
(63) Continuation of application No. 12/086,020, filed on Nov. 17, 2009, now abandoned.
(60) Provisional application No. 60/742,992, filed on Dec. 6, 2005.
(60) Provisional application No. 60/758,355, filed on Jan. 11, 2006.

(51) Int. Cl. C07K 16/46

(52) U.S. Cl. 530/387.3

(57) ABSTRACT

Disclosed are ligands that have binding specificity for vascular endothelial growth factor (VEGF), for epidermal growth factor receptor (EGFR), or for VEGF and EGFR. Also disclosed are methods of using these ligands. In particular, the use of these ligands for cancer therapy is described.

### RELATED APPLICATION

This application claims the benefit of U.S. Provisional Application No. 60/742,992, filed on Dec. 6, 2005, and the benefit of U.S. Provisional Application No. 60/758,355, filed on Jan. 11, 2006. The entire teachings of the above applications are incorporated herein by reference.

### BACKGROUND OF THE INVENTION

Cancer is a leading cause of mortality and morbidity. Approaches to treating cancer include surgical intervention to remove tumors and chemotherapy. These approaches can successfully cure some patients. However, even patients that appear to have been cured often suffer a recurrence of the cancer necessitating further therapy. Chemotherapeutic agents generally are nonselective agents that are toxic to cells, such as proliferating cells. Accordingly, such agents may effectively kill cancer cells but also kill healthy cells producing several deleterious side effects.

Certain cancer cells express or overexpress certain cellular components such as cell surface proteins, or express different cellular components when compared to normal cells. One approach to address the short comings of surgical and chemotherapeutic approaches to cancer therapy and diagnosis involves targeting cancer cells, for example using antibodies or antibody fragments that bind to proteins that are expressed or overexpressed on cancerous cells. A number of such target proteins have been identified. Among such proteins is the epidermal growth factor receptor (EGFR).

EGFR is a member of the ErbB1 family and transduces signals that lead to cellular proliferation and survival, and the elaboration of growth and angiogenic factors upon binding epidermal growth factor (EGF) and or transforming growth factor alpha (TGF alpha). Accordingly, EGFR has been demonstrated to be involved in tumor growth, metastasis and angiogenesis. Further, many cancers express EGFR, such as bladder cancer, ovarian cancer, colorectal cancer, breast cancer, lung cancer (e.g., non-small cell lung carcinoma), gastric cancer, pancreatic cancer, prostate cancer, head and neck cancer, renal cancer and gall bladder cancer. ERBITUX (cetuximab; Imclone Systems Inc) is a chimeric mouse/human antibody that binds human EGFR that has been approved for treating certain EGFR-expressing cancers in combination with irinotecan.

An important pathophysiological process that facilitates tumor formation, metastasis and recurrence is tumor angiogenesis. This process is mediated by the elaboration of angiogenic factors by the tumor, such as vascular endothelial growth factor (VEGF), which induce the formation of blood vessels that deliver nutrients to the tumor. Accordingly, another approach to treating certain cancers is to inhibit tumor angiogenesis mediated by VEGF, thereby starving the tumor. AVASTIN (bevacizumab; Genetech, Inc.) is a humanized antibody that binds human VEGF that has been approved for treating colorectal cancer. An antibody referred to as antibody 2C3 (ATCC Accession No. PTA 1595) is reported to bind VEGF and inhibit binding of VEGF to epidemial growth factor receptor 2.

Targering EGFR or VEGF with currently available therapeutics is not effective in all patients, or for all cancers (e.g., EGFR-expressing cancers). Thus, a need exists for improved agents for treating cancer and other pathological conditions.

### SUMMARY OF THE INVENTION

The invention relates to ligands that have binding specificity for VEGF (e.g., human VEGF), ligands that have binding specificity for EGFR (e.g., human EGFR), and to ligands that have binding specificity for VEGF and EGFR (e.g., human VEGF and human EGFR). For example, the ligand can comprise a polypeptide domain having a binding site with binding specificity for VEGF, a polypeptide domain having a binding site with binding specificity EGFR, or comprise a polypeptide domain having a binding site with binding specificity for VEGF and a polypeptide domain having a binding site with binding specificity EGFR.

In one aspect, the invention relates to a ligand that has binding specificity for VEGF and for EGFR. Such ligands comprise at least one protein moiety that has a binding site with binding specificity for VEGF and at least one protein moiety that has a binding site with binding specificity for EGFR. The protein moiety that has a binding site with binding specificity for VEGF and the protein moiety that has a binding site with binding specificity for EGFR can each be any suitable binding moiety. The protein moieties can be a peptide moiety, polypeptide moiety or protein moiety. For example, the protein moieties can be provided by an antibody fragment that has a binding site with binding specificity for VEGF or EGFR, such as an immunoglobulin single variable domain that has binding specificity for VEGF or EGFR.

The ligand can comprise a protein moiety that has a binding site with binding specificity for VEGF that competes for binding to VEGF with AVASTIN (bevacizumab; Genentech, Inc.) and/or antibody 2C3 (ATCC Accession No. PTA 1595). The ligand can comprise a protein moiety that has a binding site with binding specificity for EGFR that competes for binding to EGFR with ERBITUX (cetuximab; Imclone Systems, Inc.) and/or VECTIBIX (panitumumab; Amgen, Inc.). In some embodiments, the ligand comprises a protein moiety that has a binding site with binding specificity for VEGF that competes for binding to VEGF with bevacizumab and/or antibody 2C3 (ATCC Accession No. PTA 1595), and further comprises a protein moiety that has a binding site with binding specificity for EGFR that competes for binding to EGFR with cetuximab.

In some embodiments, the ligand comprises a protein moiety that has a binding site with binding specificity for VEGF (e.g., an immunoglobulin single variable domain) that competes for binding to VEGF with an anti-VEGF domain antibody (dAb) selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), TAR15-26-549 (SEQ ID NO:198), TAR15-26-550 (SEQ ID NO:539), and TAR15-26-551 (SEQ ID NO:540).

In some embodiments, the ligand comprises a protein moiety that has a binding site with binding specificity for VEGF (e.g., an immunoglobulin single variable domain) that competes for binding to VEGF with TAR15-26-555 (SEQ ID NO:704).

Additionally, or in other embodiments, the ligand can comprise a protein moiety that has a binding site with binding specificity for EGFR (e.g., an immunoglobulin single variable domain) that competes for binding to EGFR with an anti-EGFR domain antibody (dAb) selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In particular embodiments, the ligand has binding specificity for VEGF and for EGFR and comprises a protein moiety that has a binding site with binding specificity for VEGF that competes for binding to VEGF with an anti-VEGF domain antibody (dAb) selected from the group consisting of TAR15-6 (SEQ ID NO:117), TAR15-8 (SEQ ID NO:119), and TAR15-26 (SEQ ID NO:123), and further comprises a protein moiety that has a binding site with binding specificity for EGFR that competes for binding to EGFR with an anti-EGFR domain antibody (dAb) selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

In particular embodiments, the ligand has binding specificity for VEGF and for EGFR and comprises a protein moiety that has a binding site with binding specificity for VEGF that competes for binding to VEGF with an anti-VEGF domain antibody (dAb) selected from the group consisting of TAR15-6 (SEQ ID NO:117), TAR15-8 (SEQ ID NO:119), and TAR15-26 (SEQ ID NO:123), and further comprises a protein moiety that has a binding site with binding specificity for EGFR that competes for binding to EGFR with an anti-EGFR domain antibody (dAb) selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

In more particular embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for VEGF competes for binding to VEGF with an anti-VEGF domain antibody (dAb) selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540).

For example, the immunoglobulin single variable domain with binding specificity for VEGF can comprise an amino acid sequence that has at least about 85% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540).

In other particular embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for EGFR competes for binding to EGFR with an anti-EGFR domain antibody (dAb) selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In other particular embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for EGFR competes for binding to EGFR with an anti-EGFR domain antibody (dAb) selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

For example, the immunoglobulin single variable domain with binding specificity for EGFR can comprise an amino acid sequence that has at least about 85% amino acid sequence identity with the amino acid sequence of a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

For example, the immunoglobulin single variable domain with binding specificity for EGFR can comprise an amino acid sequence that has at least about 85% amino acid sequence identity with the amino acid sequence of a dAb selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

In some embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for VEGF competes for binding to VEGF with an anti-VEGF domain antibody (dAb) selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540); and wherein an immunoglobulin single variable domain with binding specificity for EGFR competes for binding to EGFR with an anti-EGFR domain antibody (dAb) selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In additional embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for VEGF competes for binding to VEGF with an anti-VEGF domain antibody (dAb) selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), TAR15-26-549 (SEQ ID NO:198), TAR15-26-550 (SEQ ID NO:539), and TAR15-26-551 (SEQ ID NO:540); and wherein an immunoglobulin single variable domain with binding specificity for EGFR competes for binding to EGFR with an anti-EGFR domain antibody (dAb) selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

For example, the ligand can comprise an immunoglobulin single variable domain with binding specificity for VEGF that comprises an amino acid sequence that has at least about 85% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18(SEQ ID NO:112), TAR15-19 (SEQ ID NO:113), TAR15-20 (SEQ ID NO:114), TAR15-26 (SEQ ID NO:115), TAR15-5 (SEQ ID NO:116), TAR15-7 (SEQ ID NO:117), TAR15-22 (SEQ ID NO:118), TAR15-8 (SEQ ID NO:119), TAR15-23(SEQ ID NO:120), TAR15-24 (SEQ ID NO:121), TAR15-25(SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-6-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-6-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:1161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:1169) TAR15-26-521 (SEQ ID NO:170), TAR15-26-522 (SEQ ID NO:171), TAR15-26-523 (SEQ ID NO:172) TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178) TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540); and further comprise an immunoglobulin single variable domain with binding specificity for EGFR that comprise an amino acid sequence that has at least about 85% amino acid sequence identity with the amino acid sequence of a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

For example, the ligand can comprise an immunoglobulin single variable domain with binding specificity for VEGF that comprises an amino acid sequence that has at least about 85% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), TAR15-26-549 (SEQ ID NO:198), TAR15-26-550 (SEQ ID NO:539), and TAR15-26-551 (SEQ ID NO:540); and further comprise an immunoglobulin single variable domain with binding specificity for EGFR that comprise an amino acid sequence that has at least about 85% amino acid sequence identity with the amino acid sequence of a dAb selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

In some embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for VEGF competes for binding to VEGF with an anti-VEGF domain antibody (dAb) selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540); and an immunoglobulin single variable domain with binding specificity for EGFR competes for binding to EGFR with cetuximab.

For example, the immunoglobulin single variable domain with binding specificity for VEGF can comprise an amino acid sequence that has at least about 85% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540).

In other embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for VEGF competes for binding to VEGF with bevacizumab and/or antibody 2C3 (ATCC Accession No. PTA 1595); and an immunoglobulin single variable domain with binding specificity for EGFR competes for binding to EGFR with an anti-EGFR domain antibody (dAb) selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In other embodiments, the ligand has binding specificity for VEGF and for EGFR and comprises at least one immunoglobulin single variable domain with binding specificity for VEGF and at least one immunoglobulin single variable domain with binding specificity for EGFR, wherein an immunoglobulin single variable domain with binding specificity for VEGF competes for binding to VEGF with bevacizumab and/or antibody 2C3 (ATCC Accession No. PTA 1595); and an immunoglobulin single variable domain with binding specificity for EGFR competes for binding to EGFR with an anti-EGFR domain antibody (dAb) selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

For example, the immunoglobulin single variable domain with binding specificity for EGFR can comprise an amino acid sequence that has at least about 85% amino acid sequence identity with the amino acid sequence of a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

For example, the immunoglobulin single variable domain with binding specificity for EGFR can comprise an amino acid sequence that has at least about 85% amino acid sequence identity with the amino acid sequence of a dAb selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

In other embodiments, the ligand that has binding specificity for VEGF and for EGFR comprises a first immunoglobulin single variable domain with binding specificity for VEGF and a second immunoglobulin single variable domain with binding specificity for EGFR, wherein said first immunoglobulin single variable domain competes for binding to VEGF with bevacizumab and/or antibody 2C3 (ATCC Accession No. PTA 1595); and said second immunoglobulin single variable domain competes for binding to EGFR with cetuximab.

In particular embodiments, the ligand has binding specificity for VEGF and for EGFR and comprises at least one immunoglobulin single variable domain with binding specificity for VEGF and at least one immunoglobulin single variable domain with binding specificity for EGFR, wherein the ligand comprises an immunoglobulin single variable domain with binding specificity for VEGF that comprises an amino acid sequence that has at least 90% amino acid sequence identity with the amino acid sequence of an anti-VEGF dAb selected from the group consisting of TAR15-6 (SEQ ID NO:117), TAR15-8 (SEQ ID NO:119), and TAR15-26 (SEQ ID NO:123), and further comprises an immunoglobulin single variable domain with binding specificity for EGFR that comprises an amino acid sequence that has at least 90% amino acid sequence identity with an amino acid sequence selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), or DOM16-39-200 (SEQ ID NO:441).

In particular embodiments, the ligand has binding specificity for VEGF and for EGFR and comprises at least one immunoglobulin single variable domain with binding specificity for VEGF and at least one immunoglobulin single variable domain with binding specificity for EGFR, wherein the ligand comprises an immunoglobulin single variable domain with binding specificity for VEGF that comprises an amino acid sequence that has at least 90% amino acid sequence identity with the amino acid sequence of an anti-VEGF dAb selected from the group consisting of TAR15-6 (SEQ ID NO:117), TAR15-8 (SEQ ID NO:119), and TAR15-26 (SEQ ID NO:123), and further comprises an immunoglobulin single variable domain with binding specificity for EGFR that comprises an amino acid sequence that has at least 90% amino acid sequence identity with an amino acid sequence selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

The ligand that has binding specificity for VEGF and for EGFR can inhibit binding of epidermal growth factor (EGF) and/or transforming growth factor alpha (TGFalpha) to EGFR, inhibit the activity of EGFR, and/or inhibit the activity of EGFR without substantially inhibiting binding of epidermal growth factor (EGF) and/or transforming growth factor alpha (TGFalpha) to EGFR. In addition, or alternatively, the ligand that has binding specificity for VEGF and for EGFR can inhibit binding of VEGF to vascular endothelial growth factor receptor 1 (VEGFR1) and/or vascular endothelial growth factor receptor 2 (VEGFR2), inhibit the activity of VEGF and/or inhibit the activity of VEGF without substantially inhibiting binding of VEGF to VEGFR1 and/or VEGFR2.

The ligand that has binding specificity for VEGF and for EGFR can contain a protein binding moiety (e.g., immunoglobulin single variable domain) with binding specificity for VEGF that binds VEGF with an affinity (KD) that is between about 100 nM and about 1 pM, as determined by surface plasmon resonance.

The ligand that has binding specificity for VEGF and for EGFR can contain a protein binding moiety (e.g., immunoglobulin single variable domain) with binding specificity for EGFR that binds EGFR with an affinity (KD) that is between about 100 nM and about 1 pM or about 10 nM to about 100 pM, as determined by surface plasmon resonance.

The ligand that has binding specificity for VEGF and for EGFR can bind VEGF with an affinity (KD) that is between about 100 nM and about 1 pM, as determined by surface plasmon resonance.

The ligand that has binding specificity for VEGF and for EGFR can bind EGFR with an affinity (KD) that is between about 100 nM and about 1 pM or about 10 nM to about 100 pM, as determined by surface plasmon resonance.

The ligand that has binding specificity for VEGF and for EGFR can comprise an immunoglobulin single variable domain with binding specificity for VEGF that is a V_{HH} and/or an immunoglobulin single variable domain with bining specificity for EGFR that is a V_{HH}.

The ligand that has binding specificity for VEGF and for EGFR can comprise an immunoglobulin single variable domain with binding specificity for VEGF and an immunoglobulin single variable domain with binding specificity for EGFR, wherein the immunoglobulin single domains are selected from the group consisting of a human V_{H} and a human V_{L}.

In some embodiments, the ligand that has binding specificity for VEGF and for EGFR can be an IgG-like format comprising two immunoglobulin single variable domains with binding specificity for VEGF, and two immunoglobulin single variable domains with binding specificity for EGFR.

In some embodiments, the ligand that has binding specificity for VEGF and for EGFR can comprise an antibody Fc region.

The invention also relates to a ligand that has binding specificity for VEGF, comprising at least one immunoglobulin single variable domain with binding specificity for VEGF, wherein an immunoglobulin single variable domain with binding specificity for VEGF competes for binding to VEGF with an anti-VEGF domain antibody (dAb) selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540).

For example, an immunoglobulin single variable domain with binding specificity for VEGF can comprise an amino acid sequence that has at least about 85% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), and TAR15-26-549 (SEQ ID NO:198), TAR15-26-550 (SEQ ID NO:539), and TAR15-26-551 (SEQ ID NO:540).

The ligand that has binding specificity for VEGF can inhibit binding of VEGF to vascular endothelial growth factor receptor 1 (VEGFR1) and/or vascular endothelial growth factor receptor 2 (VEGFR2), inhibit the activity of VEGF and/or inhibit the activity of VEGF without substantially inhibiting binding of VEGF to VEGFR1 and/or VEGFR2.

The ligand that has binding specificity for VEGF can contain an immunoglobulin single variable domain with binding specificity for VEGF that binds VEGF with an affinity (KD) that is between about 100 nM and about 1 pM, as determined by surface plasmon resonance.

The ligand that has binding specificity for VEGF can bind VEGF with an affinity (KD) that is between about 100 nM and about 1 pM, as determined by surface plasmon resonance.

The ligand that has binding specificity for VEGF can comprise an immunoglobulin single variable domain with binding specificity for VEGF that is a V_{HH}.

The ligand that has binding specificity for VEGF can comprise an immunoglobulin single variable domain with binding specificity for VEGF that is selected from the group consisting of a human V_{H} and a human V_{L}.

In some embodiments, the ligand that has binding specificity for VEGF is an IgG-like format comprising at least two immunoglobulin single variable domains with binding specificity for VEGF.

In some embodiments, the ligand that has binding specificity for VEGF comprises an antibody Fc region.

The invention also relates to a ligand that has binding specificity for EGFR comprising at least one immunoglobulin single variable domain with binding specificity for EGFR, wherein an immunoglobulin single variable domain with binding specificity for EGFR competes for binding to EGFR with an anti-EGFR domain antibody (dAb) selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

The invention also relates to a ligand that has binding specificity for EGFR comprising at least one immunoglobulin single variable domain with binding specificity for EGFR, wherein an immunoglobulin single variable domain with binding specificity for EGFR competes for binding to EGFR with an anti-EGFR domain antibody (dAb) selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

For example, the immunoglobulin single variable domain with binding specificity for EGFR can comprise an amino acid sequence that has at least about 85% amino acid sequence identity with the amino acid sequence of a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

For example, the immunoglobulin single variable domain with binding specificity for EGFR can comprise an amino acid sequence that has at least about 85% amino acid sequence identity with the amino acid sequence of a dAb selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

The ligand that has binding specificity for EGFR can inhibit binding of epidermal growth factor (EGF) and/or transforming growth factor alpha (TGFalpha) to EGFR, inhibit the activity of EGFR, and/or inhibit the activity of EGFR without substantially inhibiting binding of epidermal growth factor (EGF) and/or transforming growth factor alpha (TGFalpha) to EGFR.

The ligand that has binding specificity for EGFR can contain an immunoglobulin single variable domain with binding specificity for EGFR that binds EGFR with an affinity (KD) that is between about 100 nM and about 1 pM or about 10 nM to about 100 pM, as determined by surface plasmon resonance.

The ligand that has binding specificity for VEGF and for EGFR can bind EGFR with an affinity (KD) that is between about 100 nM and about 1 pM or about 10 nM to about 100 pM, as determined by surface plasmon resonance.

The ligand that has binding specificity for EGFR can comprise an immunoglobulin single variable domain with binding specificity for EGFR that is a V_{HH}.

The ligand that has binding specificity for EGFR can comprise an immunoglobulin single variable domain with binding specificity for EGFR that is selected from the group consisting of a human V_{H} and a human V_{L}.

In some embodiments, the ligand that has binding specificity for EGFR is an IgG-like format comprising at least two immunoglobulin single variable domains with binding specificity for EGFR.

In some embodiments, the ligand that has binding specificity for EGFR comprises an antibody Fc region.

In some embodiments, the ligand comprises a single immunoglobulin variable domain polypeptide that antagonizes (inhibits) human EGFR binding to a receptor, wherein said single immunoglobulin variable domain polypeptide comprises a CDR3 sequence that is the same sequence of CDR3 of an anti-EGFR dAb disclosed herein.

In other embodiments, the ligand comprises a single immunoglobulin variable domain polypeptide that binds EGFR, wherein the polypeptide has an amino acid sequence that is identical to the amino acid sequence of an anti-EGFR dAb disclosed herein, or differs from the amino acid sequence of an anti-EGFR dAb disclosed herein at no more than 25 amino acid positions and has a CDR1 sequence that has at least 50% identity to the CDR1 sequence of the anti-EGFR dAb.

In other embodiments, the ligand comprises single immunoglobulin variable domain polypeptide that binds to EGFR, wherein the polypeptide has an amino acid sequence that is identical to the amino acid sequence of anti-EGFR dAb disclosed herein, or differs from the amino acid sequence of anti-EGFR dAb disclosed herein at no more than 25 amino acid positions and has a CDR2 sequence that has at least 50% identity to the CDR2 sequence of the anti-EGFR dAb.

In other embodiments, the ligand comprises an immunoglobulin single variable domain polypeptide that binds EGFR, wherein the polypeptide has an amino acid sequence that is identical to the amino acid sequence of anti-EGFR dAb disclosed herein, or differs from the amino acid sequence of anti-EGFR dAb disclosed herein at no more than 25 amino acid positions and has a CDR3 sequence that has at least 50% identity to the CDR3 sequence of the anti-EGFR dAb.

In other embodiments, the ligand comprises an immunoglobulin single variable domain polypeptide that binds EGFR, wherein the polypeptide has an amino acid sequence that is identical to the amino acid sequence of an anti-EGFR dAb disclosed herein, or differs from the amino acid sequence of an anti-EGFR dAb disclosed herein at no more than 25 amino acid positions and has a CDR1 sequence that has at least 50% identity to the CDR1 sequence of the anti-EGFR dAb and has a CDR2 sequence has at least 50% identity to the CDR2 sequence of the anti-EGFR dAb.

In other embodiments, the ligand comprises an immunoglobulin single variable domain polypeptide that binds EGFR, wherein the polypeptide has an amino acid sequence that is identical to the amino acid sequence of an anti-EGFR dAb disclosed herein, or differs from the amino acid sequence of an anti-EGFR dAb disclosed herein at no more than 25 amino acid positions and has a CDR2 sequence that has at least 50% identity to the CDR2 sequence of the anti-EGFR dAb and has a CDR3 sequence that has at least 50% identity to the CDR3 sequence of the anti-EGFR dAb.

In other embodiments, the ligand comprises an immunoglobulin single variable domain polypeptide that binds EGFR, wherein the polypeptide has an amino acid sequence that is identical to the amino acid sequence of an anti-EGFR dAb disclosed herein, or differs from the amino acid sequence of an anti-EGFR dAb disclosed herein at no more than 25 amino acid positions and has a CDR1 sequence that has at least 50% identity to the CDR1 sequence of the anti-EGFR dAb and has a CDR3 sequence that has at least 50% identity to the CDR3 sequence of the anti-EGFR dAb.

In other embodiments, the ligand comprises an immunoglobulin single variable domain polypeptide that binds EGFR, wherein the polypeptide has an amino acid sequence that is identical to the amino acid sequence of an anti-EGFR dAb disclosed herein, or differs from the amino acid sequence of an anti-EGFR dAb disclosed herein at no more than 25 amino acid positions and has a CDR1 sequence that has at least 50% identity to the CDR1 sequence of the anti-EGFR dAb and has a CDR2 sequence that has at least 50% identity to the CDR2 sequence of the anti-EGFR dAb and has a CDR3 sequence that has at least 50% identity to the CDR3 sequence of the anti-EGFR dAb.

In another embodiment, the invention is an EGFR antagonist having a CDR1 sequence that has at least 50% identity to the CDR1 sequence of an anti-EGFR dAb disclosed herein.

In another embodiment, the invention is an EGFR antagonist having a CDR2 sequence that has at least 50% identity to the CDR2 sequence of an anti-EGFR dAb disclosed herein.

In another embodiment, the invention is an EGFR antagonist having a CDR3 sequence that has at least 50% identity to the CDR3 sequence of an anti-EGFR dAb disclosed herein.

In another embodiment, the invention is an EGFR antagonist having a CDR1 sequence that has at least 50% identity to the CDR1 sequence of an anti-EGFR dAb disclosed herein and a CDR2 sequence that has at least 50% identity to the CDR2 sequence of the anti-EGFR dAb.

In another embodiment, the invention is an EGFR antagonist having a CDR2 sequence that has at least 50% identity to the CDR2 sequence of an anti-EGFR dAb disclosed herein and a CDR3 sequence that has at least 50% identity to the CDR3 sequence of the anti-EGFR dAb.

In another embodiment, the invention is an EGFR antagonist having a CDR1 sequence that has at least 50% identity to the CDR1 sequence of an anti-EGFR dAb disclosed herein and a CDR3 sequence that has at least 50% identity to the CDR3 sequence of the anti-EGFR dAb.

In another embodiment, the invention is an EGFR antagonist having a CDR1 sequence that has at least 50% identity to the CDR1 sequence of an anti-EGFR dAb disclosed herein and a CDR2 sequence that has at least 50% identity to the CDR2 sequence of the anti-EGFR dAb and a CDR3 sequence that has at least 50% identity to the CDR3 sequence of the anti-EGFR dAb.

In some embodiments, the ligand comprises a single immunoglobulin variable domain polypeptide that antagonizes (inhibits) human VEGF binding to a receptor, wherein said single immunoglobulin variable domain polypeptide comprises a CDR3 sequence that is the same sequence of CDR3 of an anti-VEGF dAb disclosed herein.

In other embodiments, the ligand comprises a single immunoglobulin variable domain polypeptide that binds VEGF, wherein the polypeptide has an amino acid sequence that is identical to the amino acid sequence of an anti-VEGF dAb disclosed herein, or differs from the amino acid sequence of an anti-VEGF dAb disclosed herein at no more than 25 amino acid positions and has a CDR1 sequence that has at least 50% identity to the CDR1 sequence of the anti-VEGF dAb.

In other embodiments, the ligand comprises single immunoglobulin variable domain polypeptide that binds to VEGF, wherein the polypeptide has an amino acid sequence that is identical to the amino acid sequence of anti-VEGF dAb disclosed herein, or differs from the amino acid sequence of anti-VEGF dAb disclosed herein at no more than 25 amino acid positions and has a CDR2 sequence that has at least 50% identity to the CDR2 sequence of the anti-VEGF dAb.

In other embodiments, the ligand comprises an immunoglobulin single variable domain polypeptide that binds VEGF, wherein the polypeptide has an amino acid sequence that is identical to the amino acid sequence of anti-VEGF dAb disclosed herein, or differs from the amino acid sequence of anti-VEGF dAb disclosed herein at no more than 25 amino acid positions and has a CDR3 sequence that has at least 50% identity to the CDR3 sequence of the anti-VEGF dAb.

In other embodiments, the ligand comprises an immunoglobulin single variable domain polypeptide that binds VEGF, wherein the polypeptide has an amino acid sequence that is identical to the amino acid sequence of an anti-VEGF dAb disclosed herein, or differs from the amino acid sequence of an anti-VEGF dAb disclosed herein at no more than 25 amino acid positions and has a CDR1 sequence that has at least 50% identity to the CDR1 sequence of the anti-VEGF dAb and has a CDR2 sequence has at least 50% identity to the CDR2 sequence of the anti-VEGF dAb.

In other embodiments, the ligand comprises an immunoglobulin single variable domain polypeptide that binds VEGF, wherein the polypeptide has an amino acid sequence that is identical to the amino acid sequence of an anti-VEGF dAb disclosed herein, or differs from the amino acid sequence of an anti-VEGF dAb disclosed herein at no more than 25 amino acid positions and has a CDR2 sequence that has at least 50% identity to the CDR2 sequence of the anti-VEGF dAb and has a CDR3 sequence that has at least 50% identity to the CDR3 sequence of the anti-VEGF dAb.

In other embodiments, the ligand comprises an immunoglobulin single variable domain polypeptide that binds VEGF, wherein the polypeptide has an amino acid sequence that is identical to the amino acid sequence of an anti-VEGF dAb disclosed herein, or differs from the amino acid sequence of an anti-VEGF dAb disclosed herein at no more than 25 amino acid positions and has a CDR1 sequence that has at least 50% identity to the CDR1 sequence of the anti-VEGF dAb and has a CDR3 sequence that has at least 50% identity to the CDR3 sequence of the anti-VEGF dAb.

In other embodiments, the ligand comprises an immunoglobulin single variable domain polypeptide that binds VEGF, wherein the polypeptide has an amino acid sequence that is identical to the amino acid sequence of an anti-VEGF dAb disclosed herein, or differs from the amino acid sequence of an anti-VEGF dAb disclosed herein at no more than 25 amino acid positions and has a CDR1 sequence that has at least 50% identity to the CDR1 sequence of the anti-VEGF dAb and has a CDR2 sequence that has at least 50% identity to the CDR2 sequence of the anti-VEGF dAb and has a CDR3 sequence that has at least 50% identity to the CDR3 sequence of the anti-VEGF dAb.

In another embodiment, the invention is an VEGF antagonist having a CDR1 sequence that has at least 50% identity to the CDR1 sequence of an anti-VEGF dAb disclosed herein.

In another embodiment, the invention is an VEGF antagonist having a CDR2 sequence that has at least 50% identity to the CDR2 sequence of an anti-VEGF dAb disclosed herein.

In another embodiment, the invention is an VEGF antagonist having a CDR3 sequence that has at least 50% identity to the CDR3 sequence of an anti-VEGF dAb disclosed herein.

In another embodiment, the invention is an VEGF antagonist having a CDR1 sequence that has at least 50% identity to the CDR1 sequence of an anti-VEGF dAb disclosed herein and a CDR2 sequence that has at least 50% identity to the CDR2 sequence of the anti-VEGF dAb.

In another embodiment, the invention is an VEGF antagonist having a CDR2 sequence that has at least 50% identity to the CDR2 sequence of an anti-VEGF dAb disclosed herein and a CDR3 sequence that has at least 50% identity to the CDR3 sequence of the anti-VEGF dAb.

In another embodiment, the invention is an VEGF antagonist having a CDR1 sequence that has at least 50% identity to the CDR1 sequence of an anti-VEGF dAb disclosed herein and a CDR3 sequence that has at least 50% identity to the CDR3 sequence of the anti-VEGF dAb.

In another embodiment, the invention is an VEGF antagonist having a CDR1 sequence that has at least 50% identity to the CDR1 sequence of an anti-VEGF dAb disclosed herein and a CDR2 sequence that has at least 50% identity to the CDR2 sequence of the anti-VEGF dAb and a CDR3 sequence that has at least 50% identity to the CDR3 sequence of the anti-VEGF dAb.

In additional embodiments, any of the ligands described herein further comprise a toxin, such as a cytotoxin, free radical generator, antimetabolite, protein, polypeptide, peptide, photoactive agent, antisense compound, chemotherapeutic, radionuclide or intrabody. In particular embodiments, the toxin is a surface active toxin (e.g., a free radical generator, a radionuclide).

In other embodiments, the ligand further comprises a half-life extending moiety, such as a polyalkylene glycol moiety, serum albumin or a fragment thereof, transferrin receptor or a transferrin-binding portion thereof, or a moiety comprising a binding site for a polypeptide that enhances half-life in vivo. In some embodiments, the half-life extending moiety is a moiety comprising a binding site for a polypeptide that enhances half-life in vivo selected from the group consisting of an affibody, an SpA domain, an LDL receptor class A domain, an EGF domain, and an avimer.

In other embodiments, the half-life extending moiety is an antibody or antibody fragment (e.g., an immunoglobulin single variable domain) comprising a binding site for serum albumin or neonatal Fc receptor.

In particular embodiments, the half-life extending moiety is an immunoglobulin single variable domain comprising a binding site for serum albumin that competes for binding to human serum albumin with a dAb selected from the group consisting of DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), and DOM7r-33 (SEQ ID NO: 517).

For example, the immunoglobulin single variable domain comprising a binding site for serum albumin can comprise an amino acid sequence that has at least 85% amino acid sequence identity with the amino acid sequence of a dAb selected from the group consisting of DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), and DOM7r-33 (SEQ ID NO: 517).

The invention also relates to an isolated or recombinant nucleic acid encoding a ligand described herein, and to a vector (e.g., recombinant vector) comprising the recombinant nucleic acid. The invention also relates to a host cell (e.g., recombinant host cell, isolated host cell) comprising a recombinant nucleic acid or vector of the invention. The invention also relates to a method for producing a ligand, comprising maintaining a host cell of the invention under conditions suitable for expression of said nucleic acid or vector, whereby a ligand is produced. In some embodiments, the method further comprises isolating the ligand.

The invention also relates to a ligand of the invention for use in therapy or diagnosis, and to the use of a ligand of the invention for the manufacture of a medicament for treatment, prevention or suppression of a disease described herein (e.g., cancer).

The invention also relates to a pharmaceutical composition for the treatment, prevention or suppression of a disease described herein (e.g., cancer) comprising as an active ingredient a ligand of the invention.

In some embodiments, the invention relates to a ligand for use in treating cancer, or cancer cells that overexpress EGFR and/or VEGF.

In other embodiments, the invention relates to use of a ligand for the manufacture of a medicament for killing cells (e.g., selectively killing cancer cells over normal cells).

In other embodiments, the invention relates to use of a ligand for the manufacture of a medicament for treating cancer cells that overexpress EGFR and/or VEGF.

The invention also relates to therapeutic methods that comprise administering a therapeutically effective amount of a ligand of the invention to a subject in need thereof. In one embodiment, the invention relates to a method for treating cancer comprising administering to a subject in need thereof a therapeutically effective amount of ligand of the invention. In some embodiments, the method further comprises administering to the subject a chemotherapeutic agent (e.g., at a low dose).

In other embodiments, the method for treating cancer comprises administering to a subject in need thereof a therapeutically effective amount of ligand of the invention and an anti-neoplastic composition, wherein said anti-neoplastic composition comprises at least one chemotherapeutic agent. The chemotherapeutic agent can be selected from the group consisting of alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitor, interferons, platinum cooridnation complexes, anthracenedione substituted urea, methyl hydrazine derivatives, adreno cortical suppressant, adrenocorticosteroides, progestins, estrogens, antiestrogen, androgens, antiandrogen, and gonadotropin-releasing hormone analog. In some embodiments, the chemotherapeutic agent is selected from the group consisting of cisplatin, dicarbazine, dactinomycin, mechlorethamine, streptozocin, cyclophosphamide, capecitabine, cannustine, lomustine, doxorubicin, daunorubicin, procarbazine, mitomycin, cytarabine, etoposide, methotrexate, 5-fluorouracil, vinbiastine, vincristine, bleomycin, paclitaxel, docetaxel, doxetaxe, aldesleukin, asparaginase, busulfan, carboplatin, cladribine, dacarbazine, floxuridine, fludarabine, hydroxyurea, ifosfamide, interferon alpha, irinotecan, leuprolide, leucovorin, megestrol, melphalan, mercaptopurine, oxaliplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol, an additional growth factor receptor antagonist, and a combination of any of the foregoing.

In some embodiments, the method is a method of treating a cancer selected from the group consisting of bladder cancer, ovarian cancer, colorectal cancer (colorectal carcinoma), breast cancer, lung cancer (non-small cell lung carcinoma), gastric cancer, pancreatic cancer, prostate cancer, head and neck cancer, renal cancer and gall bladder cancer.

The invention also relates to a method of administering to a subject anti-VEGF treatment and anti-EGFR treatment, the method comprising simultaneous administration of an anti-VEGF treatment and an anti-EGFR treatment by administering to said subject a therapeutically effective amount of a ligand that has binding specificity for VEGF and EGFR.

The invention also relates to a composition (e.g., pharmaceutical composition) comprising a ligand of the invention and a physiologically or pharmaceutically acceptable carrier. In some embodiments, the composition comprises a vehicle for intravenous, intramuscular, intraperitoneal, intraarterial, intrathecal, intraarticular subcutaneous administration, pulmonary, intranasal, vaginal, or rectal administration.

The invention also relates to a drug delivery device comprising the composition (e.g., pharmaceutical composition) of the invention or a ligand of the invention. In one embodiment, the drug delivery device is for simultaneously administering to a subject anti-VEGF treatment and anti-EGFR treatment, and the device comprising a ligand that has binding specificity for VEGF and EGFR. In some embodiments, the drug device comprises a plurality of therapeutically effective doses of ligand.

In other embodiments, the drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, a catheter.

The invention also relates to a ligand that has binding specificity for vascular endothelial growth factor (VEGF) and epidermal growth factor receptor (EGFR), comprising at least one protein moiety that has a binding site with binding specificity for VEGF, at least one protein moiety that has a binding site with binding specificity for EGFR, and an Fc region of an antibody. Such ligands can consist of a single polypeptide. In other embodiments, two ligands that contain Fc regions are bonded together, for example through a disulfide bond (e.g., in the hinge region), to form a dimer.

In some embodiments, the ligand that has binding specificity for VEGF can comprise a protein moiety that has a binding site with binding specificity for VEGF and an Fc region of an antibody. In some embodiments, the protein moiety having binding specificity for VEGF is fused to an Fc region of an antibody.

In other embodiments, the ligand that has binding specificity for EGFR can comprise a protein moiety that has a binding site with binding specificity for EGFR and an Fc region of an antibody. In some embodiments, the protein moiety having binding specificity for EGFR is fused to an Fc region of an antibody. For example, the ligand can comprise two protein moieties that have binding sites with binding specificity for EGFR and an Fc region of an antibody.

Additionally, or in other embodiments, the ligand that has binding specificity for VEGF and EGFR, comprises a single variable domain with binding specificity for VEGF, a single variable domain with binding specificity for EGFR, and optionally a linker. In such embodiments, the single variable domain with binding specificity for EGFR can be bonded via the linker to the immunoglobulin single variable domain with binding specificity for VEGF. Suitable linkers include SEQ ID NO:706, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:723 and SEQ ID NO:724. The ligand can also comprise an Fc region of an antibody if desired. When the ligand further comprises an Fc region of an antibody, a linker can bind an immunoglobulin variable domain to the Fc region. In other embodiments, two ligands that contain Fc regions are bonded together, for example through a disulfide bond (e.g., in the hinge region), to form a dimer.

Additionally, or in other embodiments, the ligand that has binding specificity for VEGF and EGFR, comprises a single variable domain with binding specificity for VEGF directly fused to a single variable domain with binding specificity for EGFR.

In embodiments where the ligand comprises a single variable domain with binding specificity for VEGF, and a single variable domain with binding specificity for EGFR, and optionally one or more linkers, the single variable domains can independently be a light chain variable domain or a heavy chain variable domain. For example, the ligand can comprise a) a single variable domain with binding specificity for VEGF that is a heavy chain variable region, and the immunoglobulin single variable domain with binding specificity for EGFR that is a light chain variable region; b) a single variable domain with binding specificity for VEGF that is a light chain variable domain, and a single variable domain with binding specificity for EGFR that is a heavy chain variable domain; c) a single variable domain with binding specificity for VEGF that is a heavy chain variable domain, and a single variable domain with binding specificity for EGFR that is a heavy chain variable domain; or d) a single variable domain with binding specificity for VEGF that is a light chain variable domain, and a single variable domain with binding specificity for EGFR that is a light chain variable domain. In particular embodiments, the heavy chain variable region is a V_{H} or V_{HH}. In further embodiments, the V_{H} is a human V_{H}. In other embodiments, the light chain variable region is a V_{K}.

In another aspect of the invention, a ligand that has binding specificity for VEGF and EGFR comprises at least one immunoglobulin single variable domain with binding specificity for VEGF and at least one immunoglobulin single variable domain with binding specificity for EGFR, wherein the immunoglobulin single variable domain with binding specificity for EGFR is bonded via a disulfide bond to the immunoglobulin single variable domain with binding specificity for VEGF. Alternatively, a ligand that has binding specificity for VEGF and EGFR can comprises at least one immunoglobulin single variable domain with binding specificity for VEGF and at least one immunoglobulin single variable domain with binding specificity for EGFR, wherein the immunoglobulin single variable domain with binding specificity for EGFR is directly fused to the immunoglobulin single variable domain with binding specificity for VEGF (i.e., a single polypeptide comprising two dAbs).

In other particular embodiments, the ligand is a fusion of a dAb to an anti-serum albumin dAb (a DOM7 dAb). For example, the ligand can have the structure, from amino-terminal to carboxy-terminal, DOM15-10-DOM16-39-anti-serum albumin dAb, DOM16-39-DOM15-10-anti-serum albumin dAb, DOM15-26-501-DOM16-39-anti-serum albumin dAb, or DOM16-39DOM15-26-501-anti-serum albumin dAb. In additional embodiments, the ligand that has a binding site with binding specificity for EGFR can compete for binding to EGFR with cetuximab and/or panitumumab and is fused to an anti-serum albumin dAb. Additionally, or in other embodiments, the ligand can comprise two or more dAbs (e.g. anti-EGFR dAbs) fused to an anti-serum albumin dAb.

### BRIEF DESCRIPTION OF THE DRAWINGS

FIG. 1A-1E illustrates twenty-seven nucleotide sequences that encode human (Homo sapiens) domain antibodies (dAbs) that specifically bind human VEGF. The nucleotide sequences presented are SEQ ID NOS:1-27, 535 and 536.

FIG. 2A-2c is a alignment of twelve nucleotide sequences that encode human dAbs that bind human VEGF. The nucleotide sequences presented are SEQ ID NO:18 and SEQ ID NOS:28-38.

FIG. 3A-3D is a alignment of twelve nucleotide sequences that encode human dAbs that bind human VEGF. The nucleotide sequences presented are SEQ ID NO:20 and SEQ ID NOS:39-49.

FIG. 4A-4J is a alignment of fifty-three nucleotide sequences that encode human dAbs that bind human VEGF. The nucleotide sequences presented are SEQ ID NO:24, 50-99, 537 and 538.

FIG. 5A-5C illustrates the amino acid sequences of dAbs encoded by several of the nucleic acid sequences shown in FIG. 1A-1E. The amino acid sequences presented are SEQ ID NOS:100-126.

FIG. 6 is an aligmnent of the amino acid sequences of the dAbs encoded by the nucleic acid sequences shown in FIG. 2A-2C. The amino acid sequences presented are SEQ ID NO:117 AND SEQ ID NOS:127-137.

FIG. 7A-7B is an aligmnent of the amino acid sequences of the dAbs encoded by the nucleic acid sequences shown in FIG. 3A-2D. The symbol ˜ has been inserted into the sequence of TAR15-8-500 to facilitate alignment. The amino acid sequences presented are SEQ ID NO:119 and SEQ ID NOS:138-148.

FIG. 8A-8D is an alignment of the amino acid sequences of the dAbs encoded by the nucleic acid sequences shown in FIG. 4A-4J. The amino acid sequences presented are SEQ ID NO:123, 149-198, 539 and 540.

FIG. 9A-90 illustrates several nucleotide sequences that encode human (Homo sapiens) domain antibodies (dAbs) that specifically bind human EGFR. The nucleotide sequences presented are SEQ ID NOS:199-324.

FIG. 10A-10I illustrates the amino acid sequences of the dAbs encoded by the nucleic acid sequences shown in FIG. 9A-90. The amino acid sequences presented are SEQ ID NOS:325-450.

FIG. 11A-11B illustrates the amino acid sequences of several Camelid V_{HH}s that bind EGFR that are disclosed in WO 2005/044858. NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), NB22 (SEQ ID NO:472).

FIG. 12A is an alignment of the amino acid sequences of three Ws that bind mouse serum albumin (MSA). The aligned amino acid sequences are from Vκs designated MSA16, which is also referred to as DOM7m-16 (SEQ ID NO: 473), MSA 12, which is also referred to as DOM7m-12 (SEQ ID NO: 474), and MSA 26, which is also referred to as DOM7m-26 (SEQ ID NO: 475).

FIG. 12B is an alignment of the amino acid sequences of six Ws that bind rat serum albumin (RSA). The aligned amino acid sequences are from Ws designated DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), and DOM7r-8 (SEQ ID NO: 481).

FIG. 12C is an alignment of the amino acid sequences of six Ws that bind human serum albumin (HSA). The aligned amino acid sequences are from Ws designated DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), and DOM7h-7 (SEQ ID NO: 487).

FIG. 12D is an alignment of the amino acid sequences of seven V_{H}s that bind human serum albumin and a consensus sequence (SEQ ID NO: 488). The aligned sequences are from V_{H}s designated DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), and DOM7h-27 (SEQ ID NO: 495).

FIG. 12E is an alignment of the amino acid sequences of three Ws that bind human serum albumin and rat serum albumin. The aligned amino acid sequences are from Ws designated DOM7h-8 (SEQ ID NO: 496), DOM7r-13 (SEQ ID NO: 497), and DOM7r-14 (SEQ ID NO: 498).

FIG. 13 is an illustration of the amino acid sequences of Ws that bind rat serum albumin (RSA). The illustrated sequences are from Ws designated DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503).

FIG. 14A-14B is an illustration of the amino acid sequences of V_{H}s that bind rat serum albumin (RSA). The illustrated sequences are from V_{H}s designated DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), and DOM7r-33 (SEQ ID NO: 517).

FIG. 15 illustrates the amino acid sequences of several Camelid V_{HH}s that bind mouse serum albumin that are disclosed in WO 2004/041862. Sequence A (SEQ ID NO: 518), Sequence B (SEQ ID NO: 519), Sequence C (SEQ ID NO: 520), Sequence D (SEQ ID NO: 521), Sequence E (SEQ ID NO: 522), Sequence F (SEQ ID NO: 523), Sequence G (SEQ ID NO: 524), Sequence H (SEQ ID NO: 525), Sequence I (SEQ ID NO:526), Sequence J (SEQ ID NO:527), Sequence K (SEQ ID NO: 528), Sequence L (SEQ ID NO:529), Sequence M (SEQ ID NO:530), Sequence N (SEQ ID NO:531), Sequence 0 (SEQ ID NO: 532), Sequence P (SEQ ID NO:533), Sequence Q (SEQ ID NO:534).

FIG. 16 is a map of a vector used to prepare IgG-like formats.

FIG. 17A-17F illustrates the amino acid sequences of human dAbs that bind human EGFR. The amino acid sequences presented are SEQ ID NOS:541-622, 725 and 726. The sequences are continuous with no gaps, the symbols ˜, ˜˜ and ˜˜˜ have been inserted to show the locations of the CDRs. CDR1 is flanked by ˜, CDR2 is flanked by ˜˜, and CDR3 is flanked by ˜˜˜.

FIG. 18A-18L illustrates nucleotide sequences that encode the dAbs shown in FIG. 17A-17F. The nucleotide sequences presented are SEQ ID NOS:623-703, 727 and 728.

FIG. 19 illustrates the amino acid sequence (SEQ ID NO:704) of a human dAb that binds VEGF, and a nucleotide sequence (SEQ ID NO:705) that encodes the dAb. The sequences are continuous with no gaps, the symbols ˜, ˜˜ and ˜˜˜ have been inserted to show the locations of the CDRs. CDR1 is flanked by ˜, CDR2 is flanked by ˜˜, and CDR3 is flanked by ˜˜˜.

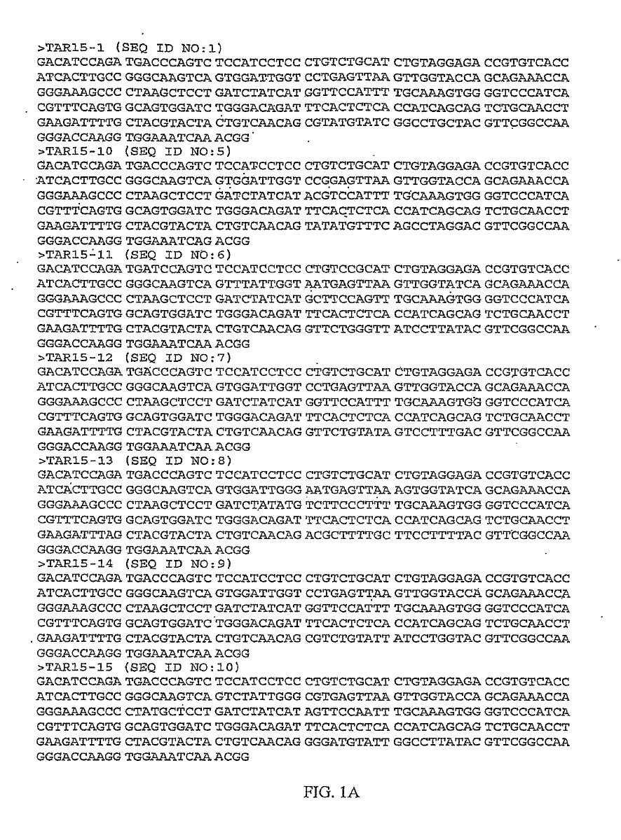

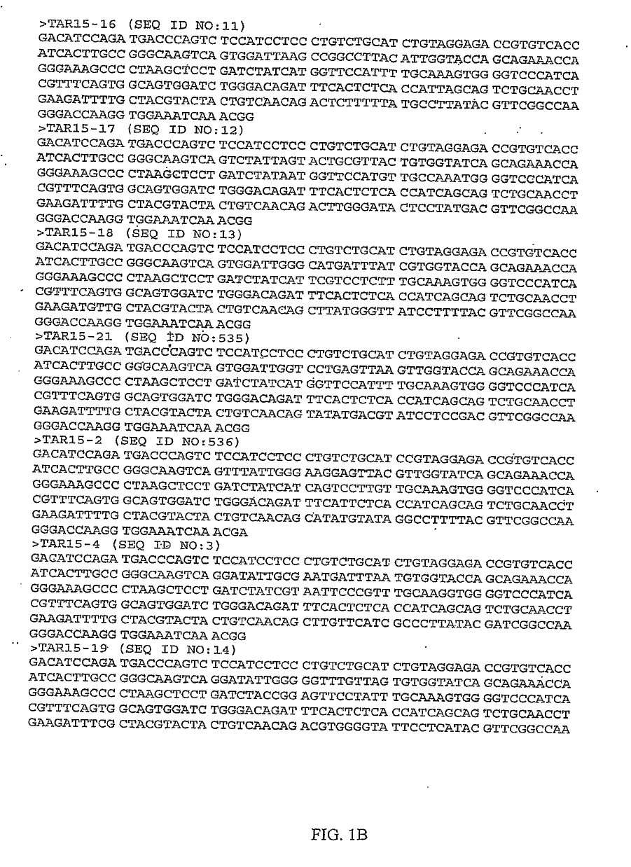

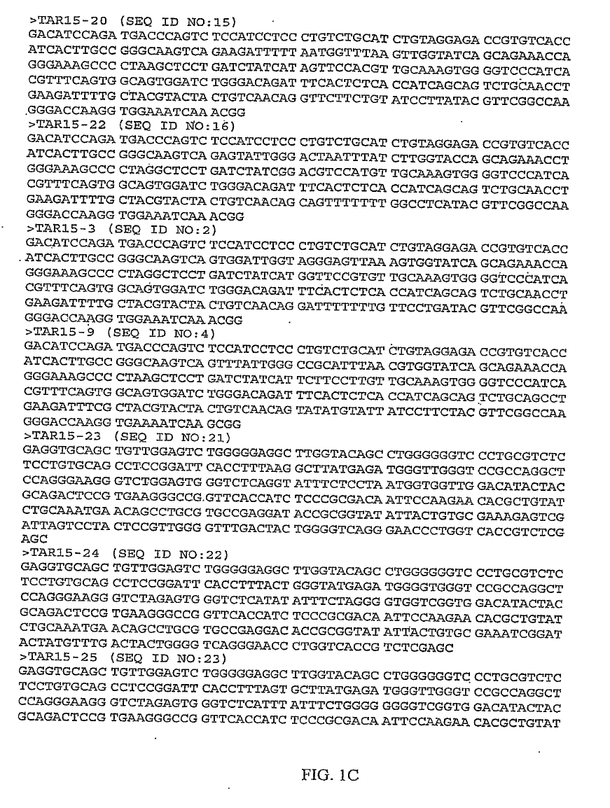

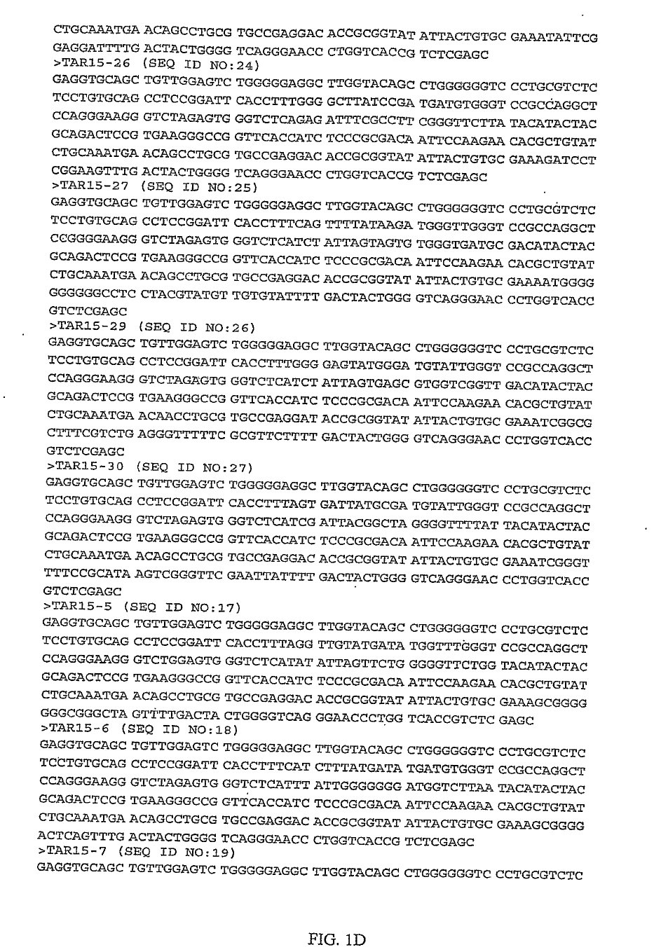

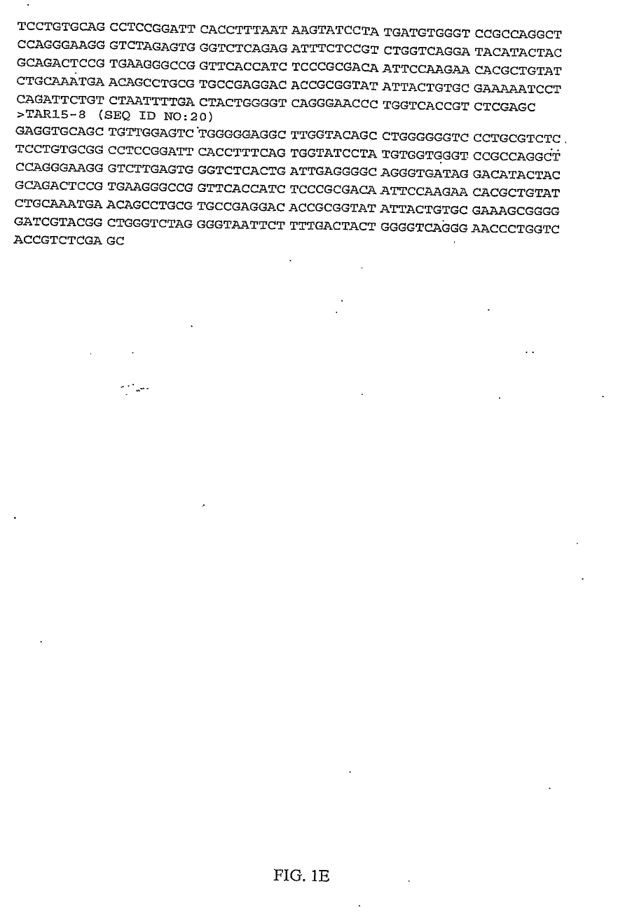

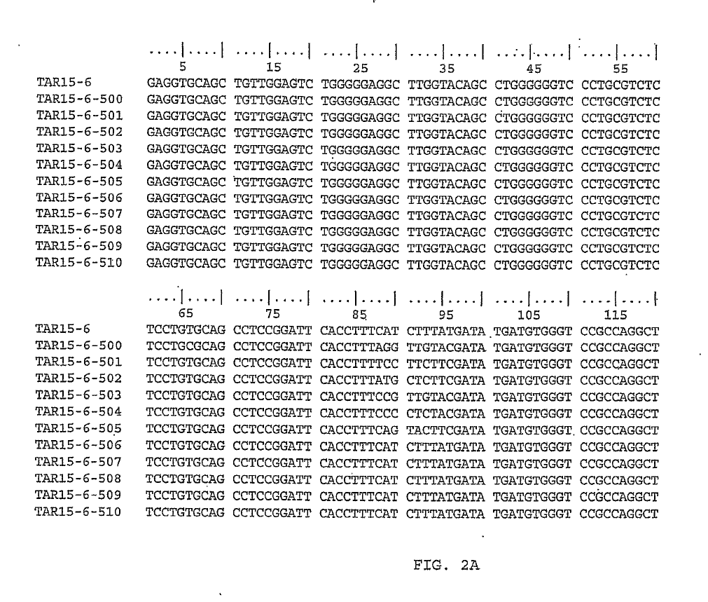

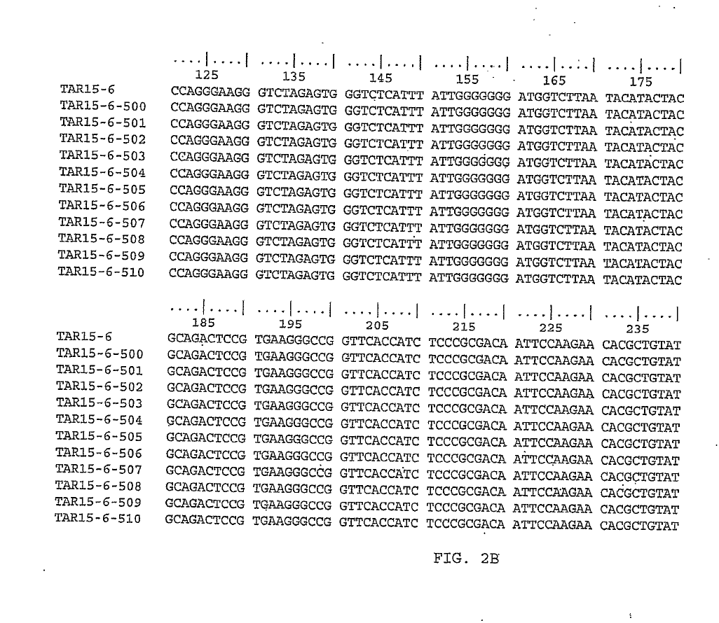

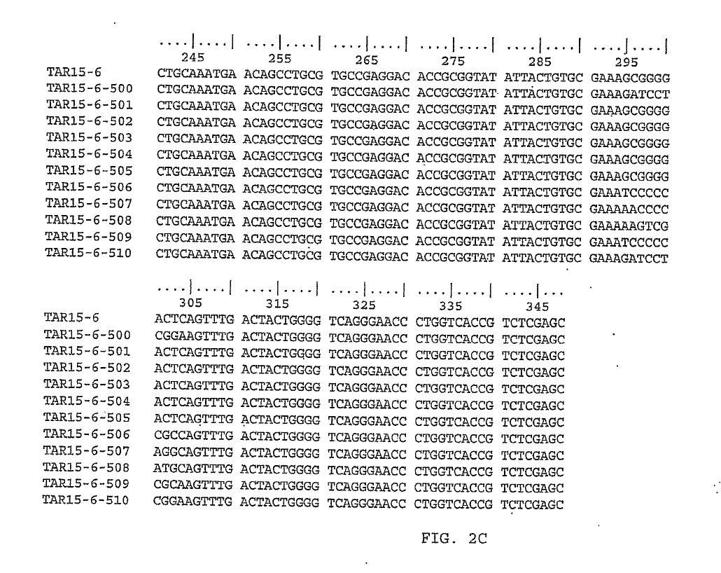

### DETAILED DESCRIPTION OF THE INVENTION

Within this specification embodiments have been described in a way which enables a clear and concise specification to be written, but it is intended and will be appreciated that embodiments may be variously combined or separated without parting from the invention.

As used herein, the term “ligand” refers to a compound that comprises at least one peptide, polypeptide or protein moiety that has a binding site with binding specificity for a desired endogenous target compound. The ligands according to the invention preferably comprise immunoglobulin variable domains which have different binding specificities, and do not contain variable domain pairs which have the same specificity. Preferably each domain which has a binding site that has binding specificity for a cell surface target is an immunoglobulin single variable domain (e.g., immunoglobulin single heavy chain variable domain (e.g., V_{H}, V_{HH}) immunoglobulin single light chain variable domain (e.g., V_{L})) that has binding specificity for a desired cell surface target (e.g., a membrane protein, such as a receptor protein). Each polypeptide domain which has a binding site that has binding specificity for a cell surface target can also comprise one or more complementarity determining regions (CDRs) of an antibody or antibody fragment (e.g., an immunoglobulin single variable domain) that has binding specificity for a desired cell surface target in a suitable format, such that the binding domain has binding specificity for the cell surface target. For example, the CDRs can be grafted onto a suitable protein scaffold or skeleton, such as an affibody, an SpA scaffold, an LDL receptor class A domain, or an EGF domain. Further, the ligand can be bivalent (heterobivalent) or multivalent (heteromultivalent) as described herein. The first and second domains lack domains that share the same specificity. Thus, “ligands” include polypeptides that comprise two dAbs wherein each dAb binds to a different cell surface target. Ligands also include polypeptides that comprise at least two dAbs that bind different cell surface targets (or the CDRs of a dAbs) in a suitable format, such as an antibody format (e.g., IgG-like format, scFv, Fab, Fab′, F(ab′)₂) or a suitable protein scaffold or skeleton, such as an affibody, an SpA scaffold, an LDL receptor class A domain, an EGF domain, avimer and multispecific ligands as described herein. The polypeptide domain which has a binding site that has binding specificity for a cell surface target (i.e., first or second cell surface target) can also be a protein domain comprising a binding site for a desired target, e.g., a protein domain is selected from an affibody, an SpA domain, an LDL receptor class A domain, an avimer (see, e.g., U.S. Patent Application Publication Nos. 2005/0053973, 2005/0089932, 2005/0164301). If desired, a “ligand” can further comprise one or more additional moieties, that can each independently be a peptide, polypeptide or protein moiety or a non-peptidic moiety (e.g., a polyalkylene glycol, a lipid, a carbohydrate). For example, the ligand can further comprise a half-life extending moiety as described herein (e.g., a polyalkylene glycol moiety, a moiety comprising albumin, an albumin fragment or albumin variant, a moiety comprising transferrin, a transferrin fragment or transferrin variant, a moiety that binds albumin, a moiety that binds neonatal Fc receptor).

As used herein, the phrase “target” refers to a biological molecule (e.g., peptide, polypeptide, protein, lipid, carbohydrate) to which a polypeptide domain which has a binding site can bind. The target can be, for example, an intracellular target (e.g., an intracellular protein target) or a cell surface target (e.g., a membrane protein, a receptor protein). Preferably, the target is VEGF or EGFR.

The phrase “immunoglobulin single variable domain” refers to an antibody variable region (V_{H}, V_{HH}, V_{L}) that specifically binds a target, antigen or epitope independently of other V domains; however, as the term is used herein, an immunoglobulin single variable domain can be present in a format (e.g., hetero-multimer) with other variable regions or variable domains where the other regions or domains are not required for antigen binding by the single immunoglobulin variable domain (i.e., where the immunoglobulin single variable domain binds antigen independently of the additional variable domains). Each “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, preferably human, but also includes rodent (for example, as disclosed in WO 00/29004, the contents of which are incorporated herein by reference in their entirety) or camelid V_{HH} dAbs. As used herein, camelid dAbs are immunoglobulin single variable domain polypeptides which are derived from species including camel, llama, alpaca, dromedary, and guanaco, and comprise heavy chain antibodies naturally devoid of light chain (V_{HH}). Similar dAbs, can be obtained for single chain antibodies from other species, such as nurse shark. Preferred ligands comprises at least two different immunoglobulin single variable domain polypeptides or at least two different dAbs.

A “human” immunoglobulin single variable domain (e.g., dAb, V_{H}, V_{L}, V_{λ}) can be derived from an antibody of human origin or from a library prepared using human antibody variable region genes. For example, as described herein, human immunoglobulin single variable domains have one or more framework regions that are encoded by a human germline antibody gene segment, or that have up to 5 amino acid differences relative to the amino acid sequence encoded by a human germline antibody gene segment. Preferably, the amino acid sequences of FW1, FW2, FW3 and FW4 are each encoded by a human germline antibody gene segment, or collectively contain up to 10 amino acid differences relative to the amino acid sequences of the corresponding framework regions encoded by the human germline antibody gene segment.

As used herein “vascular endothelial growth factor” (VEGF) refers to naturally occurring or endogenous mammalian VEGF-A proteins and to proteins having an amino acid sequence which is the same as that of a naturally occurring or endogenous corresponding mammalian VEGF-A protein (e.g., recombinant proteins, synthetic proteins (i.e., produced using the methods of synthetic organic chemistry)). Accordingly, as defined herein, the term includes mature VEGF-A protein, polymorphic or allelic variants, and other isoforms of a VEGF-A (e.g., produced by alternative splicing or other cellular processes), and modified or unmodified forms of the foregoing (e.g., lipidated, glycosylated). Alternative splicing of RNA encoding human (Homo sapiens) VEGF-A yield several isoforms of human VEGF-A that differ in the number of amino acids in the protein sequence. For example, isoforms referred to as VEGF-121, VEGF-165, VEGF-189 and VEGF-206 are produced in humans. (See, e.g., Ferrara, N., Endocrine Reviews, 25(4):581-611 (2004).) These isoforms and other naturally occurring isoforms are expressly encompassed by the term “VEGF”. Naturally occurring or endogenous VEGF-A include wild type proteins such as mature VEGF-A, polymorphic or allelic variants and other isoforms which occur naturally in mammals (e.g., humans, non-human primates). Such proteins can be recovered or isolated from a source which naturally produces VEGF-A, for example. These proteins and proteins having the same amino acid sequence as a naturally occurring or endogenous corresponding VEGF, are referred to by the name of the corresponding mammal. For example, where the corresponding mammal is a human, the protein is designated as a human VEGF.

A ligand (e.g., immunoglobulin single variable domain) that inhibits binding of VEGF to VEGFR1 or VEGFR2 inhibits binding in the VEGFR1 binding assay or VEGFR2 assay described herein by at least about 40%, at least about 50%, at least about 60%, at least about 70%, at least about 80%, at least about 85%, at least about 90%, or at least about 95% when the ligand is assayed at a concentration of about 1 nM, about 10 nM, about 50 nM, about 100 nM, about 1 μM, about 10 μM or about 100 μM. A ligand that inhibits binding of VEGF to VEGFR1 or VEGFR2, can also or alternatively, inhibit binding in the VEGFR1 binding assay or VEGFR2 assay with an IC50 of about 1 μM or less, about 500 nM or less, about 100 nM or less, about 75 nM or less, about 50 nM or less, about 10 nM or less or about 1 nM or less.

A ligand (e.g., immunoglobulin single variable domain) that inhibits activity of VEGF inhibits viability in the VEGF bioassay described herein by at least about 20%, at least about 30%, at least about 40%, at least about 50%, at least about 60%, at least about 70%, at least about 80%, at least about 85%, at least about 90%, or at least about 95%.

A ligand (e.g., immunoglobulin single variable domain) that does not substantially inhibit binding of VEGF to VEGFR1 or VEGFR2 does not significantly inhibit binding in the VEGFR1 binding assay or VEGFR2 assay described herein. For example, such a ligand might inhibit binding of VEGF in the VEGFR1 binding assay or VEGFR2 assay described herein with an 1050 of about 1 mM or higher, or inhibit binding by no more than about 20%, no more than about 15%, no more than about 10% or no more than about 5%.

As used herein “epideinial growth factor receptor” (EGFR, ErbB1, HER1) refers to naturally occurring or endogenous mammalian EGFR proteins and to proteins having an amino acid sequence which is the same as that of a naturally occurring or endogenous corresponding mammalian EGFR protein (e.g., recombinant proteins, synthetic proteins (i.e., produced using the methods of synthetic organic chemistry)). Accordingly, as defined herein, the term includes mature EGFR protein, polymorphic or allelic variants, and other isoforms of a EGFR (e.g., produced by alternative splicing or other cellular processes), and modified or unmodified forms of the foregoing (e.g., lipidated, glycosylated). Naturally occurring or endogenous EGFR include wild type proteins such as mature EGFR, polymorphic or allelic variants and other isoforms which occur naturally in mammals (e.g., humans, non-human primates). Such proteins can be recovered or isolated from a source which naturally produces EGFR, for example. These proteins and proteins having the same amino acid sequence as a naturally occurring or endogenous corresponding EGFR, are referred to by the name of the corresponding mammal. For example, where the corresponding mammal is a human, the protein is designated as a human EGFR.

A ligand (e.g., immunoglobulin single variable domain) that inhibits binding of EGF and/or TGF alpha to EGFR inhibits binding in the EGFR binding assay or EGFR kinase assay described herein with an IC50 of about 1 μM or less, about 500 nM or less, about 100 nM or less, about 75 nM or less, about 50 nM or less, about 10 nM or less or about 1 nM or less.

A ligand (e.g., immunoglobulin single variable domain) that inhibits activity of EGFR inhibits kinase activity of EGFR in the EGFR kinase assay described herein with an 1050 of about 1 μM or less, about 500 nM or less, about 100 nM or less, about 75 nM or less, about 50 nM or less, about 10 nM or less or about 1 nM or less.

A ligand (e.g., immunoglobulin single variable domain) that does not substantially inhibit binding of EGF or TGF alpha to EGFR does not significantly inhibit binding of EGF and/or TGF alpha to EGFR in the receptor binding assay or kinase assay described herein. For example, such a ligand might inhibit binding of EGF or TGF alpha to EGFR in the receptor binding assay or kinase assay described herein with an 1050 of about 1 mM or higher.

“Affinity” and “avidity” are terms of art that describe the strength of a binding interaction. With respect to the ligands of the invention, avidity refers to the overall strength of binding between the targets (e.g., first cell surface target and second cell surface target) on the cell and the ligand. Avidity is more than the sum of the individual affinities for the individual targets.

As used herein, “toxin moiety” refers to a moiety that comprises a toxin. A toxin is an agent that has deleterious effects on or alters cellular physiology (e.g., causes cellular necrosis, apoptosis or inhibits cellular division).

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds VEGF and an immunoglobulin single variable domain that binds EGFR) administered to a subject over the course of one day (24 hours) (daily dose), two days, one week, two weeks, three weeks or one or more months (e.g., by a single administration, or by two or more administrations). The interval between doses can be any desired amount of time.

As used herein “complementary” refers to when two immunoglobulin domains belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on (for example) an immunoglobulin domain and a fibronectin domain are not complementary.

As used herein, “immunoglobulin” refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signaling (for example, receptor molecules, such as the PDGF receptor). The present invention is applicable to all immunoglobulin superfamily molecules which possess binding domains. Preferably, the present invention relates to antibodies.

As used herein “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain. Thus, each ligand comprises at least two different domains.

“Repertoire” A collection of diverse variants, for example polypeptide variants which differ in their primary sequence. A library used in the present invention will encompass a repertoire of polypeptides comprising at least 1000 members.

“Library” The term library refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As described herein an “antigen’ is a molecule that is bound by a binding domain according to the present invention. Typically, antigens are bound by antibody ligands and are capable of raising an antibody response in vivo. It may be a polypeptide, protein, nucleic acid or other molecule. Generally, the dual-specific ligands according to the invention are selected for target specificity against two particular targets (e.g., antigens). In the case of conventional antibodies and fragments thereof, the antibody binding site defined by the variable loops (L1, L2, L3 and H1, H2, H3) is capable of binding to the antigen.

An “epitope” is a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

“Universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity though variation in the hypervariable regions alone.

The phrase, “half-life,” refers to the time taken for the serum concentration of the ligand to reduce by 50%, in vivo, for example due to degradation of the ligand and/or clearance or sequestration of the dual-specific ligand by natural mechanisms. The ligands of the invention are stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus a ligand specific for HSA and two target molecules is compared with the same ligand wherein the specificity to HSA is not present, that is does not bind HSA but binds another molecule. For example, it may bind a third target on the cell. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible.

As referred to herein, the term “competes” means that the binding of a first target to its cognate target binding domain is inhibited when a second target is bound to its cognate target binding domain. For example, binding may be inhibited sterically, for example by physical blocking of a binding domain or by alteration of the structure or environment of a binding domain such that its affinity or avidity for a target is reduced. A protein moiety competes for binding to a target (e.g., EGFR, VEGF, serum albumin) with another agent, when the protein moiety inhibits binding of the other agent to the target in a competitive binding assay (e.g., a competitive ELISA or other suitable binding assay). For example, the protein moiety can inhibit binding of another agent that binds a target (e.g., EGFR, VEGF, serum albumin) in a competitive binding assay by at least about 25%, at least about 30%, at least about 40%, at least about 50%, at least about 60%, at least about 70%, at least about 80%, at least about 90%, or at least about 95%.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45 C., followed by two washes in 0.2×SSC, 0.1% SDS at least at 50 C. (the temperature of the washes can be increased to 55 C. for low stringency conditions); (2) medium stringency hybridization conditions in 6×SSC at about 45 C., followed by one or more washes in 0.2×SSC, 0.1% SDS at 60 C.; (3) high stringency hybridization conditions in 6×SSC at about 45 C., followed by one or more washes in 0.2×SSC, 0.1% SDS at 65 C.; and preferably (4) very high stringency hybridization conditions are 0.5M sodium phosphate, 7% SDS at 65 C., followed by one or more washes at 0.2×SSC, 1% SDS at 65 C. Very high stringency conditions (4) are the preferred conditions and the ones that should be used unless otherwise specified.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are preferably prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

The invention relates to ligands that have binding specificity for VEGF (e.g., human VEGF), ligands that have binding specificity for EGFR (e.g., human EGFR), and to ligands that have binding specificity for VEGF and EGFR (e.g., human VEGF and human EGFR). For example, the ligand can comprise a polypeptide domain having a binding site with binding specificity for VEGF, a polypeptide domain having a binding site with binding specificity for EGFR, or comprise a polypeptide domain having a binding site with binding specificity for VEGF and a polypeptide domain having a binding site with binding specificity for EGFR.

The ligands of the invention provide several advantages. For example, as described herein, the ligand can be tailored to have a desired in vivo serum half-life. Thus, the ligands can be used to control, reduce, or eliminate general toxicity of therapeutic agents, such as cytotoxin used to treat cancer. Further, dAbs are much smaller than conventional antibodies, and can be administered to achieve better tissue penetration than conventional antibodies. Thus, dAbs and ligands that comprise a dAb provide advantages over conventional antibodies when administered to treat cancer, for example by targeting solid tumors. Moreover, many cancers overexpress EGFR, and ligands that have binding specificity for EGFR and VEGF can be administered to target VEGF-inhibitory activity to tumors or the environment of cancer cells. This approach provides two beneficial activities directly at the site of a tumor or cancer, i.e., direct anti-cancer activity by binding to EGFR and inhibiting binding of ligands (e.g., EGF, TGF alpha) to the receptor, and inhibition of angiogenesis that supports tumor formation and development. Accordingly, ligands that have binding specificity for VEGF and EGFR can be administered to a patient with cancer (e.g., EGFR-expressing cancer) to provide superior therapy using a single therapeutic agent.

Further, signals transduced through EGFR can lead to the production of angiogenic factors, such as VEGF. Cancer cells (e.g., in a tumor) that express or overexpress EGFR can produce a high level of VEGF that acts locally to induce formation of tumor vasculature. Accordingly, the ligands of the invention that have binding specificity for VEGF and EGFR can be administered to a subject to target delivery of the VEGF inhibitory activity of the ligand to cells that overexpress EGFR. Accordingly, anti-angiogenic therapy can be delivered specifically to sites where VEGF is being produced (e.g., to cells that overexpress EGFR).

In some embodiments, the ligand has binding specificity for VEGF and comprises an (at least one) immunoglobulin single variable domain with binding specificity for VEGF. In other embodiments, the ligand has binding specificity for EGFR and comprises an (at least one) immunoglobulin single variable domain with binding specificity for EGFR. In certain embodiments, the ligand has binding specificity for VEGF and EGFR, and comprises an (at least one) immunoglobulin single variable domain with binding specificity for VEGF and an (at least one) immunoglobulin single variable domain with binding specificity for EGFR.

The ligand of the invention can be formatted as described herein For example, the ligand of the invention can be formatted to tailor in vivo serum half-life. If desired, the ligand can further comprise a toxin or a toxin moiety as described herein. In some embodiments, the ligand comprises a surface active toxin, such as a free radical generator (e.g., selenium containing toxin) or a radionuclide. In other embodiments, the toxin or toxin moiety is a polypeptide domain (e.g., a dAb) having a binding site with binding specificity for an intracellular target. In particular embodiments, the ligand is an IgG-like format that has binding specificity for VEGF and EGFR (e.g., human VEGF and human EGFR).

### Ligand Formats

The ligand of the invention can be formatted as a monospecific, dual specific or multispecific ligand as described herein. See, also WO 03/002609, the entire teachings of which are incorporated herein by reference, regarding ligand formatting. Such dual specific ligands comprise immunoglobulin single variable domains that have different binding specificities. Such dual specific ligands can comprise combinations of heavy and light chain domains. For example, the dual specific ligand may comprise a V_{H} domain and a V_{L} domain, which may be linked together in the form of an scFv (e.g., using a suitable linker such as Gly₄Ser), or formatted into a bispecific antibody or antigen-binding fragment thereof (e.g. F(ab′)₂ fragment). The dual specific ligands do not comprise complementary V_{H}/V_{L} pairs which form a conventional two chain antibody antigen-binding site that binds antigen or epitope co-operatively. Instead, the dual format ligands comprise a V_{H}/V_{L} complementary pair, wherein the V domains have different binding specificities.

In addition, the dual specific ligands may comprise one or more C_{H} or C_{L} domains if desired. A hinge region may also be included if desired. Such combinations of domains may, for example, mimic natural antibodies, such as IgG or IgM, or fragments thereof, such as Fv, scFv, Fab or F(ab′)₂ molecules. Other structures, such as a single aim of an IgG molecule comprising V_{H}, V_{L}, C_{H}1 and C_{L} domains, are envisaged. Preferably, the dual specific ligand of the invention comprises only two variable domains although several such ligands may be incorporated together into the same protein, for example two such ligands can be incorporated into an IgG or a multimeric immunoglobulin, such as IgM. Alternatively, in another embodiment a plurality of dual specific ligands are combined to form a multimer. For example, two different dual specific ligands are combined to create a tetra-specific molecule. It will be appreciated by one skilled in the art that the light and heavy variable regions of a dual-specific ligand produced according to the method of the present invention may be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

In some embodiments, the linker may be a “natural linker” that contains carboxy-terminal amino acids of an antibody variable domain and amino-terminal amino acids of an antibody constant domain. For example, a natural linker can contain the carboxy-terminal amino acids of Vk and amino-terminal amino acids of Ck (e.g. KVEIKRTVAAPS (SEQ ID NO:706)). In other embodiments, the linker can contain fewer Lys and Arg residues than the natural linker (e.g., LVTVSSAST (SEQ ID NO:707) or (LVTVSSGGGGSGGGS (SEQ ID NO:708)). If desired, the linker can be mutated to substitute some or all of the positively charged residues (e.g., in a natural linker), such as Lys and/or Arg with residues that are not positively charged at physiological pH. For example, Lys and/or Arg residues can be replaced with Asn, Leu, Gln or Ser. This type of linker provides the advantage of reducing protease sensitivity (e.g., serine protease, cysteine protease, matrix metalloprotease, pepsin, trypsin, elastase, chymotrypsin, carboxypeptidase, cathepsin (e.g., cathepsin G), proteinase 3). Examples of such linkers include, GQGTNVEINRTVAAPS (SEQ ID NO:710), GQGTNVEINQTVAAPS (SEQ ID NO:711), GQGTNVEIQRTVAAPS (SEQ ID NO:712), or GQGTLVTVSSTVAAPS (SEQ ID NO:713).

Proteases (e.g., a serine protease, cysteine protease, matrix metalloprotease, pepsin, trypsin, elastase, chymotrypsin, carboxypeptidase, cathepsin (e.g., cathepsin G), proteinase 3) function in the normal turn over and metabolism of proteins. However, in certain physiological states, such as inflammatory states (e.g., COPD) and cancer, the amount of proteases present in a tissue, organ or animal (e.g., in the lung, in or adjacent to a tumor) can increase. This increase in proteases can result in accelerated degradation and inactivation of endogenous proteins and of therapeutic or diagnostic peptides, polypeptides and proteins that are administered. In fact, some agents that have potential for in vivo use (e.g., use in treating, diagnosing or preventing disease) have only limited efficacy because they are rapidly degraded and inactivated by proteases.

The invention relates to ligand comprising a linker that is resistant to protease degradation. The protease resistant ligands of the invention provide several advantages. For example, a protease resistant ligand can be administered to a subject and remain active in vivo longer than protease sensitive agents. Accordingly, protease resistant ligand will remain functional for a period of time that is sufficient to produce biological effects.

A ligand or linker that is resistant to protease degradation is not substantially degraded by a protease when incubated with the protease under conditions suitable for protease activity for at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, at least about 36 hours, or at least about 48 hours. A ligand or linker is not substantially degraded when no more than about 25%, no more than about 20%, no more than about 15%, no more than about 14%, no more than about 13%, no more than about 12%, no more than about 11%, no more than about 10%, no more than about 9%, no more than about 8%, no more than about 7% no more than about 6%, no more than about 5%, no more than about 4%, no more than about 3%, no more than about 2%, no more than about 1%, or substantially none of the ligand or linker is degraded by protease after incubation with the protease for at least about 2 hours. Protein degradation can be assessed using any suitable method, for example, by SDS-PAGE.

Protease resistance can be assessed using any suitable method. For example, a protease can be added to a solution of ligand or linker in a suitable buffer (e.g., PBS) to produce a ligand or linker/protease solution, such as a solution of at least about 0.01% (w/w) protease, about 0.01% to about 5% (w/w) protease, about 0.05% to about 5% (w/w) protease, about 0.1% to about 5% (w/w) protease, about 0.5% to about 5% (w/w) protease, about 1% to about 5% (w/w) protease, at least about 0.01% (w/w) protease, at least about 0.02% (w/w) protease, at least about 0.03% (w/w) protease, at least about 0.04% (w/w) protease, at least about 0.05% (w/w) protease, at least about 0.06% (w/w) protease, at least about 0.07% (w/w) protease, at least about 0.08% (w/w) protease, at least about 0.09% (w/w) protease, at least about 0.1% (w/w) protease, at least about 0.2% (w/w) protease, at least about 0.3% (w/w) protease, at least about 0.4% (w/w) protease, at least about 0.5% (w/w) protease, at least about 0.6% (w/w) protease, at least about 0.7% (w/w) protease, at least about 0.8% (w/w) protease, at least about 0.9% (w/w) protease, at least about 1% (w/w) protease, at least about 2% (w/w) protease, at least about 3% (w/w) protease, at least about 4% (w/w) protease, or about 5% (w/w) protease. The ligand or linker/protease mixture can be incubated at a suitable temperature for protease activity (e.g., at 37° C.) and samples can be taken at time intervals (e.g., at 1 hour, 2 hours, 3 hours, etc.) and the protease reaction stopped. The samples can then be analyzed for protein degradation using any suitable method, such as SDS-PAGE analysis. The results can be used to establish a time course of degradation.

Ligands can be formatted as bi- or multispecific antibodies or antibody fragments or into bi- or multispecific non-antibody structures. Suitable formats include, any suitable polypeptide structure in which an antibody variable domain or one or more of the CDRs thereof can be incorporated so as to confer binding specificity for antigen on the structure. A variety of suitable antibody formats are known in the art, such as, bispecific IgG-like formats (e.g., chimeric antibodies, humanized antibodies, human antibodies, single chain antibodies, heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., V_{H}, V_{L}, V_{HH}), a dAb), and modified versions of any of the foregoing (e.g., modified by the covalent attachment of polyalkylene glycol (e.g., polyethylene glycol, polypropylene glycol, polybutylene glycol) or other suitable polymer). See, PCT/GB03/002804, filed Jun. 30, 2003, which designated the United States, (WO 2004/081026) regarding PEGylated single variable domains and dAbs, suitable methods for preparing same, increased in vivo half-life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

The ligand can be formatted using a suitable linker such as (Gly₄Ser)ₙ, where n=from 1 to 8, e.g., 2, 3, 4, 5, 6 or 7. If desired, ligands, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides. In some embodiments, the ligand comprises one, two or more dAbs of the same or different binding specificities, and C_{H}2, C_{H}3, C_{H}2-C_{H}3, hinge-C_{H}2, hinge-C_{H}3, hinge-C_{H}2-C_{H}3, a portion of hinge-C_{H}2, a portion of hinge-C_{H}3, or a portion of hinge-C_{H}2-C_{H}3. In such embodiments, C_{H}2, C_{H}3, C_{H}2-C_{H}3, hinge-C_{H}2, hinge-C_{H}3, hinge-C_{H}2-C_{H}3, the portion of hinge-C_{H}2, the portion of hinge-C_{H}3, and the portion of hinge-C_{H}2-C_{H}3 can be from any desired antibody, such as a human IgG, such as a human IgG1 or a human IgG4.

In some embodiments, the ligand of the invention comprises an anti-EGFR dAb or an anti-VEGF dAb that is fused (e.g., directly or through a linker) to an Fc region of an antibody. In some embodiments, the ligand is an Fc fusion that comprises an anti-VEGF dAb that is disulfide bonded to an anti-EGFR dAb. In particular examples, the ligand comprises two or more dAbs (e.g., two dAbs that bind EGFR, two dAbs that bind VEGF, a dAb that binds EGFR and a dAb that binds VEGF) and an Fe region, and the ligand has the structure, from amino terminus to carboxy terminus, V_{H}-V_{H}-Fc, V_{L}-V_{L}-Fc, V_{H}-V_{L}-Fc, V_{L}-V_{H}-Fc. For example, the ligand can have the structure V_{H}-V_{K}-hinge-CH2-CH3, V_{K}-V_{H}-hinge-CH2-CH3, V_{K}-V_{K}-hinge-CH2-CH3, or V_{H}-V_{H}-hinge-CH2-CH3. If desired, the V_{H} shown in any of the foregoing formulae can be a V_{HH}. Two ligands that contain Fc regions can be bonded together to form a dimer, for example, through a disulfide bond (e.g., in the hinge region).

Generally the orientation of the polypeptide domains that have a binding site with binding specificity for a target (e.g., dAbs), and whether the ligand comprises a linker, is a matter of design choice. However, some orientations, with or without linkers, may provide preferred binding characteristics in comparison to other orientations. All orientations (e.g., dAb1-dAb2-Fc; dAb2-dAb1-Fc) are encompassed by the invention, and ligands that contain an orientation that provides desired binding characteristics can be easily identified by routine screening.

Ligands and dAb monomers can also be combined and/or formatted into non-antibody multi-ligand structures to form multivalent complexes, which bind target molecules with the same antigen, thereby providing superior avidity. For example natural bacterial receptors such as SpA can be used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides. Protein scaffolds may be combined; for example, CDRs may be grafted on to a CTLA4 scaffold and used together with immunoglobulin V_{H} or V_{L} domains to form a ligand. Likewise, fibronectin, lipocallin and other scaffolds may be combined

A variety of suitable methods for preparing any desired format are known in the art. For example, antibody chains and formats (e.g., bispecific IgG-like formats, chimeric antibodies, humanized antibodies, human antibodies, single chain antibodies, homodimers and heterodimers of antibody heavy chains and/or light chains) can be prepared by expression of suitable expression constructs and/or culture of suitable cells (e.g., hybridomas, heterohybridomas, recombinant host cells containing recombinant constructs encoding the format). Further, formats such as antigen-binding fragments of antibodies or antibody chains (e.g., bispecific binding fragments, such as a Fv fragment (e.g., single chain Fv (scFv), a disulfide bonded Fv), a Fab fragment, a Fab′ fragment, a F(ab′)₂ fragment), can be prepared by expression of suitable expression constructs or by enzymatic digestion of antibodies, for example using papain or pepsin.

The ligand can be formatted as a multispecific ligand, for example as described in WO 03/002609, the entire teachings of which are incorporated herein by reference. Such multispecific ligands possess more than one epitope binding specificity. Generally, the multi-specific ligand comprises two or more epitope binding domains, such as dAbs or non-antibody protein domain comprising a binding site for an epitope, e.g., an affibody, an SpA domain, an LDL receptor class A domain, an EGF domain, an avimer. Multispecific ligands can be formatted further as described herein.

In some embodiments, the ligand is an IgG-like format. Such formats have the conventional four chain structure of an IgG molecule (2 heavy chains and two light chains), in which one or more of the variable regions (V_{H} and or V_{L}) have been replaced with a dAb or single variable domain of a desired specificity. Preferably, each of the variable regions (2 V_{H} regions and 2 V_{L} regions) is replaced with a dAb or single variable domain. The dAb(s) or single variable domain(s) that are included in an IgG-like format can have the same specificity or different specificities. In some embodiments, the IgG-like format is tetravalent and can have one, two, three or four specificities. For example, the IgG-like format can be monospecific and comprises 4 dAbs that have the same specificity; bispecific and comprises 3 dAbs that have the same specificity and another dAb that has a different specificity; bispecific and comprise two dAbs that have the same specificity and two dAbs that have a common but different specificity; trispecific and comprises first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)₂, Fab′, Fv, scF_{V}) can be prepared. In addition, a particular constant region of Fc portion (e.g., of an IgG, such as IgG1), variant or portion thereof can be selected in order to tailor effector function. For example, if complement activation and/or antibody dependent cellular cytotoxicity (ADCC) function is desired, the ligand can be an IgG1-like format. If desired, the IgG-like format can comprise a mutated constant region (variant IgG heavy chain constant region) to minimize binding to Fc receptors and/or ability to fix complement. (see e.g. Winter et al, GB 2,209,757 B; Morrison et al., WO 89/07142; Morgan et al., WO 94/29351, Dec. 22, 1994).

In some embodiments, the IgG-like formats can comprise an anti-EGFR dAb (e.g., DOM16-39-542, DOM16-39-618 or DOM16-39-619), an anti-VEGF dAb (e.g., DOM15-26-501), or an anti-EGFR dAb and an anti-VEGF dAb.

The ligands of the invention can be formatted as a fusion protein that contains a first immunoglobulin single variable domain that is fused directly to a second immunoglobulin single variable domain. If desired such a format can further comprise a half-life extending moiety. For example, the ligand can comprise a first immunoglobulin single variable domain that is fused directly to a second immunoglobulin single variable domain that is fused directly to an immunoglobulin single variable domain that binds serum albumin. For example, the ligand can be an in line fusion of two or more protein moieties that have a binding site with binding specificity for EGFR that competes for binding to EGFR with an anti-EGFR domain antibody (e.g., any of the DOM16 dAbs disclosed herein) and fused to an anti-serum albumin dAb (e.g., any of the DOM7 dAbs disclosed herein). In some embodiments, the protein moieties that have a binding site with binding specificity for EGFR (e.g., anti-EGFR dAbs) have different epitopic specificities. In other examples, the ligand is an in line fusion protein comprising a protein moiety that has a binding site with binding specificity for EGFR (e.g., anti-EGFR dAb), a protein moiety that has a binding site with binding specificity for VEGF, and an anti-serum albumin dAb.

In particular embodiments, such an in line fusion comprises DOM16-39-618 dAb and/or DOM16-39-619 and an anti-serum albumin dAb (e.g., DOM16-39-6,8-DOM7h-14, DOM7h-14-DOM16-39-618, DOM16-39-6,9-DOM7h-14, DOM7h-14-DOM16-39-619). In other embodiments, the in line fusion comprises a protein moiety (e.g., dAb) that has at least about 80%, 85%, 87%, 90%, 91%, 92%, 93%, 94%, 95%, 96%, 97%, 98% or 99% amino acid sequence identity with the amino acid sequence of DOM16-39-618, a protein moiety that has at least about 80%, 85%, 87%, 90%, 91%, 92%, 93%, 94%, 95%, 96%, 97%, 98% or 99% amino acid sequence identity with the amino acid sequence of DOM16-39-619, and/or a protein moiety (e.g., dAb) that has at least about 80%, 85%, 87%, 90%, 91%, 92%, 93%, 94%, 95%, 96%, 97%, 98% or 99% amino acid sequence identity with the amino acid sequence of an anti-serum albumin dAb disclosed herein, such as DOM7h-14.

In other particular embodiments, the ligand comprises an anti-VEGF dAb, an anti-EGFR dAb, and an anti-serum albumin dAb (e.g., DOM15-10-DOM16-39-anti-serum albumin dAb, DOM16-39-DOM15-10-anti-serum albumin dAb, DOM15-26-501-DOM16-39-anti-serum albumin dAb, DOM16-39-DOM15-26-501-anti-serum albumin dAb). In other embodiments, the in line fusion comprises a protein moiety (e.g., dAb) that has at least about 80%, 85%, 87%, 90%, 91%, 92%, 93%, 94%, 95%, 96%, 97%, 98% or 99% amino acid sequence identity with the amino acid sequence of an anti-VEGF dAb disclosed herein, such as DOM15-10 or DOM15-25-501, and/or a protein moiety that has at least about 80%, 85%, 87%, 90%, 91%, 92%, 93%, 94%, 95%, 96%, 97%, 98% or 99% amino acid sequence identity with the amino acid sequence of an anti-EGFR dAb disclosed herein, such as DOM16-39, and/or a protein moiety (e.g., dAb) that has at least about 80%, 85%, 87%, 90%, 91%, 92%, 93%, 94%, 95%, 96%, 97%, 98% or 99% amino acid sequence identity with the amino acid sequence of an anti-serum albumin dAb disclosed herein, such as DOM7h-14.

Generally the orientation of the polypeptide domains that have a binding site with binding specificity for a target, and whether the ligand comprises a linker, is a matter of design choice. However, some orientations, with or without linkers, may provide better binding characteristics than other orientations. All orientations (e.g., dAb1-linker-dAb2; dAb2-linker-dAb1) are encompassed by the invention are ligands that contain an orientation that provides desired binding characteristics can be easily identified by screening.

### Half-Life Extended Formats

The ligands and dAb monomers disclosed herein can be formatted to extend their in vivo serum half-life. Increased in vivo half-life is useful in in vivo applications of immunoglobulins, especially antibodies and most especially antibody fragments of small size such as dAbs. Such fragments (Fvs, disulphide bonded Fvs, Fabs, scFvs, dAbs) are rapidly cleared from the body, which can limit clinical applications.

A ligand can be formatted as a larger antigen-binding fragment of an antibody or as an antibody (e.g., formatted as a Fab, Fab′, F(ab)₂, F(ab′)₂, IgG, scFv) that has larger hydrodynamic size. Ligands can also be formatted to have a larger hydrodynamic size, for example, by attachment of a polyalkyleneglycol group (e.g. polyethyleneglycol (PEG) group, polypropylene glycol, polybutylene glycol), serum albumin, transferrin, transferrin receptor or at least the transferrin-binding portion thereof, an antibody Fc region, or by conjugation to an antibody domain. In some embodiments, the ligand (e.g., dAb monomer) is PEGylated. Preferably the PEGylated ligand (e.g., dAb monomer) binds VEGF and/or EGFR with substantially the same affinity or avidity as the same ligand that is not PEGylated. For example, the ligand can be a PEGylated ligand comprising a dAb that binds VEGF or EGFR with an affinity or avidity that differs from the avidity of ligand in unPEGylated form by no more than a factor of about 1000, preferably no more than a factor of about 100, more preferably no more than a factor of about 10, or with affinity or avidity substantially unchanged relative to the unPEGylated form. See, PCT/GB03/002804, filed Jun. 30, 2003, which designated the United States, (WO 2004/081026) regarding PEGylated single variable domains and dAbs, suitable methods for preparing same, increased in vivo half-life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

Hydrodynamic size of the ligands (e.g., dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g., the size of a PEG moiety attached to a dAb monomer), can be varied depending on the desired application. For example, where ligand is intended to leave the circulation and enter into peripheral tissues, it is desirable to keep the hydrodynamic size of the ligand low to facilitate extravazation from the blood stream. Alternatively, where it is desired to have the ligand remain in the systemic circulation for a longer period of time the size of the ligand can be increased, for example by formatting as an Ig like protein or by addition of a 30 to 60 kDa PEG moiety (e.g., linear or branched PEG 30 to 40 kDa PEG, such as addition of two 20 kDa PEG moieties.) The size of the ligand format can be tailored to achieve a desired in vivo serum half-life, for example to control exposure to a toxin and/or to reduce side effects of toxic agents.

The hydrodynamic size of ligand (e.g., dAb monomer) and its serum half-life can also be increased by conjugating or linking the ligand to a binding domain (e.g., antibody or antibody fragment) that binds an antigen or epitope that increases half-life in vivo, as described herein. For example, the ligand (e.g., dAb monomer) can be conjugated or linked to an anti-serum albumin or anti-neonatal Fc receptor antibody or antibody fragment, e.g., an anti-SA or anti-neonatal Fc receptor dAb, Fab, Fab′ or scFv, or to an anti-SA affibody or anti-neonatal Fc receptor affibody.

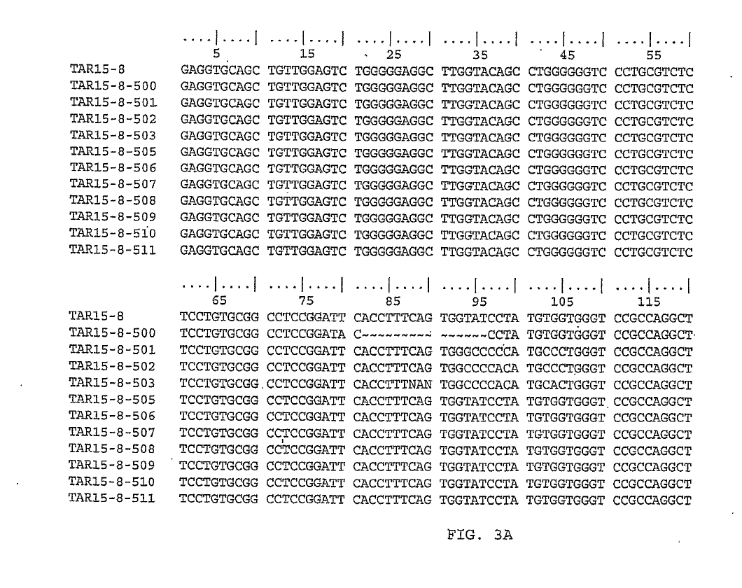

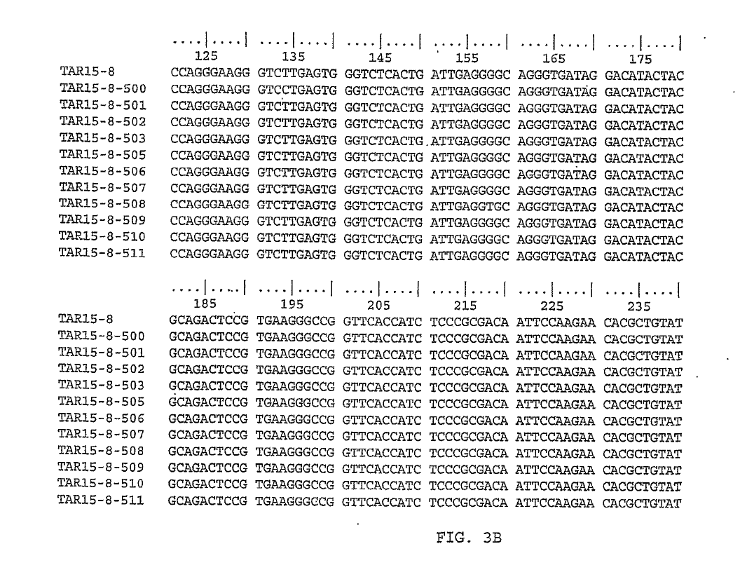

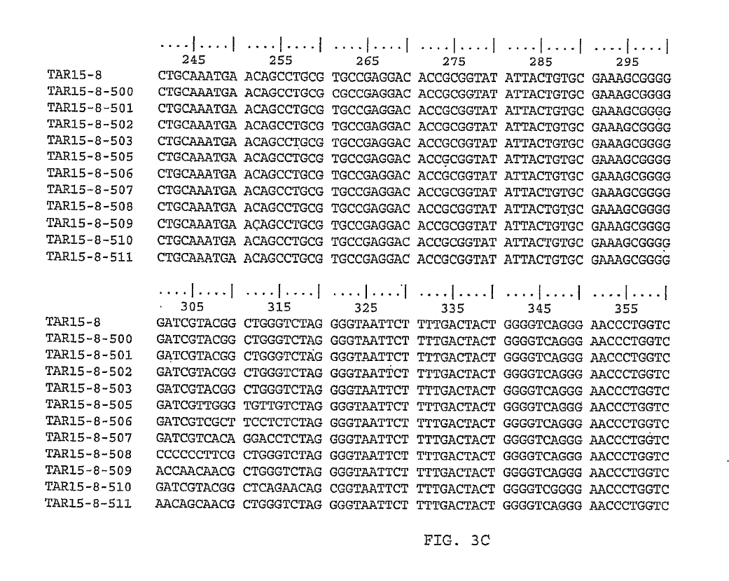

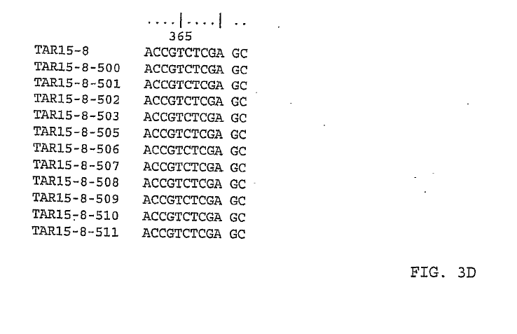

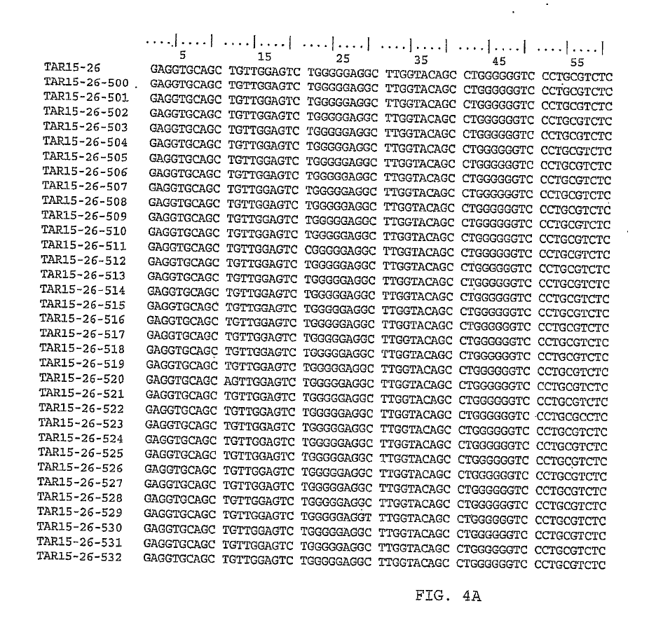

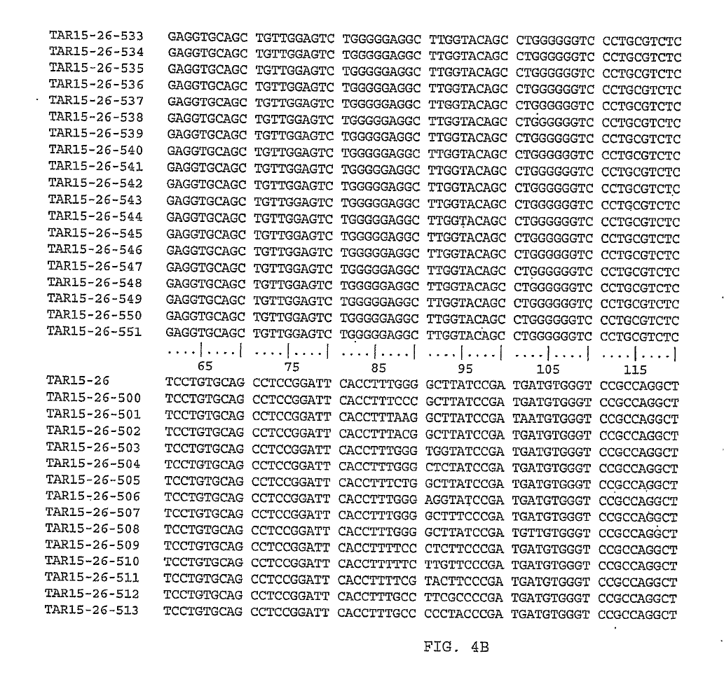

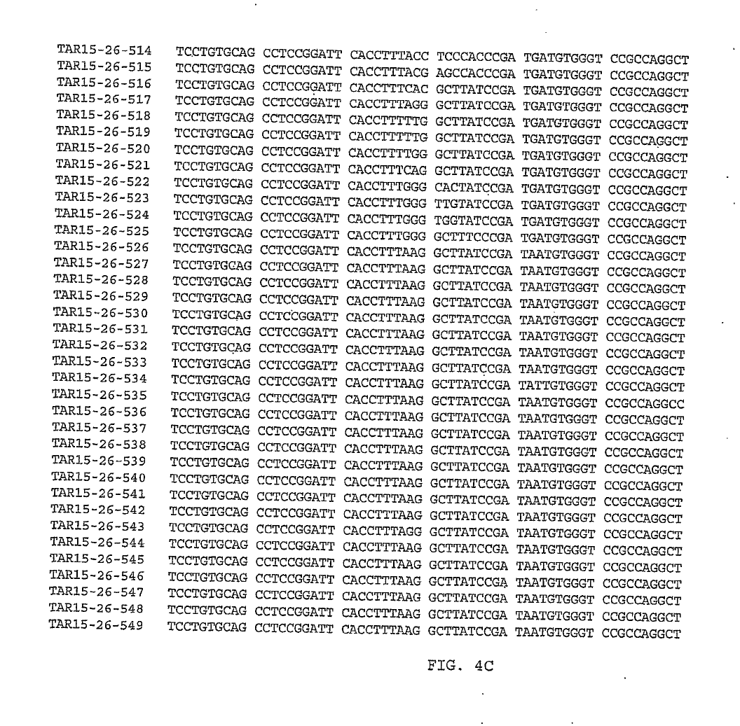

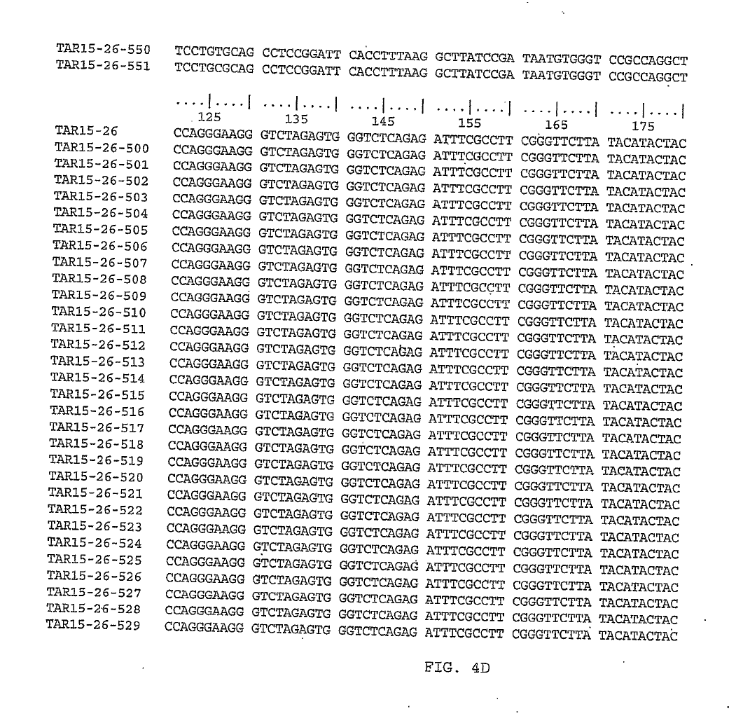

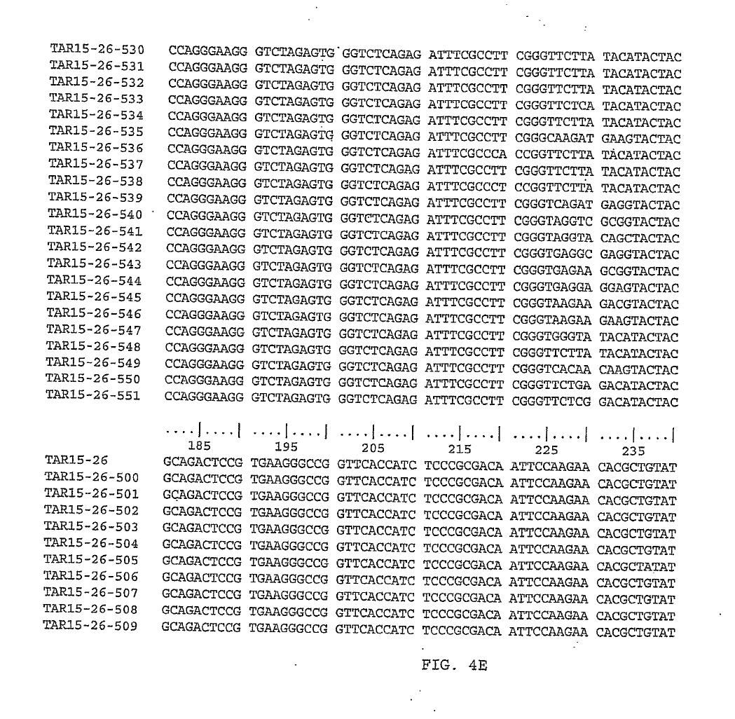

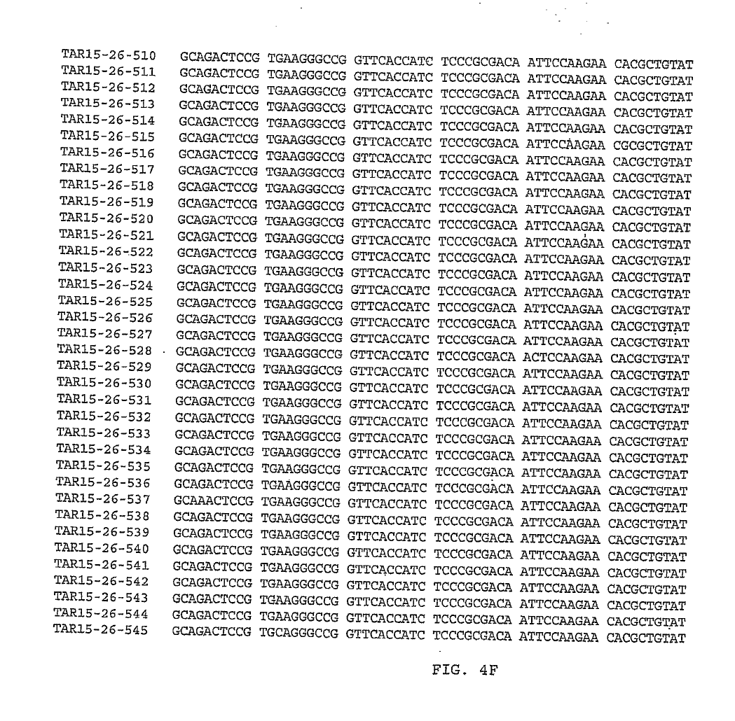

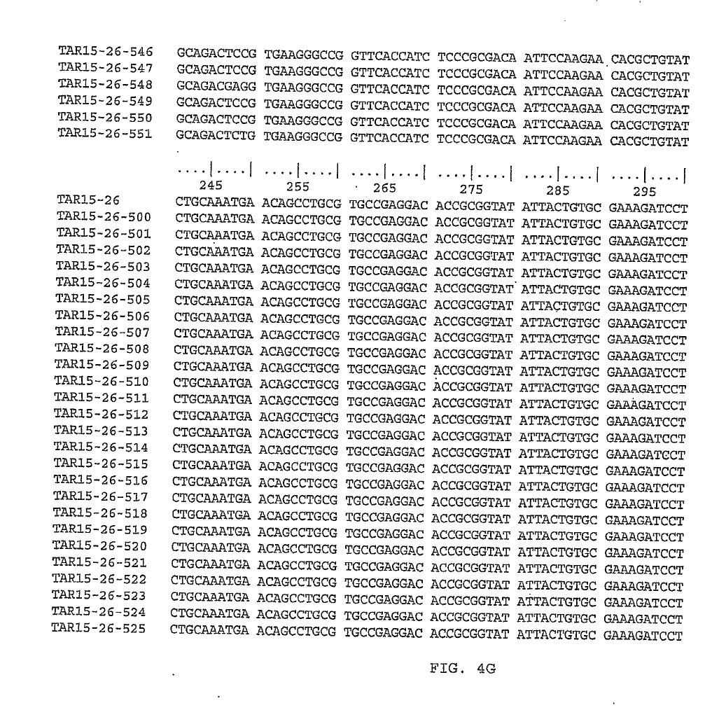

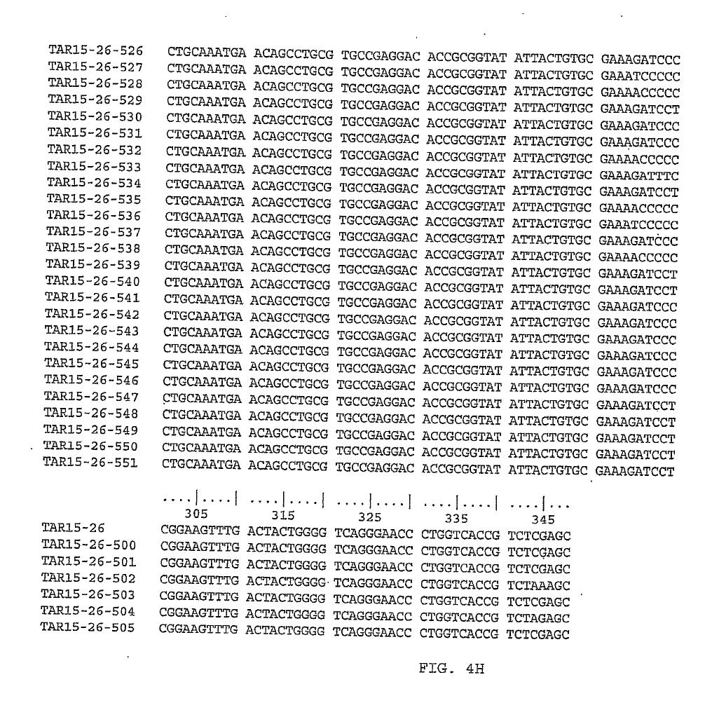

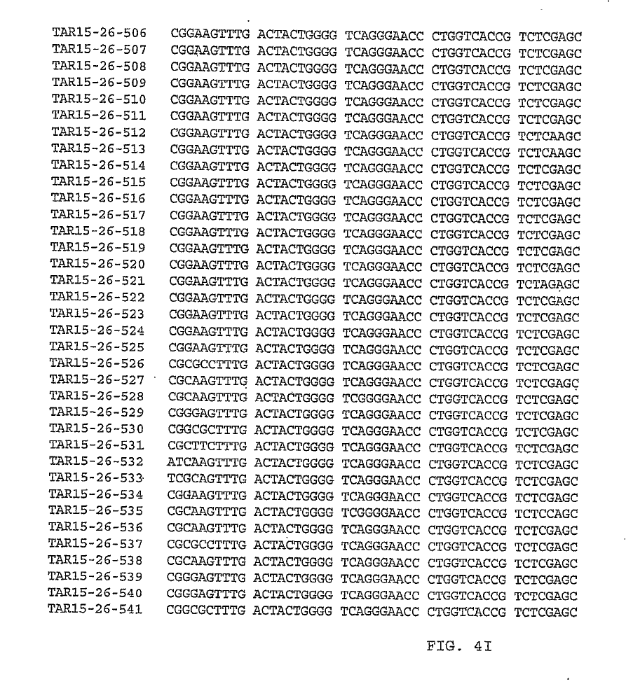

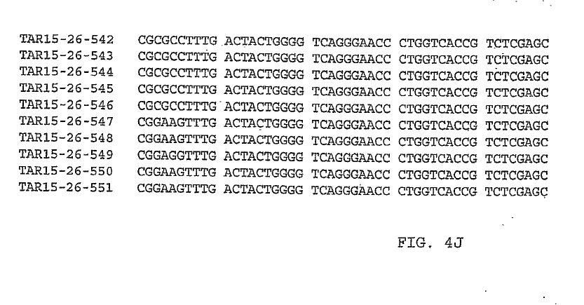

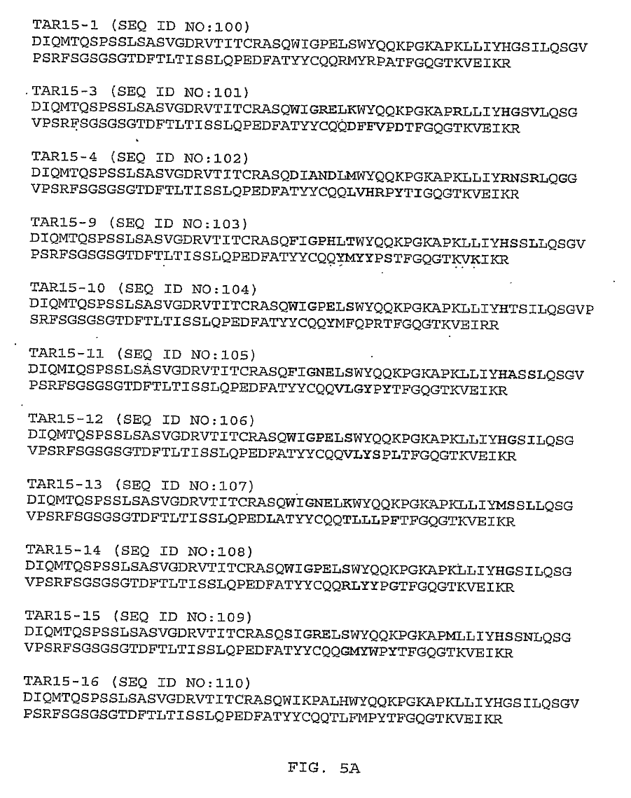

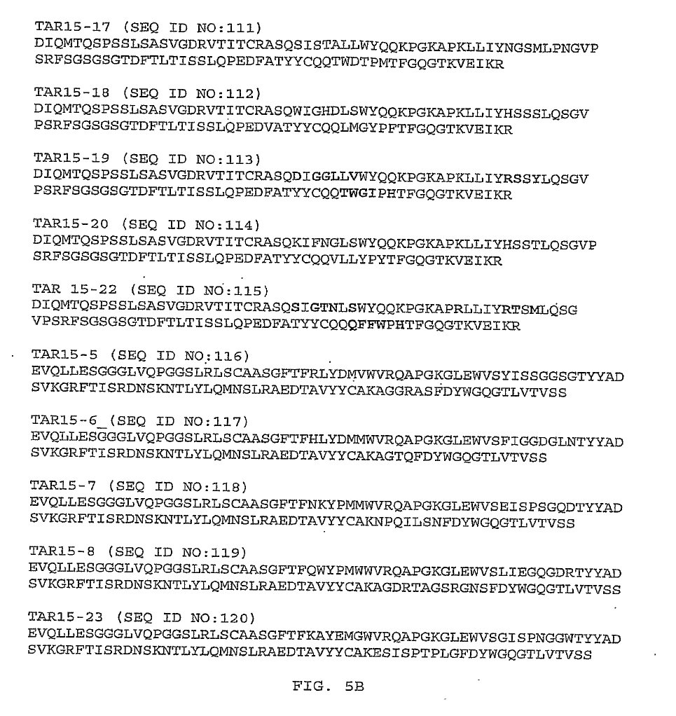

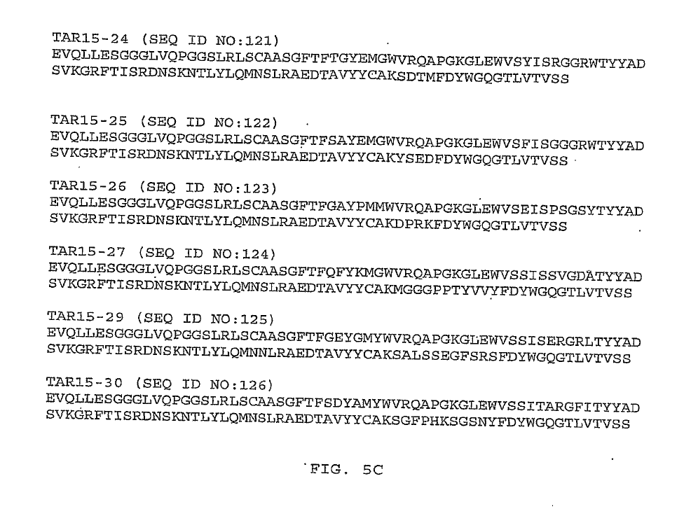

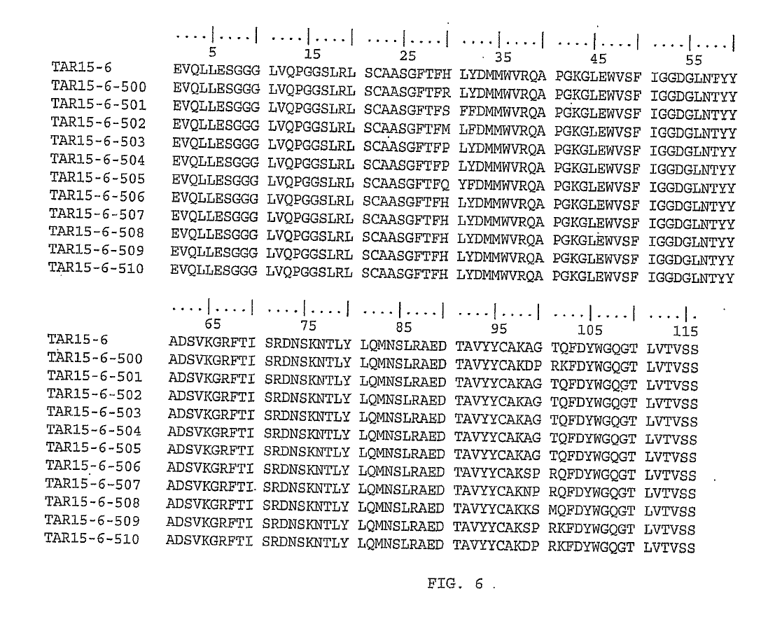

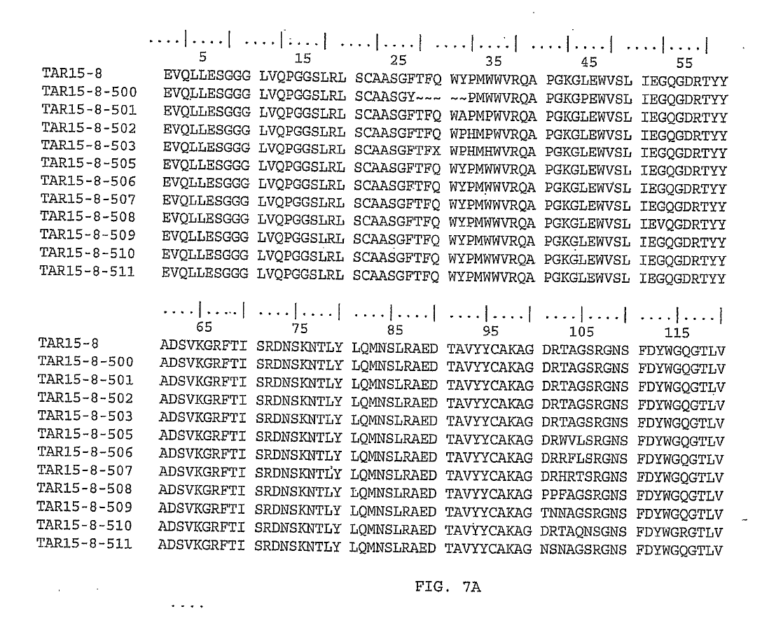

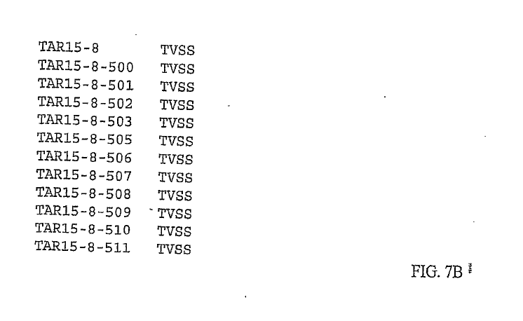

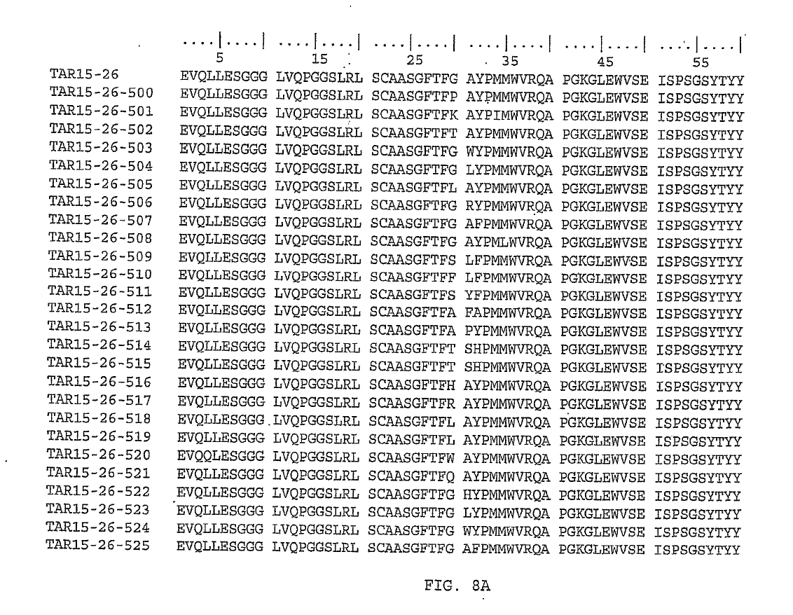

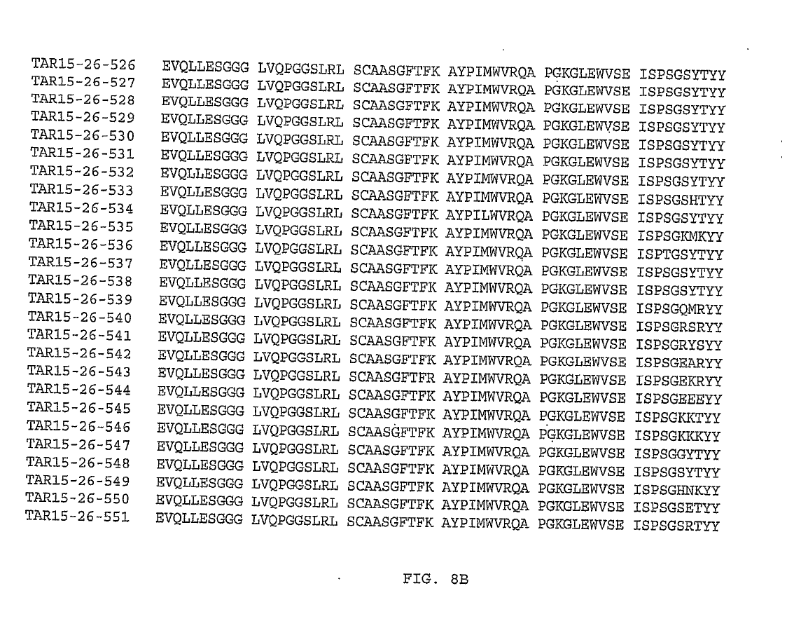

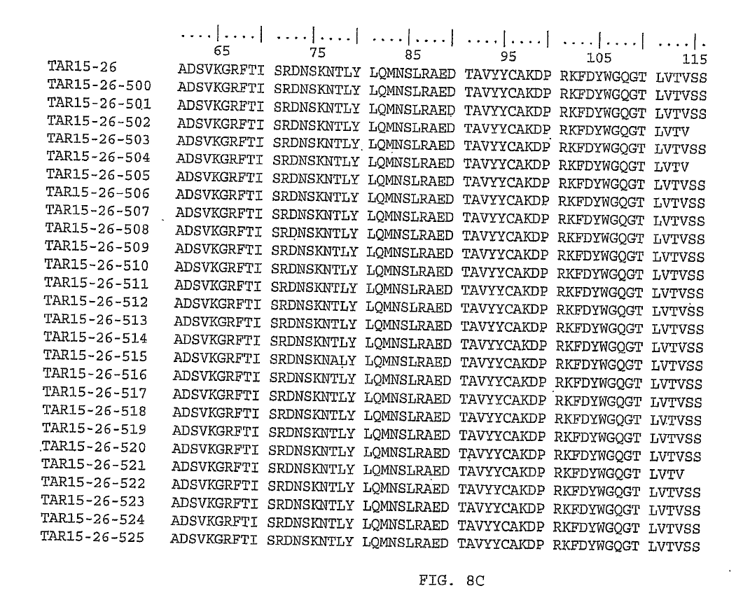

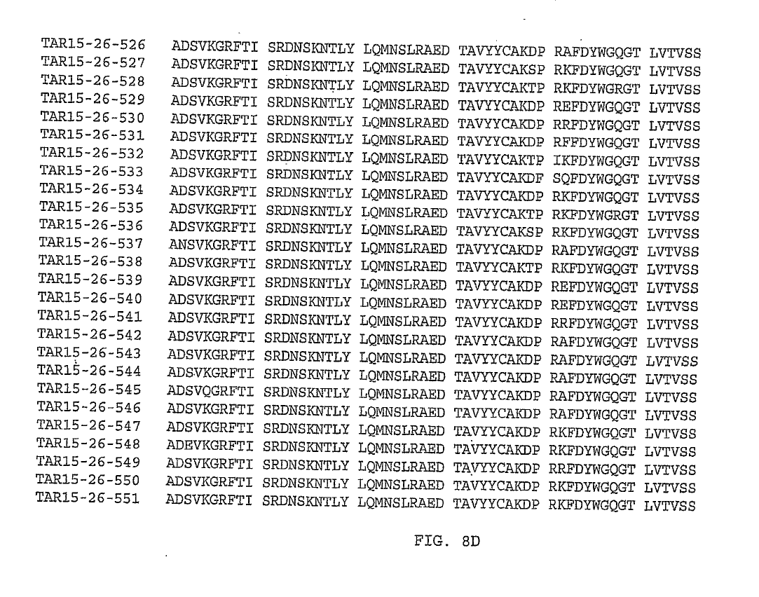

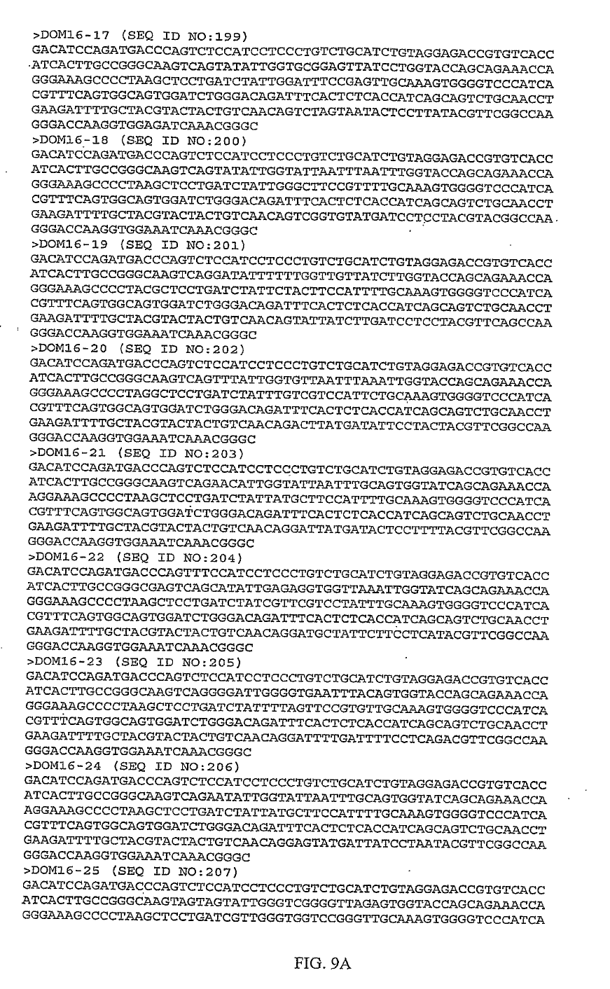

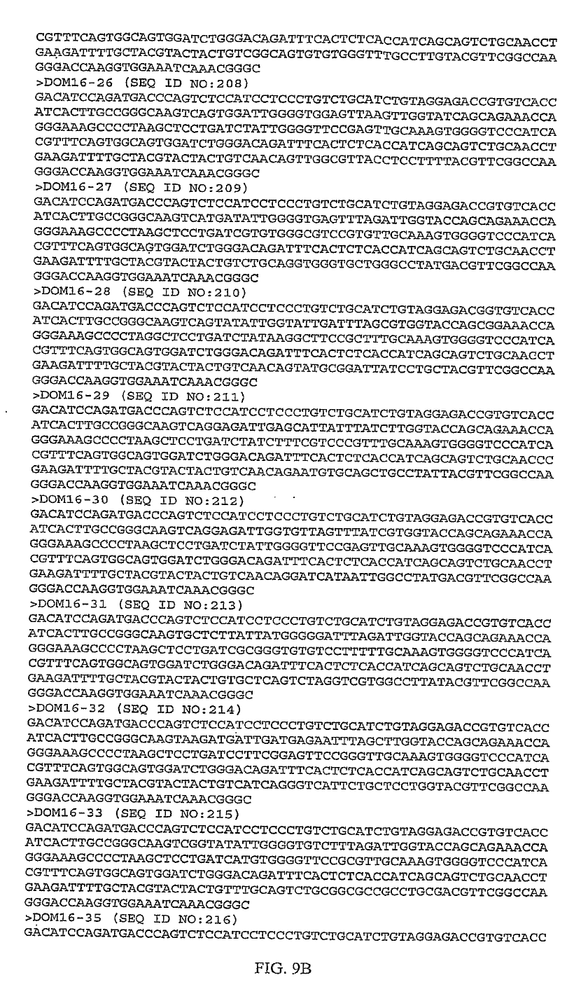

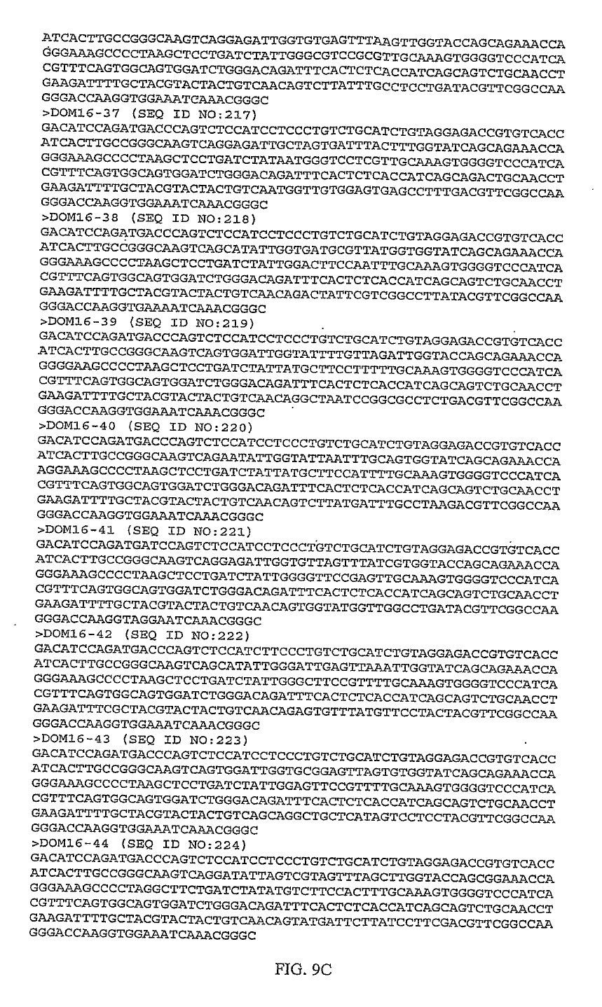

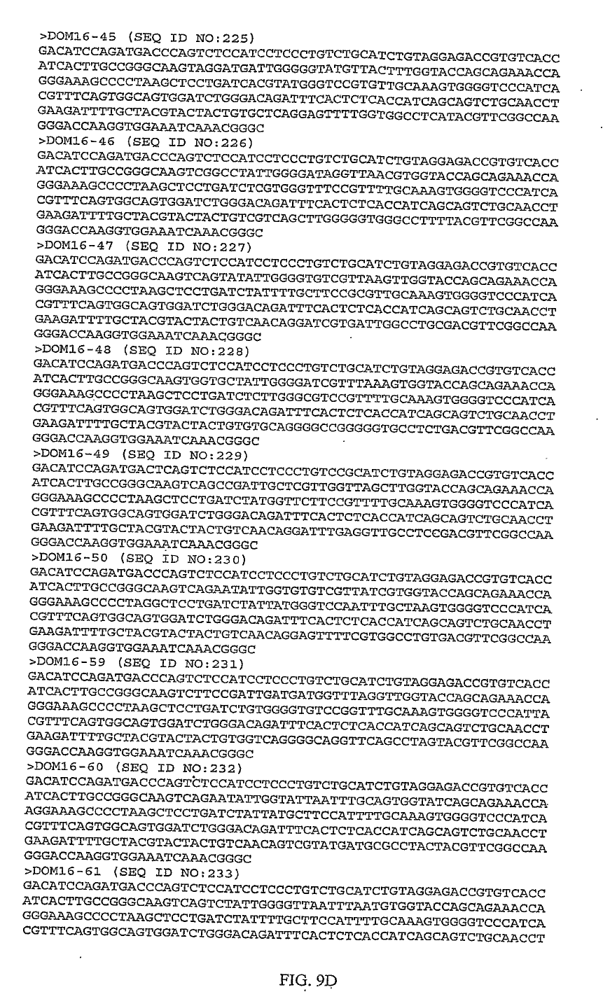

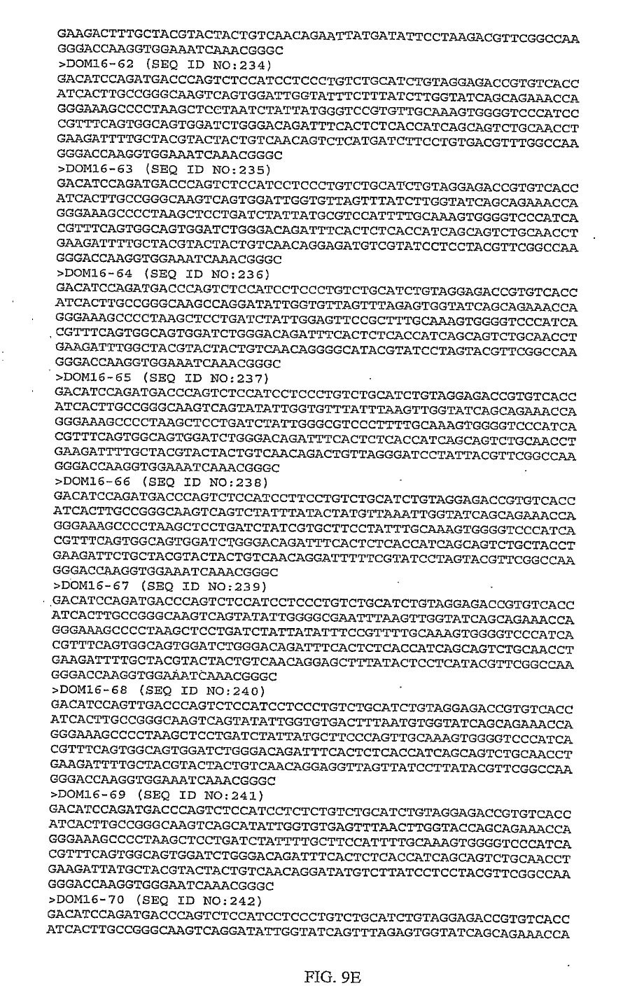

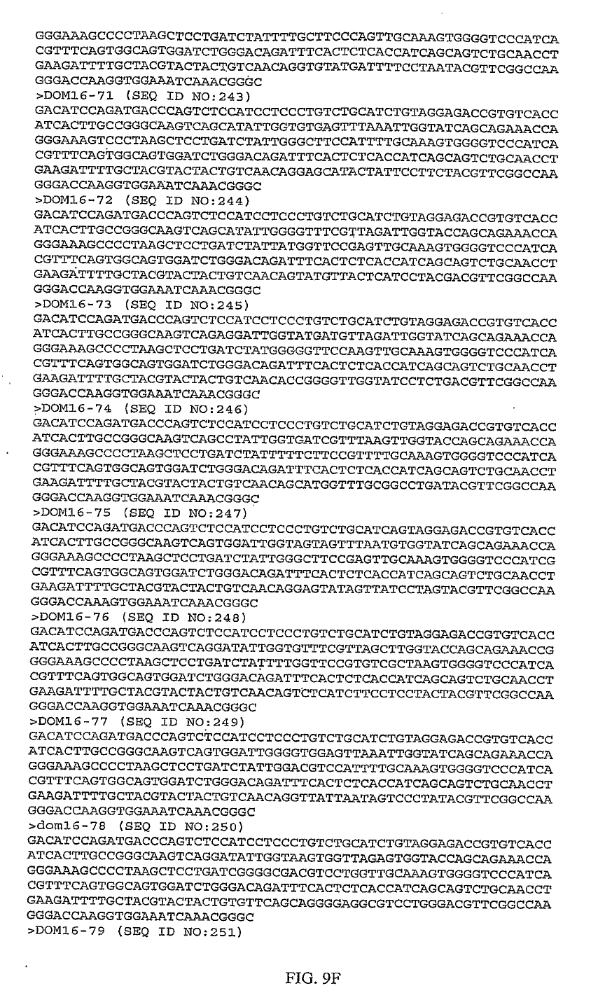

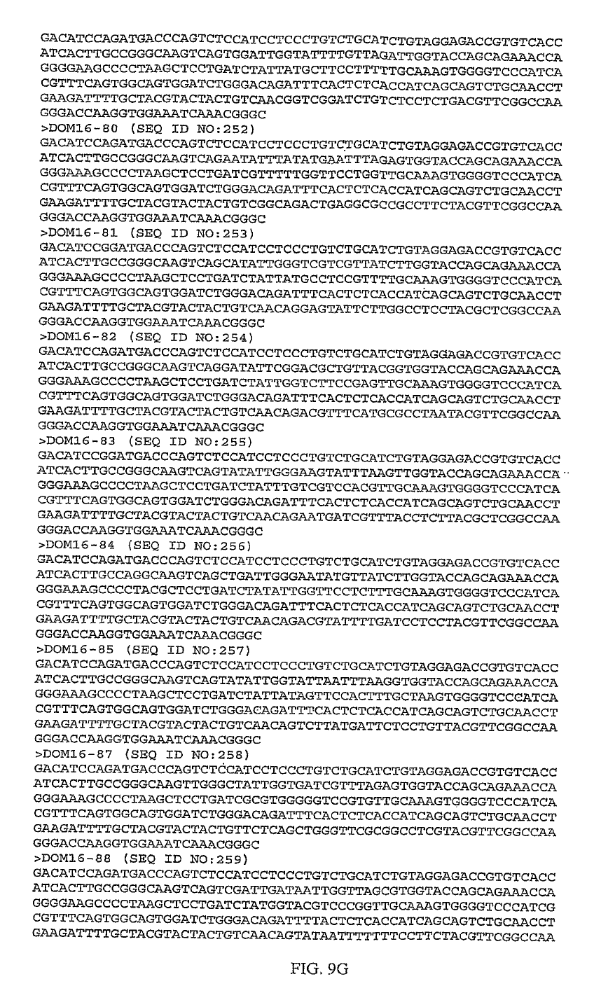

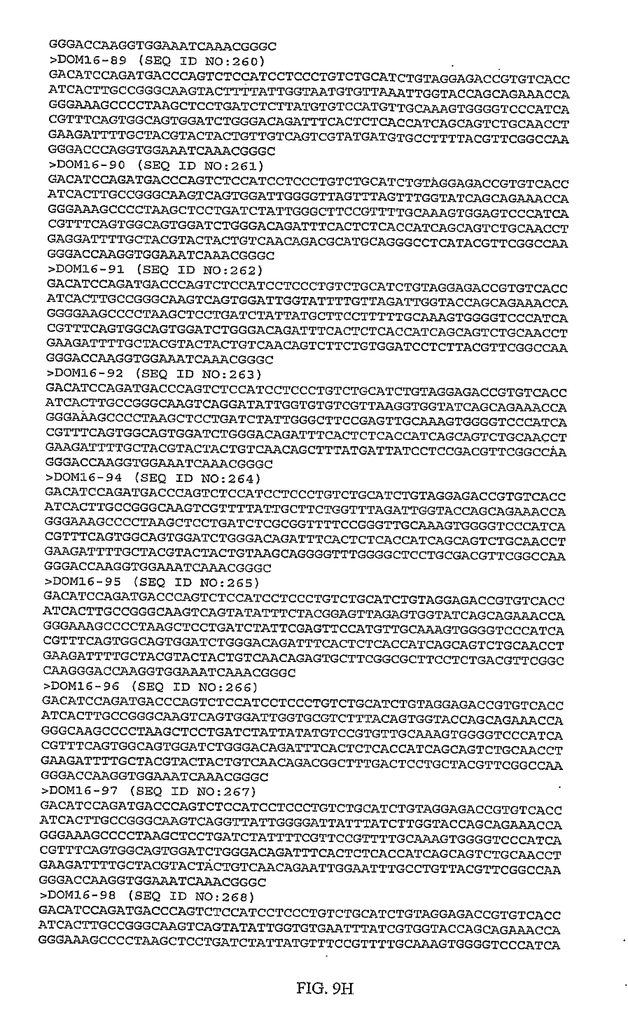

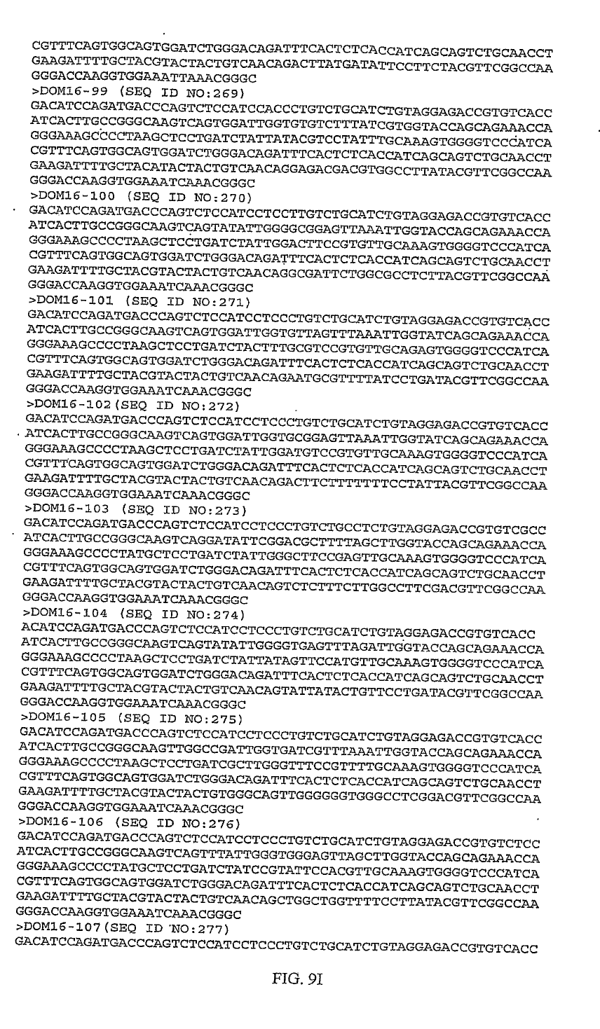

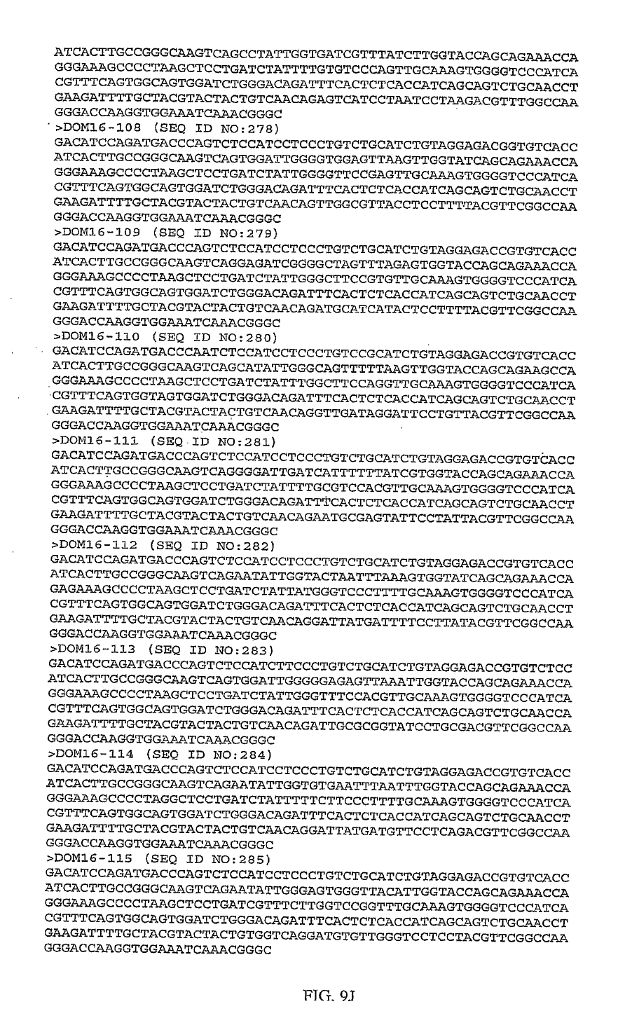

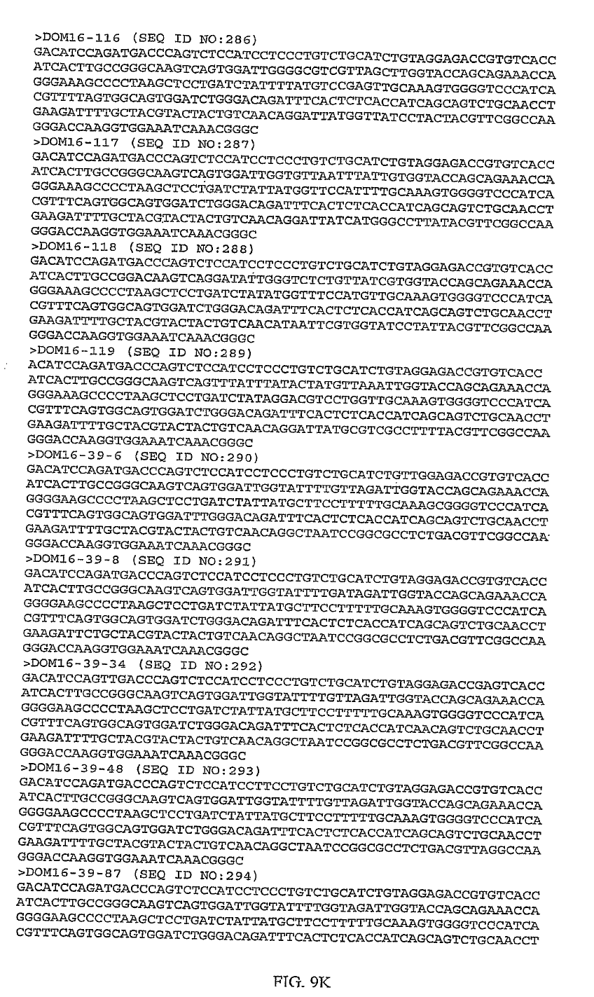

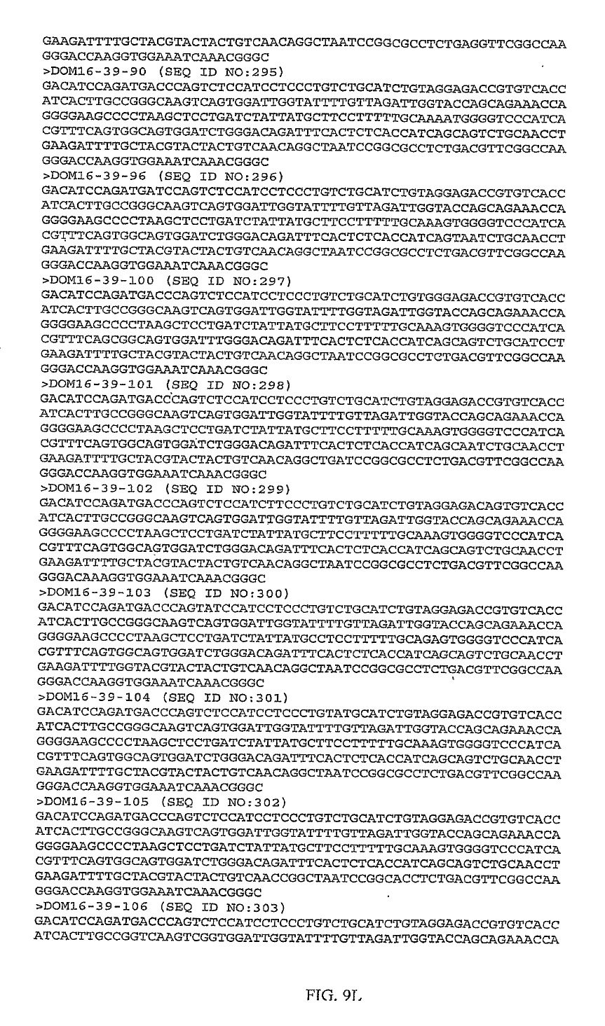

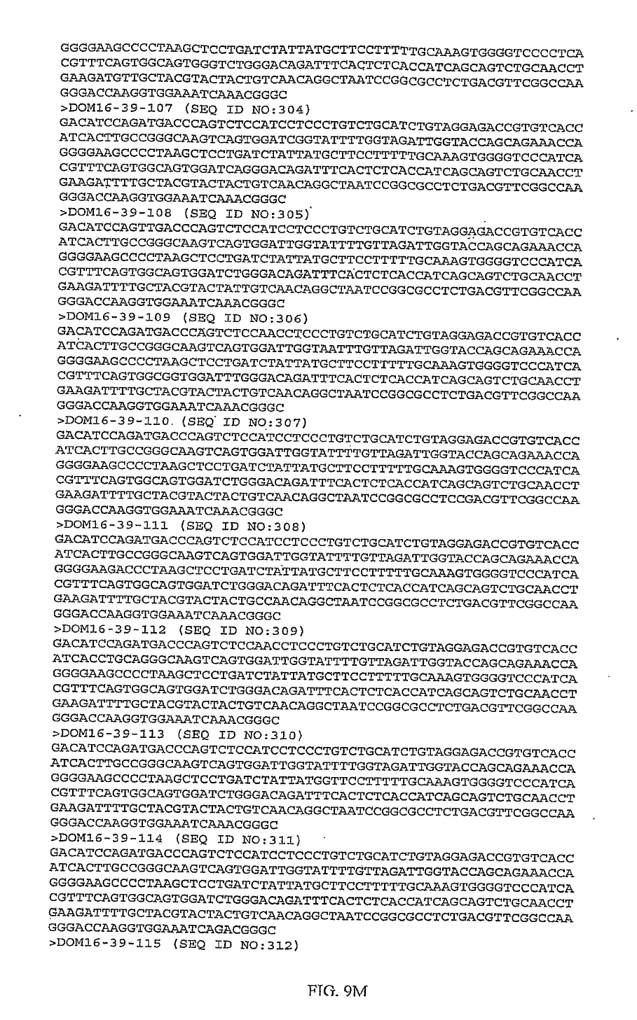

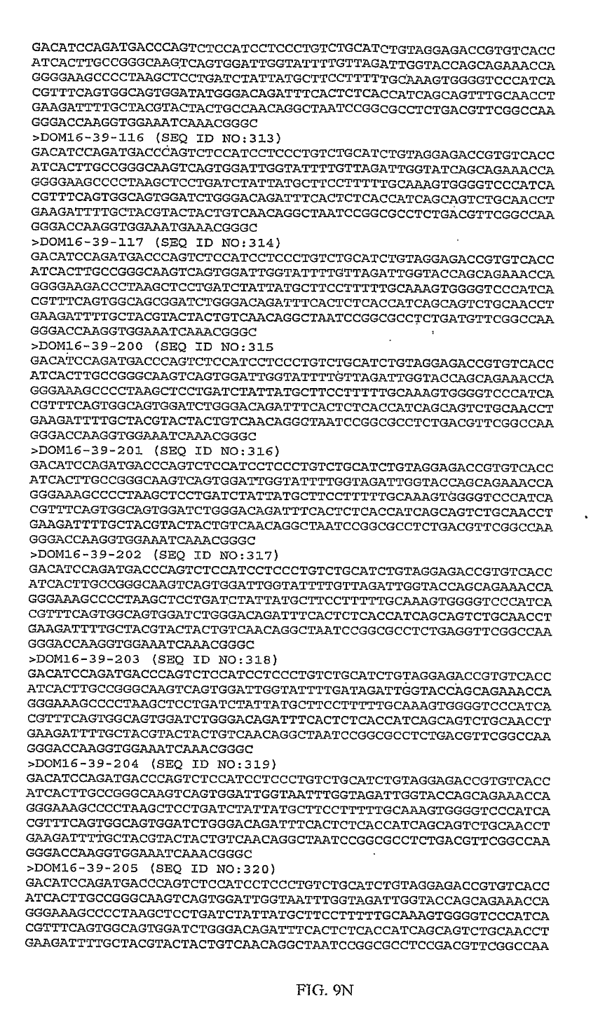

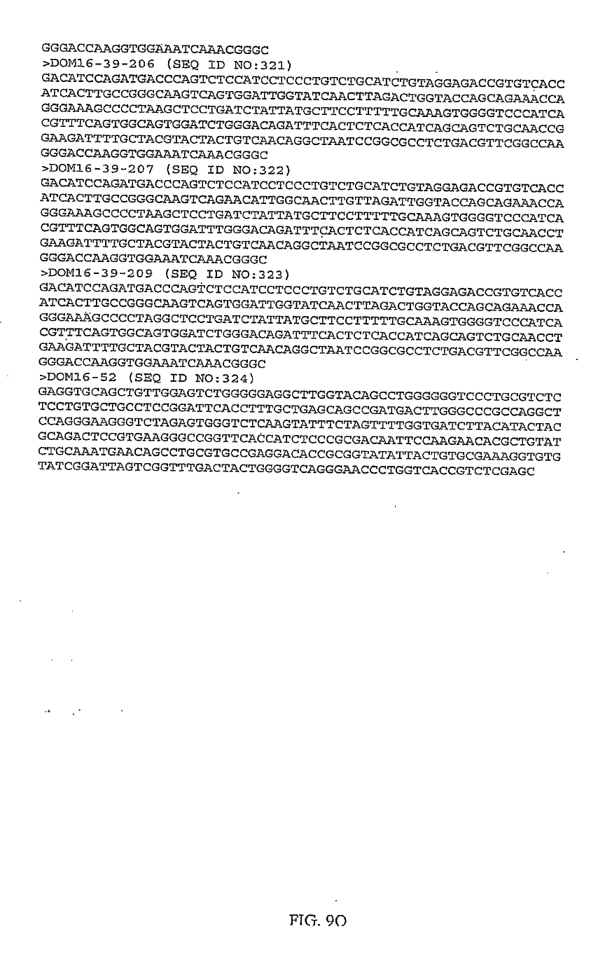

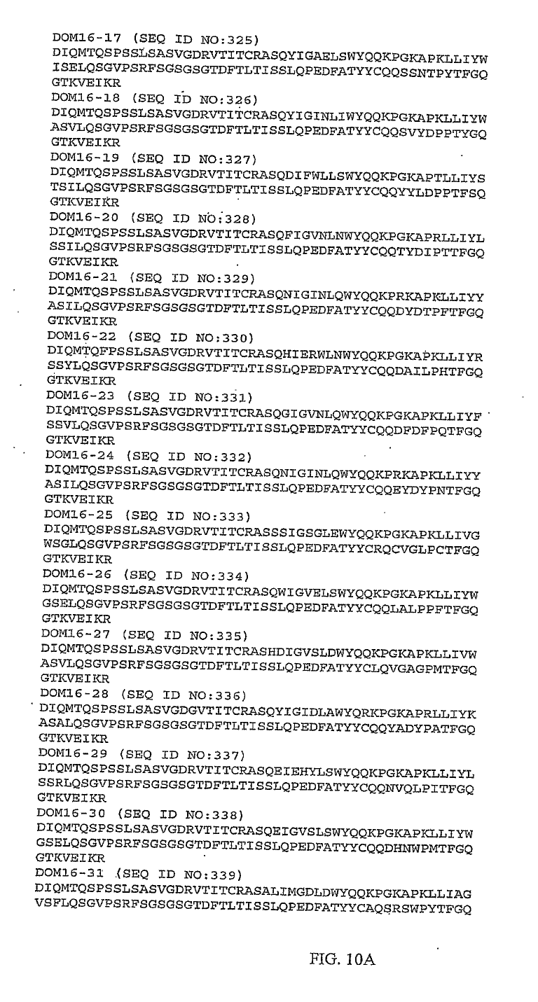

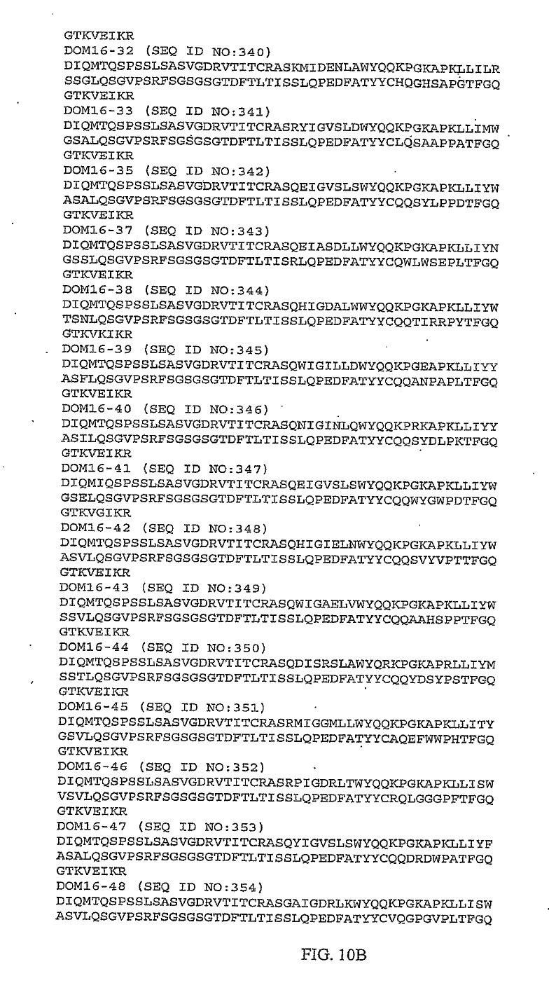

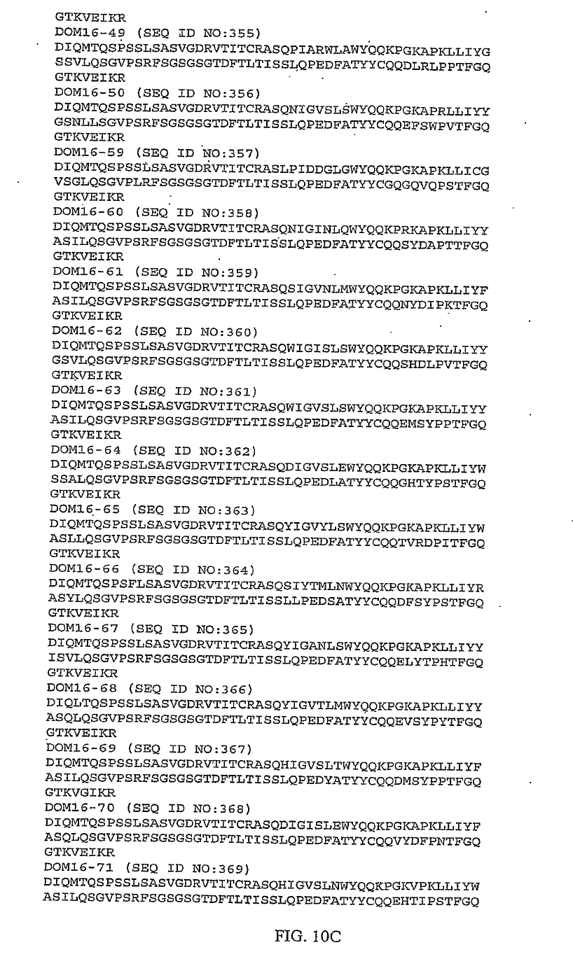

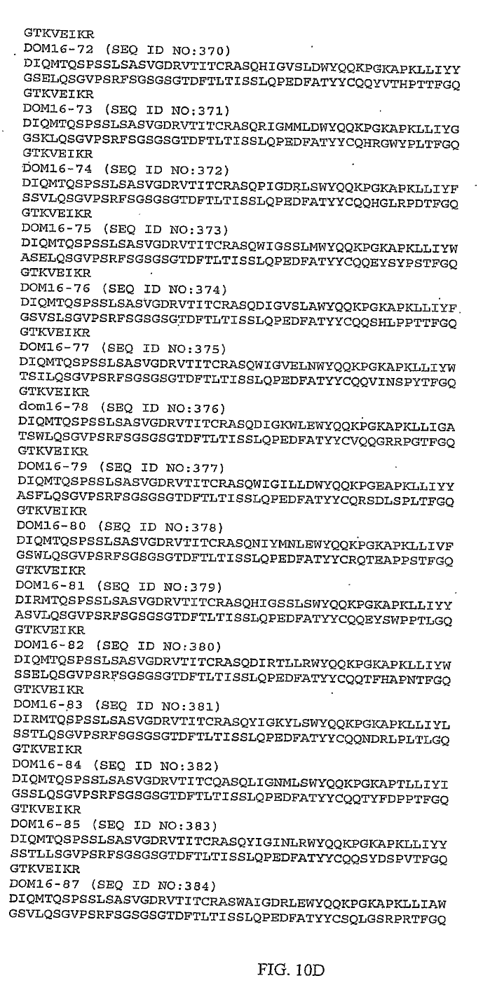

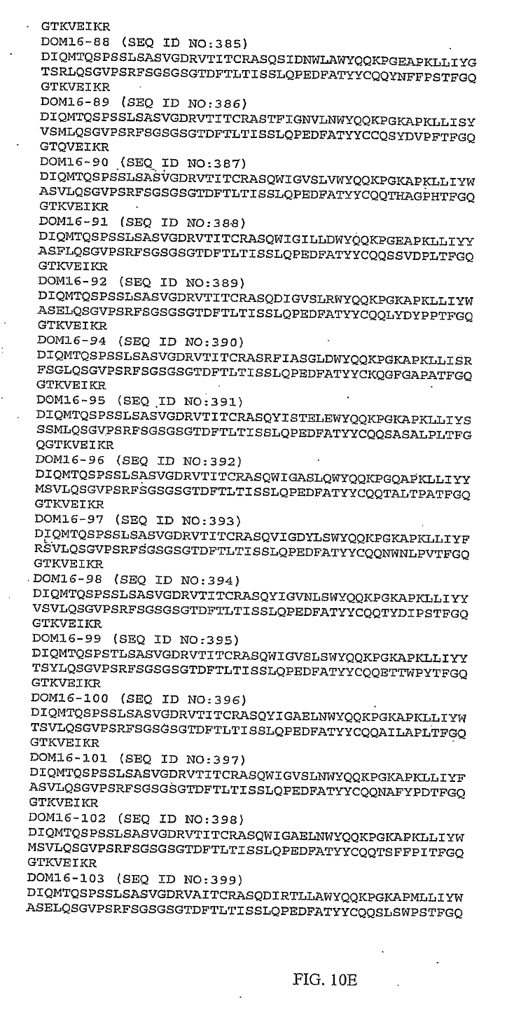

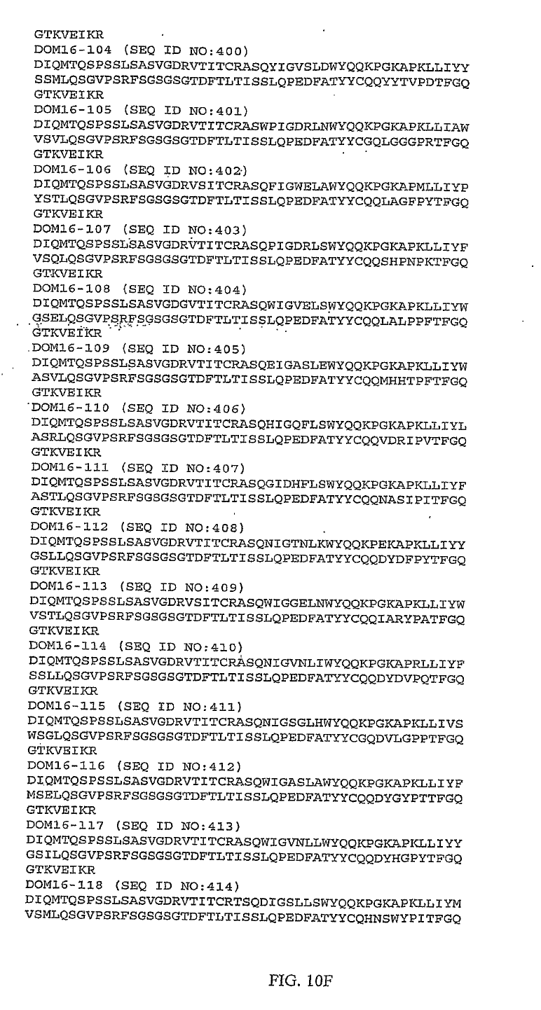

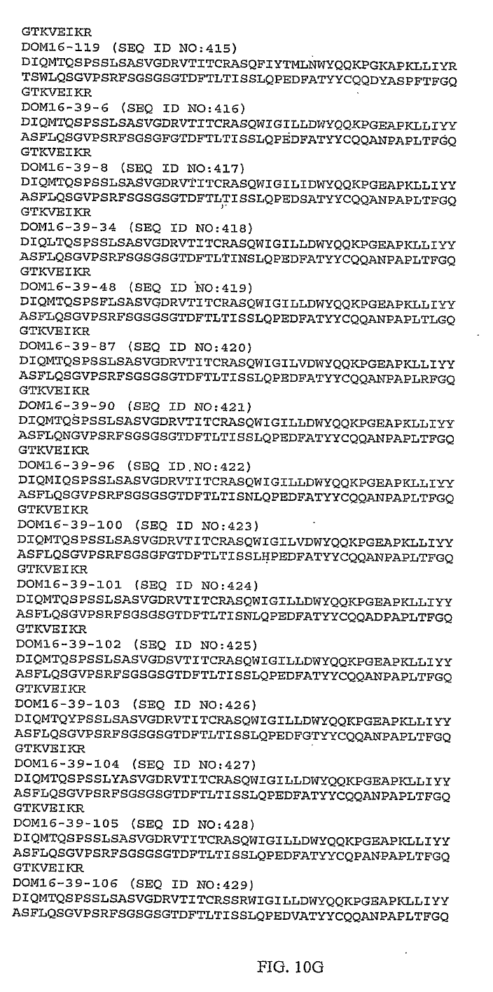

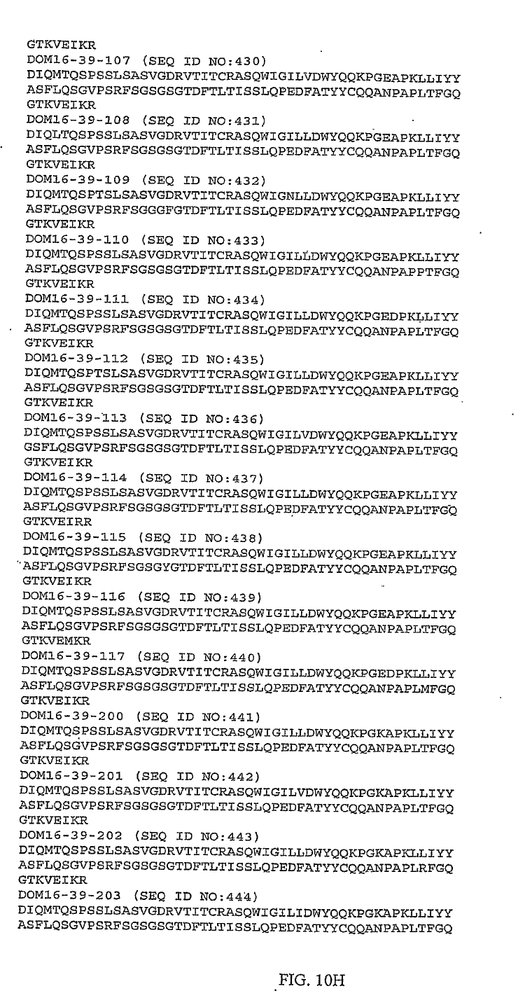

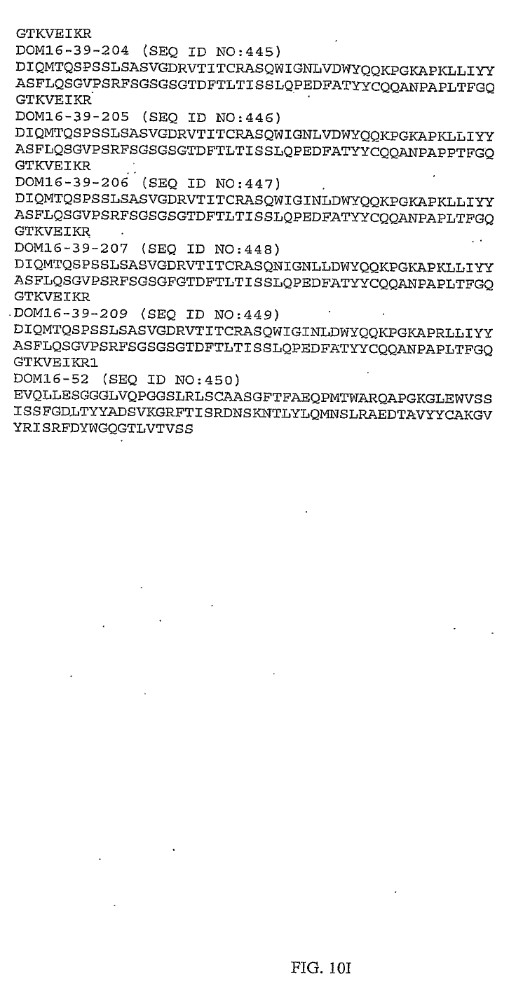

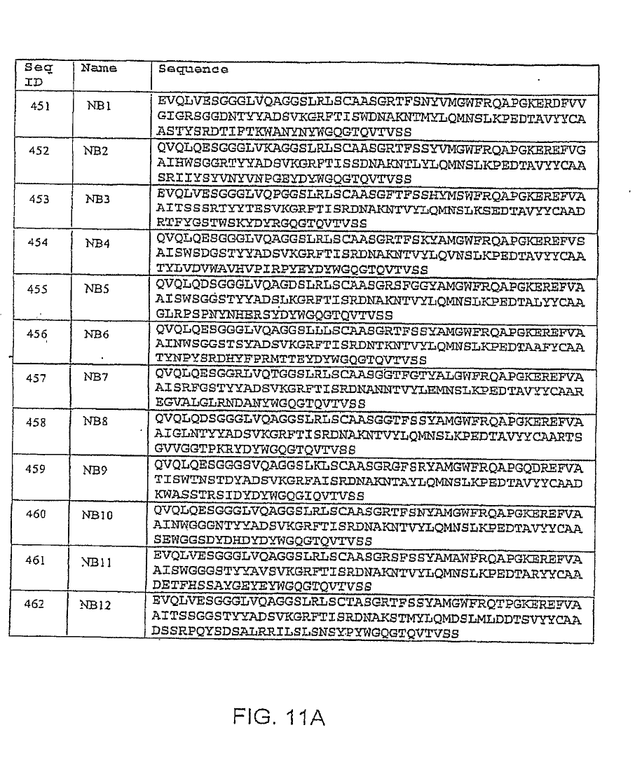

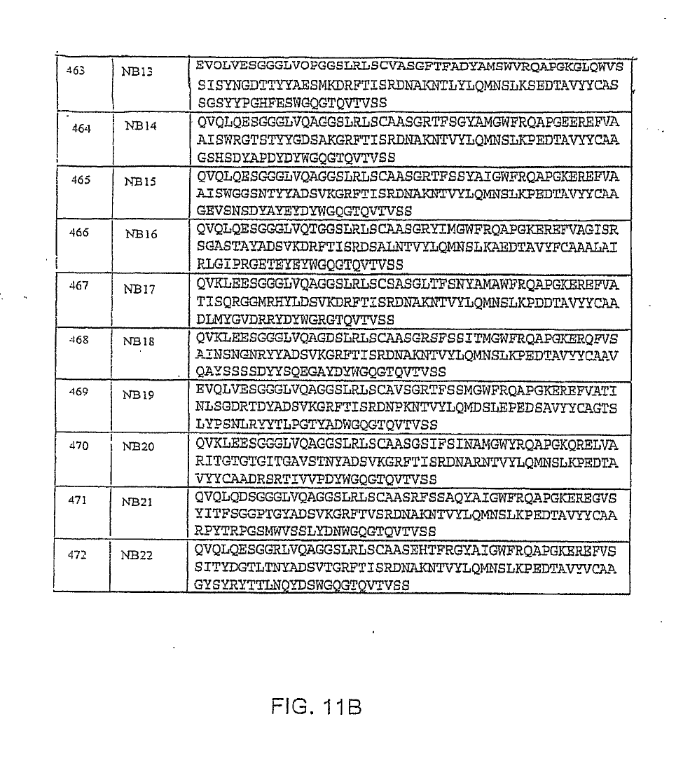

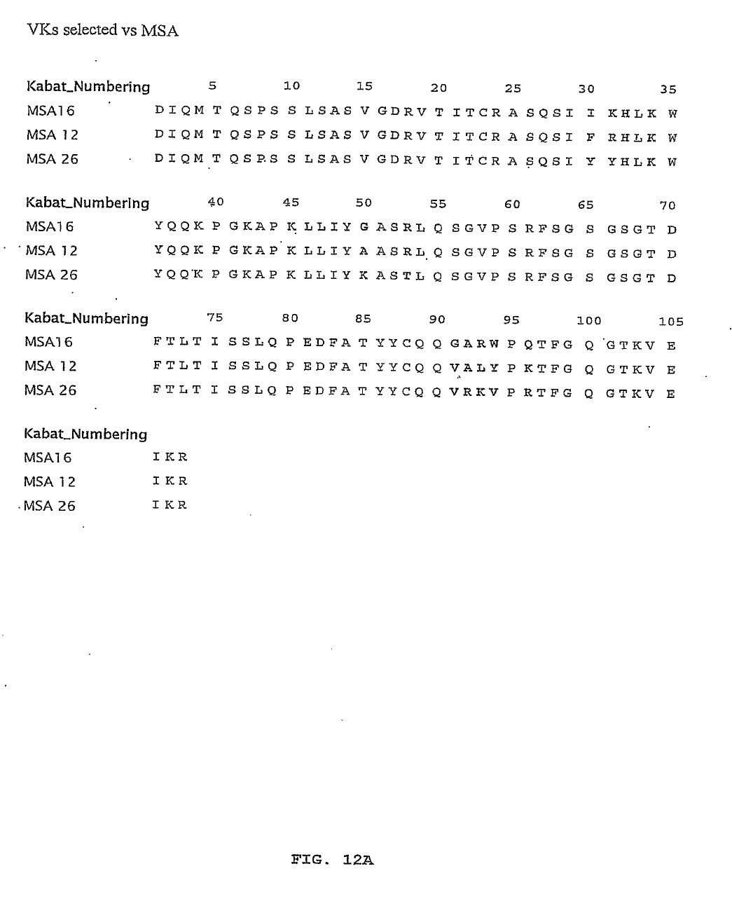

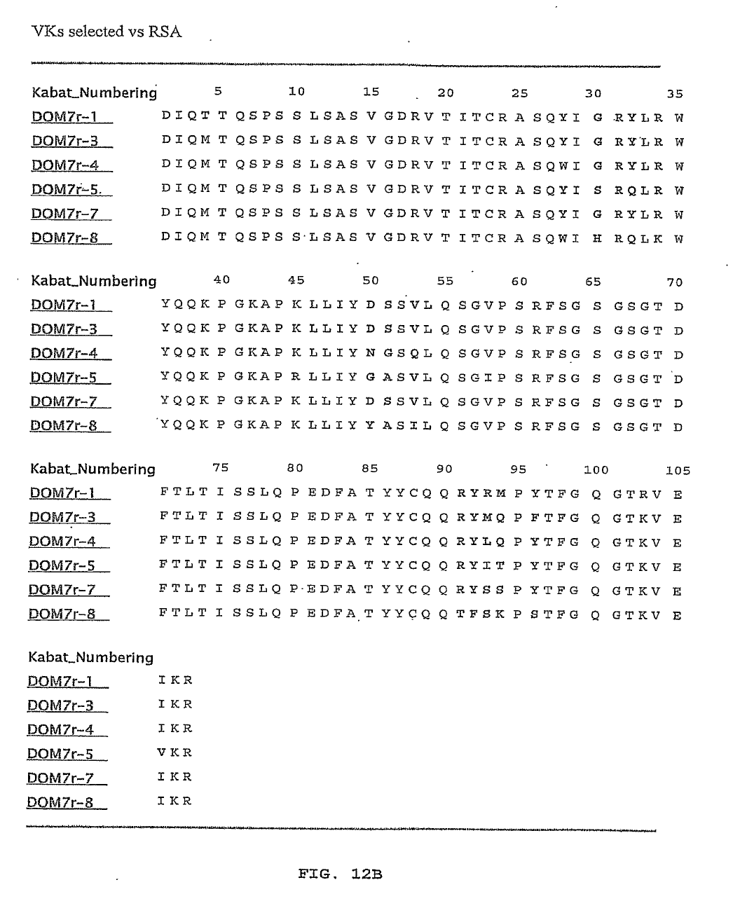

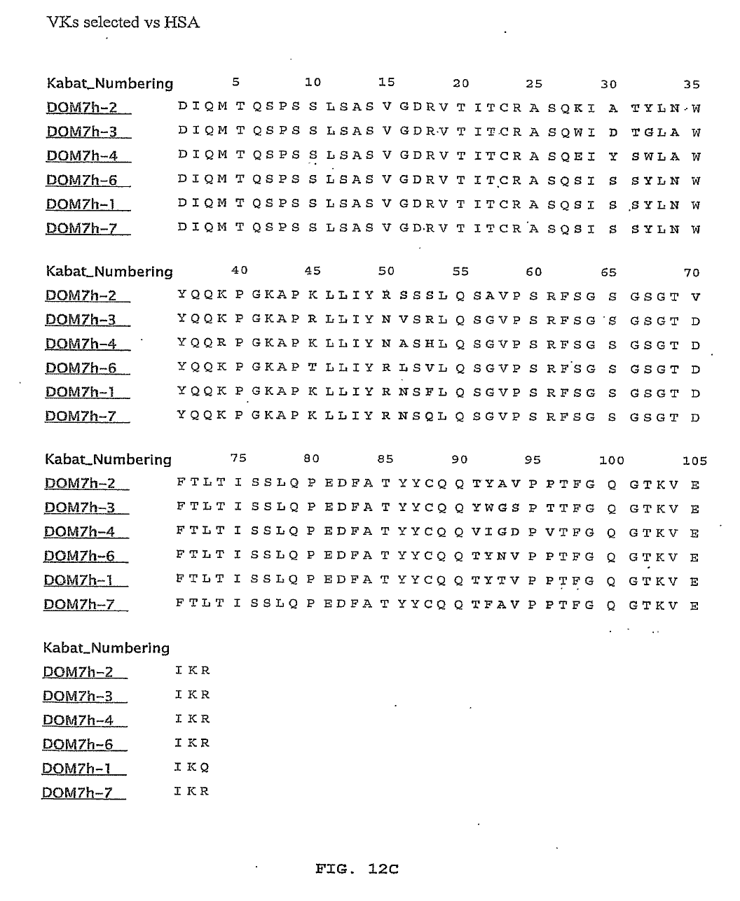

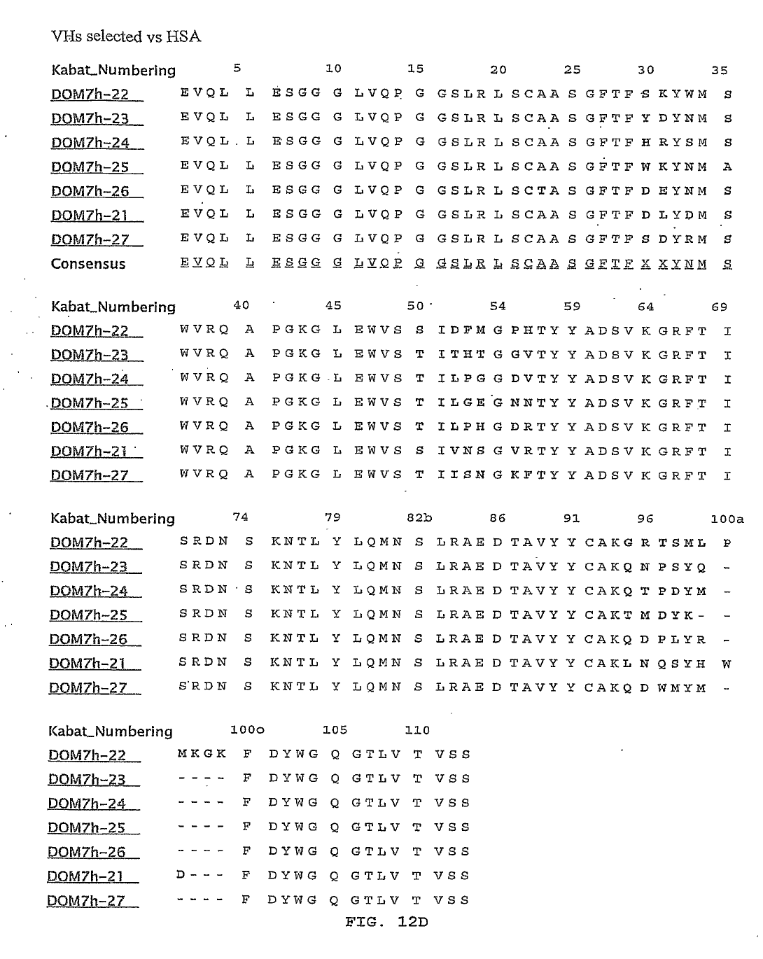

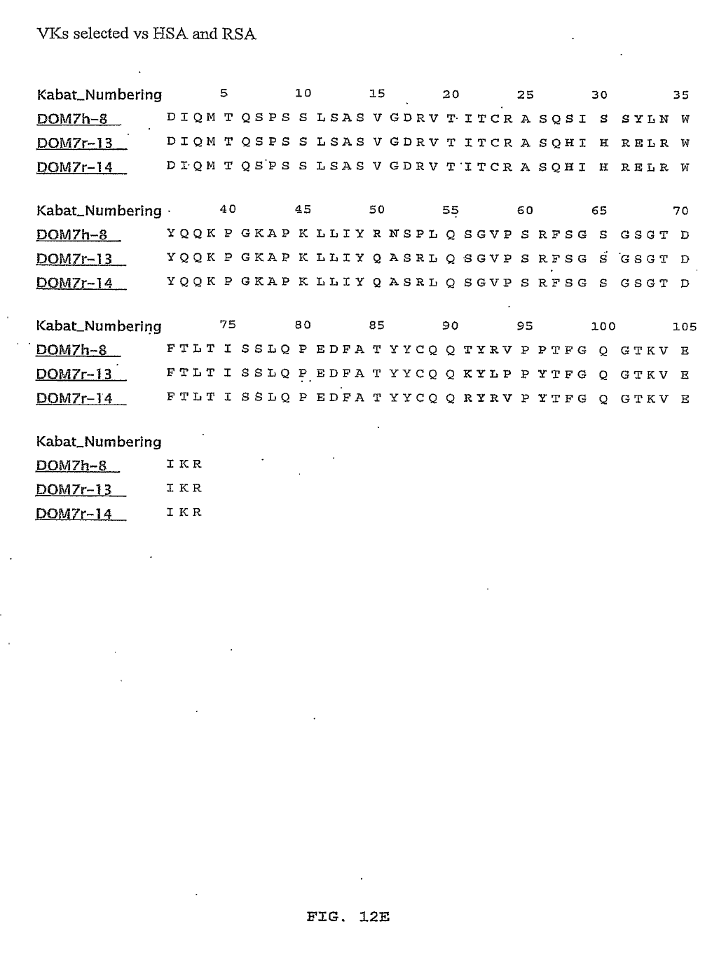

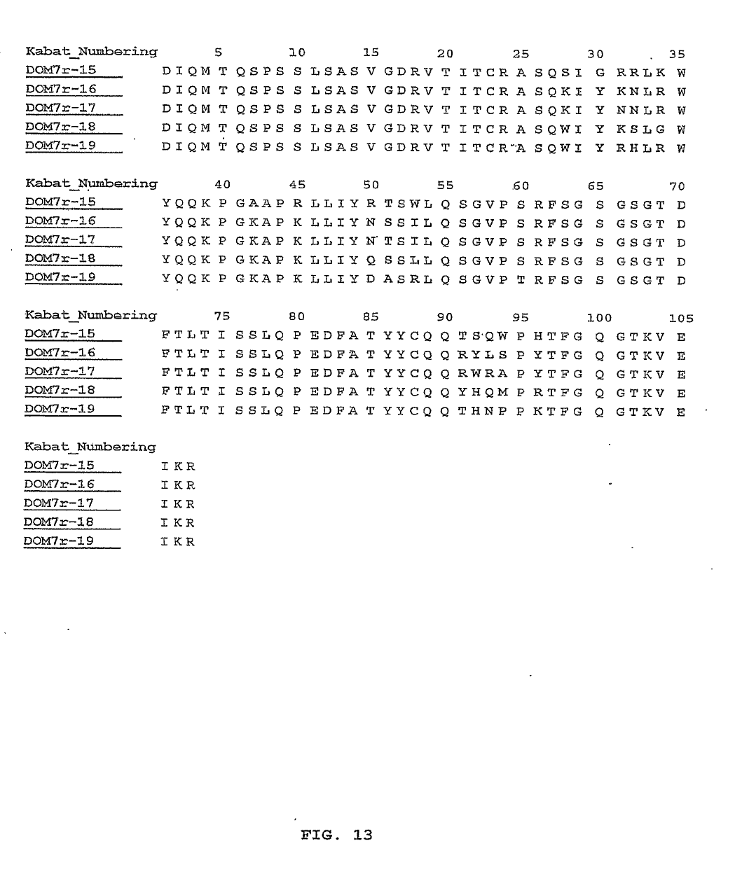

Examples of suitable albumin, albumin fragments or albumin variants for use in a ligand according to the invention are described in WO 2005/077042A2, which is incorporated herein by reference in its entirety. In particular, the following albumin, albumin fragments or albumin variants can be used in the present invention:
SEQ ID NO:1 as disclosed in WO 2005/077042A2, (this sequence being explicitly incorporated into the present disclosure by reference);
Albumin fragment or variant comprising or consisting of amino acids 1-387 of SEQ ID NO:1 in WO 20051077042A2;
Albumin, or fragment or variant thereof, comprising an amino acid sequence selected from the group consisting of (a) amino acids 54 to 61 of SEQ ID NO:1 in WO 2005/077042A2; (b) amino acids 76 to 89 of SEQ ID NO:1 in WO 2005/077042A2; (c) amino acids 92 to 100 of SEQ ID NO:1 in WO 2005/077042A2; (d) amino acids 170 to 176 of SEQ ID NO:1 in WO 2005/077042A2; (e) amino acids 247 to 252 of SEQ ID NO:1 in WO 2005/077042A2; (f) amino acids 266 to 277 of SEQ ID NO:1 in WO 2005/077042A2; (g) amino acids 280 to 288 of SEQ ID NO:1 in WO 2005/077042A2; (h) amino acids 362 to 368 of SEQ ID NO:1 in WO 2005/077042A2; (i) amino acids 439 to 447 of SEQ ID NO:1 in WO 2005/077042A2 (j) amino acids 462 to 475 of SEQ ID NO:1 in WO 2005/077042A2; (k) amino acids 478 to 486 of SEQ ID NO:1 in WO 2005/077042A2; and (l) amino acids 560 to 566 of SEQ ID NO:1 in WO 2005/077042A2.
   Further examples of suitable albumin, fragments and analogs for use in a ligand according to the invention are described in WO 03/076567A2, which is incorporated herein by reference in its entirety. In particular, the following albumin, fragments or variants can be used in the present invention:
Human serum albumin as described in WO 03/076567A2, e.g., in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, e.g., HA(1-373., HA(1-388), HA(1-389), HA(1-369), and HA(1-419) and fragments between 1-369 and 1-419;
An albumin fragment or variant as described in EP 399666, e.g., HA(1-177) and HA(1-200) and fragments between HA(1-X), where X is any number from 178 to 199.

Where a (one or more) half-life extending moiety (e.g., albumin, transferrin and fragments and analogues thereof) is used in the ligands of the invention, it can be conjugated to the ligand using any suitable method, such as, by direct fusion to the target-binding moiety (e.g., dAb or antibody fragment), for example by using a single nucleotide construct that encodes a fusion protein, wherein the fusion protein is encoded as a single polypeptide chain with the half-life extending moiety located N- or C-terminally to the cell surface target binding moieties. Alternatively, conjugation can be achieved by using a peptide linker between moieties, e.g., a peptide linker as described in WO 03/076567A2 or WO 2004/003019 (these linker disclosures being incorporated by reference in the present disclosure to provide examples for use in the present invention). Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fe transport.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and 13-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhance serum half-life in vivo also include proteins localized to the kidney (e.g., polycystin, type IV collagen, organic anion transporter K1, Heymann's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (0:263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes phanuacokinetic parameters such as t alpha and t beta half lives and area under the curve (AUC).

Ligands that Contain a Toxin Moiety or Toxin

The invention also relates to ligands that comprise a toxin moiety or toxin.

Suitable toxin moieties comprise a toxin (e.g., surface active toxin, cytotoxin). The toxin moiety or toxin can be linked or conjugated to the ligand using any suitable method. For example, the toxin moiety or toxin can be covalently bonded to the ligand directly or through a suitable linker. Suitable linkers can include noncleavable or cleavable linkers, for example, pH cleavable linkers that comprise a cleavage site for a cellular enzyme (e.g., cellular esterases, cellular proteases such as cathepsin B). Such cleavable linkers can be used to prepare a ligand that can release a toxin moiety or toxin after the ligand is internalized.

A variety of methods for linking or conjugating a toxin moiety or toxin to a ligand can be used. The particular method selected will depend on the toxin moiety or toxin and ligand to be linked or conjugated. If desired, linkers that contain terminal functional groups can be used to link the ligand and toxin moiety or toxin. Generally, conjugation is accomplished by reacting toxin moiety or toxin that contains a reactive functional group (or is modified to contain a reactive functional group) with a linker or directly with a ligand. Covalent bonds formed by reacting an toxin moiety or toxin that contains (or is modified to contain) a chemical moiety or functional group that can, under appropriate conditions, react with a second chemical group thereby forming a covalent bond. If desired, a suitable reactive chemical group can be added to ligand or to a linker using any suitable method. (See, e.g., Hermanson, G. T., Bioconjugate Techniques, Academic Press: San Diego, Calif. (1996).) Many suitable reactive chemical group combinations are known in the art, for example an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

Suitable toxin moieties and toxins include, for example, a maytansinoid (e.g., maytansinol, e.g., DM1, DM4), a taxane, a calicheamicin, a duocannycin, or derivatives thereof. The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. Nos. 5,208,020 and 6,333,410, the contents of which are incorporated herein by reference. Maytansinol can be coupled to antibodies and antibody fragments using, e.g., an N-succinimidyl 3-(2-pyridyldithio) proprionate (also known as N-succinimidyl4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2 iminothiolane, or S-acetylsuccinic anhydride. The taxane can be, for example, a taxol, taxotere, or novel taxane (see, e.g., WO 01/38318). The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include I1-BR, I2-BR, I3-BR, I4-BR, J1-BR, J2-BR and K1-BR. Iodo-complex calicheamicins include I1-I, I2-I, I3-I, J1-I, J2-I, L1-I and K1-BR. Calicheamicin and mutants, analogs and mimics thereof are described, for example, in U.S. Pat. Nos. 4,970,198; 5,264,586; 5,550,246; 5,712,374, and 5,714,586, the contents of which are incorporated herein by reference. Duocarmycin analogs (e.g., KW-2189, DC88, DC89 CBI-TMI, and derivatives thereof) are described, for example, in U.S. Pat. No. 5,070,092, U.S. Pat. No. 5,187,186, U.S. Pat. No. 5,641,780, U.S. Pat. No. 5,641,780, U.S. Pat. No. 4,923,990, and U.S. Pat. No. 5,101,038, the contents of which are incorporated herein by reference.

Examples of other toxins include, but are not limited to antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065 (see U.S. Pat. Nos. 5,475,092, 5,585,499, 5,846,545), melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, mitomycin, puromycin anthramycin (AMC)), duocannycin and analogs or derivatives thereof, and anti-mitotic agents (e.g., vincristine, vinblastine, taxol, auristatins (e.g., auristatin E) and maytansinoids, and analogs or homologs thereof.

The toxin can also be a surface active toxin, such as a toxin that is a free radical generator (e.g. selenium containing toxin moieties), or radionuclide containing moiety. Suitable radionuclide containing moieties, include for example, moieties that contain radioactive iodine (¹³¹I or ¹²⁵I), yttrium (⁹⁰Y), lutetium (¹⁷⁷Lu), actinium (²²⁵ Ac), praseodymium, astatine (²¹¹At), rhenium (¹⁸⁶Re), bismuth (²¹² Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

The toxin can be a protein, polypeptide or peptide, from bacterial sources, e.g., diphtheria toxin, pseudomonas exotoxin (PE) and plant proteins, e.g., the A chain of ricin (RTA), the ribosome inactivating proteins (RIPs) gelonin, pokeweed antiviral protein, saporin, and dodecandron are contemplated for use as toxins.

Antisense compounds of nucleic acids designed to bind, disable, promote degradation or prevent the production of the mRNA responsible for generating a particular target protein can also be used as a toxin. Antisense compounds include antisense RNA or DNA, single or double stranded, oligonucleotides, or their analogs, which can hybridize specifically to individual mRNA species and prevent transcription and/or RNA processing of the mRNA species and/or translation of the encoded polypeptide and thereby effect a reduction in the amount of the respective encoded polypeptide. Ching, et al., Proc. Natl. Acad. Sci. U.S.A. 86: 10006-10010 (1989); Broder, et al., Ann. Int. Med. 113: 604-618 (1990); Loreau, et al., FEBS Letters 274: 53-56 (1990); Useful antisense therapeutics include for example: Veglin™ (VasGene) and OGX-011 (Oncogenix).

Toxins can also be photoactive agents. Suitable photoactive agents include porphyrin-based materials such as porfimer sodium, the green porphyrins, chlorin E6, hematoporphyrin derivative itself, phthalocyanines, etiopurpurins, texaphrin, and the like.

The toxin can be an antibody or antibody fragment that binds an intracellular target (e.g., an intrabody), such as a dAb that binds an intracellular target. Such antibodies or antibody fragments (dAbs) can be directed to defined subcellular compartments or targets. For example, the antibodies or antibody fragments (dAbs) can bind an intracellular target selected from erbB2, EGFR, BCR-ABL, p21Ras, Caspase3, Caspase7, Bcl-2, p53, Cyclin E, ATF-1/CREB, HPV16 E7, HP1, Type IV collagenases, cathepsin L as well as others described in Kontermann, R. E., Methods, 34:163-170 (2004), incorporated herein by reference in its entirety.

Polypeptide Domains that Bind VEGF

The invention provides polypeptide domains (e.g., immunoglobulin single variable domains, dAb monomers) that have a binding site with binding specificity for VEGF. In preferred embodiments, the polypeptide domain (e.g., dAb) binds to VEGF with an affinity (KD; KD=K_{off}(kd)/Kₒₙ(ka)) of 300 nM to 1 pM (i.e., 3×10⁻⁷ to 5×10⁻¹²M), preferably 50 nM to 1 pM, more preferably 5 nM to 1 pM and most preferably 1 nM to 1 pM, for example and K_{D} of 1×10⁻⁷M or less, preferably 1×10⁻⁸M or less, more preferably 1×10⁻⁹ M or less, advantageously 1×10⁻¹⁰M or less and most preferably 1×10⁻¹¹M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

In some embodiments, the polypeptide domain that has a binding site with binding specificity for VEGF competes for binding to VEGF with a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), TAR15-26-549 (SEQ ID NO:198), TAR15-26-550 (SEQ ID NO:539), and TAR15-26-551 (SEQ ID NO:540).

In some embodiments, the polypeptide domain that has a binding site with binding specificity for VEGF competes for binding to VEGF with TAR15-26-555 (SEQ ID NO:704).

In some embodiments, the polypeptide domain that has a binding site with binding specificity for VEGF comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), TAR15-26-549 (SEQ ID NO:198), TAR15-26-550 (SEQ ID NO:539), and TAR15-26-551 (SEQ ID NO:540).

In some embodiments, the polypeptide domain that has a binding site with binding specificity for VEGF comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of TAR15-26-555 (SEQ ID NO:704).

In preferred embodiments, the polypeptide domain that has a binding site with binding specificity for VEGF comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of TAR15-6 (SEQ ID NO:117), TAR15-8 (SEQ ID NO:119), and TAR15-26 (SEQ ID NO:123). For example, the polypeptide domain that has a binding site with binding specificity for VEGF can comprise TAR15-6 (SEQ ID NO:117), TAR15-8 (SEQ ID NO:119), or TAR15-26 (SEQ ID NO:123).

In some embodiments, the polypeptide domain that has a binding site with binding specificity for VEGF competes with any of the dAbs disclosed herein for binding to VEGF.

Preferably the polypeptide domain that has a binding site with binding specificity for VEGF is an immunoglobulin single variable domain. The polypeptide domain that has a binding site with binding specificity for VEGF can comprise any suitable immunoglobulin variable domain, and preferably comprises a human variable domain or a variable domain that comprises human framework regions. In certain embodiments, the polypeptide domain that has a binding site with binding specificity for VEGF comprises a universal framework, as described herein.

The universal framework can be a V_{L} framework (Vλ or Vκ), such as a framework that comprises the framework amino acid sequences encoded by the human germline DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprise the framework amino acid sequence encoded by the human germline J_{κ}1, J_{Kκ}2, J_{κ}3, J_{κ}4, or J_{κ}5 immunoglobulin gene segment.

In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segment. If desired, the V_{H} framework can further comprise the framework amino acid sequence encoded by the human germline J_{H}1, J_{H}2, J_{H}4, J_{H}4b, J_{H}5 and J_{H}6 immunoglobulin gene segment.

In certain embodiments, the polypeptide domain that has a binding site with binding specificity for VEGF comprises one or more framework regions comprising an amino acid sequence that is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In other embodiments, the amino acid sequences of FW1, FW2, FW3 and FW4 of the polypeptide domain that has a binding site with binding specificity for VEGF are the same as the amino acid sequences of corresponding framework regions encoded by a human germline antibody gene segment, or the amino acid sequences of FW1, FW2, FW3 and FW4 collectively contain up to 10 amino acid differences relative to the amino acid sequences of corresponding framework regions encoded by said human germline antibody gene segment.

In other embodiments, the polypeptide domain that has a binding site with binding specificity for VEGF comprises FW1, FW2 and FW3 regions, and the amino acid sequence of said FW1, FW2 and FW3 regions are the same as the amino acid sequences of corresponding framework regions encoded by human germline antibody gene segments.

In particular embodiments, the polypeptide domain that has a binding site with binding specificity for VEGF comprises the DPK9 V_{L} framework, or a V_{H} framework selected from the group consisting of DP47, DP45 and DP38. The polypeptide domain that has a binding site with binding specificity for VEGF can comprise a binding site for a generic ligand, such as protein A, protein L and protein G.

In certain embodiments, the polypeptide domain that has a binding site with binding specificity for VEGF is substantially resistant to aggregation. For example, in some embodiments, less than about 10%, less than about 9%, less than about 8%, less than about 7%, less than about 6%, less than about 5%, less than about 4%, less than about 3%, less than about 2% or less than about 1% of the polypeptide domain that has a binding site with binding specificity for VEGF aggregates when a 1-5 mg/ml, 5-10 mg/ml, 10-20 mg/ml, 20-50 mg/ml, 50-100 mg/ml, 100-200 mg/ml or 200-500 mg/ml solution of ligand or dAb in a solvent that is routinely used for drug formulation such as saline, buffered saline, citrate buffer saline, water, an emulsion, and, any of these solvents with an acceptable excipient such as those approved by the FDA, is maintained at about 22° C., 22-25° C., 25-30° C., 30-37° C., 37-40° C., 40-50° C., 50-60° C., 60-70° C., 70-80° C., 15-20° C., 10-15° C., 5-10° C., 2-5° C., 0-2° C., −10° C. to 0° C., −20° C. to −10° C., −40° C. to −20° C., −60° C. to −40° C., or −80° C. to −60° C., for a period of about time, for example, 10 minutes, 1 hour, 8 hours, 24 hours, 2 days, 3 days, 4 days, 1 week, 2 weeks, 3 weeks, 1 month, 2 months, 3 months, 4 months, 6 months, 1 year, or 2 years.

Aggregation can be assessed using any suitable method, such as, by microscopy, assessing turbidity of a solution by visual inspection or spectroscopy or any other suitable method. Preferably, aggregation is assessed by dynamic light scattering. Polypeptide domains that have a binding site with binding specificity for VEGF that are resistant to aggregation provide several advantages. For example, such polypeptide domains that have a binding site with binding specificity for VEGF can readily be produced in high yield as soluble proteins by expression using a suitable biological production system, such as E. coli, and can be formulated and/or stored at higher concentrations than conventional polypeptides, and with less aggregation and loss of activity.

In addition, the polypeptide domain that has a binding site with binding specificity for VEGF that are resistant to aggregation can be produced more economically than other antigen- or epitope-binding polypeptides (e.g., conventional antibodies). For example, generally, preparation of antigen- or epitope-binding polypeptides intended for in vivo applications includes processes (e.g., gel filtration) that remove aggregated polypeptides. Failure to remove such aggregates can result in a preparation that is not suitable for in vivo applications because, for example, aggregates of an antigen-binding polypeptide that is intended to act as an antagonist can function as an agonist by inducing cross-linking or clustering of the target antigen. Protein aggregates can also reduce the efficacy of therapeutic polypeptide by inducing an immune response in the subject to which they are administered.

In contrast, the aggregation resistant polypeptide domain that has a binding site with binding specificity for VEGF of the invention can be prepared for in vivo applications without the need to include process steps that remove aggregates, and can be used in in vivo applications without the aforementioned disadvantages caused by polypeptide aggregates.

In some embodiments, the polypeptide domain that has a binding site with binding specificity for VEGF unfolds reversibly when heated to a temperature (Ts) and cooled to a temperature (Tc), wherein Ts is greater than the melting temperature (Tm) of the polypeptide domain that has a binding site with binding specificity for VEGF, and Tc is lower than the melting temperature of the polypeptide domain that has a binding site with binding specificity for VEGF. For example, a polypeptide domain that has a binding site with binding specificity for VEGF can unfold reversibly when heated to 80° C. and cooled to about room temperature. A polypeptide that unfolds reversibly loses function when unfolded but regains function upon refolding. Such polypeptides are distinguished from polypeptides that aggregate when unfolded or that improperly refold (misfolded polypeptides), i.e., do not regain function.

Polypeptide unfolding and refolding can be assessed, for example, by directly or indirectly detecting polypeptide structure using any suitable method. For example, polypeptide structure can be detected by circular dichroism (CD) (e.g., far-UV CD, near-UV CD), fluorescence (e.g., fluorescence of tryptophan side chains), susceptibility to proteolysis, nuclear magnetic resonance (NMR), or by detecting or measuring a polypeptide function that is dependent upon proper folding (e.g., binding to target ligand, binding to generic ligand). In one example, polypeptide unfolding is assessed using a functional assay in which loss of binding function (e.g., binding a generic and/or target ligand, binding a substrate) indicates that the polypeptide is unfolded.

The extent of unfolding and refolding of a polypeptide domain that has a binding site with binding specificity for VEGF can be determined using an unfolding or denaturation curve. An unfolding curve can be produced by plotting temperature as the ordinate and the relative concentration of folded polypeptide as the abscissa. The relative concentration of folded polypeptide domain that has a binding site with binding specificity for VEGF can be determined directly or indirectly using any suitable method (e.g., CD, fluorescence, binding assay). For example, a polypeptide domain that has a binding site with binding specificity for VEGF solution can be prepared and ellipticity of the solution determined by CD. The ellipticity value obtained represents a relative concentration of folded ligand (e.g., dAb monomer) of 100%. The polypeptide domain that has a binding site with binding specificity for VEGF in the solution is then unfolded by incrementally raising the temperature of the solution and ellipticity is determined at suitable increments (e.g., after each increase of one degree in temperature). The polypeptide domain that has a binding site with binding specificity for VEGF in solution is then refolded by incrementally reducing the temperature of the solution and ellipticity is determined at suitable increments. The data can be plotted to produce an unfolding curve and a refolding curve. The unfolding and refolding curves have a characteristic sigmoidal shape that includes a portion in which the polypeptide domain that has a binding site with binding specificity for VEGF molecules are folded, an unfolding/refolding transition in which polypeptide domain that has a binding site with binding specificity for VEGF molecules are unfolded to various degrees, and a portion in which polypeptide domain that has a binding site with binding specificity for VEGF are unfolded. The y-axis intercept of the refolding curve is the relative amount of refolded polypeptide domain that has a binding site with binding specificity for VEGF recovered. A recovery of at least about 50%, or at least about 60%, or at least about 70%, or at least about 75%, or at least about 80%, or at least about 85%, or at least about 90%, or at least about 95% is indicative that the ligand or dAb monomer unfolds reversibly.

In a preferred embodiment, reversibility of unfolding of a polypeptide domain that has a binding site with binding specificity for VEGF is determined by preparing a polypeptide domain that has a binding site with binding specificity for VEGF solution and plotting heat unfolding and refolding curves. The polypeptide domain that has a binding site with binding specificity for VEGF solution can be prepared in any suitable solvent, such as an aqueous buffer that has a pH suitable to allow polypeptide domain that has a binding site with binding specificity for VEGF to dissolve (e.g., pH that is about 3 units above or below the isoelectric point (pI)). The polypeptide domain that has a binding site with binding specificity for VEGF solution is concentrated enough to allow unfolding/folding to be detected. For example, the ligand or dAb monomer solution can be about 0.1 μM to about 100 μM, or preferably about 1 μM to about 10 μM.

If the melting temperature (Tm) of a polypeptide domain that has a binding site with binding specificity for VEGF is known, the solution can be heated to about ten degrees below the Tm (Tm-10) and folding assessed by ellipticity or fluorescence (e.g., far-UV CD scan from 200 nm to 250 nm, fixed wavelength CD at 235 nm or 225 nm; tryptophan fluorescent emission spectra at 300 to 450 nm with excitation at 298 nm) to provide 100% relative folded ligand or dAb monomer. The solution is then heated to at least ten degrees above Tm (Tm+10) in predetermined increments (e.g., increases of about 0.1 to about 1 degree), and ellipticity or fluorescence is determined at each increment. Then, the polypeptide domain that has a binding site with binding specificity for VEGF is refolded by cooling to at least Tm-10 in predetermined increments and ellipticity or fluorescence determined at each increment. If the melting temperature of a polypeptide domain that has a binding site with binding specificity for VEGF is not known, the solution can be unfolded by incrementally heating from about 25° C. to about 100° C. and then refolded by incrementally cooling to at least about 25° C., and ellipticity or fluorescence at each heating and cooling increment is determined. The data obtained can be plotted to produce an unfolding curve and a refolding curve, in which the y-axis intercept of the refolding curve is the relative amount of refolded protein recovered. In some embodiments, the polypeptide domain that has a binding site with binding specificity for VEGF does not comprise a Camelid immunoglobulin variable domain, or one or more framework amino acids that are unique to immunoglobulin variable domains encoded by Camelid germline antibody gene segments.

Preferably, the polypeptide domain that has a binding site with binding specificity for VEGF is secreted in a quantity of at least about 0.5 mg/L when expressed in E. coli or in Pichia species (e.g., P. pastoris). In other preferred embodiments, a polypeptide domain that has a binding site with binding specificity for VEGF is secreted in a quantity of at least about 0.75 mg/L, at least about 1 mg/L, at least about 4 mg/L, at least about 5 mg/L, at least about 10 mg/L, at least about 15 mg/L, at least about 20 mg/L, at least about 25 mg/L, at least about 30 mg/L, at least about 35 mg/L, at least about 40 mg/L, at least about 45 mg/L, or at least about 50 mg/L, or at least about 100 mg/L, or at least about 200 mg/L, or at least about 300 mg/L, or at least about 400 mg/L, or at least about 500 mg/L, or at least about 600 mg/L, or at least about 700 mg/L, or at least about 800 mg/L, at least about 900 mg/L, or at least about 1 g/L when expressed in E. coli or in Pichia species (e.g., P. P. pastoris). In other preferred embodiments, a polypeptide domain that has a binding site with binding specificity for VEGF is secreted in a quantity of at least about 1 mg/L to at least about 1 g/L, at least about 1 mg/L to at least about 750 mg/L, at least about 100 mg/L to at least about 1 g/L, at least about 200 mg/L to at least about 1 g/L, at least about 300 mg/L to at least about 1 g/L, at least about 400 mg/L to at least about 1 g/L, at least about 500 mg/L to at least about 1 g/L, at least about 600 mg/L to at least about 1 g/L, at least about 700 mg/L to at least about 1 g/L, at least about 800 mg/L to at least about 1 g/L, or at least about 900 mg/L to at least about 1 g/L when expressed in E. coli or in Pichia species (e.g., P. pastoris). Although, a polypeptide domain that has a binding site with binding specificity for VEGF described herein can be secretable when expressed in E. coli or in Pichia species (e.g., P. pastoris), they can be produced using any suitable method, such as synthetic chemical methods or biological production methods that do not employ E. coli or Pichia species.

Polypeptide Domains that Bind EGFR

The invention provides polypeptide domains (e.g., dAb) that have a binding site with binding specificity for EGFR. In preferred embodiments, the polypeptide domain (e.g., dAb) binds to EGFR with an affinity (KD; KD=K_{off}(kd)/Kₒₙ(ka)) of 300 nM to 1 pM (i.e., 3×10⁻⁷ to 5×10⁻¹²M), preferably 100 nM to 1 pM, or 50 nM to 10 pM, more preferably 10 nM to 100 pM and most preferably about 1 nM, for example and K_{D} of 1×10⁻⁷M or less, preferably 1×10⁻⁸M or less, more preferably about 1×10⁻⁹ M or less, 1×10⁻¹⁰ M or less or 1×10⁻¹¹M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

In some embodiments, the polypeptide domain that has a binding site with binding specificity for EGFR competes for binding to EGFR with a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In some embodiments, the polypeptide domain that has a binding site with binding specificity for EGFR competes for binding to EGFR with a dAb selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

In some embodiments, the polypeptide domain that has a binding site with binding specificity for EGFR comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In some embodiments, the polypeptide domain that has a binding site with binding specificity for EGFR comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM16-39-210 (SEQ ID NO:541), DOM16-39-211 (SEQ ID NO:542), DOM16-39-212 (SEQ ID NO:543), DOM16-39-213 (SEQ ID NO:544), DOM16-39-214 (SEQ ID NO:545), DOM16-39-215 (SEQ ID NO:546), DOM16-39-216 (SEQ ID NO:547), DOM16-39-217 (SEQ ID NO:548), DOM16-39-218 (SEQ ID NO:549), DOM16-39-219 (SEQ ID NO:550), DOM16-39-220 (SEQ ID NO:551), DOM16-39-221 (SEQ ID NO:552), DOM16-39-222 (SEQ ID NO:553), DOM16-39-223 (SEQ ID NO:554), DOM16-39-224 (SEQ ID NO:555), DOM16-39-225 (SEQ ID NO:556), DOM16-39-226 (SEQ ID NO:557), DOM16-39-227 (SEQ ID NO:558), DOM16-39-228 (SEQ ID NO:559), DOM16-39-229 (SEQ ID NO:560), DOM16-39-230 (SEQ ID NO:561), DOM16-39-231 (SEQ ID NO:562), DOM16-39-232 (SEQ ID NO:563), DOM16-39-233 (SEQ ID NO:564), DOM16-39-234 (SEQ ID NO:565), DOM16-39-235 (SEQ ID NO:566), DOM16-39-500 (SEQ ID NO:725), DOM16-39-502 (SEQ ID NO:726), DOM16-39-503 (SEQ ID NO:567), DOM16-39-504 (SEQ ID NO:568), DOM16-39-505 (SEQ ID NO:569), DOM16-39-506 (SEQ ID NO:570), DOM16-39-507 (SEQ ID NO:571), DOM16-39-508 (SEQ ID NO:572), DOM16-39-509 (SEQ ID NO:573), DOM16-39-510 (SEQ ID NO:574), DOM16-39-511 (SEQ ID NO:575), DOM16-39-512 (SEQ ID NO:576), DOM16-39-521 (SEQ ID NO:577), DOM16-39-522 (SEQ ID NO:578), DOM16-39-523 (SEQ ID NO:579), DOM16-39-524 (SEQ ID NO:580), DOM16-39-527 (SEQ ID NO:581), DOM16-39-525 (SEQ ID NO:582), DOM16-39-526 (SEQ ID NO:583), DOM16-39-540 (SEQ ID NO:584), DOM16-39-541 (SEQ ID NO:585), DOM16-39-542 (SEQ ID NO:586), DOM16-39-543 (SEQ ID NO:587), DOM16-39-544 (SEQ ID NO:588), DOM16-39-545 (SEQ ID NO:589), DOM16-39-550 (SEQ ID NO:590), DOM16-39-551 (SEQ ID NO:591), DOM16-39-552 (SEQ ID NO:592), DOM16-39-553 (SEQ ID NO:593), DOM16-39-554 (SEQ ID NO:594), DOM16-39-555 (SEQ ID NO:595), DOM16-39-561 (SEQ ID NO:596), DOM16-39-562 (SEQ ID NO:597), DOM16-39-563 (SEQ ID NO:598), DOM16-39-564 (SEQ ID NO:599), DOM16-39-571 (SEQ ID NO:600), DOM16-39-572 (SEQ ID NO:601), DOM16-39-573 (SEQ ID NO:602), DOM16-39-574 (SEQ ID NO:603), DOM16-39-580 (SEQ ID NO:604), DOM16-39-591 (SEQ ID NO:605), DOM16-39-592 (SEQ ID NO:606), DOM16-39-593 (SEQ ID NO:607), DOM16-39-601 (SEQ ID NO:608), DOM16-39-602 (SEQ ID NO:609), DOM16-39-603 (SEQ ID NO:610), DOM16-39-604 (SEQ ID NO:611), DOM16-39-605 (SEQ ID NO:612), DOM16-39-607 (SEQ ID NO:613), DOM16-39-611 (SEQ ID NO:614), DOM16-39-612 (SEQ ID NO:615), DOM16-39-613 (SEQ ID NO:616), DOM16-39-614 (SEQ ID NO:617), DOM16-39-615 (SEQ ID NO:618), DOM16-39-616 (SEQ ID NO:619), DOM16-39-617 (SEQ ID NO:620), DOM16-39-618 (SEQ ID NO:621), and DOM16-39-619 (SEQ ID NO:622).

In preferred embodiments, the polypeptide domain that has a binding site with binding specificity for EGFR comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of DOM16-39 (SEQ ID NO:345). For example, the polypeptide domain that has a binding site with binding specificity for EGFR can comprise the amino acid sequence of DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), or DOM16-39-200 (SEQ ID NO:441).

In other preferred embodiments, the polypeptide domain that has a binding site with binding specificity for EGFR comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

In some embodiments, the polypeptide domain that has a binding site with binding specificity for EGFR competes with any of the dAbs disclosed herein for binding to EGFR.

Preferably, the polypeptide domain that has a binding site with binding specificity for EGFR is an immunoglobulin single variable domain. The polypeptide domain that has a binding site with binding specificity for EGFR can comprise any suitable immunoglobulin variable domain, and preferably comprises a human variable domain or a variable domain that comprises human framework regions. In certain embodiments, the polypeptide domain that has a binding site with binding specificity for EGFR comprises a universal framework, as described herein.

In certain embodiments, the polypeptide domain that has a binding site with binding specificity for EGFR resists aggregation, unfolds reversibly, comprises a framework region and/or is secreted as described above for the polypeptide domain that has a binding site with binding specificity for VEGF.

dAb Monomers that Bind Serum Albumin

The ligands of the invention can further comprise a dAb monomer that binds serum albumin (SA) with a K_{d} of 1 nM to 500 M (i.e., ×10⁻⁹ to 5×10⁻⁴), preferably 100 nM to 10 μM. Preferably, for a ligand comprising an anti-SA dAb, the binding (e.g. K_{d} and/or K_{off} as measured by surface plasmon resonance, e.g., using BiaCore) of the ligand to its target(s) is from 1 to 100000 times (preferably 100 to 100000, more preferably 1000 to 100000, or 10000 to 100000 times) stronger than for SA. Preferably, the serum albumin is human serum albumin (HSA). In one embodiment, the first dAb (or a dAb monomer) binds SA (e.g., HSA) with a K_{d} of approximately 50, preferably 70, and more preferably 100, 150 or 200 nM.

In certain embodiments, the dAb monomer that binds SA resists aggregation, unfolds reversibly and/or comprises a framework region as described above for dAb monomers that bind CD38.

In particular embodiments, the antigen-binding fragment of an antibody that binds serum albumin is a dAb that binds human serum albumin. In certain embodiments, the dAb binds human serum albumin and competes for binding to albumin with a dAb selected from the group consisting of DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), and DOM7r-33 (SEQ ID NO: 517).

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), and DOM7r-33 (SEQ ID NO: 517).

For example, the dAb that binds human serum albumin can comprise an amino acid sequence that has at least about 90%, or at least about 95%, or at least about 96%, or at least about 97%, or at least about 98%, or at least about 99% amino acid sequence identity with DOM7h-2 (SEQ ID NO:482), DOM7h-3 (SEQ ID NO:483), DOM7h-4 (SEQ ID NO:484), DOM7h-6 (SEQ ID NO:485), DOM7h-1 (SEQ ID NO:486), DOM7h-7 (SEQ ID NO:487), DOM7h-8 (SEQ ID NO:496), DOM7r-13 (SEQ ID NO:497), DOM7r-14 (SEQ ID NO:498), DOM7h-22 (SEQ ID NO:489), DOM7h-23 (SEQ ID NO:490), DOM7h-24 (SEQ ID NO:491), DOM7h-25 (SEQ ID NO:492), DOM7h-26 (SEQ ID NO:493), DOM7h-21 (SEQ ID NO:494), and DOM7h-27 (SEQ ID NO:495).

Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In more particular embodiments, the dAb is a V_{K} dAb that binds human serum albumin and has an amino acid sequence selected from the group consisting of DOM7h-2 (SEQ ID NO:482), DOM7h-3 (SEQ ID NO:483), DOM7h-4 (SEQ ID NO:484), DOM7h-6 (SEQ ID NO:485), DOM7h-1 (SEQ ID NO:486), DOM7h-7 (SEQ ID NO:487), DOM7h-8 (SEQ ID NO:496), DOM7r-13 (SEQ ID NO:497), and DOM7r-14 (SEQ ID NO:498), or a V_{H} dAb that has an amino acid sequence selected from the group consisting of: DOM7h-22 (SEQ ID NO:489), DOM7h-23 (SEQ ID NO:490), DOM7h-24 (SEQ ID NO:491), DOM7h-25 (SEQ ID NO:492), DOM7h-26 (SEQ ID NO:493), DOM7h-21 (SEQ ID NO:494), DOM7h-27 (SEQ ID NO:495). In other embodiments, the antigen-binding fragment of an antibody that binds serum albumin is a dAb that binds human serum albumin and comprises the CDRs of any of the foregoing amino acid sequences.

Suitable Camelid V_{HH} that bind serum albumin include those disclosed in WO 2004/041862 (Ablynx N.V.) and herein, such as Sequence A (SEQ ID NO:518), Sequence B (SEQ ID NO:519), Sequence C (SEQ ID NO:520), Sequence D (SEQ ID NO:521), Sequence E (SEQ ID NO:522), Sequence F (SEQ ID NO:523), Sequence G (SEQ ID NO:524), Sequence H (SEQ ID NO:525), Sequence I (SEQ ID NO:526), Sequence J (SEQ ID NO:527), Sequence K (SEQ ID NO:528), Sequence L (SEQ ID NO:529), Sequence M (SEQ ID NO:530), Sequence N (SEQ ID NO:531), Sequence O (SEQ ID NO:532), Sequence P (SEQ ID NO:533), Sequence Q (SEQ ID NO:534). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with any one of SEQ ID NOS:518-534.

Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In some embodiments, the ligand comprises an anti-serum albumin dAb that competes with any anti-serum albumin dAb disclosed herein for binding to serum albumin (e.g., human serum albumin).

### Nucleic Acid Molecules, Vectors and Host Cells

The invention also provides isolated and/or recombinant nucleic acid molecules encoding ligands (e.g., dual-specific ligands and multispecific ligands) as described herein.

Nucleic acids referred to herein as “isolate” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (see e.g., Daugherty, B. L. et al., Nucleic Acids Res., 19(9): 2471-2476 (1991); Lewis, A. P. and J. S. Crowe, Gene, 101: 297-302 (1991)).

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes.

In certain embodiments, the isolated and/or recombinant nucleic acid comprises a nucleotide sequence encoding a ligand, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb that binds VEGF disclosed herein, or a dAb that binds EGFR disclosed herein.

For example, in some embodiments, the isolated and/or recombinant nucleic acid comprises a nucleotide sequence encoding a ligand that has binding specificity for VEGF, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of TAR15-1 (SEQ ID NO:100), TAR15-3 (SEQ ID NO:101), TAR15-4 (SEQ ID NO:102), TAR15-9 (SEQ ID NO:103), TAR15-10 (SEQ ID NO:104), TAR15-11 (SEQ ID NO:105), TAR15-12 (SEQ ID NO:106), TAR15-13 (SEQ ID NO:107), TAR15-14 (SEQ ID NO:108), TAR15-15 (SEQ ID NO:109), TAR15-16 (SEQ ID NO:110), TAR15-17 (SEQ ID NO:111), TAR15-18 (SEQ ID NO:112), TAR15-19 (SEQ ID NO:113), TAR15-20 (SEQ ID NO:114), TAR15-22 (SEQ ID NO:115), TAR15-5 (SEQ ID NO:116), TAR15-6 (SEQ ID NO:117), TAR15-7 (SEQ ID NO:118), TAR15-8 (SEQ ID NO:119), TAR15-23 (SEQ ID NO:120), TAR15-24 (SEQ ID NO:121), TAR15-25 (SEQ ID NO:122), TAR15-26 (SEQ ID NO:123), TAR15-27 (SEQ ID NO:124), TAR15-29 (SEQ ID NO:125), TAR15-30 (SEQ ID NO:126), TAR15-6-500 (SEQ ID NO:127), TAR15-6-501 (SEQ ID NO:128), TAR15-6-502 (SEQ ID NO:129), TAR15-6-503 (SEQ ID NO:130), TAR15-6-504 (SEQ ID NO:131), TAR15-6-505 (SEQ ID NO:132), TAR15-6-506 (SEQ ID NO:133), TAR15-6-507 (SEQ ID NO:134), TAR15-6-508 (SEQ ID NO:135), TAR15-6-509 (SEQ ID NO:136), TAR15-6-510 (SEQ ID NO:137), TAR15-8-500 (SEQ ID NO:138), TAR15-8-501 (SEQ ID NO:139), TAR15-8-502 (SEQ ID NO:140), TAR15-8-503 (SEQ ID NO:141), TAR15-8-505 (SEQ ID NO:142), TAR15-8-506 (SEQ ID NO:143), TAR15-8-507 (SEQ ID NO:144), TAR15-8-508 (SEQ ID NO:145), TAR15-8-509 (SEQ ID NO:146), TAR15-8-510 (SEQ ID NO:147), TAR15-8-511 (SEQ ID NO:148), TAR15-26-500 (SEQ ID NO:149), TAR15-26-501 (SEQ ID NO:150), TAR15-26-502 (SEQ ID NO:151), TAR15-26-503 (SEQ ID NO:152), TAR15-26-504 (SEQ ID NO:153), TAR15-26-505 (SEQ ID NO:154), TAR15-26-506 (SEQ ID NO:155), TAR15-26-507 (SEQ ID NO:156), TAR15-26-508 (SEQ ID NO:157), TAR15-26-509 (SEQ ID NO:158), TAR15-26-510 (SEQ ID NO:159), TAR15-26-511 (SEQ ID NO:160), TAR15-26-512 (SEQ ID NO:161), TAR15-26-513 (SEQ ID NO:162), TAR15-26-514 (SEQ ID NO:163), TAR15-26-515 (SEQ ID NO:164), TAR15-26-516 (SEQ ID NO:165), TAR15-26-517 (SEQ ID NO:166), TAR15-26-518 (SEQ ID NO:167), TAR15-26-519 (SEQ ID NO:168), TAR15-26-520 (SEQ ID NO:169), TAR15-26-521 (SEQ ID NO:170), TAR15-26-522 (SEQ ID NO:171), TAR15-26-523 (SEQ ID NO:172), TAR15-26-524 (SEQ ID NO:173), TAR15-26-525 (SEQ ID NO:174), TAR15-26-526 (SEQ ID NO:175), TAR15-26-527 (SEQ ID NO:176), TAR15-26-528 (SEQ ID NO:177), TAR15-26-529 (SEQ ID NO:178), TAR15-26-530 (SEQ ID NO:179), TAR15-26-531 (SEQ ID NO:180), TAR15-26-532 (SEQ ID NO:181), TAR15-26-533 (SEQ ID NO:182), TAR15-26-534 (SEQ ID NO:183), TAR15-26-535 (SEQ ID NO:184), TAR15-26-536 (SEQ ID NO:185), TAR15-26-537 (SEQ ID NO:186), TAR15-26-538 (SEQ ID NO:187), TAR15-26-539 (SEQ ID NO:188), TAR15-26-540 (SEQ ID NO:189), TAR15-26-541 (SEQ ID NO:190), TAR15-26-542 (SEQ ID NO:191), TAR15-26-543 (SEQ ID NO:192), TAR15-26-544 (SEQ ID NO:193), TAR15-26-545 (SEQ ID NO:194), TAR15-26-546 (SEQ ID NO:195), TAR15-26-547 (SEQ ID NO:196), TAR15-26-548 (SEQ ID NO:197), TAR15-26-549 (SEQ ID NO:198), TAR15-26-550 (SEQ ID NO:539), and TAR15-26-551 (SEQ ID NO:540).

For example, in some embodiments, the isolated and/or recombinant nucleic acid comprises a nucleotide sequence encoding a ligand that has binding specificity for VEGFA, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of SEQ ID NO:705 (TAR15-26-555).

In other embodiments, the isolated and/or recombinant nucleic acid comprises a nucleotide sequence encoding a ligand that has binding specificity for EGFR, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM16-17 (SEQ ID NO:325), DOM16-18 (SEQ ID NO:326), DOM16-19 (SEQ ID NO:327), DOM16-20 (SEQ ID NO:328), DOM16-21 (SEQ ID NO:329), DOM16-22 (SEQ ID NO:330), DOM16-23 (SEQ ID NO:331), DOM16-24 (SEQ ID NO:332), DOM16-25 (SEQ ID NO:333), DOM16-26 (SEQ ID NO:334), DOM16-27 (SEQ ID NO:335), DOM16-28 (SEQ ID NO:336), DOM16-29 (SEQ ID NO:337), DOM16-30 (SEQ ID NO:338), DOM16-31 (SEQ ID NO:339), DOM16-32 (SEQ ID NO:340), DOM16-33 (SEQ ID NO:341), DOM16-35 (SEQ ID NO:342), DOM16-37 (SEQ ID NO:343), DOM16-38 (SEQ ID NO:344), DOM16-39 (SEQ ID NO:345), DOM16-40 (SEQ ID NO:346), DOM16-41 (SEQ ID NO:347), DOM16-42 (SEQ ID NO:348), DOM16-43 (SEQ ID NO:349), DOM16-44 (SEQ ID NO:350), DOM16-45 (SEQ ID NO:351), DOM16-46 (SEQ ID NO:352), DOM16-47 (SEQ ID NO:353), DOM16-48 (SEQ ID NO:354), DOM16-49 (SEQ ID NO:355), DOM16-50 (SEQ ID NO:356), DOM16-59 (SEQ ID NO:357), DOM16-60 (SEQ ID NO:358), DOM16-61 (SEQ ID NO:359), DOM16-62 (SEQ ID NO:360), DOM16-63 (SEQ ID NO:361), DOM16-64 (SEQ ID NO:362), DOM16-65 (SEQ ID NO:363), DOM16-66 (SEQ ID NO:364), DOM16-67 (SEQ ID NO:365), DOM16-68 (SEQ ID NO:366), DOM16-69 (SEQ ID NO:367), DOM16-70 (SEQ ID NO:368), DOM16-71 (SEQ ID NO:369), DOM16-72 (SEQ ID NO:370), DOM16-73 (SEQ ID NO:371), DOM16-74 (SEQ ID NO:372), DOM16-75 (SEQ ID NO:373), DOM16-76 (SEQ ID NO:374), DOM16-77 (SEQ ID NO:375), DOM16-78 (SEQ ID NO:376), DOM16-79 (SEQ ID NO:377), DOM16-80 (SEQ ID NO:378), DOM16-81 (SEQ ID NO:379), DOM16-82 (SEQ ID NO:380), DOM16-83 (SEQ ID NO:381), DOM16-84 (SEQ ID NO:382), DOM16-85 (SEQ ID NO:383), DOM16-87 (SEQ ID NO:384), DOM16-88 (SEQ ID NO:385), DOM16-89 (SEQ ID NO:386), DOM16-90 (SEQ ID NO:387), DOM16-91 (SEQ ID NO:388), DOM16-92 (SEQ ID NO:389), DOM16-94 (SEQ ID NO:390), DOM16-95 (SEQ ID NO:391), DOM16-96 (SEQ ID NO:392), DOM16-97 (SEQ ID NO:393), DOM16-98 (SEQ ID NO:394), DOM16-99 (SEQ ID NO:395), DOM16-100 (SEQ ID NO:396), DOM16-101 (SEQ ID NO:397), DOM16-102 (SEQ ID NO:398), DOM16-103 (SEQ ID NO:399), DOM16-104 (SEQ ID NO:400), DOM16-105 (SEQ ID NO:401), DOM16-106 (SEQ ID NO:402), DOM16-107 (SEQ ID NO:403), DOM16-108 (SEQ ID NO:404), DOM16-109 (SEQ ID NO:405), DOM16-110 (SEQ ID NO:406), DOM16-111 (SEQ ID NO:407), DOM16-112 (SEQ ID NO:408), DOM16-113 (SEQ ID NO:409), DOM16-114 (SEQ ID NO:410), DOM16-115 (SEQ ID NO:411), DOM16-116 (SEQ ID NO:412), DOM16-117 (SEQ ID NO:413), DOM16-118 (SEQ ID NO:414), DOM16-119 (SEQ ID NO:415), DOM16-39-6 (SEQ ID NO:416), DOM16-39-8 (SEQ ID NO:417), DOM16-39-34 (SEQ ID NO:418), DOM16-39-48 (SEQ ID NO:419), DOM16-39-87 (SEQ ID NO:420), DOM16-39-90 (SEQ ID NO:421), DOM16-39-96 (SEQ ID NO:422), DOM16-39-100 (SEQ ID NO:423), DOM16-39-101 (SEQ ID NO:424), DOM16-39-102 (SEQ ID NO:425), DOM16-39-103 (SEQ ID NO:426), DOM16-39-104 (SEQ ID NO:427), DOM16-39-105 (SEQ ID NO:428), DOM16-39-106 (SEQ ID NO:429), DOM16-39-107 (SEQ ID NO:430), DOM16-39-108 (SEQ ID NO:431), DOM16-39-109 (SEQ ID NO:432), DOM16-39-110 (SEQ ID NO:433), DOM16-39-111 (SEQ ID NO:434), DOM16-39-112 (SEQ ID NO:435), DOM16-39-113 (SEQ ID NO:436), DOM16-39-114 (SEQ ID NO:437), DOM16-39-115 (SEQ ID NO:438), DOM16-39-116 (SEQ ID NO:439), DOM16-39-117 (SEQ ID NO:440), DOM16-39-200 (SEQ ID NO:441), DOM16-39-201 (SEQ ID NO:442), DOM16-39-202 (SEQ ID NO:443), DOM16-39-203 (SEQ ID NO:444), DOM16-39-204 (SEQ ID NO:445), DOM16-39-205 (SEQ ID NO:446), DOM16-39-206 (SEQ ID NO:447), DOM16-39-207 (SEQ ID NO:448), DOM16-39-209 (SEQ ID NO:449), DOM16-52 (SEQ ID NO:450), NB1 (SEQ ID NO:451), NB2 (SEQ ID NO:452), NB3 (SEQ ID NO:453), NB4 (SEQ ID NO:454), NB5 (SEQ ID NO:455), NB6 (SEQ ID NO:456), NB7 (SEQ ID NO:457), NB8 (SEQ ID NO:458), NB9 (SEQ ID NO:459), NB10 (SEQ ID NO:460), NB11 (SEQ ID NO:461), NB12 (SEQ ID NO:462), NB13 (SEQ ID NO:463), NB14 (SEQ ID NO:464), NB15 (SEQ ID NO:465), NB16 (SEQ ID NO:466), NB17 (SEQ ID NO:467), NB18 (SEQ ID NO:468), NB19 (SEQ ID NO:469), NB20 (SEQ ID NO:470), NB21 (SEQ ID NO:471), and NB22 (SEQ ID NO:472).

In other embodiments, the isolated and/or recombinant nucleic acid comprises a nucleotide sequence encoding a ligand that has binding specificity for EGFR, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with an amino acid sequence selected from the group consisting of SEQ ID NOS:623-703, 727 and 728.

In other embodiments, the isolated and/or recombinant nucleic acid encoding a ligand that has binding specificity for VEGF, as described herein, comprises a nucleotide sequence that has at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence encoding an anti-VEGF dAb selected from the group consisting of TAR15-1 (SEQ ID NO:1), TAR15-3 (SEQ ID NO:2), TAR15-4 (SEQ ID NO:3), TAR15-9 (SEQ ID NO:4), TAR15-10 (SEQ ID NO:5), TAR15-11 (SEQ ID NO:6), TAR15-12 (SEQ ID NO:7), TAR15-13 (SEQ ID NO:8), TAR15-14 (SEQ ID NO:9), TAR15-15 (SEQ ID NO:10), TAR15-16 (SEQ ID NO:11), TAR15-17 (SEQ ID NO:12), TAR15-18 (SEQ ID NO:13), TAR15-19 (SEQ ID NO:14), TAR15-20 (SEQ ID NO:15), TAR 15-22 (SEQ ID NO:16), TAR15-5 (SEQ ID NO:17), TAR15-6 (SEQ ID NO:18), TAR15-7 (SEQ ID NO:19), TAR15-8 (SEQ ID NO:20), TAR15-23 (SEQ ID NO:21), TAR15-24 (SEQ ID NO:22), TAR15-25 (SEQ ID NO:23), TAR15-26 (SEQ ID NO:24), TAR15-27 (SEQ ID NO:25), TAR15-29 (SEQ ID NO:26), TAR15-30 (SEQ ID NO:27), TAR15-6-500 (SEQ ID NO:28), TAR15-6-501 (SEQ ID NO:29), TAR15-6-502 (SEQ ID NO:30), TAR15-6-503 (SEQ ID NO:31), TAR15-6-504 (SEQ ID NO:32), TAR15-6-505 (SEQ ID NO:33), TAR15-6-506 (SEQ ID NO:34), TAR15-6-507 (SEQ ID NO:35), TAR15-6-508 (SEQ ID NO:36), TAR15-6-509 (SEQ ID NO:37), TAR15-6-510 (SEQ ID NO:38), TAR15-8-500 (SEQ ID NO:39), TAR15-8-501 (SEQ ID NO:40), TAR15-8-502 (SEQ ID NO:41), TAR15-8-503 (SEQ ID NO:42), TAR15-8-505 (SEQ ID NO:43), TAR15-8-506 (SEQ ID NO:44), TAR15-8-507 (SEQ ID NO:45), TAR15-8-508 (SEQ ID NO:46), TAR15-8-509 (SEQ ID NO:47), R15-8-510 (SEQ ID NO:48), TAR15-8-511 (SEQ ID NO:49), TAR15-26-500 (SEQ ID NO:50), TAR15-26-501 (SEQ ID NO:51), TAR15-26-502 (SEQ ID NO:52), TAR15-26-503 (SEQ ID NO:53), TAR15-26-504 (SEQ ID NO:54), TAR15-26-505 (SEQ ID NO:55), TAR15-26-506 (SEQ ID NO:56), TAR15-26-507 (SEQ ID NO:57), TAR15-26-508 (SEQ ID NO:58), TAR15-26-509 (SEQ ID NO:59), TAR15-26-510 (SEQ ID NO:60), TAR15-26-511 (SEQ ID NO:61), TAR15-26-512 (SEQ ID NO:62), TAR15-26-513 (SEQ ID NO:63), TAR15-26-514 (SEQ ID NO:64), TAR15-26-515 (SEQ ID NO:65), TAR15-26-516 (SEQ ID NO:66), TAR15-26-517 (SEQ ID NO:67), TAR15-26-518 (SEQ ID NO:68), TAR15-26-519 (SEQ ID NO:69), TAR15-26-520 (SEQ ID NO:70), TAR15-26-521 (SEQ ID NO:71), TAR15-26-522 (SEQ ID NO:72), TAR15-26-523 (SEQ ID NO:73), TAR15-26-524 (SEQ ID NO:74), TAR15-26-525 (SEQ ID NO:75), TAR15-26-526 (SEQ ID NO:76), TAR15-26-527 (SEQ ID NO:77), TAR15-26-528 (SEQ ID NO:78), TAR15-26-529 (SEQ ID NO:79), TAR15-26-530 (SEQ ID NO:80), TAR15-26-531 (SEQ ID NO:81), TAR15-26-532 (SEQ ID NO:82), TAR15-26-533 (SEQ ID NO:83), TAR15-26-534 (SEQ ID NO:84), TAR15-26-535 (SEQ ID NO:85), TAR15-26-536 (SEQ ID NO:86), TAR15-26-537 (SEQ ID NO:87), TAR15-26-538 (SEQ ID NO:88), TAR15-26-539 (SEQ ID NO:89), TAR15-26-540 (SEQ ID NO:90), TAR15-26-541 (SEQ ID NO:91), TAR15-26-542 (SEQ ID NO:92), TAR15-26-543 (SEQ ID NO:93), TAR15-26-544 (SEQ ID NO:94), TAR15-26-545 (SEQ ID NO:95), TAR15-26-546 (SEQ ID NO:96), TAR15-26-547 (SEQ ID NO:97), TAR15-26-548 (SEQ ID NO:98), TAR15-26-549 (SEQ ID NO:99), TAR15-21 (SEQ ID NO:535), TAR15-2 (SEQ ID NO:536), TAR15-26-550 (SEQ ID NO:537), and TAR15-26-551 (SEQ ID NO:538). Preferably, nucleotide sequence identity is determined over the whole length of the nucleotide sequence that encodes the selected anti-VEGF dAb.

In other embodiments, the isolated and/or recombinant nucleic acid encoding a ligand that has binding specificity for VEGF, as described herein, comprises a nucleotide sequence that has at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence encoding TAR15-26-555 (SEQ ID NO:705).

In other embodiments, the isolated and/or recombinant nucleic acid encoding a ligand that has binding specificity for EGFR, as described herein, comprises a nucleotide sequence that has at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence encoding an anti-VEGF dAb selected from the group consisting of DOM16-17 (SEQ ID NO:199), DOM16-18 (SEQ ID NO:200), DOM16-19 (SEQ ID NO:201), DOM16-20 (SEQ ID NO:202), DOM16-21 (SEQ ID NO:203), DOM16-22 (SEQ ID NO:204), DOM16-23 (SEQ ID NO:205), DOM16-24 (SEQ ID NO:206), DOM16-25 (SEQ ID NO:207), DOM16-26 (SEQ ID NO:208), DOM16-27 (SEQ ID NO:209), DOM16-28 (SEQ ID NO:210), DOM16-29 (SEQ ID NO:211), DOM16-30 (SEQ ID NO:212), DOM16-31 (SEQ ID NO:213), DOM16-32 (SEQ ID NO:214), DOM16-33 (SEQ ID NO:215), DOM16-35 (SEQ ID NO:216), DOM16-37 (SEQ ID NO:217), DOM16-38 (SEQ ID NO:218), DOM16-39 (SEQ ID NO:219), DOM16-40 (SEQ ID NO:220), DOM16-41 (SEQ ID NO:221), DOM16-42 (SEQ ID NO:222), DOM16-43 (SEQ ID NO:223), DOM16-44 (SEQ ID NO:224), DOM16-45 (SEQ ID NO:225), DOM16-46 (SEQ ID NO:226), DOM16-47 (SEQ ID NO:227), DOM16-48 (SEQ ID NO:228), DOM16-49 (SEQ ID NO:229), DOM16-50 (SEQ ID NO:230), DOM16-59 (SEQ ID NO:231), DOM16-60 (SEQ ID NO:232), DOM16-61 (SEQ ID NO:233), DOM16-62 (SEQ ID NO:234), DOM16-63 (SEQ ID NO:235), DOM16-64 (SEQ ID NO:236), DOM16-65 (SEQ ID NO:237), DOM16-66 (SEQ ID NO:238), DOM16-67 (SEQ ID NO:239), DOM16-68 (SEQ ID NO:240), DOM16-69 (SEQ ID NO:241), DOM16-70 (SEQ ID NO:242), DOM16-71 (SEQ ID NO:243), DOM16-72 (SEQ ID NO:244), DOM16-73 (SEQ ID NO:245), DOM16-74 (SEQ ID NO:246), DOM16-75 (SEQ ID NO:247), DOM16-76 (SEQ ID NO:248), DOM16-77 (SEQ ID NO:249), DOM16-78 (SEQ ID NO:250), DOM16-79 (SEQ ID NO:251), DOM16-80 (SEQ ID NO:252), DOM16-81 (SEQ ID NO:253), DOM16-82 (SEQ ID NO:254), DOM16-83 (SEQ ID NO:255), DOM16-84 (SEQ ID NO:256), DOM16-85 (SEQ ID NO:257), DOM16-87 (SEQ ID NO:258), DOM16-88 (SEQ ID NO:259), DOM16-89 (SEQ ID NO:260), DOM16-90 (SEQ ID NO:261), DOM16-91 (SEQ ID NO:262), DOM16-92 (SEQ ID NO:263), DOM16-94 (SEQ ID NO:264), DOM16-95 (SEQ ID NO:265), DOM16-96 (SEQ ID NO:266), DOM16-97 (SEQ ID NO:267), DOM16-98 (SEQ ID NO:268), DOM16-99 (SEQ ID NO:269), DOM16-100 (SEQ ID NO:270), DOM16-101 (SEQ ID NO:271), DOM16-102 (SEQ ID NO:272), DOM16-103 (SEQ ID NO:273), DOM16-104 (SEQ ID NO:274), DOM16-105 (SEQ ID NO:275), DOM16-106 (SEQ ID NO:276), DOM16-107 (SEQ ID NO:277), DOM16-108 (SEQ ID NO:278), DOM16-109 (SEQ ID NO:279), DOM16-110 (SEQ ID NO:280), DOM16-111 (SEQ ID NO:281), DOM16-112 (SEQ ID NO:282), DOM16-113 (SEQ ID NO:283), DOM16-114 (SEQ ID NO:284), DOM16-115 (SEQ ID NO:285), DOM16-116 (SEQ ID NO:286), DOM16-117 (SEQ ID NO:287), DOM16-118 (SEQ ID NO:288), DOM16-119 (SEQ ID NO:289), DOM16-39-6 (SEQ ID NO:290), DOM16-39-8 (SEQ ID NO:291), DOM16-39-34 (SEQ ID NO:292), DOM16-39-48 (SEQ ID NO:293), DOM16-39-87 (SEQ ID NO:294), DOM16-39-90 (SEQ ID NO:295), DOM16-39-96 (SEQ ID NO:296), DOM16-39-100 (SEQ ID NO:297), DOM16-39-101 (SEQ ID NO:298), DOM16-39-102 (SEQ ID NO:299), DOM16-39-103 (SEQ ID NO:300), DOM16-39-104 (SEQ ID NO:301), DOM16-39-105 (SEQ ID NO:302), DOM16-39-106 (SEQ ID NO:303), DOM16-39-107 (SEQ ID NO:304), DOM16-39-108 (SEQ ID NO:305), DOM16-39-109 (SEQ ID NO:306), DOM16-39-110 (SEQ ID NO:307), DOM16-39-111 (SEQ ID NO:308), DOM16-39-112 (SEQ ID NO:309), DOM16-39-113 (SEQ ID NO:310), DOM16-39-114 (SEQ ID NO:311), DOM16-39-115 (SEQ ID NO:312), DOM16-39-116 (SEQ ID NO:313), DOM16-39-117 (SEQ ID NO:314), DOM16-39-200 (SEQ ID NO:315), DOM16-39-201 (SEQ ID NO:316), DOM16-39-202 (SEQ ID NO:317), DOM16-39-203 (SEQ ID NO:318), DOM16-39-204 (SEQ ID NO:319), DOM16-39-205 (SEQ ID NO:320), DOM16-39-206 (SEQ ID NO:321), DOM16-39-207 (SEQ ID NO:322), DOM16-39-209 (SEQ ID NO:323), and DOM16-52 (SEQ ID NO:324). Preferably, nucleotide sequence identity is determined over the whole length of the nucleotide sequence that encodes the selected anti-EGFR dAb.

In other embodiments, the isolated and/or recombinant nucleic acid encoding a ligand that has binding specificity for EGFR, as described herein, comprises a nucleotide sequence that has at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence encoding an anti-EGFR dAb selected from the group consisting of SEQ ID NOS:623-703, 727 and 728.

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g., plasmids, phagmids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic (e.g., lac, tac, T3, T7 promoters for E. coli) and eukaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g., lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In preferred embodiments, the host cell is a non-human host cell.

The invention also provides a method for producing a ligand (e.g., dual-specific ligand, multispecific ligand) of the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

### Preparation of Immunoglobulin Based Ligands

Ligands (e.g., dual specific ligands, multispecific ligands) according to the invention can be prepared according to previously established techniques, used in the field of antibody engineering, for the preparation of scFv, “phage” antibodies and other engineered antibody molecules. Techniques for the preparation of antibodies are for example described in the following reviews and the references cited therein: Winter & Milstein, (1991) Nature 349:293-299; Pluckthun (1992) Immunological Reviews 130:151-188; Wright et al., (1992) Crit. Rev. Immunol. 12:125-168; Holliger, P. & Winter, G. (1993) Curr. Opin. Biotechnol. 4, 446-449; Carter, et al. (1995) J. Hematother. 4, 463-470; Chester, K. A. & Hawkins, R. E. (1995) Trends Biotechnol. 13, 294-300; Hoogenboom, H. R. (1997) Nat. Biotechnol. 15, 125-126; Fearon, D. (1997) Nat. Biotechnol. 15, 618-619; Plückthun, A. & Pack, P. (1997) Immunotechnology 3, 83-105; Carter, P. & Merchant, A. M. (1997) Curr. Opin. Biotechnol. 8, 449-454; Holliger, P. & Winter, G. (1997) Cancer Immunol. Immunother. 45, 128-130.

Suitable techniques employed for selection of antibody variable domains with a desired specificity employ libraries and selection procedures which are known in the art. Natural libraries (Marks et al. (1991) J. Mol. Biol., 222: 581; Vaughan et al. (1996) Nature Biotech., 14: 309) which use rearranged V genes harvested from human B cells are well known to those skilled in the art. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97) are prepared by cloning immunoglobulin V genes, usually using PCR. Errors in the PCR process can lead to a high degree of randomisation. V_{H} and/or V_{L} libraries may be selected against target antigens or epitopes separately, in which case single domain binding is directly selected for, or together.

### Library Vector Systems

A variety of selection systems are known in the art which are suitable for use in the present invention. Examples of such systems are described below.

Bacteriophage lambda expression systems may be screened directly as bacteriophage plaques or as colonies of lysogens, both as previously described Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. Whilst such expression systems can be used to screen up to 10⁶ different members of a library, they are not really suited to screening of larger numbers (greater than 10⁶ members). Of particular use in the construction of libraries are selection display systems, which enable a nucleic acid to be linked to the polypeptide it expresses. As used herein, a selection display system is a system that permits the selection, by suitable display means, of the individual members of the library by binding the generic and/or target.

Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encode them) for the in vitro selection and amplification of specific antibody fragments that bind a target antigen (McCafferty et al., WO 92/01047). The nucleotide sequences encoding the variable regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature, 348: 552; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci. U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci. U.S.A., 85: 5879-5883; Chaudhary et al. (1990) Proc. Natl. Acad. Sci. U.S.A., 87: 1066-1070; McCafferty et al. (1990) supra; Clackson et al. (1991) Nature, 352: 624; Marks et al. (1991) J. Mol. Biol., 222: 581; Chiswell et al. (1992) Trends Biotechnol., 10: 80; Marks et al. (1992) J. Biol. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Other systems for generating libraries of polypeptides involve the use of cell-free enzymatic machinery for the in vitro synthesis of the library members. In one method, RNA molecules are selected by alternate rounds of selection against a target and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection.

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### Library Construction

Libraries intended for selection, may be constructed using techniques known in the art, for example as set forth above, or may be purchased from commercial sources. Libraries which are useful in the present invention are described, for example, in WO99/20749. Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. Mutagenesis of nucleic acid sequences encoding structurally optimized polypeptides is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalyzed by a thermostable, DNA-dependent DNA polymerase to amplify the target sequence of interest, is well known in the art. The construction of various antibody libraries has been discussed in Winter et al. (1994) Ann. Rev. Immunology 12, 433-55, and references cited therein.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler. The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenised, mismatch is required, at least in the first round of synthesis. The ability to optimise the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

### Combining Single Variable Domains

Domains useful in the invention, once selected, may be combined by a variety of methods known in the art, including covalent and non-covalent methods. Preferred methods include the use of polypeptide linkers, as described, for example, in connection with scFv molecules (Bird et al., (1988) Science 242:423-426). Discussion of suitable linkers is provided in Bird et al. Science 242, 423-426; Hudson et al, Journal Immunol Methods 231 (1999) 177-189; Hudson et al, Proc Nat Acad Sci USA 85, 5879-5883. Linkers are preferably flexible, allowing the two single domains to interact. One linker example is a (Gly₄ Ser)ₙ linker, where n=1 to 8, e.g., 2, 3, 4, 5 or 7. The linkers used in diabodies, which are less flexible, may also be employed (Holliger et al., (1993) Proc Nat Acad Sci USA 90:6444-6448). In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilize V_{H}-V_{H}, V_{L}-V_{L} or V_{H}-V_{L} dimers (Reiter et al., (1994) Protein Eng. 7:697-704) or by remodelling the interface between the variable domains to improve the “fit” and thus the stability of interaction (Ridgeway et al., (1996) Protein Eng. 7:617-621; Zhu et al., (1997) Protein Science 6:781-788). Other techniques for joining or stabilizing variable domains of immunoglobulins, and in particular antibody V_{H} domains, may be employed as appropriate.

### Characterisation of Ligands

The binding of a dual-specific ligand to the cell or the binding of each binding domain to each specific target can be tested by methods which will be familiar to those skilled in the art and include ELISA. In a preferred embodiment of the invention binding is tested using monoclonal phage ELISA. Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

### Structure of Ligands

In the case that each variable domain is selected from V-gene repertoires, selected for instance using phage display technology as herein described, then these variable domains comprise a universal framework region, such that they may be recognized by a specific generic dual-specific ligand as herein defined. The use of universal frameworks, generic ligands and the like is described in WO99/20749.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In general, nucleic acid molecules and vector constructs required for selection, preparation and formatting dual-specific ligands may be constructed and manipulated as set forth in standard laboratory manuals, such as Sambrook et al. (1989) Molecular Cloning: A Laboratory Manual, Cold Spring Harbor, USA.

The manipulation of nucleic acids useful in the present invention is typically carried out in recombinant vectors. As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes and episomal vectors. Such vectors may be used for simple cloning and mutagenesis; alternatively a gene expression vector is employed. A vector of use according to the invention may be selected to accommodate a polypeptide coding sequence of a desired size, typically from 0.25 kilobase (kb) to 40 kb or more in length A suitable host cell is transformed with the vector after in vitro cloning manipulations. Each vector contains various functional components, which generally include a cloning (or “polylinker”) site, an origin of replication and at least one selectable marker gene. If the given vector is an expression vector, it additionally possesses one or more of the following: enhancer element, promoter, transcription termination and signal sequences, each positioned in the vicinity of the cloning site, such that they are operatively linked to the gene encoding a dual-specific ligand according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since the replication of vectors encoding a dual-specific ligand according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Delgarno sequence operably linked to the coding sequence.

The preferred vectors are expression vectors that enables the expression of a nucleotide sequence corresponding to a polypeptide library member. Thus, selection with the first and/or second antigen or epitope can be performed by separate propagation and expression of a single clone expressing the polypeptide library member or by use of any selection display system. As described above, the preferred selection display system is bacteriophage display. Thus, phage or phagemid vectors may be used, (e.g., pIT1 or pIT2. Leader sequences useful in the invention include pelB, stII, ompA, phoA, bla and pelA. One example are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors encoding dual-specific ligands according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

### Skeletons

Skeletons may be based on immunoglobulin molecules or may be non-immunoglobulin in origin as set forth above. Each domain of the dual-specific ligand may be a different skeleton. Preferred immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

### Protein Scaffolds

Each binding domain comprises a protein scaffold and one or more CDRs which are involved in the specific interaction of the domain with one or more epitopes. Advantageously, an epitope binding domain according to the present invention comprises three CDRs. Suitable protein scaffolds include any of those selected from the group consisting of the following: those based on immunoglobulin domains, those based on fibronectin, those based on affibodies, those based on CTLA4, those based on chaperones such as GroEL, those based on lipocallin and those based on the bacterial Fc receptors SpA and SpD. Those skilled in the art will appreciate that this list is not intended to be exhaustive.

### Scaffolds for Use in Constructing Ligands

### Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

Libraries of ligands and/or binding domains can be designed in which certain loop lengths and key residues have been chosen to ensure that the main-chain conformation of the members is known. Advantageously, these are real conformations of immunoglobulin superfamily molecules found in nature, to minimize the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on dual-specific ligand sequences and to choose residues for diversification which do not affect the canonical structure. It is known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that V_{κ} and V_{λ} domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the ligands of the invention possess a single known main-chain conformation.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin domain are considered and then a naturally occurring variable domain is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing ligands (e.g., ds-dAbs) or libraries thereof the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of 1-13 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS1 (79% of the expressed repertoire), H2-CS 3 (46%), L1-CS 2 of V_{κ} (39%), L2-CS 1 (100%), L3-CS 1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Rabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it is observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

### Diversification of the Canonical Sequence

Having selected several known main-chain conformations or, preferably a single known main-chain conformation, dual-specific ligands (e.g., ds-dAbs) or libraries for use in the invention can be constructed by varying each binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or are preferably selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomization has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

Preferably, only the residues that are directly involved in creating or modifying the desired function of each domain of the dual-specific ligand molecule are diversified. For many molecules, the function of each domain will be to bind a target and therefore diversity should be concentrated in the target binding site, while avoiding changing residues which are crucial to the overall packing of the molecule or to maintaining the chosen main-chain conformation.

### Diversification of the Canonical Sequence as it Applies to Antibody Domains

In the case of antibody based ligands (e.g., ds-dAbs), the binding site for each target is most often the antigen binding site. Thus, preferably only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Rabat et al. (1991, supra), some seven residues compared to the two diversified in the library for use according to the invention. This represents a significant improvement in terms of the functional diversity required to create a range of antigen binding specificities.

In nature, antibody diversity is the result of two processes: somatic recombination of getadine V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire can be created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

Naive repertoires of binding domains for the construction of dual-specific ligands in which some or all of the residues in the antigen binding site are varied are known in the art. (See, WO 2004/058821, WO 2004/003019, and WO 03/002609). The “primary” library mimics the natural primary repertoire, with diversity restricted to residues at the centre of the antigen binding site that are diverse in the germline V gene segments (germline diversity) or diversified during the recombination process (junctional diversity). Those residues which are diversified include, but are not limited to, H50, H52, H52a, H53, H55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated. Those residues which are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use in the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favors certain amino acid residues. If the amino acid composition of the ten most diverse positions in each of the V_{H}, V_{K} and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens or epitopes and may help to explain the required promiscuity of antibodies in the primary repertoire.

Since it is preferable to mimic this distribution of amino acids, the distribution of amino acids at the positions to be varied preferably mimics that seen in the antigen binding site of antibodies. Such bias in the substitution of amino acids that permits selection of certain polypeptides (not just antibody polypeptides) against a range of target antigens is easily applied to any polypeptide repertoire.

There are various methods for biasing the amino acid distribution at the position to be varied (including the use of tri-nucleotide mutagenesis, see WO97/08320), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

### Therapeutic and Diagnostic Compositions and Uses

The invention provides compositions comprising the ligands of the invention and a pharmaceutically acceptable carrier, diluent or excipient, and therapeutic and diagnostic methods that employ the ligands or compositions of the invention. The ligands according to the method of the present invention may be employed in in vivo therapeutic and prophylactic applications, in vivo diagnostic applications and the like.

Therapeutic and prophylactic uses of ligands of the invention involve the administration of ligands according to the invention to a recipient mammal, such as a human. The ligands bind to targets with high affinity and/or avidity. In some embodiments, such as IgG-like ligands, the ligands can allow recruitment of cytotoxic cells to mediate killing of cancer cells, for example by antibody dependent cellular cytoxicity.

Substantially pure ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

For example, the ligands of the present invention will typically find use in preventing, suppressing or treating disease states. For example, ligands can be administered to treat, suppress or prevent a chronic inflammatory disease, allergic hypersensitivity, cancer, bacterial or viral infection, autoimmune disorders (which include, but are not limited to, Type I diabetes, asthma, multiple sclerosis, rheumatoid arthritis, juvenile rheumatoid arthritis, psoriatic arthritis, spondylarthropathy (e.g., ankylosing spondylitis), systemic lupus erythematosus, inflammatory bowel disease (e.g., Crohn's disease, ulcerative colitis), myasthenia gravis and Behcet's syndrome, psoriasis, endometriosis, and abdominal adhesions (e.g., post abdominal surgery).

The ligands are useful for treating infectious diseases in which cells infected with an infectious agent contain higher levels of cell surface EGFR than uninfected cells or that contain one or more cell surface targets that are not present on in infected cells, such as a protein that is encoded by the infectious agent (e.g., bacteria, virus).

Ligands according to the invention that are able to bind to EGFR can be internalized by cells that express EGFR (e.g., endocytosed), and can deliver therapeutic agents (e.g., a toxin) intracellularly (e.g., deliver a dAb that binds an intracellular target). In addition, ligands provide a means by which a binding domain (e.g., a dAb monomer) that is specifically able to bind to an intracellular target can be delivered to an intracellular environment. This strategy requires, for example, a binding domain with physical properties that enable it to remain functional inside the cell. Alternatively, if the final destination intracellular compartment is oxidising, a well folding ligand may not need to be disulphide free.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest. Treatment includes ameliorating symptoms associated with the disease, and also preventing or delaying the onset of the disease and also lessening the severity or frequency of symptoms of the disease.

The term “cancer” refers to the pathological condition in mammals that is typically characterized by dysregulated cellular proliferation or survival. Examples of cancer include, but are not limited to, carcinoma, lymphoma, blastoma, sarcoma, and leukemia and lymphoid malignancies. More particular examples of cancers include squamous cell cancer (e.g. epithelial squamous cell cancer), lung cancer (e.g., small-cell lung carcinoma, non-small cell lung carcinoma, adenocarcinoma of the lung, squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, gall bladder cancer, hepatoma, breast cancer, colon cancer, rectal cancer, colorectal cancer, multiple myeloma, chronic myelogenous leukemia, acute myelogenous leukemia, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, and the like. Cancers characterized by expression of EGFR on the surface of cancerous cells (EGFR-expressing cancers) include, for example, bladder cancer, ovarian cancer, colorectal cancer, breast cancer, lung cancer (e.g., non-small cell lung carcinoma), gastric cancer, pancreatic cancer, prostate cancer, head and neck cancer, renal cancer and gall bladder cancer.

Animal model systems which can be used to assess efficacy of the ligands of the inventon in preventing treating or suppressing disease (e.g., cancer) are available. Suitable models of cancer include, for example, xenograft and orthotopic models of human cancers in animal models, such as the SCID-hu myeloma model (Epstein J, and Yaccoby, S., Methods Mol. Med. 113:183-90 (2005), Tassone P, et al., Clin Cancer Res. 11(11):4251-8 (2005)), mouse models of human lung cancer (e.g., Meuwissen R and Berns A, Genes Dev.19(6):643-64 (2005)), and mouse models of metastatic cancers (e.g., Kubota T., J Cell Biochem. 56(1):4-8 (1994)).

Generally, the present ligands will be utilized in purified form together with pharmacologically appropriate carriers. Typically, these carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The ligands of the present invention may be used as separately administered compositions or in conjunction with other agents. The ligands can be administered and or formulated together with one or more additional therapeutic or active agents. When a ligand is administered with an additional therapeutic agent, the ligand can be administered before, simultaneously with or subsequent to administration of the additional agent. Generally, the ligand and additional agent are administered in a manner that provides an overlap of therapeutic effect. Additional agents that can be administered or formulated with the ligand of the invention include, for example, various immunotherapeutic drugs, such as cylcosporine, methotrexate, adriamycin or cisplatinum, antibiotics, antimycotics, anti-viral agents and immunotoxins. For example, when the antagonist is administered to prevent, suppress or treat lung inflammation or a respiratory disease, it can be administered in conjunction with phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short acting anticholinergics (e.g., ipratropium bromide and oxitropium bromide), long-acting anticholinergics (e.g., tiotropium), theophylline (e.g. short acting formulation, long acting formulation), inhaled steroids (e.g., beclomethasone, beclometasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol).

The ligands of the invention can be coadministered (e.g., to treat cancer, an inflammatory disease or other disease) with a variety of suitable co-therapeutic agents, including cytokines, analgesics/antipyretics, antiemetics, and chemotherapeutics. Further suitable co-therapeutic agents include immunosuppressive agents selected from the group consisting of cyclosporine, azathioprine, mycophenolic acid, mycophenolate mofetil, corticosteroids, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, and anti-thymocyte globulin, anti-inflammatory agents selected from the group consisting of aspirin, other salicylates, steroidal drugs, NSAIDs (nonsteroidal anti-inflammatory drugs), Cox-2 inhibitors, and DMARDs (disease modifying antirheumatic drugs); anti-psoriasis agents selected from the group consisting of coal tar, A vitamin, anthralin, calcipotrien, tarazotene, corticosteroids, methotrexate, retinoids, cyclosporine, etanercept, alefacept, efaluzimab, 6-thioguanine, mycophenolate mofetil, tacrolimus (FK-506), and hydroxyurea.

Cytokines include, without limitation, a lymphokine, tumor necrosis factors, tumor necrosis factor-like cytokine, lymphotoxin, interferon, macrophage inflammatory protein, granulocyte monocyte colony stimulating factor, interleukin (including, without limitation, interleukin-1, interleukin-2, interleukin-6, interleukin-12, interleukin-15, interleukin-18), growth factors, which include, without limitation, (e.g., growth hormone, insulin-like growth factor 1 and 2 (IGF-1 and IGF-2), granulocyte colony stimulating factor (GCSF), platelet derived growth factor (PGDF), epidermal growth factor (EGF), and agents for erythropoiesis stimulation, e.g., recombinant human erythropoietin (Epoetin alfa), EPO, a hormonal agonist, hormonal antagonists (e.g., flutamide, tamoxifen, leuprolide acetate (LUPRON)), and steroids (e.g., dexamethasone, retinoid, betamethasone, cortisol, cortisone, prednisone, dehydrotestosterone, glucocorticoid, mineralocorticoid, estrogen, testosterone, progestin).

Analgesics/antipyretics can include, without limitation, e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like.

Antiemetics can also be coadministered to prevent or treat nausea and vomiting e.g., suitable antiemetics include meclizine hydrochloride, nabilone, prochlorperazine, dimenhydrinate, promethazine hydrochloride, thiethylperazine, scopolamine, and the like.

Chemotherapeutic agents, as that term is used herein, include, but are not limited to, for example antimicrotubule agents, e.g., taxol (paclitaxel), taxotere (docetaxel); alkylating agents, e.g., cyclophosphamide, carmustine, lomustine, and chlorambucil; cytotoxic antibiotics, e.g., dactinomycin, doxorubicin, mitomycin-C, and bleomycin; antimetabolites, e.g., cytarabine, gemcitatin, methotrexate, and 5-fluorouracil; antimiotics, e.g., vincristine vinca alkaloids, e.g., etoposide, vinblastine, and vincristine; and others such as cisplatin, dacarbazine, procarbazine, and hydroxyurea; and combinations thereof.

The ligands of the invention can be used to treat cancer in combination with another therapeutic agent. For example, a ligand of the invention can be administered in combination with a chemotherapeutic agent or an antineoplastic composition comprising a (at least one) chemotherapeutic agent. Advantageously, in such a therapeutic approach, the amount of chemotherapeutic agent that must be administered to be effective can be reduced. Thus the invention provides a method of treating cancer comprising administering to a patient in need thereof a therapeutically effective amount of a ligand of the invention and a chemotherapeutic agent, wherein the chemotherapeutic agent is administered at a low dose. Generally the amount of chemotherapeutic agent that is coadministered with a ligand of the invention is about 80%, or about 70%, or about 60%, or about 50%, or about 40%, or about 30%, or about 20%, or about 10% or less, of the dose of chemotherapeutic agent alone that is normally administered to a patient. Thus, cotherapy is particularly advantageous when the chemotherapeutic agent causes deleterious or undesirable side effects that may be reduced or eliminated at a lower doses.

Pharmaceutical compositions can include “cocktails” of various cytotoxic or other agents in conjunction with ligands of the present invention, or even combinations of ligands according to the present invention having different specificities, such as ligands selected using different target antigens or epitopes, whether or not they are pooled prior to administration.

The route of administration of pharmaceutical compositions according to the invention may be any suitable route, such as any of those commonly known to those of ordinary skill in the art. For therapy, including without limitation immunotherapy, the ligands of the invention can be administered to any patient in accordance with standard techniques. The administration can be by any appropriate mode, including parenterally, intravenously, intramuscularly, intraperitoneally, transdermally, intrathecally, intraarticularly, via the pulmonary route, or also, appropriately, by direct infusion (e.g., with a catheter). The dosage and frequency of administration will depend on the age, sex and condition of the patient, concurrent administration of other drugs, counterindications and other parameters to be taken into account by the clinician. Administration can be local (e.g., local delivery to the lung by pulmonary administration, (e.g., intranasal administration) or local injection directly into a tumor) or systemic as indicated.

The ligands of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the ligands can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's health, but generally range from 0.005 to 5.0 mg of ligand per kilogram of body weight, with doses of 0.05 to 2.0 mg/kg/dose being more commonly used. For prophylactic applications, compositions containing the present ligands or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a ligand is administered to treat, suppress or prevent a disease, it can be administered up to four times per day, twice weekly, once weekly, once every two weeks, once a month, or once every two months, at a dose of, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the dual-specific ligand is administered to treat, suppress or prevent a chronic inflammatory disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

In particular embodiments, the ligand of the invention is administered at a dose that provides saturation of EGFR or a desired serum concentration in vivo. The skilled physician can determine appropriate dosing to achieve saturation, for example by titrating ligand and monitoring the amount of free binding sites on EGFR expressing cells or the serum concentration of ligand. Therapeutic regiments that involve administering a therapeutic agent to achieve target saturation or a desired serum concentration of agent are common in the art, particularly in the field of oncology.

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status—score ranges from 32 to 224, with higher scores indicating a better quality of life), the Quality of Life Rheumatoid Arthritis Scale, or other accepted clinical assessment scale as known in the field. A sustained (e.g., one day or more, preferably longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing ligands according to the present invention may be utilized in prophylactic and therapeutic settings to aid in the alteration, inactivation, killing or removal of a select target cell population in a mammal. In addition, the ligands and selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g. antibodies, cell-surface receptors or binding proteins thereof whereby the undesired cells are killed or otherwise removed from the blood for return to the mammal in accordance with standard techniques.

In one embodiment, the invention relates to a method for delivering anti-angiogenic therapy (anti-VEGF therapy) to a site containing cells that express or overexpress EGFR, comprising administering an effective amount of a ligand that has binding specificity for VEGF and for EGFR to a subject in need thereof.

The invention also relates to use of a ligand that has binding specificity for VEGF and for EGFR for delivering anti-angiogenic therapy (anti-VEGF therapy) to a site containing cells that express or overexpress EGFR. The invention also relates to use of a ligand that has binding specificity for VEGF and for EGFR for the manufacture of a medicament for delivering anti-angiogenic therapy (anti-VEGF therapy) to a site containing cells that express or overexpress EGFR, or for inhibiting angiogenesis at a site containing cells that express of overexpress EGFR.

In particular embodiments, the invention relates to a method of treating cancer comprising administering to a subject in need thereof a therapeutically effective amount of a ligand, as described herein, that has binding specificity for VEGF and for EGFR. In particular embodiments, the patient has an EGFR-expressing cancer, such as, bladder cancer, ovarian cancer, colorectal cancer, breast cancer, lung cancer (e.g., non-small cell lung carcinoma), gastric cancer, pancreatic cancer, prostate cancer, head and neck cancer, renal cancer and gall bladder cancer.

In other embodiments, the invention relates to a method for treating cancer, comprising administering to a subject in need thereof a therapeutically effective amount of ligand, as described herein, (e.g., a ligand that has binding specificity for VEGF, a ligand that has binding specificity for EGFR, a ligand that has binding specificity for VEGF and EGFR) and an anti-neoplastic composition, wherein said anti-neoplastic composition comprises at least one chemotherapeutic agent selected from the group consisting of alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitor, interferons, platinum cooridnation complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. In some embodiments, the chemotherapeutic agent is selected from the group consisting of cisplatin, dicarbazine, dactinomycin, mechlorethamine, streptozocin, cyclophosphamide, capecitabine, carmustine, lomustine, doxorubicin, daunorubicin, procarbazine, mitomycin, cytarabine, etoposide, methotrexate, 5-fluorouracil, vinbiastine, vincristine, bleomycin, paclitaxel, docetaxel, doxetaxe, aldesleukin, asparaginase, busulfan, carboplatin, cladribine, dacarbazine, floxuridine, fludarabine, hydroxyurea, ifosfamide, interferon alpha, irinotecan, leuprolide, leucovorin, megestrol, melphalan, mercaptopurine, oxaliplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol, an additional growth factor receptor antagonist, and a combination of any of the foregoing.

### Assays for Evaluating Ligands

The ligands of the invention can be assayed using any suitable in vitro or in vivo assay. For example, using the receptor binding assays or bioassays described herein.

### Bioassay for VEGF Activity:

This bioassay measures the ability of ligands (e.g., dAbs) to neutralize the VEGF induced proliferation of HUVE (human vascular endothelial) cells. HUVE cells plated in 96-well plates are incubated for 72 hours with pre-equilibrated VEGF and dAb protein. Cell number is then measured using a cell viability dye.

The assay is performed as follows. HUVE cells are trypsinized from a sub-confluent 175 cm² flask. Medium is aspirated off, the cells are washed with 5 ml trypsin and then incubated with 2 ml trypsin at room temperature for 5 min. The cells are gently dislodged from the base of the flask by knocking against your hand. 8 ml of induction medium are then added to the flask, pipetting the cells to disperse any clumps. Viable cells are counted using trypan blue stain.

Cells are spun down and washed 2× in induction medium, spinning cells down and aspirating the medium after each wash. After the final aspiration the cells are diluted to 10⁵ cells/ml (in induction medium) and plated at 100 μl per well into a 96 well plate (10,000 cells/well). The plate is incubated for >2 h @ 37 C. to allow attachment of cells.

60 μl dAb protein and 60 μl induction media containing 40 ng/ml VEGF₁₆₅ (for a final concentration of 10 ng/ml) is added to a v-bottom 96-well plate and sealed with film. The dAb/VEGF mixture is then incubated at 37 C. for 0.5-1 hour.

The dAb/VEGF plate is removed from the incubator and 100 μl of solution added to each well of the HUVEC containing plate (final volume of 200 μl). This plate is then returned to the 37° C. incubator for a period of at least 72 hours.

Control wells include the following: wells containing cells, but no VEGF; wells containing cells, a positive control neutralizing anti-VEGF antibody and VEGF; and control wells containing cells and VEGF only.

Cell viability is assessed by adding 20 μl per well Celltiter96 reagent, and the plate is incubated at 37° C. for 2-4 h until a brown colour develops. The reaction is stopped by the addition of 20 μl per well of 10% (w/v) SDS. The absorbance is then read at 490 nm using a Wallac microplate reader.

The absorbance of the no VEGF control wells is subtracted from all other values. Absorbance is proportional to cell number. The control wells containing control anti-VEGF antibodies should also exhibit minimum cell proliferation. The wells containing VEGF only should exhibit maximum cell proliferation.

### EXAMPLES

### Example 1

### VEGF Receptor Binding Assays

VEGF is a specific mitogen for endothelial cells in vitro and a potent angiogenic factor in vivo, with high levels of the protein being expressed in various types of tumours. It is a 45 kDa glycoprotein that is active as a homodimer. Several isoforms have been described which occur through alternative mRNA splicing. Of these isoforms VEGF-121 and VEGF-165 appear to be the most abundant.

The specific action of VEGF on endothelial cells is mainly regulated by two types of receptor tyrosine kinases (RTK), VEGF R1 (Flt-1), and VEGF R2 (KDR/Flk-1). However, it appears that the VEGF activities such as mitogenicity, chemotaxis, and induction of morphological changes are mediated by VEGF R2, even though both receptors undergo phosphorylation upon binding of VEGF.

### VEGF Receptor 2 Binding Assay

This method describes a VEGF receptor binding assay for measuring the ability of ligands (e.g., dAbs) to prevent binding of VEGF-165 to VEGF Receptor 2.

A recombinant human VEGF R2/Fc chimera was used in this assay, comprising the extracellular domain of human VEGF R2 fused to the Fc region of human IgG₁. Briefly, the receptor was captured on an ELISA plate, then the plate was blocked to prevent nonspecific binding. A mixture of VEGF-165 and ligand was then added, the plate was washed and receptor bound VEGF-165 detected using a biotinylated anti-VEGF antibody and an Horse-radish Peroxidase (HRP) conjugated anti-biotin antibody. The plate was developed using a colorimetric substrate and the OD read at 450 nm. If the dAb blocked VEGF binding to the receptor then no colour was detected.

The assay was performed as follows. A 96 well Nunc Maxisorp assay plate was coated overnight at 4° C. with 100 μl per well of recombinant human VEGF R2/Fc (R&D Systems, Cat. No: 357-KD-050) at a concentration of 0.5 μg/ml in carbonate buffer. Wells were washed 3 times with 0.05% Tween/PBS and 3 times with PBS. 200 μl per well of 2% BSA in PBS was added to block the plate and the plate was incubated for a minimum of 1 hour at room temperature.

Wells were washed (as above), then 50 μl per well of ligand was added to each well. 50 μl of VEGF, at a concentration of 6 ng/ml in diluent (for a final concentration of 3 ng/ml), was then added to each well and the plate was incubated for 2 hours at room temperature (for assay of supernatants; 80 μl of supernatant was added to each well then 20 μl of VEGF at a concentration of 15 ng/ml).

The following controls were included: 0 ng/ml VEGF (diluent only); 3 ng/ml VEGF (R&D Systems, Cat No: 293-VE-050); 3 ng/ml VEGF with 0.1 μg/ml anti-VEGF neutralizing antibody (R&D Systems cat#MAB293).

The plate was washed (as above) and then 100 μl biotinylated anti-VEGF antibody (R&D Systems, Cat No: BAF293), 0.5 μg/ml in diluent, was added and incubated for 2 hours at room temperature.

Wells were washed (as above) then 100 μl HRP conjugated anti-biotin antibody (1:5000 dilution in diluent; Stratech, Cat No: 200-032-096) was added. The plate was then incubated for 1 hour at room temperature.

The plate was washed (as above) ensuring any traces of Tween-20 were removed to limit background in the subsequent peroxidase assay and to help the prevention of bubbles in the assay plate wells that might give inaccurate OD readings.

100 μl of SureBlue 1-Component TMB MicroWell Peroxidase solution was added to each well, and the plate was left at room temperature for up to 20 minutes. A deep blue soluble product developed as bound HRP labelled conjugate reacted with the substrate. The reaction was stopped by the addition of 100 μl 1M hydrochloric acid (the blue colour turned yellow). The OD, at 450 nm, of the plate was read in a 96-well plate reader within 30 minutes of acid addition. The OD450 nm is proportional to the amount of bound streptavidin-HRP conjugate.

For some assays protein L was added. Protein L cross links two dAb monomers.

Expected results from the controls are as follows: 0 ng/ml VEGF should give a low signal of <0.15 OD; 3 ng/ml VEGF should give a signal of >0.5 OD; and 3 ng/ml VEGF pre-incubated with 0.1 μg/ml neutralizing antibody should give a signal <0.2 OD.

### VEGF Receptor 1 Binding Assay

This assay measures the binding of VEGF-165 to VEGF R1 and the ability of ligands to block this interaction. A recombinant human VEGF R1/Fc chimera was used here, comprising the extracellular domain of human VEGF R1 fused to the Fc region of human IgG₁. The receptor was captured on an ELISA plate then the plate was blocked to prevent non-specific binding. A mixture of VEGF-165 and ligand was then added, the plate was washed and receptor bound VEGF-165 detected using a biotinylated anti-VEGF antibody and an HRP conjugated anti-biotin antibody. The plate was developed using a colorimetric substrate and the OD read at 450 nm.

The assay was performed as follows. A 96-well Nunc Maxisorp assay plate was coated overnight at 4° C. with 100 μl per well of recombinant human VEGF R1/Fc (R&D Systems, Cat No: 321-FL-050) @ 0.1 μg/ml in carbonate buffer. Wells were washed 3 times with 0.05% Tween/PBS and 3 times with PBS.

200 μl per well of 2% BSA in PBS was added to block the plate and the plate was incubated for a minimum of 1 hour at room temperature.

Wells were washed (as above), then 50 μl per well of purified dAb protein was added to each well. 50 μl of VEGF, at a concentration of 1 ng/ml in diluent (for a final concentration of 500 pg/ml), was then added to each well and the plate incubated for 1 hour at room temperature (assay of supernatants; 80 μl of supernatant was added to each well then 20 μl of VEGF @ 2.5 ng/ml).

The following controls were included: 0 ng/ml VEGF (diluent only); 500 pg/ml VEGF; and 500 pg/ml VEGF with 1 μg/ml anti-VEGF antibody (R&D Systems cat#MAB293).

The plate was washed (as above) and then 100 μl biotinylated anti-VEGF antibody, 50 ng/ml in diluent, was added and incubated for 1 hour at room temperature.

Wells were washed (as above) then 100 μl HRP conjugated anti-biotin antibody was added (1:5000 dilution in diluent). The plate was then incubated for 1 hour at room temperature.

The plate was washed (as above), ensuring any traces of Tween-20 were removed to limit background in the subsequent peroxidase assay and to help the prevention of bubbles in the assay plate wells that might give inaccurate OD readings.

100 μl of SureBlue 1-Component TMS MicroWell Peroxidase solution was added to each well, and the plate was left at room temperature for up to 20 minutes. A deep blue soluble product developed as bound HRP labelled conjugate reacted with the substrate. The reaction was stopped by the addition of 100 μl 1M hydrochloric acid. The OD, at 450 nm, of the plate was read in a 96-well plate reader within 30 minutes of acid addition. The OD450 nm is proportional to the amount of bound streptavidin-HRP conjugate.

Expected result from the controls: 0 ng/ml VEGF should give a low signal of <0.15 OD; 500 pg/ml VEGF should give a signal of >0.8 OD; and 500 pg/ml VEGF pre-incubated with 1 μg/ml neutralizing antibody should give a signal <0.3 OD

TABLE 1

| | | RBA (VEGFR2) | RBA (VEGFR2) |
|---|---|---|---|
| | | IC50 − | IC50 + |
| | | protein L | protein L |
| dAb | | (nM) | (nM) |
| TAR15-1 | VK | 171 | 7.4 |
| TAR15-10 | VK | 12.2 | 0.3 |
| TAR15-16 | VK | 31 | 1.7 |
| TAR15-17 | VK | 38 | 0.5 |
| TAR15-18 | VK | 174 | 0.4 |
| TAR15-20 | VK | 28 | 0.3 |

The TAR15-1 had a Kd of 50-80 nM when tested at various concentrations on a low density BIAcore chip. Other VK dAbs were passed over the low density chip at one concentration (50 nM). Different dAbs showed different binding kinetics.

TABLE 2

| | | More than 50% reduction in |
|---|---|---|
| dAb | | supernatant RBA (VEGFR2)* |
| TAR15-5 | VH | + |
| TAR15-6 | VH | + |
| TAR15-7 | VH | + |
| TAR15-8 | VH | + |
| TAR15-23 | VH | + |
| TAR15-24 | VH | + |
| TAR15-25 | VH | + |
| TAR15-26 | VH | + |
| TAR15-27 | VH | + |
| TAR15-29 | VH | + |
| TAR15-30 | VH | + |

*dAb was assayed at 50 nM

VH dAbs were passed over the low density VEGF chip on a BIAcore at one concentration (50 nM). Different dAbs showed different binding kinetics.

### Example 2

### EGFR Binding

### EGFR Binding Assay

25 ul of ligand (e.g., dAb) were plated into a 96-well plate and then 25 ul streptavidin-Alexa Fluor (1 ug/ml) (Molecular Probes) and 25 ul A431 cells (ATCC No. CRL-1555) (8×10⁵/ml) were added. All reagents were prepared in PBS/1% BSA. The plate was incubated for 30 minutes at room temperature.

Without disturbing the cells, 25 ul biotinylated EGF (Invitrogen) at 40 ng/ml was added to each well, and the plate was incubated for three hours at room temperature. Fluoresecence was measured using the AB8200 Cellular Detection System (Applied Biosystems).

Ligands (e.g., dAbs) that inhibited the binding of biotinylated EGF to EGFR expressed on A431 cells resulted in lower fluorescence counts. Wells without ligand provided a reference of the maximum fluorescence (i.e., biotinylated EGF binding) and wells without ligand or biotinylated EGF provided a reference of the background level of fluorescence. These controls were included in all assays.

Results obtained in this assay using certain anti-EGFR dAbs are presented in the Table 3.

### EGFR Kinase Assay

In a 96-well plate, 5×10⁴ A431 cells (ATCC No. CRL-1555) were plated per well in RPMI-1640 supplemented with 10% foetal calf serum. The plate was incubated overnight at 37° C./5% CO₂ to allow the cells to adhere, then the medium was replaced with RPMI-1640. The plate was incubated for 4 hours at 37° C./5% CO₂. The ligand (prepared in RPMI-1640) was added to the wells and the plate was incubated for 45 minutes at 37° C./5% CO₂. EGF (Invitrogen) was added to the wells to give a final concentration of 100 ng/ml and the plate was incubated for 10 minutes at room temperature. The wells were washed twice with ice cold PBS. Cold lysis buffer (1% NP-40, 20 mM Tris, 137 mM NaCl, 10% glycerol, 2 mM EDTA, 1 mM sodium orthovanadate, 10 ug/ml aprotinin, 10 ug/ml leupeptin) was added and the plate was incubated on ice for 10 minutes.

The supernatants were transferred to an ELISA plate which had been coated overnight with anti-EGFR antibody (R&D Systems) at 1 ug/ml in carbonate buffer. The ELISA plate was incubated for 2 hours at room temperature. The plate was washed three times with PBS/0.05% Tween 20. Anti-phosphotyrosine antibody conjugated to horse-radish peroxidase (Upstate Biotechnology) at 1 ug/ml was added and the plate was incubated for 1 hour at room temperature. The plate was washed three times with PBS/Tween and three times with PBS. The reaction was developed with SureBlue TMB 1-component microwell peroxidase substrate (KPL) and the reaction was stopped with 1M HCl after 25 minutes. The absorbance was read using a Wallac plate reader.

Results obtained in this assay using certain anti-EGFR dAbs are presented in the Table 3.

TABLE 3

| | | Receptor Binding | | | |
|---|---|---|---|---|---|
| | KD | Assay | | Kinase Assay | |
| dAb | (nM) | IC50 (nM)* | | IC50 (nM)* | |
| DOM16-39 | 27.3 | 28.68 to 112.6 | (56.84) | 31.16 to 100.9 | (56.07) |
| DOM16-200 | 15.3 | 12.47 to 37.88 | (21.74) | 30.29 to 111.9 | (58.21) |
| DOM16-39-87 | 6.81 | 4.471 to 10.39 | (6.8) | 11.95 to 252.4 | (54.92) |
| DOM16-39-100 | 1.24 | 1.007 to 2.757 | (1.67) | 9.142 t0 17.56 | (12.67) |
| DOM16-39-107 | 7.09 | 1.472 to 4.208 | (2.49) | 12.00 to 34.99 | (20.49) |
| DOM16-39-109 | 1.01 | 0.746 to 1.472 | (1.05) | 6.817 to 11.08 | (8.69) |
| DOM16-39-115 | 6.90 | 1.085 to 6.886 | (2.73) | 21.52 to 83.34 | (42.35) |
| ERBITUX | — | 1.422 to 5.388 | (2.77) | 3.875 to 7.689 | (5.46) |
| (cetuximab, Imclone | | | | | |
| Systems, Inc.) | | | | | |

*the data presented are the lowest to highest values obtained and the (average)

### Example 3

### IgG-Like Formats that have Binding Specificity for VEGF and EGFR

### Vectors

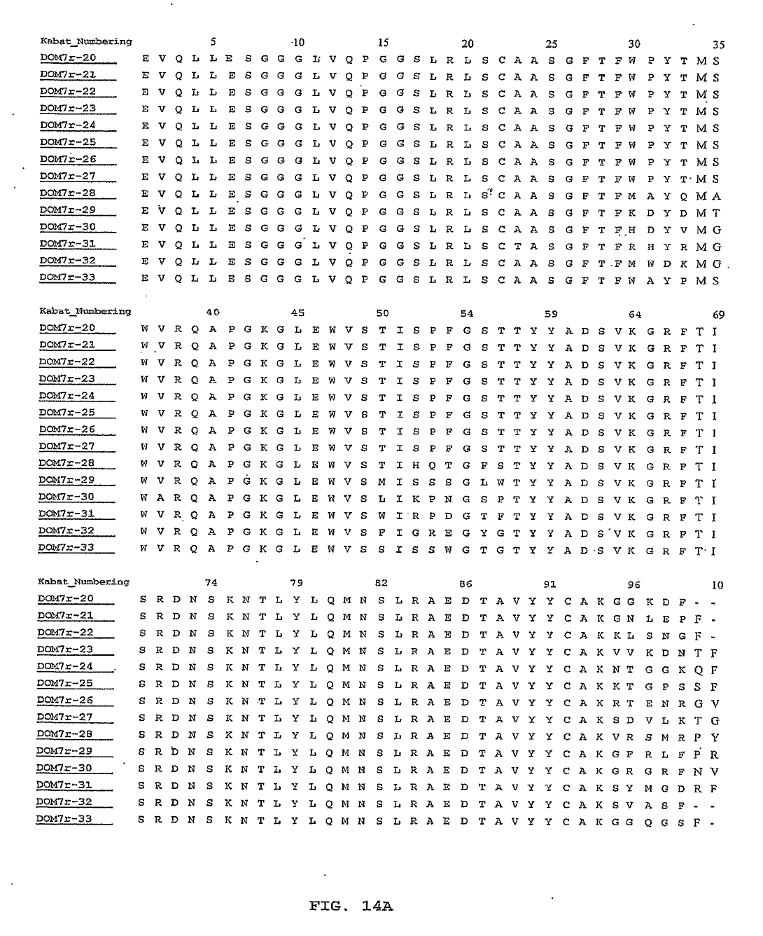

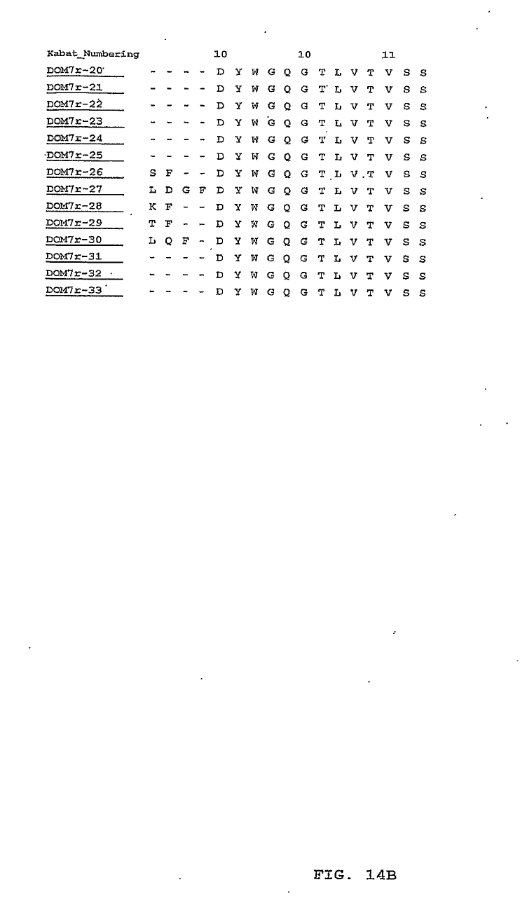

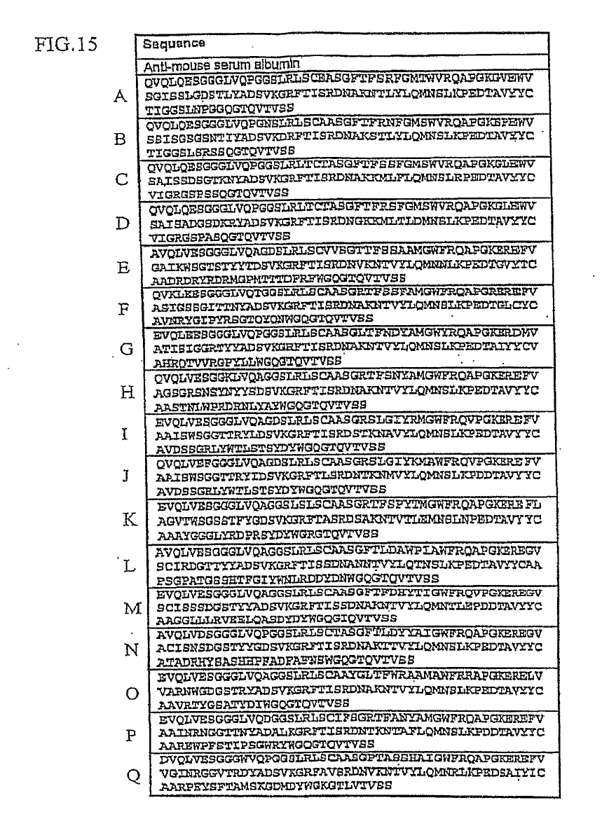

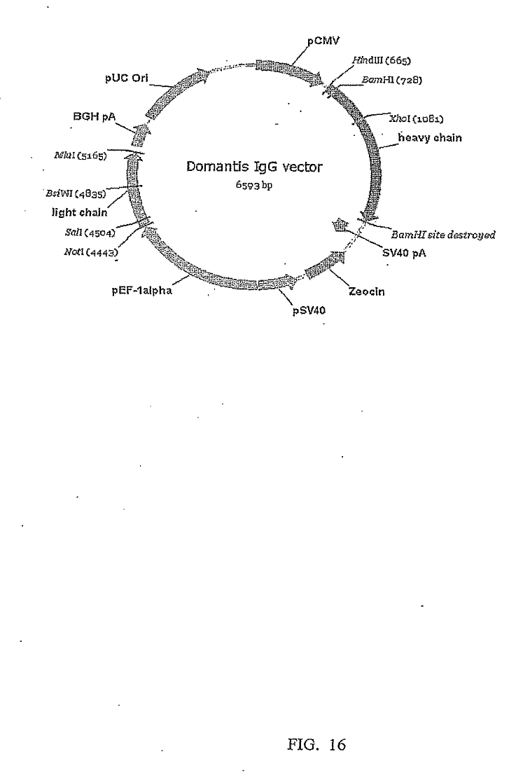

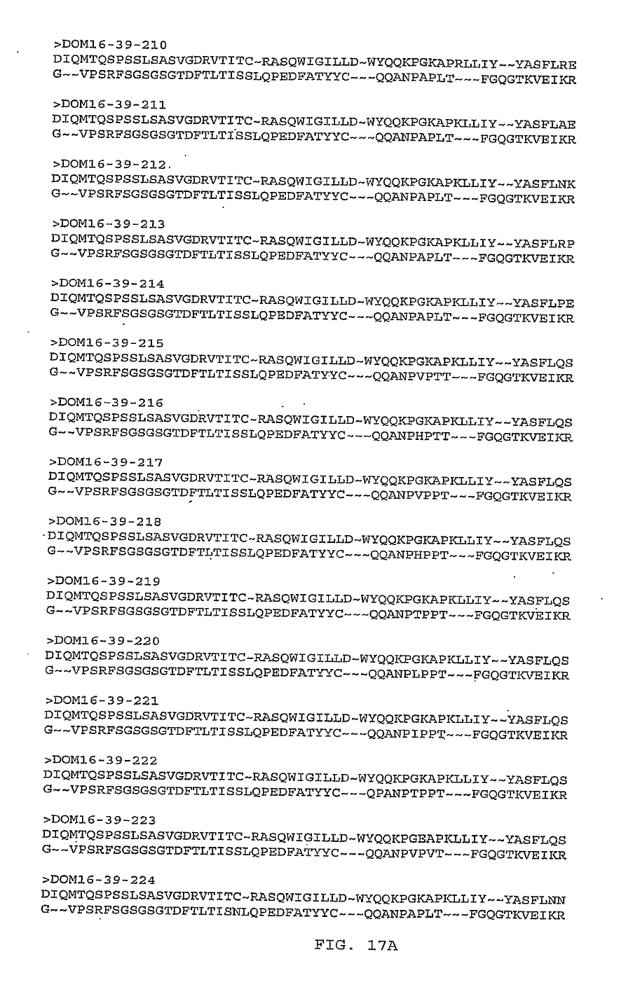

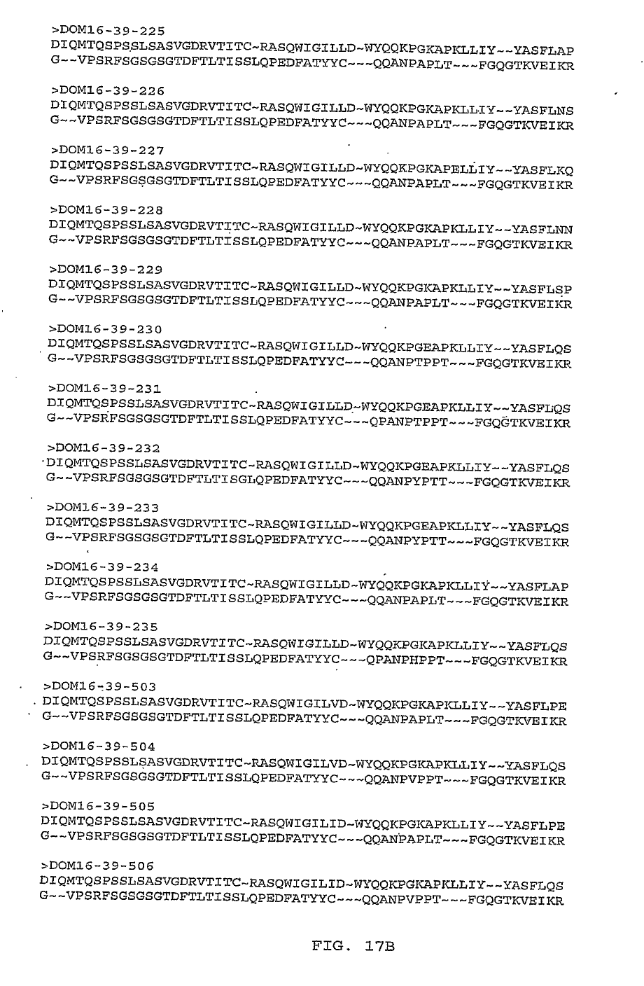

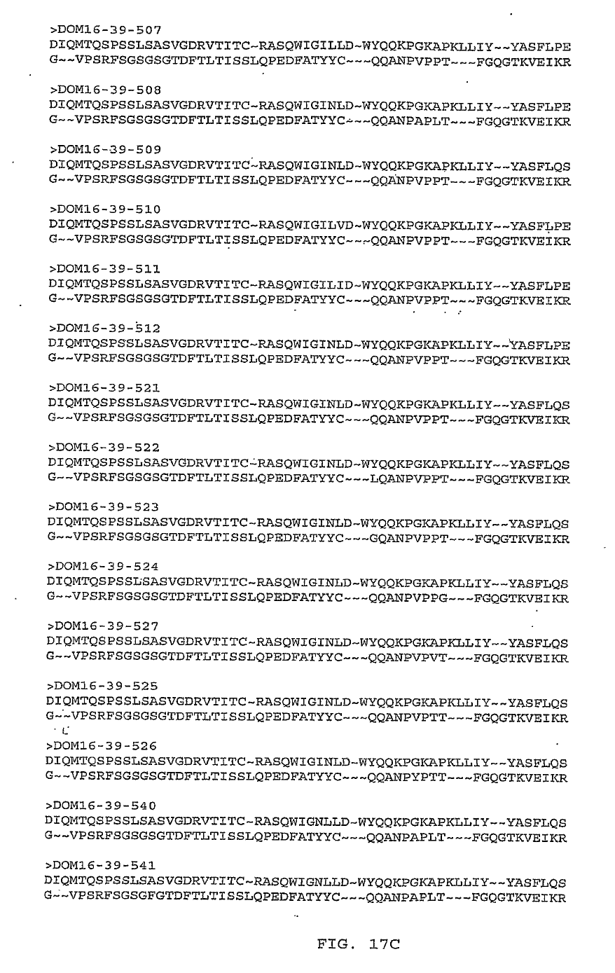

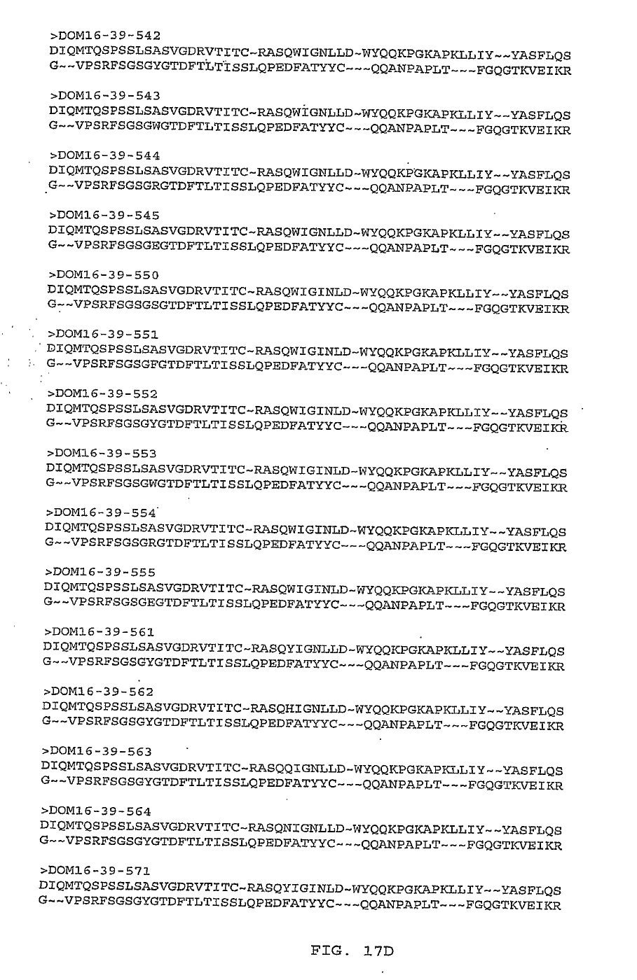

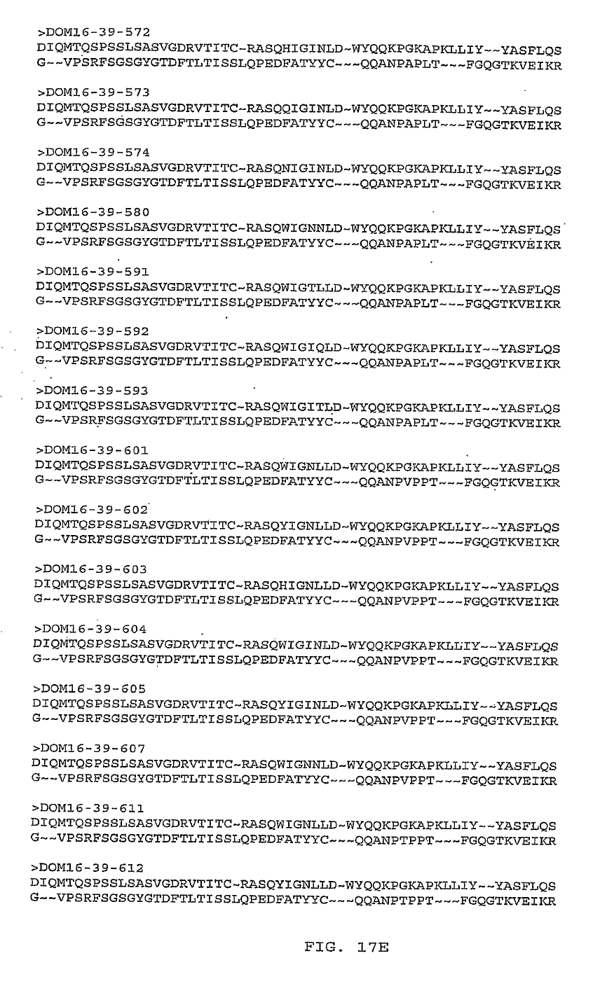

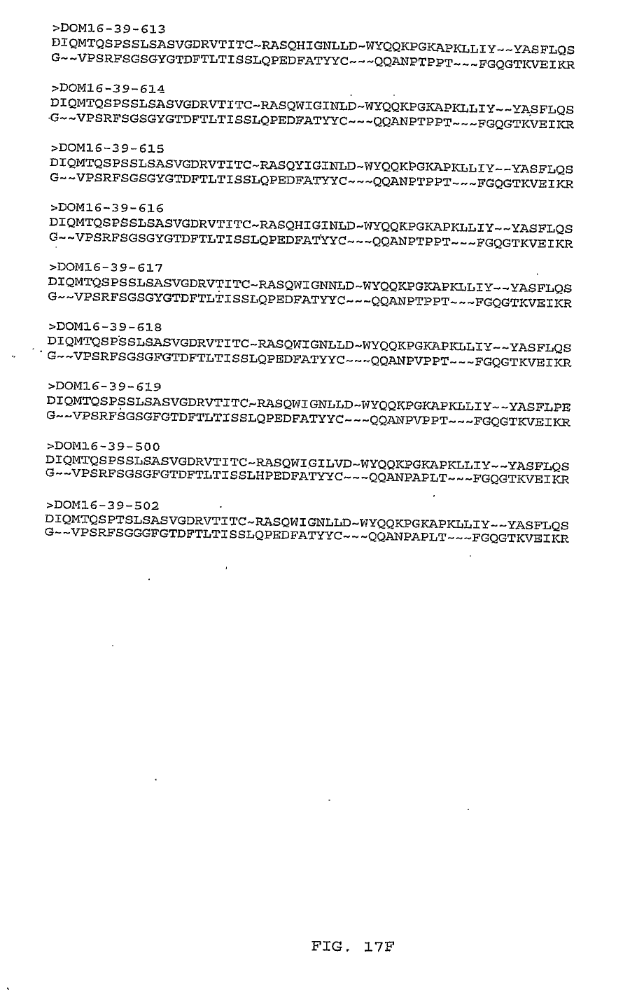

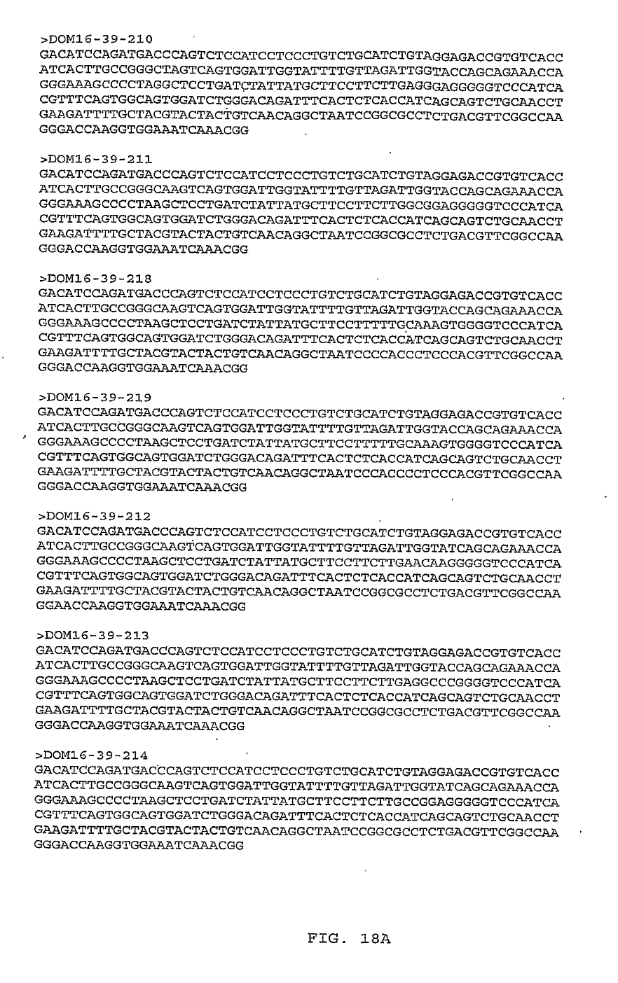

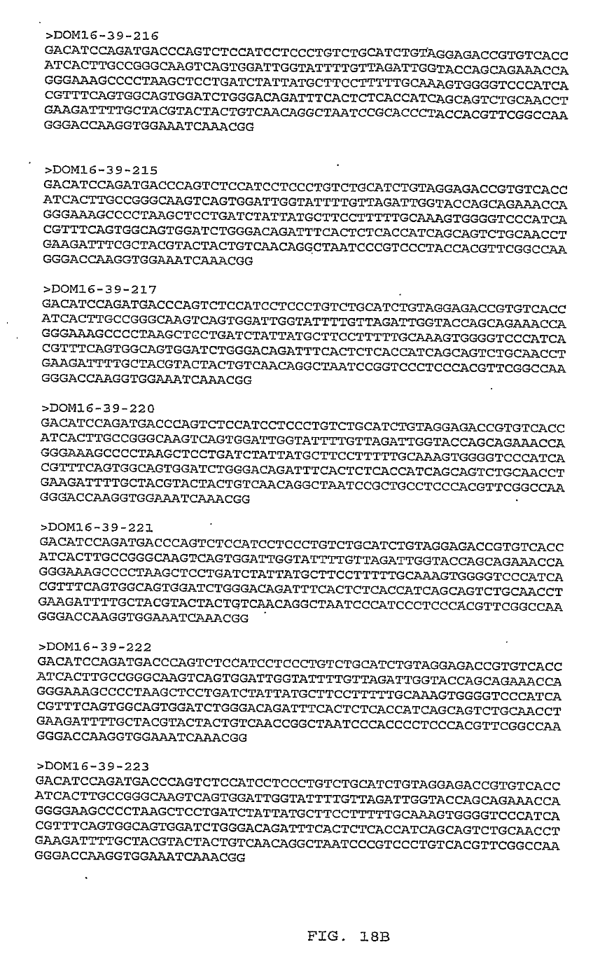

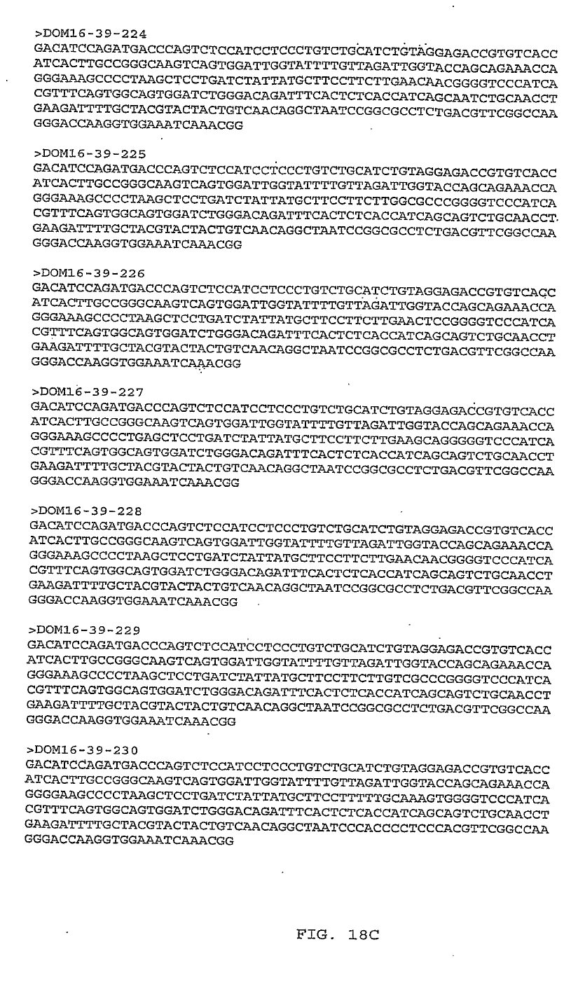

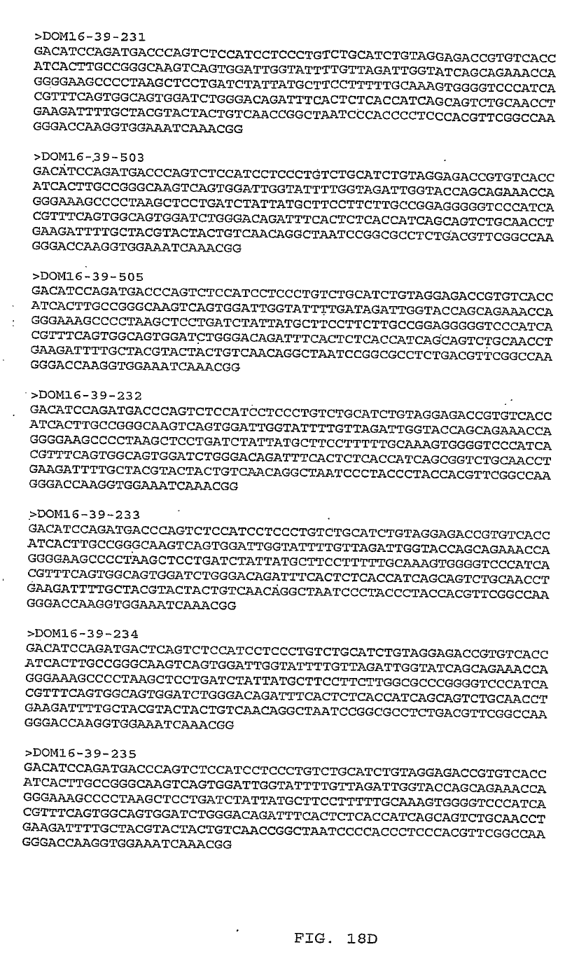

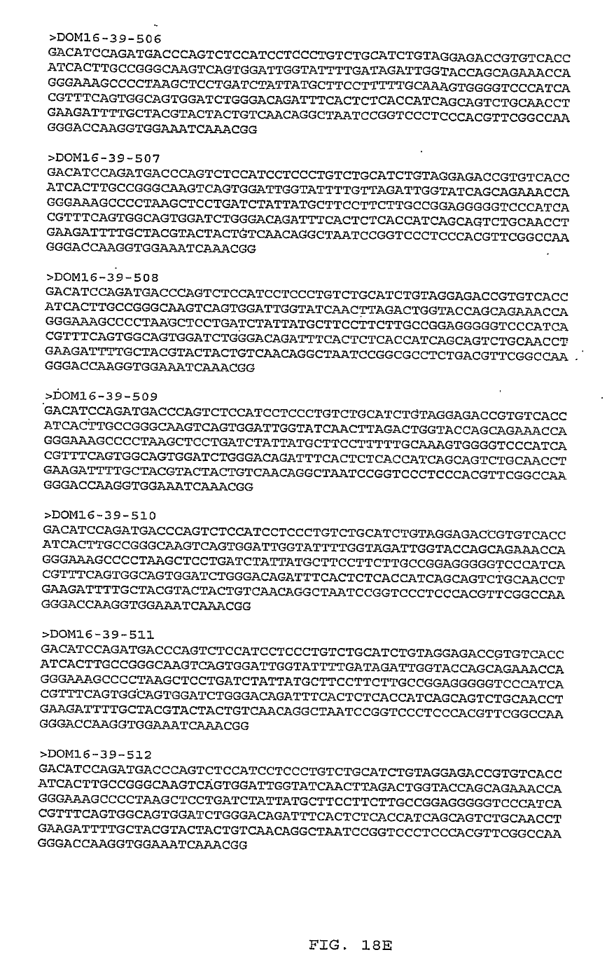

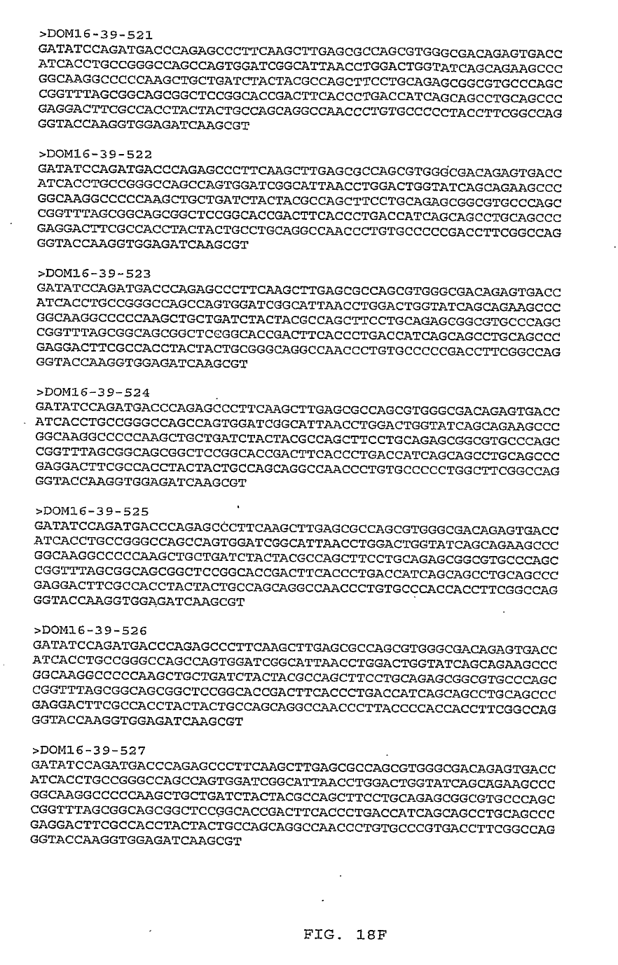

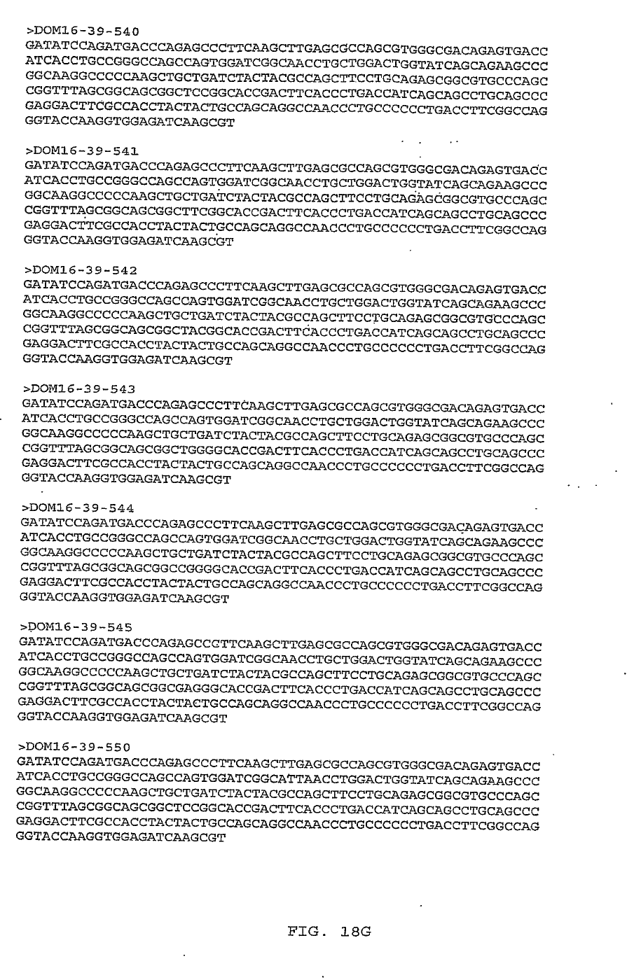

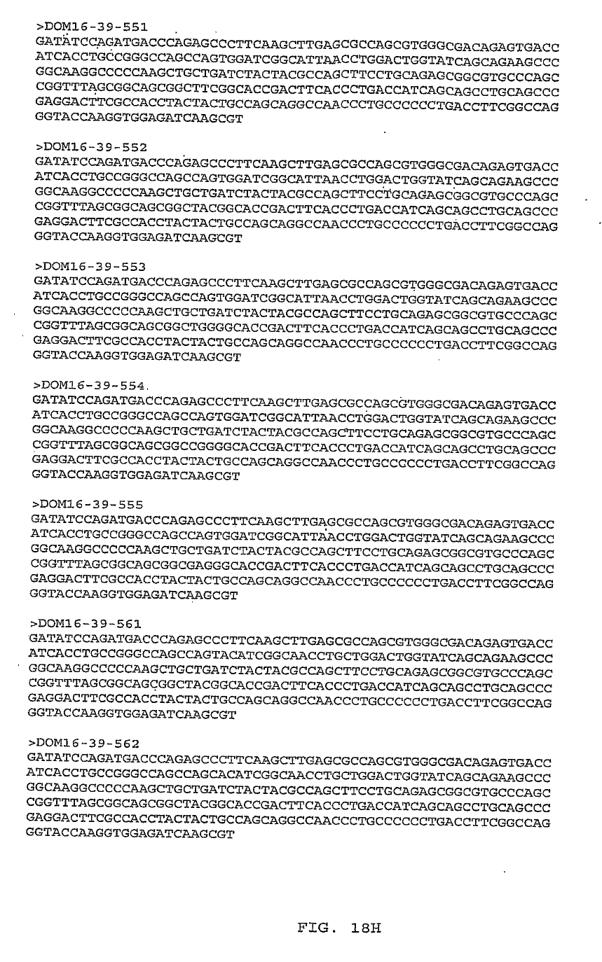

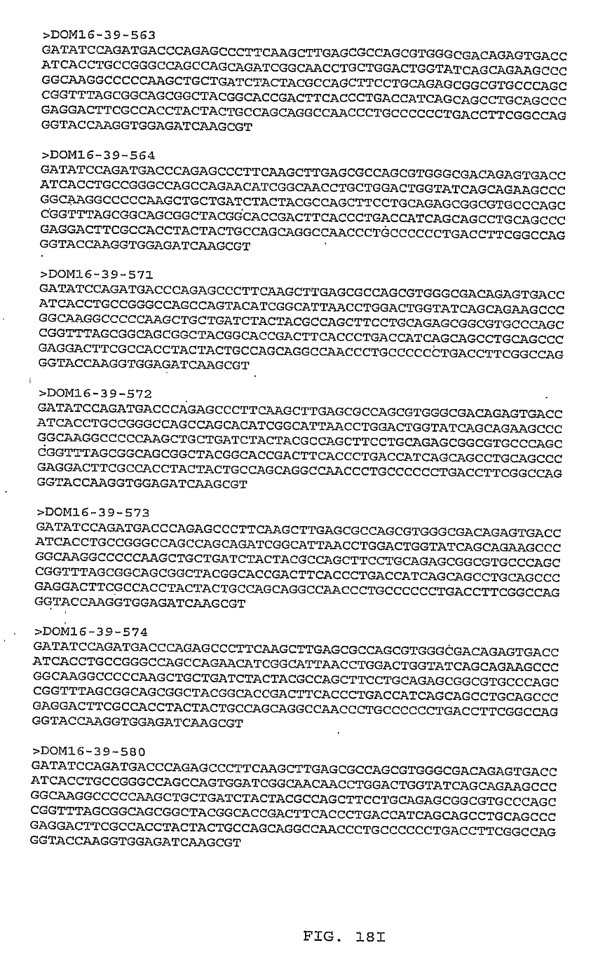

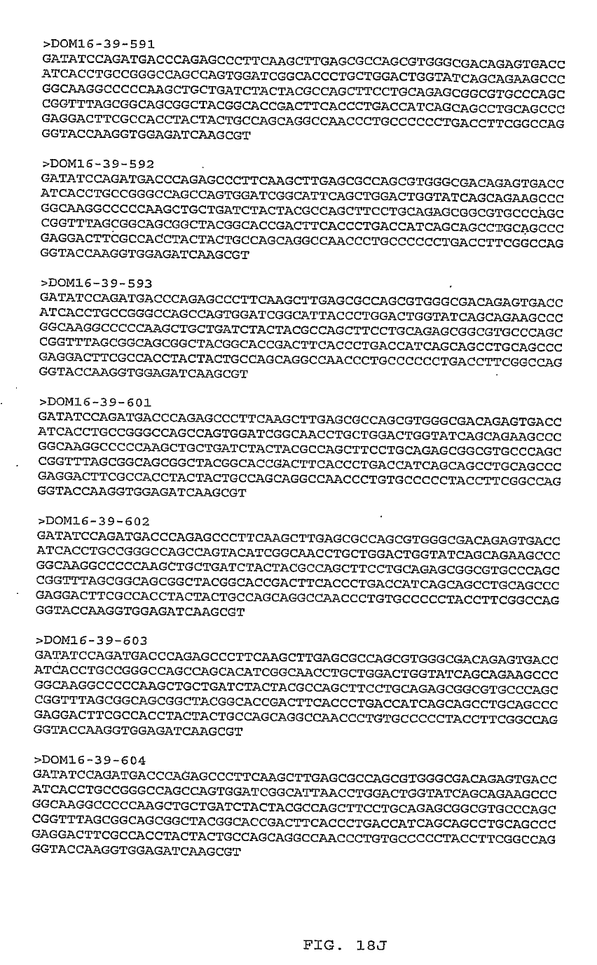

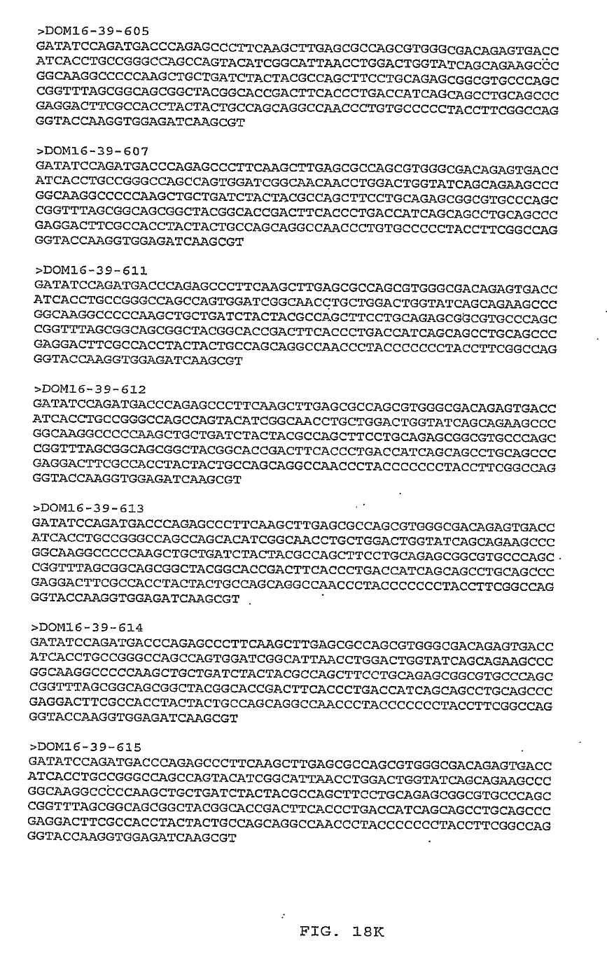

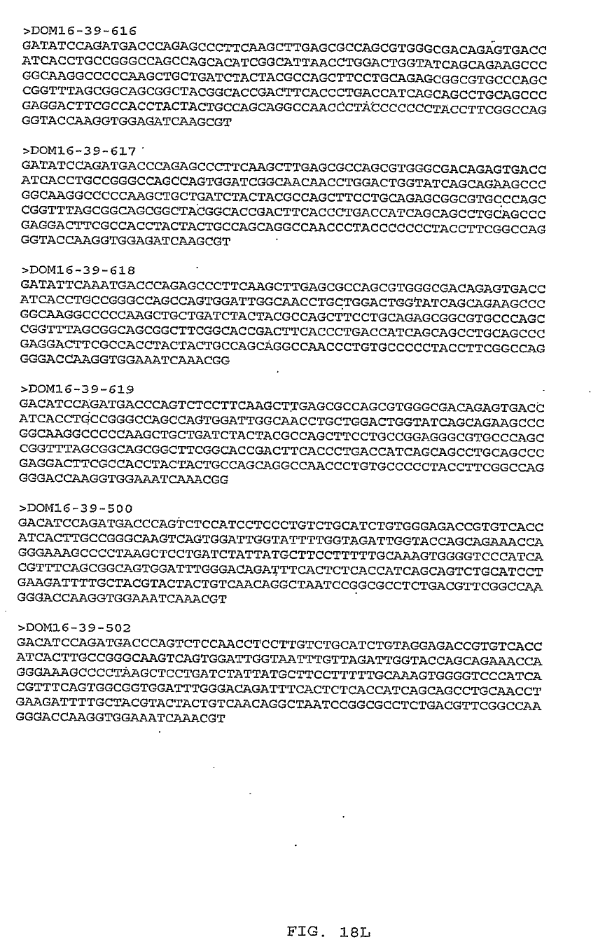

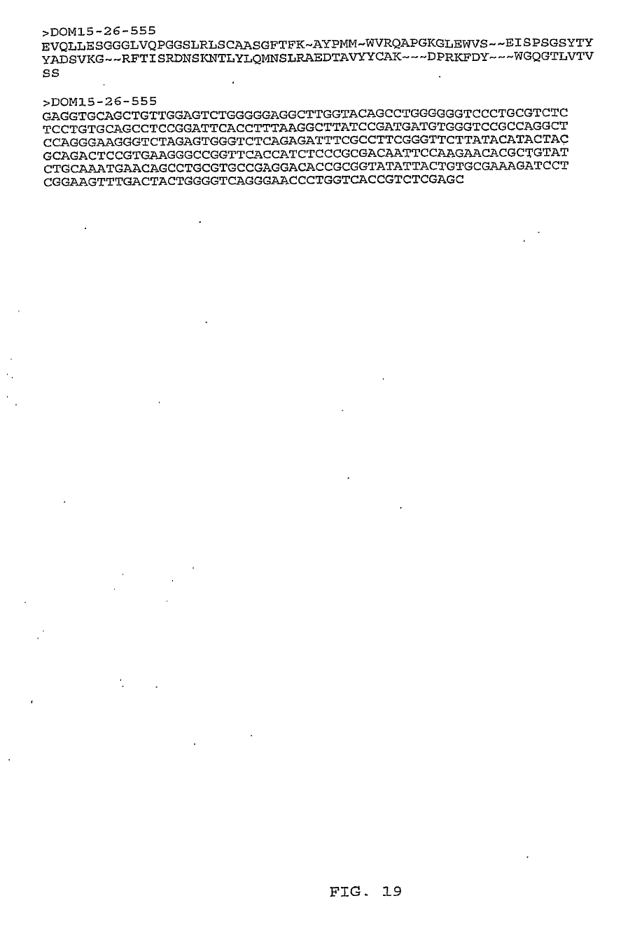

The pBudCE4.1 backbone (Invitrogen) was used for cloning immunoglobulin constant regions, such as the IgG1 heavy chain constant region and light chain kappa constant region (see FIG. 16 for overview). An Ig Kappa chain leader was used to facilitate secretion of the expressed protein. Ig constant regions (human IgG1 and CK) were produced by GeneArt (Germany).

The heavy chain constant region and signal peptide were cloned into pBudCE4.1 as a Hind III/BgIII fragment into the HindIII/BamH I restriction sites.

The light chain constant region and signal peptide were cloned into pBudCE4.1 as a NotI/MluI fragment.

### Cloning of dAb in IgG Vectors and Production of IgG-Like Format

VK dAb (specific to VEGF or EGFR) was cloned into IgG vector as a SalI/BsiWI fragment. VH dAb (specific to VEGF or EGFR was cloned into IgG vector as a BamHI/XhoI fragment.

The plasmid was then transfected into HEK293T cells (ATCC CRL-11268) and IgG was expressed transiently for five days. The IgG produced was purified using streamline Protein A.

Purified IgG was checked on a reducing and non-reducing SDS gel and bands of expected size were observed.

Several dAbs that bind VEGF or EGFR were formatted into IgG-like formats that have binding specificity for VEGF and EGFR. The IgG-like formats were prepared by producing constructs that encoded an IgG heavy chain wherein VH is a dAb, and a Kappa light chain wherein VK is a dAb. The IgG-like formats that were prepared are shown in Table 4, and the results obtained for some of the IgG-like formats in assays are presented in Table 5. (Dummy VH and Dummy VK are germ line sequences that do not bind VEGF or EGFR).

TABLE 4

| IgG-like | Heavy Chain | Light Chain |
|---|---|---|
| format No. | V region | V Region |
| 1 | DOM16-39 VK | DOM16-39 VK |
| 2 | DOM16-32 VK | DOM15-10 VK |
| 3 | DOM16-39 VK | DOM15-10 VK |
| 4 | DOM16-72 VK | DOM15-10 VK |
| 5 | DOM15-26 VH | DOM16-32 VK |
| 6 | DOM15-26 VH | DOM16-39 VK |
| 7 | DOM15-26 VH | DOM16-72 VK |
| 8 | DOM15-26 VH | DOM15-10 VK |
| 9 | DOM16-52 VH | DOM15-10 VK |
| 10 | DOM15-26 VH | DOM16-52 VH |
| 11 | DOM15-10 VK | DOM15-26 VH |
| 12 | DOM15-10 VK | DOM15-10 VK |
| 13 | DOM16-200 VK | DOM16-200 VK |
| 14 | DOM15-10 VK | DOM16-200 VK |
| 15 | DOM15-10 VK | DOM16-32 VK |
| 16 | DOM15-10 VK | DOM16-72 VK |
| 17 | DOM15-10 VK | DOM16-39 VK |
| 18 | DOM15-26 VH | DOM16-200 VK |
| 19 | DOM15-26 VH | Dummy VK |
| 20 | DOM15-26 VH | dCDR1/DOM16-200 VK |
| 21 | DOM15-26 VH | dCDR2/DOM16-200 VK |
| 22 | DOM15-26 VH | dCDR3/DOM16-200 VK |
| 23 | DOM15-26-501 VH | DOM16-200 VK |
| 24 | DOM15-6-506VH | DOM16-200 VK |
| 25 | DOM15-8-505 VH | DOM16-200 VK |
| 26 | DOM15-26 VH | DOM15-26 VH |
| 27 | DOM15-26-534 VH | DOM16-200 VK |
| 28 | DOM15-26-501 VH | DOM16-39-500 VK |
| 29 | DOM15-26-501 VH | DOM16-39-201 VK |
| 29a | DOM15-26-501 VH | DOM16-39-501 VK |
| 30 | DOM15-26-501 VH | DOM16-39-502 VK |
| 31 | DOM15-26-534 VH | DOM16-39-501 VK |
| 32 | Dummy VH | DOM16-200 VK |
| 33 | Dummy VH | DOM16-39-201 VK |
| 34 | DOM15-26-501 VH | DOM16-39-204 VK |
| 35 | DOM15-26-501 VH | DOM16-39-206 VK |
| 36 | DOM15-26-501 VH | DOM16-39-207 VK |
| 37 | DOM15-26-501 VH | DOM16-39-209 VK |
| 38 | DOM15-26-501 VH | DOM16-39-203 VK |
| 39 | DOM15-26-501 VH | DOM16-39-214 VK |
| 40 | DOM15-26-501 VH | DOM16-39-217 VK |
| 41 | DOM15-26-501 VH | Dummy VK VK |

TABLE 5

| | | | VEGF | | EGFR | EGFR |
|---|---|---|---|---|---|---|
| | | | (bio- | VEGF | (cell | (Kinase |
| | | | assay) | (RBA) | RBA) | assay) |
| | | | ND50 | IC50 | EC50 | ND50 |
| IgG | CH | CK | (nM) | (nM) | (nM) | (nM) |
| 1 | DOM16-39 | DOM16-39 | | | 17 | 22 |
| | VK | VK | | | | |
| 3 | DOM16-39 | DOM15-10 | | 1.5 | | |
| | VK | VK | | | | |
| 4 | DOM16-72 | DOM15-10 | | | | |
| | VK | VK | | | | |
| 6 | DOM15-26 | DOM16-39 | 1 | | 126 | 22 |
| | VH | VK | | | | |
| 10 | DOM15-26 | DOM16-52 | 0.2 | 0.05 | | |
| | VH | VH | | | | |
| 11 | DOM15-10 | DOM15-26 | | 0.03 | | |
| | VK | VH | | | | |
| 18 | DOM15-26 | DOM16-200 | | 0.4 | 5 | |
| | VH | VK | | | | |
| 19 | DOM15-26 | Dummy VK | | 4.8 | | |
| | VH | | | | | |
| 20 | DOM15-26 | dCDR1/ | | 4.1 | | |
| | VH | DOM16-200 | | | | |
| | | VK | | | | |
| 21 | DOM15-26 | dCDR2/ | | 0.1 | | |
| | VH | DOM16-200 | | | | |
| | | VK | | | | |
| 23 | DOM15-26- | DOM16-200 | 0.16 | 0.16 | 23 | |
| | 501 VH | VK | | | | |
| 24 | DOM15-6- | DOM16-200 | | 12 | 26 | |
| | 506VH | VK | | | | |
| 25 | DOM15-8- | DOM16-200 | | | 34 | |
| | 505 VH | VK | | | | |
| 27 | DOM15-26- | DOM16-200 | | 0.5 | 137 | |
| | 534 VH | VK | | | | |
| 28 | DOM15-26- | DOM16-39- | | 0.8 | 43 | |
| | 501 VH | 500 VK | | | | |
| 30 | DOM15-26- | DOM16-39- | | 0.2 | 17 | |
| | 501 VH | 502 VK | | | | |

### Example 4

### Dual Specific Inline Formats

Domain antibodies that bind VEGF or EGFR were incorporated into fusion polypeptides that contained an anti-VEGFR dAb and an anti-EGFR dAb in a single polypeptide chain. Some of the fusion polypeptides also included an antibody Fc region (—CH2—CH3 of human IgG1). Specific examples of the fusion polypeptides that were cloned and expressed include TAR15-10 fused to DOM16-39-206 and to Fc (SEQ ID NO:715); DOM16-39-206 fused to TAR15-10 and to Fc (SEQ ID NO:716); DOM16-39-206 fused to TAR15-26-501 and to Fc (SEQ ID NO:717); TAR15-26-501 fused to DOM16-39-206 and to Fc (SEQ ID NO:718); TAR15-10 fused to DOM16-39-206 (SEQ ID NO:719); DOM16-39-206 fused to TAR15-10 (SEQ ID NO:720); DOM16-39-206 fused to TAR15-26-501 (SEQ ID NO:721); and TAR15-26-501 fused to DOM16-39-206 (SEQ ID NO:722). The positions of the foregoing fusions are listed as they appear in the fusion proteins from amino terminus to carboxy terminus.

DNA encoding dAbs was PCR amplified and cloned into expression vectors using standard methods. Inline fusion polypeptides were produced by expressing the expression vectors in Pichia (fusion that did not contain an Fc region) or in HEK 293T cells (Fc region containing fusions). Inline fusions were batch bound and affinity purified on streamline protein A and streamline protein L resins for HEK 293T cells (Fc-tagged) and Pichia expressed constructs respectively.

The portions of several fusions that contain Fc are listed in Table 6 as they appear in the fusion proteins, from amino terminus to carboxy terminus. Accordingly, the structure of the fusion proteins can be appreciated by reading the table from left to right. The first fusion protein presented in Table 6 has the structure, from amino terminus to carboxy terminus, DOM15-10-Linker 1-DOM16-39-206-Linker 2-Fc. Binding activities of the fusions were assessed using the EGFR binding assay described in Example 2, and the VEGF receptor 2 binding assay described in Example 1.

General robustness and resistance to degradation were tested by subjecting the inline fusions to proteolysis with trypsin. A solution of dual specific ligand and trypsin (1/25 (w/w) trypsin to ligand) was prepared and incubated at 30° C. Samples were taken at 0 minutes (i.e., before addition of trypsin), 60 minutes, 180 minutes, and 24 hours. At the given time points, the reaction was stopped by the addition of complete protease inhibitor cocktail at 2× final concentration (Roche code: 11 836 145 001) with PAGE loading dye, followed by flash freezing the samples in liquid nitrogen. Samples were analyzed by SDS-PAGE, and protein bands were visualized to reveal a time course for the protease degradation of the fusions.

These experiments showed that inline fusions having a “natural” linker (KVEIKRTVAAPS (SEQ ID NO:706), which contains the carboxy-terminal amino acids of Vk and amino-terminal amino acids of Ck, were susceptible to rapid proteolysis, with degradation evident as soon as the 10 minute time point. SDS-PAGE analysis revealed that degradation occurred at the linkers between dAbs and at the linkers between dAb and Fc.

New linkers were designed that contain fewer Lys and Arg residues, which are cleavage points for trypsin and are abundant in the natural linker. Fusions that contained the engineered linkers (LVTVSSAST (SEQ ID NO:707)) or (LVTVSSGGGGSGGGS (SEQ ID NO:708)) showed much improved resistance to trypsin proteolysis.

Additional binding assays were performed to assess the potency of the inline fusions that contained the engineered linkers. The results revealed engineered linkers did not have any substantial adverse effect on potency.

TABLE 6
Fusion polypeptides that contain Fc

| | | | | Assay | Assay |
|---|---|---|---|---|---|
| | | | | dAb1 | dAb2 |
| dAb1 | Linker 1 | dAb2 | Linker 2 | (nM) | (nM) |
| DOM15- | KVEIKRTVAAPS | DOM16- | KVEIKRTVAAPS | 0.45 | 23.8 |
| 10 (V_{K}) | | 39-206 | | | |
| | | (V_{K}) | | | |
| DOM16- | KVEIKRTVAAPS | DOM15- | KVEIKRTVAAPS | 3.7 | 0.88 |
| 39-206 | | 10 (V_{K}) | | | |
| (V_{K}) | | | | | |
| DOM16- | KVEIKRTVAAPS | DOM15- | LVTVSSASTKGPS | 20.7 | 21.3 |
| 39-206 | | 26-501 | | | |
| (V_{K}) | | (V_{H}) | | | |
| DOM15- | LVTVSSASTKGPS | DOM16- | KVEIKRTVAAPS | 5.7 | 7.7 |
| 26-501 | | 39-206 | | | |
| (V_{H}) | | (V_{K}) | | | |
| DOM16- | LVTVSSAST | DOM15- | LVTVSSAST | 0.68 | 10.8 |
| 39-601 | | 10 (V_{K}) | | | |
| (V_{K}) | | | | | |
| DOM16- | KVEIKRTVAAPS | DOM15- | KVEIKRTVAAPS | 0.77 | 2.9 |
| 39-601 | | 10 (V_{K}) | | | |
| (V_{K}) | | | | | |
| DOM15- | LVTVSSAST | DOM16- | LVTVSSAST | 1.2 | 4.2 |
| 10 (V_{K}) | | 39-601 | | | |
| | | (V_{K}) | | | |
| DOM16- | LVTVSSGGGGSGGGS | DOM15- | LVTVSSGGGGSGGGS | 5.7 | 0.2 |
| 39-601 | | 10 (V_{K}) | | | |
| (V_{K}) | | | | | |
| DOM15- | LVTVSSGGGGSGGGS | DOM16- | LVTVSSGGGGSGGGS | 0.8 | 3.1 |
| 10 (V_{K}) | | 39-601 | | | |
| | | (V_{K}) | | | |
| DOM15- | KVEIKRTVAAPS | DOM16- | KVEIKRTVAAPS | 0.2 | 2.9 |
| 10 (V_{K}) | | 39-601 | | | |
| | | (V_{K}) | | | |

### Example 5

### Additional Engineered Linkers

Several designed mutations were introduced to the C-terminal region of Vk dAbs expressed on the light chain of IgG-like formats to reduce protease sensitivity. The “natural linker” was GQGTKVEIKRTVAAPS (SEQ ID NO:709 which contains the carboxy-terminal amino acids of Vk and amino-terminal amino acids of Ck). Variant linkers 1-3 were designed with amino acid replacements that replaced some or all of the positively charged residues in the natural linker with the most conservative substitutions that are not positively charged at physiological pH. It is likely that the arginine residue in the natural linker is less amenable to alteration due to ionic interactions it forms within the CL domain.

Variant linker 1 (GQGTNVEINRTVAAPS (SEQ ID NO:710)) substitutes both lysines in the natural linker with asparagines. Variant linker 1, and variant linker 2 (GQGTNVEINQTVAAPS (SEQ ID NO:711)), which additionally changes the arginine in the natural linker to glutamine, introduce an N-glycosylation site (N×T) into the linker. SDS-PAGE analysis of IgG-like formats containing variant linker 1 or variant linker 2 showed that the light chain had a higher molecular weight, consistent with an N-glycosylation event. Variant linker 3 (GQGTNVEIQRTVAAPS (SEQ ID NO:712) removes the N-glycosylation site while leaving the arginine in the natural linker in place. Variant linker 4 (GQGTLVTVSSTVAAPS (SEQ ID NO:713)) replaces the six C-terminal amino acids of the Vk domain with the corresponding residues from a VH domain, and is devoid of positive charges.

Protease resistance (trypsin resistance assessed as described in Example 4) of IgG-like formats that contain variant linkers 1-4 revealed that IgG-like formats that contained engineered variant linkers were more protease resistant than an IgG-like format that contained the natural linker.

### Example 6

### DOM16 dAb-Anti-Serum Albumin Dab Fusions

DOM16 dAb-anti-serum albumin dAb fusions were designed and expressed as a fusion of an anti-EGFR dAb to an anti-serum albumin dAb (a DOM7 dAb). The portions of the fusions are listed in Table 7 as they appear in the fusion proteins, from amino terminus to carboxy terminus. Accordingly, the structure of the fusion proteins can be appreciated by reading the table from left to right. The first fusion protein presented in Table 7 has the structure, from amino terminus to carboxy terminus, DOM16-39-618(S12P)-Linker-DOM7h-14. DOM16-39-618 contains a Serine to Proline mutation at position 12, which stops binding to protein L and prevents light chain aggregation. The iDOM7 dAbs are mutated so as to abrogate binding to albumin, and thus are inactivated.

TABLE 7

| N terminal dAb | Linker | C terminal dAb |
|---|---|---|
| DOM16-39-618 (S12P) | TVAAPS | DOM7h-14 |
| DOM16-39-618 (S12P) | TVAAPS | iDOM7h-14 |
| DOM7h-14 | TVAAPS | DOM16-39-618 (S12P) |
| DOM7h-14 | TVAAPS | DOM16-39-618 (S12P) |
| DOM16-39-618 (S12P) | TVAAPS | DOM7r-16 |
| DOM16-39-618 (S12P) | TVAAPS | iDOM7r-16 |
| DOM7r-16 | TVAAPS | DOM16-39-618 (S12P) |
| DOM7r-16 | TVAAPS | DOM16-39-618 (S12P) |

### Example 7

### EGFR Epitope Mapping

Epitope mapping was conducted in competitive binding assays using anti-EGFR dAbs, EGF and ERBITUX (cetuximab; Imclone Systems). The binding studies were performed using a BIAcore biosensor. DOM16-39-200 was used as a reference in this study. DOM16-39-200, and other dAbs designated DOM-16-39-x, are affinity matured variants of DOM16-39. Accordingly, all dAbs in the DOM-16-39 series will have substantially the same epitopic specificity, because affinity maturation produces dAbs that bind with higher affinity, but does not alter the specificity of the dAb.

The results showed that DOM16-72, DOM16-79 and DOM16-112 competed with DOM16-39-200 for binding to EGFR, indicating that these dAbs bind overlapping epitopes. However, DOM16-32, DOM16-52 and DOM16-80 were shown to bind to a different epitope. ERBITUX (cetuximab; Imclone Systems) is known to inhibit binding of EGF to EGFR (cetuximab and EGF bind overlapping epitopes on EGFR). The results of this study also demonstrated that DOM16-39-200 and cetuximab competed for binding to EGFR, indicating that the DOM16-39-200 epitope overlaps with the cetuximab epitope, and with the binding site for EGF.

While this invention has been particularly shown and described with references to preferred embodiments thereof, it will be understood by those skilled in the art that various changes in form and details may be made therein without departing from the scope of the invention encompassed by the appended claims.

1. A ligand that has binding specificity for vascular endothelial growth factor (VEGF) and epidermal growth factor receptor (EGFR), comprising at least one protein moiety that has a binding site with binding specificity for VEGF and at least one protein moiety that has a binding site with binding specificity for EGFR.

2. The ligand of claim 1, wherein each said protein moiety that has a binding site with binding specificity for EGFR competes for binding to EGFR with an anti-EGFR domain antibody (dAb) selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

3. The ligand of claim 1, wherein each said protein moiety that has a binding site with binding specificity for EGFR competes for binding to EGFR with an anti-EGFR domain antibody (dAb) selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

4. The ligand of claim 1, wherein each said protein moiety that has a binding site with binding specificity for VEGF competes for binding to VEGF with an anti-VEGF domain antibody (dAb) selected from the group consisting of TAR15-6 (SEQ ID NO:117), TAR15-8 (SEQ ID NO:119), and TAR15-26 (SEQ ID NO:123); and wherein each said protein moiety that has a binding site with binding specificity for EGFR competes for binding to EGFR with an anti-EGFR domain antibody (dAb) selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

5. The ligand of claim 1, wherein each said protein moiety that has a binding site with binding specificity for VEGF competes for binding to VEGF with an anti-VEGF domain antibody (dAb) selected from the group consisting of TAR15-6 (SEQ ID NO:117), TAR15-8 (SEQ ID NO:119), and TAR15-26 (SEQ ID NO:123); and wherein each said protein moiety that has a binding site with binding specificity for EGFR competes for binding to EGFR with an anti-EGFR domain antibody (dAb) selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

6. The ligand of claim 1, wherein each said protein moiety that has a binding site with binding specificity for VEGF competes for binding to VEGF with bevacizumab.

7. The ligand of claim 1, wherein each said protein moiety that has a binding site with binding specificity for EGFR competes for binding to EGFR with cetuximab.

8. The ligand of claim 1, wherein each said protein moiety that has a binding site with binding specificity for VEGF competes for binding to VEGF with bevacizumab; and wherein each said protein moiety that has a binding site with binding specificity for EGFR competes for binding to EGFR with cetuximab.

9. The ligand of claim 1, wherein each protein moiety that has a binding site with binding specificity for VEGF and each protein moiety that has a binding site with binding specificity for EGFR is provided by an antibody fragment.

10. The ligand of claim 9, wherein said antibody fragment is an immunoglobulin single variable domain.

11. A ligand that has binding specificity for vascular endothelial growth factor (VEGF) and epidermal growth factor receptor (EGFR), comprising at least one immunoglobulin single variable domain with binding specificity for VEGF and at least one immunoglobulin single variable domain with binding specificity for EGFR, wherein each said immunoglobulin single variable domain with binding specificity for EGFR competes for binding to EGFR with an anti-EGFR domain antibody (dAb) selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

12. A ligand that has binding specificity for vascular endothelial growth factor (VEGF) and epidermal growth factor receptor (EGFR), comprising at least one immunoglobulin single variable domain with binding specificity for VEGF and at least one immunoglobulin single variable domain with binding specificity for EGFR, wherein each said immunoglobulin single variable domain with binding specificity for EGFR competes for binding to EGFR with an anti-EGFR domain antibody (dAb) selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

13. The ligand of claim 11, wherein each said immunoglobulin single variable domain with binding specificity for EGFR is fused to an Fc region of an antibody.

14. The ligand of claim 11, wherein each said immunoglobulin single variable domain with binding specificity for VEGF is fused to an Fc region of an antibody.

15. The ligand of claim 11, wherein each said immunoglobulin single variable domain with binding specificity for EGFR comprises an amino acid sequence that has at least about 85% amino acid sequence identity with the amino acid sequence of a dAb selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

16. The ligand of claim 12, wherein each said immunoglobulin single variable domain with binding specificity for EGFR comprises an amino acid sequence that has at least about 85% amino acid sequence identity with the amino acid sequence of a dAb selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

17. A ligand that has binding specificity for vascular endothelial growth factor (VEGF) and epidermal growth factor receptor (EGFR), comprising at least one immunoglobulin single variable domain with binding specificity for VEGF and at least one immunoglobulin single variable domain with binding specificity for EGFR, wherein
each said immunoglobulin single variable domain with binding specificity for VEGF competes for binding to VEGF with an anti-VEGF domain antibody (dAb) selected from the group consisting of TAR15-6 (SEQ ID NO:117), TAR15-8 (SEQ ID NO:119), and TAR15-26 (SEQ ID NO:123); and
each said immunoglobulin single variable domain with binding specificity for EGFR competes for binding to EGFR with an anti-EGFR domain antibody (dAb) selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

18. A ligand that has binding specificity for vascular endothelial growth factor (VEGF) and epidermal growth factor receptor (EGFR), comprising at least one immunoglobulin single variable domain with binding specificity for VEGF and at least one immunoglobulin single variable domain with binding specificity for EGFR, wherein
each said immunoglobulin single variable domain with binding specificity for VEGF competes for binding to VEGF with an anti-VEGF domain antibody (dAb) selected from the group consisting of TAR15-6 (SEQ ID NO:117), TAR15-8 (SEQ ID NO:119), and TAR15-26 (SEQ ID NO:123); and
each said immunoglobulin single variable domain with binding specificity for EGFR competes for binding to EGFR with an anti-EGFR domain antibody (dAb) selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

19. The ligand of claim 17, wherein each said immunoglobulin single variable domain with binding specificity for VEGF comprises an amino acid sequence that has at least about 85% amino acid sequence identity with the amino acid sequence of a dAb selected from the group consisting of TAR15-6 (SEQ ID NO:117), TAR15-8 (SEQ ID NO:119), and TAR15-26 (SEQ ID NO:123); and
each said immunoglobulin single variable domain with binding specificity for EGFR competes for binding to EGFR with an anti-EGFR domain antibody (dAb) selected from the group consisting of DOM16-39 (SEQ ID NO:345), DOM16-39-87 (SEQ ID NO:420), DOM16-39-100 (SEQ ID NO:423), DOM16-39-107 (SEQ ID NO:430), DOM16-39-109 (SEQ ID NO:432), DOM16-39-115 (SEQ ID NO:438), and DOM16-39-200 (SEQ ID NO:441).

20. The ligand of claim 18, wherein each said immunoglobulin single variable domain with binding specificity for VEGF comprises an amino acid sequence that has at least about 85% amino acid sequence identity with the amino acid sequence of a dAb selected from the group consisting of TAR15-6 (SEQ ID NO:117), TAR15-8 (SEQ ID NO:119), and TAR15-26 (SEQ ID NO:123); and
each said immunoglobulin single variable domain with binding specificity for EGFR competes for binding to EGFR with an anti-EGFR domain antibody (dAb) selected from the group consisting of DOM16-39-521 (SEQ ID NO:577), DOM16-39-541 (SEQ ID NO:585), DOM16-39-542 (SEQ ID NO:586), DOM16-39-551 (SEQ ID NO:591), DOM16-39-601 (SEQ ID NO:608), DOM16-39-604 (SEQ ID NO:611), DOM16-39-618 (SEQ ID NO:621), and DOM16-39-619 (SEQ ID NO:622).

21-196. (canceled)


SEQUENCE LISTING (728 sequences)

SEQ ID NO: 1 - DNA, 324 bp, Homo sapiens
gacatccagatgacccagtctccatcctccctgtctgcatctgtaggagaccgtgtcaccatcacttgccgggcaagtcagtggattggtcctgagttaagttggtaccagcagaaaccagggaaagcccctaagctcctgatctatcatggttccattttgcaaagtggggtcccatcacgtttcagtggcagtggatctgggacagatttcactctcaccatcagcagtctgcaacctgaagattttgctacgtactactgtcaacagcgtatgtatcggcctgctacgttcggccaagggaccaaggtggaaatcaaacgg

SEQ ID NO: 2 - DNA, 324 bp, Homo sapiens
gacatccagatgacccagtctccatcctccctgtctgcatctgtaggagaccgtgtcaccatcacttgccgggcaagtcagtggattggtagggagttaaagtggtatcagcagaaaccagggaaagcccctaggctcctgatctatcatggttccgtgttgcaaagtggggtcccatcacgtttcagtggcagtggatctgggacagatttcactctcaccatcagcagtctgcaacctgaagattttgctacgtactactgtcaacaggatttttttgttcctgatacgttcggccaagggaccaaggtggaaatcaaacgg

SEQ ID NO: 3 - DNA, 324 bp, Homo sapiens
gacatccagatgacccagtctccatcctccctgtctgcatctgtaggagaccgtgtcaccatcacttgccgggcaagtcaggatattgcgaatgatttaatgtggtaccagcagaaaccagggaaagcccctaagctcctgatctatcgtaattcccgtttgcaaggtggggtcccatcacgtttcagtggcagtggatctgggacagatttcactctcaccatcagcagtctgcaacctgaagattttgctacgtactactgtcaacagcttgttcatcgcccttatacgatcggccaagggaccaaggtggaaatcaaacgg

SEQ ID NO: 4 - DNA, 324 bp, Homo sapiens
gacatccagatgacccagtctccatcctccctgtctgcatctgtaggagaccgtgtcaccatcacttgccgggcaagtcagtttattgggccgcatttaacgtggtatcagcagaaaccagggaaagcccctaagctcctgatctatcattcttccttgttgcaaagtggggtcccatcacgtttcagtggcagtggatctgggacagatttcactctcaccatcagcagtctgcagcctgaagatttcgctacgtactactgtcaacagtatatgtattatccttctacgttcggccaagggaccaaggtgaaaatcaagcgg

SEQ ID NO: 5 - DNA, 324 bp, Homo sapiens
gacatccagatgacccagtctccatcctccctgtctgcatctgtaggagaccgtgtcaccatcacttgccgggcaagtcagtggattggtccggagttaagttggtaccagcagaaaccagggaaagcccctaagctcctgatctatcatacgtccattttgcaaagtggggtcccatcacgtttcagtggcagtggatctgggacagatttcactctcaccatcagcagtctgcaacctgaagattttgctacgtactactgtcaacagtatatgtttcagcctaggacgttcggccaagggaccaaggtggaaatcagacgg

SEQ ID NO: 6 - DNA, 324 bp, Homo sapiens
gacatccagatgatccagtctccatcctccctgtccgcatctgtaggagaccgtgtcaccatcacttgccgggcaagtcagtttattggtaatgagttaagttggtatcagcagaaaccagggaaagcccctaagctcctgatctatcatgcttccagtttgcaaagtggggtcccatcacgtttcagtggcagtggatctgggacagatttcactctcaccatcagcagtctgcaacctgaagattttgctacgtactactgtcaacaggttctgggttatccttatacgttcggccaagggaccaaggtggaaatcaaacgg

SEQ ID NO: 7 - DNA, 324 bp, Homo sapiens
gacatccagatgacccagtctccatcctccctgtctgcatctgtaggagaccgtgtcaccatcacttgccgggcaagtcagtggattggtcctgagttaagttggtaccagcagaaaccagggaaagcccctaagctcctgatctatcatggttccattttgcaaagtggggtcccatcacgtttcagtggcagtggatctgggacagatttcactctcaccatcagcagtctgcaacctgaagattttgctacgtactactgtcaacaggttctgtatagtcctttgacgttcggccaagggaccaaggtggaaatcaaacgg

SEQ ID NO: 8 - DNA, 324 bp, Homo sapiens
gacatccagatgacccagtctccatcctccctgtctgcatctgtaggagaccgtgtcaccatcacttgccgggcaagtcagtggattgggaatgagttaaagtggtatcagcagaaaccagggaaagcccctaagctcctgatctatatgtcttcccttttgcaaagtggggtcccatcacgtttcagtggcagtggatctgggacagatttcactctcaccatcagcagtctgcaacctgaagatttagctacgtactactgtcaacagacgcttttgcttccttttacgttcggccaagggaccaaggtggaaatcaaacgg

SEQ ID NO: 9 - DNA, 324 bp, Homo sapiens
gacatccagatgacccagtctccatcctccctgtctgcatctgtaggagaccgtgtcaccatcacttgccgggcaagtcagtggattggtcctgagttaagttggtaccagcagaaaccagggaaagcccctaagctcctgatctatcatggttccattttgcaaagtggggtcccatcacgtttcagtggcagtggatctgggacagatttcactctcaccatcagcagtctgcaacctgaagattttgctacgtactactgtcaacagcgtctgtattatcctggtacgttcggccaagggaccaaggtggaaatcaaacgg

SEQ ID NO: 10 - DNA, 324 bp, Homo sapiens
gacatccagatgacccagtctccatcctccctgtctgcatctgtaggagaccgtgtcaccatcacttgccgggcaagtcagtctattgggcgtgagttaagttggtaccagcagaaaccagggaaagcccctatgctcctgatctatcatagttccaatttgcaaagtggggtcccatcacgtttcagtggcagtggatctgggacagatttcactctcaccatcagcagtctgcaacctgaagattttgctacgtactactgtcaacaggggatgtattggccttatacgttcggccaagggaccaaggtggaaatcaaacgg

SEQ ID NO: 11 - DNA, 324 bp, Homo sapiens
gacatccagatgacccagtctccatcctccctgtctgcatctgtaggagaccgtgtcaccatcacttgccgggcaagtcagtggattaagccggccttacattggtaccagcagaaaccagggaaagcccctaagctcctgatctatcatggttccattttgcaaagtggggtcccatcacgtttcagtggcagtggatctgggacagatttcactctcaccattagcagtctgcaacctgaagattttgctacgtactactgtcaacagactctttttatgccttatacgttcggccaagggaccaaggtggaaatcaaacgg

SEQ ID NO: 12 - DNA, 324 bp, Homo sapiens
gacatccagatgacccagtctccatcctccctgtctgcatctgtaggagaccgtgtcaccatcacttgccgggcaagtcagtctattagtactgcgttactgtggtatcagcagaaaccagggaaagcccctaagctcctgatctataatggttccatgttgccaaatggggtcccatcacgtttcagtggcagtggatctgggacagatttcactctcaccatcagcagtctgcaacctgaagattttgctacgtactactgtcaacagacttgggatactcctatgacgttcggccaagggaccaaggtggaaatcaaacgg

SEQ ID NO: 13 - DNA, 324 bp, Homo sapiens
gacatccagatgacccagtctccatcctccctgtctgcatctgtaggagaccgtgtcaccatcacttgccgggcaagtcagtggattgggcatgatttatcgtggtaccagcagaaaccagggaaagcccctaagctcctgatctatcattcgtcctctttgcaaagtggggtcccatcacgtttcagtggcagtggatctgggacagatttcactctcaccatcagcagtctgcaacctgaagatgttgctacgtactactgtcaacagcttatgggttatccttttacgttcggccaagggaccaaggtggaaatcaaacgg

SEQ ID NO: 14 - DNA, 324 bp, Homo sapiens
gacatccagatgacccagtctccatcctccctgtctgcatctgtaggagaccgtgtcaccatcacttgccgggcaagtcaggatattgggggtttgttagtgtggtatcagcagaaaccagggaaagcccctaagctcctgatctaccggagttcctatttgcaaagtggggtcccatcacgtttcagtggcagtggatctgggacagatttcactctcaccatcagcagtctgcaacctgaagatttcgctacgtactactgtcaacagacgtggggtattcctcatacgttcggccaagggaccaaggtggaaatcaaacgg

SEQ ID NO: 15 - DNA, 324 bp, Homo sapiens
gacatccagatgacccagtctccatcctccctgtctgcatctgtaggagaccgtgtcaccatcacttgccgggcaagtcagaagatttttaatggtttaagttggtatcagcagaaaccagggaaagcccctaagctcctgatctatcatagttccacgttgcaaagtggggtcccatcacgtttcagtggcagtggatctgggacagatttcactctcaccatcagcagtctgcaacctgaagattttgctacgtactactgtcaacaggttcttctgtatccttatacgttcggccaagggaccaaggtggaaatcaaacgg

SEQ ID NO: 16 - DNA, 324 bp, Homo sapiens
gacatccagatgacccagtctccatcctccctgtctgcatctgtaggagaccgtgtcaccatcacttgccgggcaagtcagagtattgggactaatttatcttggtaccagcagaaacctgggaaagcccctaggctcctgatctatcggacgtccatgttgcaaagtggggtcccatcacgtttcagtggcagtggatctgggacagatttcactctcaccatcagcagtctgcaacctgaagattttgctacgtactactgtcaacagcagtttttttggcctcatacgttcggccaagggaccaaggtggaaatcaaacgg

SEQ ID NO: 17 - DNA, 354 bp, Homo sapiens
gaggtgcagctgttggagtctgggggaggcttggtacagcctggggggtccctgcgtctctcctgtgcagcctccggattcacctttaggttgtatgatatggtttgggtccgccaggctccagggaagggtctggagtgggtctcatatattagttctgggggttctggtacatactacgcagactccgtgaagggccggttcaccatctcccgcgacaattccaagaacacgctgtatctgcaaatgaacagcctgcgtgccgaggacaccgcggtatattactgtgcgaaagcgggggggcgggctagttttgactactggggtcagggaaccctggtcaccgtctcgagc

SEQ ID NO: 18 - DNA, 348 bp, Homo sapiens
gaggtgcagctgttggagtctgggggaggcttggtacagcctggggggtccctgcgtctctcctgtgcagcctccggattcacctttcatctttatgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagcggggactcagtttgactactggggtcagggaaccctggtcaccgtctcgagc

SEQ ID NO: 19 - DNA, 357 bp, Homo sapiens
gaggtgcagctgttggagtctgggggaggcttggtacagcctggggggtccctgcgtctctcctgtgcagcctccggattcacctttaataagtatcctatgatgtgggtccgccaggctccagggaagggtctagagtgggtctcagagatttctccgtctggtcaggatacatactacgcagactccgtgaagggccggttcaccatctcccgcgacaattccaagaacacgctgtatctgcaaatgaacagcctgcgtgccgaggacaccgcggtatattactgtgcgaaaaatcctcagattctgtctaattttgactactggggtcagggaaccctggtcaccgtctcgagc

SEQ ID NO: 20 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtacggctgggtctaggggtaattcttttgactactggggtcagggaaccctggtcaccgtctcgagc

SEQ ID NO: 21 - DNA, 363 bp, Homo sapiens
gaggtgcagctgttggagtctgggggaggcttggtacagcctggggggtccctgcgtctctcctgtgcagcctccggattcacctttaaggcttatgagatgggttgggtccgccaggctccagggaagggtctggagtgggtctcaggtatttctcctaatggtggttggacatactacgcagactccgtgaagggccggttcaccatctcccgcgacaattccaagaacacgctgtatctgcaaatgaacagcctgcgtgccgaggataccgcggtatattactgtgcgaaagagtcgattagtcctactccgttggggtttgactactggggtcagggaaccctggtcaccgtctcgagc

SEQ ID NO: 22 - DNA, 348 bp, Homo sapiens
gaggtgcagctgttggagtctgggggaggcttggtacagcctggggggtccctgcgtctctcctgtgcagcctccggattcacctttactgggtatgagatggggtgggtccgccaggctccagggaagggtctagagtgggtctcatatatttctaggggtggtcggtggacatactacgcagactccgtgaagggccggttcaccatctcccgcgacaattccaagaacacgctgtatctgcaaatgaacagcctgcgtgccgaggacaccgcggtatattactgtgcgaaatcggatactatgtttgactactggggtcagggaaccctggtcaccgtctcgagc

SEQ ID NO: 23 - DNA, 348 bp, Homo sapiens
gaggtgcagctgttggagtctgggggaggcttggtacagcctggggggtccctgcgtctctcctgtgcagcctccggattcacctttagtgcttatgagatgggttgggtccgccaggctccagggaagggtctagagtgggtctcatttatttctggggggggtcggtggacatactacgcagactccgtgaagggccggttcaccatctcccgcgacaattccaagaacacgctgtatctgcaaatgaacagcctgcgtgccgaggacaccgcggtatattactgtgcgaaatattcggaggattttgactactggggtcagggaaccctggtcaccgtctcgagc

SEQ ID NO: 24 - DNA, 348 bp, Homo sapiens
gaggtgcagctgttggagtctgggggaggcttggtacagcctggggggtccctgcgtctctcctgtgcagcctccggattcacctttggggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 25 - DNA, 369 bp, Homo sapiens
gaggtgcagctgttggagtctgggggaggcttggtacagcctggggggtccctgcgtctctcctgtgcagcctccggattcacctttcagttttataagatgggttgggtccgccaggctccggggaagggtctagagtgggtctcatctattagtagtgtgggtgatgcgacatactacgcagactccgtgaagggccggttcaccatctcccgcgacaattccaagaacacgctgtatctgcaaatgaacagcctgcgtgccgaggacaccgcggtatattactgtgcgaaaatggggggggggcctcctacgtatgttgtgtattttgactactggggtcagggaaccctggtcaccgtctcgagc

SEQ ID NO: 26 - DNA, 369 bp, Homo sapiens
gaggtgcagctgttggagtctgggggaggcttggtacagcctggggggtccctgcgtctctcctgtgcagcctccggattcacctttggggagtatgggatgtattgggtccgccaggctccagggaagggtctagagtgggtctcatctattagtgagcgtggtcggttgacatactacgcagactccgtgaagggccggttcaccatctcccgcgacaattccaagaacacgctgtatctgcaaatgaacaacctgcgtgccgaggataccgcggtatattactgtgcgaaatcggcgctttcgtctgagggtttttcgcgttcttttgactactggggtcagggaaccctggtcaccgtctcgagc

SEQ ID NO: 27 - DNA, 369 bp, Homo sapiens
gaggtgcagctgttggagtctgggggaggcttggtacagcctggggggtccctgcgtctctcctgtgcagcctccggattcacctttagtgattatgcgatgtattgggtccgccaggctccagggaagggtctagagtgggtctcatcgattacggctaggggttttattacatactacgcagactccgtgaagggccggttcaccatctcccgcgacaattccaagaacacgctgtatctgcaaatgaacagcctgcgtgccgaggacaccgcggtatattactgtgcgaaatcgggttttccgcataagtcgggttcgaattattttgactactggggtcagggaaccctggtcaccgtctcgagc

SEQ ID NO: 28 - DNA, 348 bp, Homo sapiens
gaggtgcagctgttggagtctgggggaggcttggtacagcctggggggtccctgcgtctctcctgcgcagcctccggattcacctttaggttgtacgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 29 - DNA, 348 bp, Homo sapiens
gaggtgcagctgttggagtctgggggaggcttggtacagcctggggggtccctgcgtctctcctgtgcagcctccggattcaccttttccttcttcgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagcggggactcagtttgactactggggtcagggaaccctggtcaccgtctcgagc

SEQ ID NO: 30 - DNA, 348 bp, Homo sapiens
gaggtgcagctgttggagtctgggggaggcttggtacagcctggggggtccctgcgtctctcctgtgcagcctccggattcacctttatgctcttcgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagcggggactcagtttgactactggggtcagggaaccctggtcaccgtctcgagc

SEQ ID NO: 31 - DNA, 348 bp, Homo sapiens
gaggtgcagctgttggagtctgggggaggcttggtacagcctggggggtccctgcgtctctcctgtgcagcctccggattcacctttccgttgtacgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagcggggactcagtttgactactggggtcagggaaccctggtcaccgtctcgagc

SEQ ID NO: 32 - DNA, 348 bp, Homo sapiens
gaggtgcagctgttggagtctgggggaggcttggtacagcctggggggtccctgcgtctctcctgtgcagcctccggattcacctttcccctctacgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagcggggactcagtttgactactggggtcagggaaccctggtcaccgtctcgagc

SEQ ID NO: 33 - DNA, 348 bp, Homo sapiens
gaggtgcagctgttggagtctgggggaggcttggtacagcctggggggtccctgcgtctctcctgtgcagcctccggattcacctttcagtacttcgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagcggggactcagtttgactactggggtcagggaaccctggtcaccgtctcgagc

SEQ ID NO: 34 - DNA, 348 bp, Homo sapiens
gaggtgcagctgttggagtctgggggaggcttggtacagcctggggggtccctgcgtctctcctgtgcagcctccggattcacctttcatctttatgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaatccccccgccagtttgactactggggtcagggaaccctggtcaccgtctcgagc

SEQ ID NO: 35 - DNA, 348 bp, Homo sapiens
gaggtgcagctgttggagtctgggggaggcttggtacagcctggggggtccctgcgtctctcctgtgcagcctccggattcacctttcatctttatgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaaaaccccaggcagtttgactactggggtcagggaaccctggtcaccgtctcgagc

SEQ ID NO: 36 - DNA, 348 bp, Homo sapiens
gaggtgcagctgttggagtctgggggaggcttggtacagcctggggggtccctgcgtctctcctgtgcagcctccggattcacctttcatctttatgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaaaagtcgatgcagtttgactactggggtcagggaaccctggtcaccgtctcgagc

SEQ ID NO: 37 - DNA, 348 bp, Homo sapiens
gaggtgcagctgttggagtctgggggaggcttggtacagcctggggggtccctgcgtctctcctgtgcagcctccggattcacctttcatctttatgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaatccccccgcaagtttgactactggggtcagggaaccctggtcaccgtctcgagc

SEQ ID NO: 38 - DNA, 348 bp, Homo sapiens
gaggtgcagctgttggagtctgggggaggcttggtacagcctggggggtccctgcgtctctcctgtgcagcctccggattcacctttcatctttatgatatgatgtgggtccgccaggctccagggaagggtctagagtgggtctcatttattgggggggatggtcttaatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 39 - DNA, 357 bp, Homo sapiens
gaggtgcagctgttggagtctgggggaggcttggtacagcctggggggtccctgcgtctctcctgtgcggcctccggataccctatgtggtgggtccgccaggctccagggaagggtcctgagtgggtctcactgattgaggggcagggtgataggacatactacgcagactccgtgaagggccggttcaccatctcccgcgacaattccaagaacacgctgtatctgcaaatgaacagcctgcgcgccgaggacaccgcggtatattactgtgcgaaagcgggggatcgtacggctgggtctaggggtaattcttttgactactggggtcagggaaccctggtcaccgtctcgagc

SEQ ID NO: 40 - DNA, 372 bp, Homo sapiens
gaggtgcagctgttggagtctgggggaggcttggtacagcctggggggtccctgcgtctctcctgtgcggcctccggattcacctttcagtgggcccccatgccctgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtacggctgggtctaggggtaattcttttgactactggggtcagggaaccctggtcaccgtctcgagc

SEQ ID NO: 41 - DNA, 372 bp, Homo sapiens
gaggtgcagctgttggagtctgggggaggcttggtacagcctggggggtccctgcgtctctcctgtgcggcctccggattcacctttcagtggccccacatgccctgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtacggctgggtctaggggtaattcttttgactactggggtcagggaaccctggtcaccgtctcgagc

SEQ ID NO: 42 - DNA, 372 bp, Homo sapiens
  unsure 88, 90: n = A,T,C or G
gaggtgcagctgttggagtctgggggaggcttggtacagcctggggggtccctgcgtctctcctgtgcggcctccggattcacctttnantggccccacatgcactgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtacggctgggtctaggggtaattcttttgactactggggtcagggaaccctggtcaccgtctcgagc

SEQ ID NO: 43 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgttgggtgttgtctaggggtaattcttttgactactggggtcagggaaccctggtcaccgtctcgagc

SEQ ID NO: 44 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtcgcttcctctctaggggtaattcttttgactactggggtcagggaaccctggtcaccgtctcgagc

SEQ ID NO: 45 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtcacaggacctctaggggtaattcttttgactactggggtcagggaaccctggtcaccgtctcgagc

SEQ ID NO: 46 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggtgcagggtgataggacatactacgcagactccgtgaagggccggttcaccatctcccgcgacaattccaagaacacgctgtatctgcaaatgaacagcctgcgtgccgaggacaccgcggtatattactgtgcgaaagcggggccccccttcgctgggtctaggggtaattcttttgactactggggtcagggaaccctggtcaccgtctcgagc

SEQ ID NO: 47 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcggggaccaacaacgctgggtctaggggtaattcttttgactactggggtcagggaaccctggtcaccgtctcgagc

SEQ ID NO: 48 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcgggggatcgtacggctcagaacagcggtaattcttttgactactggggtcggggaaccctggtcaccgtctcgagc

SEQ ID NO: 49 - DNA, 372 bp, Homo sapiens
gaggtgcagctgttggagtctgggggaggcttggtacagcctggggggtccctgcgtctctcctgtgcggcctccggattcacctttcagtggtatcctatgtggtgggtccgccaggctccagggaagggtcttgagtgggtctcactgattgaggggcagggtgataggacatactacgcagactccgtgaagggccggttcaccatctcccgcgacaattccaagaacacgctgtatctgcaaatgaacagcctgcgtgccgaggacaccgcggtatattactgtgcgaaagcggggaacagcaacgctgggtctaggggtaattcttttgactactggggtcagggaaccctggtcaccgtctcgagc

SEQ ID NO: 50 - DNA, 348 bp, Homo sapiens
gaggtgcagctgttggagtctgggggaggcttggtacagcctggggggtccctgcgtctctcctgtgcagcctccggattcacctttcccgcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 51 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 52 - DNA, 348 bp, Homo sapiens
gaggtgcagctgttggagtctgggggaggcttggtacagcctggggggtccctgcgtctctcctgtgcagcctccggattcacctttacggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctaaagc

SEQ ID NO: 53 - DNA, 348 bp, Homo sapiens
gaggtgcagctgttggagtctgggggaggcttggtacagcctggggggtccctgcgtctctcctgtgcagcctccggattcacctttgggtggtatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 54 - DNA, 348 bp, Homo sapiens
gaggtgcagctgttggagtctgggggaggcttggtacagcctggggggtccctgcgtctctcctgtgcagcctccggattcacctttgggctctatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctagagc

SEQ ID NO: 55 - DNA, 348 bp, Homo sapiens
gaggtgcagctgttggagtctgggggaggcttggtacagcctggggggtccctgcgtctctcctgtgcagcctccggattcacctttctggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctatatctgcaaatgaacagcctgcgtgccgaggacaccgcggtatattactgtgcgaaagatcctcggaagtttgactactggggtcagggaaccctggtcaccgtctcgagc

SEQ ID NO: 56 - DNA, 348 bp, Homo sapiens
gaggtgcagctgttggagtctgggggaggcttggtacagcctggggggtccctgcgtctctcctgtgcagcctccggattcacctttgggaggtatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 57 - DNA, 348 bp, Homo sapiens
gaggtgcagctgttggagtctgggggaggcttggtacagcctggggggtccctgcgtctctcctgtgcagcctccggattcacctttggggctttcccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 58 - DNA, 348 bp, Homo sapiens
gaggtgcagctgttggagtctgggggaggcttggtacagcctggggggtccctgcgtctctcctgtgcagcctccggattcacctttggggcttatccgatgttgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 59 - DNA, 348 bp, Homo sapiens
gaggtgcagctgttggagtctgggggaggcttggtacagcctggggggtccctgcgtctctcctgtgcagcctccggattcaccttttccctcttcccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 60 - DNA, 348 bp, Homo sapiens
gaggtgcagctgttggagtctgggggaggcttggtacagcctggggggtccctgcgtctctcctgtgcagcctccggattcacctttttcttgttcccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 61 - DNA, 348 bp, Homo sapiens
gaggtgcagctgttggagtccgggggaggcttggtacagcctggggggtccctgcgtctctcctgtgcagcctccggattcaccttttcgtacttcccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 62 - DNA, 348 bp, Homo sapiens
gaggtgcagctgttggagtctgggggaggcttggtacagcctggggggtccctgcgtctctcctgtgcagcctccggattcacctttgccttcgccccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcaagc

SEQ ID NO: 63 - DNA, 348 bp, Homo sapiens
gaggtgcagctgttggagtctgggggaggcttggtacagcctggggggtccctgcgtctctcctgtgcagcctccggattcacctttgccccctacccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcaagc

SEQ ID NO: 64 - DNA, 348 bp, Homo sapiens
gaggtgcagctgttggagtctgggggaggcttggtacagcctggggggtccctgcgtctctcctgtgcagcctccggattcacctttacctcccacccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 65 - DNA, 348 bp, Homo sapiens
gaggtgcagctgttggagtctgggggaggcttggtacagcctggggggtccctgcgtctctcctgtgcagcctccggattcacctttacgagccacccgatgatgtgggtccgccaggctccagggaagggtctagagtgggtctcagagatttcgccttcgggttcttatacatactacgcagactccgtgaagggccggttcaccatctcccgcgacaattccaagaacgcgctgtatctgcaaatgaacagcctgcgtgccgaggacaccgcggtatattactgtgcgaaagatcctcggaagtttgactactggggtcagggaaccctggtcaccgtctcgagc

SEQ ID NO: 66 - DNA, 348 bp, Homo sapiens
gaggtgcagctgttggagtctgggggaggcttggtacagcctggggggtccctgcgtctctcctgtgcagcctccggattcacctttcacgcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 67 - DNA, 348 bp, Homo sapiens
gaggtgcagctgttggagtctgggggaggcttggtacagcctggggggtccctgcgtctctcctgtgcagcctccggattcacctttagggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 68 - DNA, 348 bp, Homo sapiens
gaggtgcagctgttggagtctgggggaggcttggtacagcctggggggtccctgcgtctctcctgtgcagcctccggattcacctttttggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 69 - DNA, 348 bp, Homo sapiens; identical to SEQ ID NO: 68

SEQ ID NO: 70 - DNA, 348 bp, Homo sapiens
gaggtgcagcagttggagtctgggggaggcttggtacagcctggggggtccctgcgtctctcctgtgcagcctccggattcaccttttgggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 71 - DNA, 348 bp, Homo sapiens
gaggtgcagctgttggagtctgggggaggcttggtacagcctggggggtccctgcgtctctcctgtgcagcctccggattcacctttcaggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctagagc

SEQ ID NO: 72 - DNA, 348 bp, Homo sapiens
gaggtgcagctgttggagtctgggggaggcttggtacagcctggggggtccctgcgcctctcctgtgcagcctccggattcacctttgggcactatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 73 - DNA, 348 bp, Homo sapiens
gaggtgcagctgttggagtctgggggaggcttggtacagcctggggggtccctgcgtctctcctgtgcagcctccggattcacctttgggttgtatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 74 - DNA, 348 bp, Homo sapiens; identical to SEQ ID NO: 53

SEQ ID NO: 75 - DNA, 348 bp, Homo sapiens; identical to SEQ ID NO: 57

SEQ ID NO: 76 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 77 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaatccccccgcaagtttgactactggggtcagggaaccctggtcaccgtctcgagc

SEQ ID NO: 78 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaactccaagaacacgctgtatctgcaaatgaacagcctgcgtgccgaggacaccgcggtatattactgtgcgaaaaccccccgcaagtttgactactggggtcggggaaccctggtcaccgtctcgagc

SEQ ID NO: 79 - DNA, 348 bp, Homo sapiens
gaggtgcagctgttggagtctgggggaggtttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcgggagtttgactactggggtcagggaaccctggtcaccgtctcgagc

SEQ ID NO: 80 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatccccggcgctttgactactggggtcagggaaccctggtcaccgtctcgagc

SEQ ID NO: 81 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatccccgcttctttgactactggggtcagggaaccctggtcaccgtctcgagc

SEQ ID NO: 82 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaaacccccatcaagtttgactactggggtcagggaaccctggtcaccgtctcgagc

SEQ ID NO: 83 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttctcatacatactacgcagactccgtgaagggccggttcaccatctcccgcgacaattccaagaacacgctgtatctgcaaatgaacagcctgcgtgccgaggacaccgcggtatattactgtgcgaaagatttctcgcagtttgactactggggtcagggaaccctggtcaccgtctcgagc

SEQ ID NO: 84 - DNA, 348 bp, Homo sapiens
gaggtgcagctgttggagtctgggggaggcttggtacagcctggggggtccctgcgtctctcctgtgcagcctccggattcacctttaaggcttatccgatattgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 85 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggccccagggaagggtctagagtgggtctcagagatttcgccttcgggcaagatgaagtactacgcagactccgtgaagggccggttcaccatctcccgcgacaattccaagaacacgctgtatctgcaaatgaacagcctgcgtgccgaggacaccgcggtatattactgtgcgaaaaccccccgcaagtttgactactggggtcggggaaccctggtcaccgtctccagc

SEQ ID NO: 86 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgcccaccggttcttatacatactacgcagactccgtgaagggccggttcaccatctcccgcgacaattccaagaacacgctgtatctgcaaatgaacagcctgcgtgccgaggacaccgcggtatattactgtgcgaaatccccccgcaagtttgactactggggtcagggaaccctggtcaccgtctcgagc

SEQ ID NO: 87 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcaaactccgtgaagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 88 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccctccggttcttatacatactacgcagactccgtgaagggccggttcaccatctcccgcgacaattccaagaacacgctgtatctgcaaatgaacagcctgcgtgccgaggacaccgcggtatattactgtgcgaaaaccccccgcaagtttgactactggggtcagggaaccctggtcaccgtctcgagc

SEQ ID NO: 89 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtcagatgaggtactacgcagactccgtgaagggccggttcaccatctcccgcgacaattccaagaacacgctgtatctgcaaatgaacagcctgcgtgccgaggacaccgcggtatattactgtgcgaaagatcctcgggagtttgactactggggtcagggaaccctggtcaccgtctcgagc

SEQ ID NO: 90 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtaggtcgcggtactacgcagactccgtgaagggccggttcaccatctcccgcgacaattccaagaacacgctgtatctgcaaatgaacagcctgcgtgccgaggacaccgcggtatattactgtgcgaaagatcctcgggagtttgactactggggtcagggaaccctggtcaccgtctcgagc

SEQ ID NO: 91 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtaggtacagctactacgcagactccgtgaagggccggttcaccatctcccgcgacaattccaagaacacgctgtatctgcaaatgaacagcctgcgtgccgaggacaccgcggtatattactgtgcgaaagatccccggcgctttgactactggggtcagggaaccctggtcaccgtctcgagc

SEQ ID NO: 92 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtgaggcgaggtactacgcagactccgtgaagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 93 - DNA, 348 bp, Homo sapiens
gaggtgcagctgttggagtctgggggaggcttggtacagcctggggggtccctgcgtctctcctgtgcagcctccggattcacctttagggcttatccgataatgtgggtccgccaggctccagggaagggtctagagtgggtctcagagatttcgccttcgggtgagaagcggtactacgcagactccgtgaagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 94 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtgaggaggagtactacgcagactccgtgaagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 95 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtaagaagacgtactacgcagactccgtgcagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 96 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtaagaagaagtactacgcagactccgtgaagggccggttcaccatctcccgcgacaattccaagaacacgctgtatctgcaaatgaacagcctgcgtgccgaggacaccgcggtatattactgtgcgaaagatccccgcgcctttgactactggggtcagggaaccctggtcaccgtctcgagc

SEQ ID NO: 97 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtgggtatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 98 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttcttatacatactacgcagacgaggtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 99 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggtcacaacaagtactacgcagactccgtgaagggccggttcaccatctcccgcgacaattccaagaacacgctgtatctgcaaatgaacagcctgcgtgccgaggacaccgcggtatattactgtgcgaaagatcctcggaggtttgactactggggtcagggaaccctggtcaccgtctcgagc

SEQ ID NO: 100 - protein, 108 aa, Homo sapiens
DIQMTQSPSSLSASVGDRVTITCRASQWIGPELSWYQQKPGKAPKLLIYHGSILQSGVPSRFSGSGSGTDFTLTISSLQPEDFATYYCQQRMYRPATFGQGTKVEIKR

SEQ ID NO: 101 - protein, 108 aa, Homo sapiens
DIQMTQSPSSLSASVGDRVTITCRASQWIGRELKWYQQKPGKAPRLLIYHGSVLQSGVPSRFSGSGSGTDFTLTISSLQPEDFATYYCQQDFFVPDTFGQGTKVEIKR

SEQ ID NO: 102 - protein, 108 aa, Homo sapiens
DIQMTQSPSSLSASVGDRVTITCRASQDIANDLMWYQQKPGKAPKLLIYRNSRLQGGVPSRFSGSGSGTDFTLTISSLQPEDFATYYCQQLVHRPYTIGQGTKVEIKR

SEQ ID NO: 103 - protein, 108 aa, Homo sapiens
DIQMTQSPSSLSASVGDRVTITCRASQFIGPHLTWYQQKPGKAPKLLIYHSSLLQSGVPSRFSGSGSGTDFTLTISSLQPEDFATYYCQQYMYYPSTFGQGTKVKIKR

SEQ ID NO: 104 - protein, 108 aa, Homo sapiens
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRR

SEQ ID NO: 105 - protein, 108 aa, Homo sapiens
DIQMIQSPSSLSASVGDRVTITCRASQFIGNELSWYQQKPGKAPKLLIYHASSLQSGVPSRFSGSGSGTDFTLTISSLQPEDFATYYCQQVLGYPYTFGQGTKVEIKR

SEQ ID NO: 106 - protein, 108 aa, Homo sapiens
DIQMTQSPSSLSASVGDRVTITCRASQWIGPELSWYQQKPGKAPKLLIYHGSILQSGVPSRFSGSGSGTDFTLTISSLQPEDFATYYCQQVLYSPLTFGQGTKVEIKR

SEQ ID NO: 107 - protein, 108 aa, Homo sapiens
DIQMTQSPSSLSASVGDRVTITCRASQWIGNELKWYQQKPGKAPKLLIYMSSLLQSGVPSRFSGSGSGTDFTLTISSLQPEDLATYYCQQTLLLPFTFGQGTKVEIKR

SEQ ID NO: 108 - protein, 108 aa, Homo sapiens
DIQMTQSPSSLSASVGDRVTITCRASQWIGPELSWYQQKPGKAPKLLIYHGSILQSGVPSRFSGSGSGTDFTLTISSLQPEDFATYYCQQRLYYPGTFGQGTKVEIKR

SEQ ID NO: 109 - protein, 108 aa, Homo sapiens
DIQMTQSPSSLSASVGDRVTITCRASQSIGRELSWYQQKPGKAPMLLIYHSSNLQSGVPSRFSGSGSGTDFTLTISSLQPEDFATYYCQQGMYWPYTFGQGTKVEIKR

SEQ ID NO: 110 - protein, 108 aa, Homo sapiens
DIQMTQSPSSLSASVGDRVTITCRASQWIKPALHWYQQKPGKAPKLLIYHGSILQSGVPSRFSGSGSGTDFTLTISSLQPEDFATYYCQQTLFMPYTFGQGTKVEIKR

SEQ ID NO: 111 - protein, 108 aa, Homo sapiens
DIQMTQSPSSLSASVGDRVTITCRASQSISTALLWYQQKPGKAPKLLIYNGSMLPNGVPSRFSGSGSGTDFTLTISSLQPEDFATYYCQQTWDTPMTFGQGTKVEIKR

SEQ ID NO: 112 - protein, 108 aa, Homo sapiens
DIQMTQSPSSLSASVGDRVTITCRASQWIGHDLSWYQQKPGKAPKLLIYHSSSLQSGVPSRFSGSGSGTDFTLTISSLQPEDVATYYCQQLMGYPFTFGQGTKVEIKR

SEQ ID NO: 113 - protein, 108 aa, Homo sapiens
DIQMTQSPSSLSASVGDRVTITCRASQDIGGLLVWYQQKPGKAPKLLIYRSSYLQSGVPSRFSGSGSGTDFTLTISSLQPEDFATYYCQQTWGIPHTFGQGTKVEIKR

SEQ ID NO: 114 - protein, 108 aa, Homo sapiens
DIQMTQSPSSLSASVGDRVTITCRASQKIFNGLSWYQQKPGKAPKLLIYHSSTLQSGVPSRFSGSGSGTDFTLTISSLQPEDFATYYCQQVLLYPYTFGQGTKVEIKR

SEQ ID NO: 115 - protein, 108 aa, Homo sapiens
DIQMTQSPSSLSASVGDRVTITCRASQSIGTNLSWYQQKPGKAPRLLIYRTSMLQSGVPSRFSGSGSGTDFTLTISSLQPEDFATYYCQQQFFWPHTFGQGTKVEIKR

SEQ ID NO: 116 - protein, 118 aa, Homo sapiens
EVQLLESGGGLVQPGGSLRLSCAASGFTFRLYDMVWVRQAPGKGLEWVSYISSGGSGTYYADSVKGRFTISRDNSKNTLYLQMNSLRAEDTAVYYCAKAGGRASFDYWGQGTLVTVSS

SEQ ID NO: 117 - protein, 116 aa, Homo sapiens
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 118 - protein, 119 aa, Homo sapiens
EVQLLESGGGLVQPGGSLRLSCAASGFTFNKYPMMWVRQAPGKGLEWVSEISPSGQDTYYADSVKGRFTISRDNSKNTLYLQMNSLRAEDTAVYYCAKNPQILSNFDYWGQGTLVTVSS

SEQ ID NO: 119 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRTAGSRGNSFDYWGQGTLVTVSS

SEQ ID NO: 120 - protein, 121 aa, Homo sapiens
EVQLLESGGGLVQPGGSLRLSCAASGFTFKAYEMGWVRQAPGKGLEWVSGISPNGGWTYYADSVKGRFTISRDNSKNTLYLQMNSLRAEDTAVYYCAKESISPTPLGFDYWGQGTLVTVSS

SEQ ID NO: 121 - protein, 116 aa, Homo sapiens
EVQLLESGGGLVQPGGSLRLSCAASGFTFTGYEMGWVRQAPGKGLEWVSYISRGGRWTYYADSVKGRFTISRDNSKNTLYLQMNSLRAEDTAVYYCAKSDTMFDYWGQGTLVTVSS

SEQ ID NO: 122 - protein, 116 aa, Homo sapiens
EVQLLESGGGLVQPGGSLRLSCAASGFTFSAYEMGWVRQAPGKGLEWVSFISGGGRWTYYADSVKGRFTISRDNSKNTLYLQMNSLRAEDTAVYYCAKYSEDFDYWGQGTLVTVSS

SEQ ID NO: 123 - protein, 116 aa, Homo sapiens
EVQLLESGGGLVQPGGSLRLSCAASGFTFGAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 124 - protein, 123 aa, Homo sapiens
EVQLLESGGGLVQPGGSLRLSCAASGFTFQFYKMGWVRQAPGKGLEWVSSISSVGDATYYADSVKGRFTISRDNSKNTLYLQMNSLRAEDTAVYYCAKMGGGPPTYVVYFDYWGQGTLVTVSS

SEQ ID NO: 125 - protein, 123 aa, Homo sapiens
EVQLLESGGGLVQPGGSLRLSCAASGFTFGEYGMYWVRQAPGKGLEWVSSISERGRLTYYADSVKGRFTISRDNSKNTLYLQMNNLRAEDTAVYYCAKSALSSEGFSRSFDYWGQGTLVTVSS

SEQ ID NO: 126 - protein, 123 aa, Homo sapiens
EVQLLESGGGLVQPGGSLRLSCAASGFTFSDYAMYWVRQAPGKGLEWVSSITARGFITYYADSVKGRFTISRDNSKNTLYLQMNSLRAEDTAVYYCAKSGFPHKSGSNYFDYWGQGTLVTVSS

SEQ ID NO: 127 - protein, 116 aa, Homo sapiens
EVQLLESGGGLVQPGGSLRLSCAASGFTFRLYDMMWVRQAPGKGLEWVSFIGGDGLNTYYADSVKGRFTISRDNSKNTLYLQMNSLRAEDTAVYYCAKDPRKFDYWGQGTLVTVSS

SEQ ID NO: 128 - protein, 116 aa, Homo sapiens
EVQLLESGGGLVQPGGSLRLSCAASGFTFSFFDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 129 - protein, 116 aa, Homo sapiens
EVQLLESGGGLVQPGGSLRLSCAASGFTFMLFDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 130 - protein, 116 aa, Homo sapiens
EVQLLESGGGLVQPGGSLRLSCAASGFTFPLYDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 131 - protein, 116 aa, Homo sapiens; identical to SEQ ID NO: 130

SEQ ID NO: 132 - protein, 116 aa, Homo sapiens
EVQLLESGGGLVQPGGSLRLSCAASGFTFQYFDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 133 - protein, 116 aa, Homo sapiens
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKSPRQFDYWGQGTLVTVSS

SEQ ID NO: 134 - protein, 116 aa, Homo sapiens
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKNPRQFDYWGQGTLVTVSS

SEQ ID NO: 135 - protein, 116 aa, Homo sapiens
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKKSMQFDYWGQGTLVTVSS

SEQ ID NO: 136 - protein, 116 aa, Homo sapiens
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKSPRKFDYWGQGTLVTVSS

SEQ ID NO: 137 - protein, 116 aa, Homo sapiens
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKDPRKFDYWGQGTLVTVSS

SEQ ID NO: 138 - protein, 119 aa, Homo sapiens
EVQLLESGGGLVQPGGSLRLSCAASGYPMWWVRQAPGKGPEWVSLIEGQGDRTYYADSVKGRFTISRDNSKNTLYLQMNSLRAEDTAVYYCAKAGDRTAGSRGNSFDYWGQGTLVTVSS

SEQ ID NO: 139 - protein, 124 aa, Homo sapiens
EVQLLESGGGLVQPGGSLRLSCAASGFTFQWAPMPWVRQAPGKGLEWVSLIEGQGDRTYYADSVKGRFTISRDNSKNTLYLQMNSLRAEDTAVYYCAKAGDRTAGSRGNSFDYWGQGTLVTVSS

SEQ ID NO: 140 - protein, 124 aa, Homo sapiens
EVQLLESGGGLVQPGGSLRLSCAASGFTFQWPHMPWVRQAPGKGLEWVSLIEGQGDRTYYADSVKGRFTISRDNSKNTLYLQMNSLRAEDTAVYYCAKAGDRTAGSRGNSFDYWGQGTLVTVSS

SEQ ID NO: 141 - protein, 124 aa, Homo sapiens
  UNSURE 30: Xaa = Any Amino Acid
EVQLLESGGGLVQPGGSLRLSCAASGFTFXWPHMHWVRQAPGKGLEWVSLIEGQGDRTYYADSVKGRFTISRDNSKNTLYLQMNSLRAEDTAVYYCAKAGDRTAGSRGNSFDYWGQGTLVTVSS

SEQ ID NO: 142 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRWVLSRGNSFDYWGQGTLVTVSS

SEQ ID NO: 143 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRRFLSRGNSFDYWGQGTLVTVSS

SEQ ID NO: 144 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRHRTSRGNSFDYWGQGTLVTVSS

SEQ ID NO: 145 - protein, 124 aa, Homo sapiens
EVQLLESGGGLVQPGGSLRLSCAASGFTFQWYPMWWVRQAPGKGLEWVSLIEVQGDRTYYADSVKGRFTISRDNSKNTLYLQMNSLRAEDTAVYYCAKAGPPFAGSRGNSFDYWGQGTLVTVSS

SEQ ID NO: 146 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGTNNAGSRGNSFDYWGQGTLVTVSS

SEQ ID NO: 147 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRTAQNSGNSFDYWGRGTLVTVSS

SEQ ID NO: 148 - protein, 124 aa, Homo sapiens
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGNSNAGSRGNSFDYWGQGTLVTVSS

SEQ ID NO: 149 - protein, 116 aa, Homo sapiens
EVQLLESGGGLVQPGGSLRLSCAASGFTFPAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 150 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 151 - protein, 114 aa, Homo sapiens
EVQLLESGGGLVQPGGSLRLSCAASGFTFTAYPMMWVRQAPGKGLEWVSEISPSGSYTYYADSVKGRFTISRDNSKNTLYLQMNSLRAEDTAVYYCAKDPRKFDYWGQGTLVTV

SEQ ID NO: 152 - protein, 116 aa, Homo sapiens
EVQLLESGGGLVQPGGSLRLSCAASGFTFGWYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 153 - protein, 114 aa, Homo sapiens
EVQLLESGGGLVQPGGSLRLSCAASGFTFGLYPMMWVRQAPGKGLEWVSEISPSGSYTYYADSVKGRFTISRDNSKNTLYLQMNSLRAEDTAVYYCAKDPRKFDYWGQGTLVTV

SEQ ID NO: 154 - protein, 116 aa, Homo sapiens
EVQLLESGGGLVQPGGSLRLSCAASGFTFLAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 155 - protein, 116 aa, Homo sapiens
EVQLLESGGGLVQPGGSLRLSCAASGFTFGRYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 156 - protein, 116 aa, Homo sapiens
EVQLLESGGGLVQPGGSLRLSCAASGFTFGAFPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 157 - protein, 116 aa, Homo sapiens
EVQLLESGGGLVQPGGSLRLSCAASGFTFGAYPMLWVRQAPGKGLEWVSEISPSGSYTYYADSVKGRFTISRDNSKNTLYLQMNSLRAEDTAVYYCAKDPRKFDYWGQGTLVTVSS

SEQ ID NO: 158 - protein, 116 aa, Homo sapiens
EVQLLESGGGLVQPGGSLRLSCAASGFTFSLFPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 159 - protein, 116 aa, Homo sapiens
EVQLLESGGGLVQPGGSLRLSCAASGFTFFLFPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 160 - protein, 116 aa, Homo sapiens
EVQLLESGGGLVQPGGSLRLSCAASGFTFSYFPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 161 - protein, 116 aa, Homo sapiens
EVQLLESGGGLVQPGGSLRLSCAASGFTFAFAPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 162 - protein, 116 aa, Homo sapiens
EVQLLESGGGLVQPGGSLRLSCAASGFTFAPYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 163 - protein, 116 aa, Homo sapiens
EVQLLESGGGLVQPGGSLRLSCAASGFTFTSHPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 164 - protein, 116 aa, Homo sapiens
EVQLLESGGGLVQPGGSLRLSCAASGFTFTSHPMMWVRQAPGKGLEWVSEISPSGSYTYYADSVKGRFTISRDNSKNALYLQMNSLRAEDTAVYYCAKDPRKFDYWGQGTLVTVSS

SEQ ID NO: 165 - protein, 116 aa, Homo sapiens
EVQLLESGGGLVQPGGSLRLSCAASGFTFHAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 166 - protein, 116 aa, Homo sapiens
EVQLLESGGGLVQPGGSLRLSCAASGFTFRAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 167 - protein, 116 aa, Homo sapiens; identical to SEQ ID NO: 154

SEQ ID NO: 168 - protein, 116 aa, Homo sapiens; identical to SEQ ID NO: 154

SEQ ID NO: 169 - protein, 116 aa, Homo sapiens
EVQQLESGGGLVQPGGSLRLSCAASGFTFWAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 170 - protein, 114 aa, Homo sapiens
EVQLLESGGGLVQPGGSLRLSCAASGFTFQAYPMMWVRQAPGKGLEWVSEISPSGSYTYYADSVKGRFTISRDNSKNTLYLQMNSLRAEDTAVYYCAKDPRKFDYWGQGTLVTV

SEQ ID NO: 171 - protein, 116 aa, Homo sapiens
EVQLLESGGGLVQPGGSLRLSCAASGFTFGHYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 172 - protein, 116 aa, Homo sapiens
EVQLLESGGGLVQPGGSLRLSCAASGFTFGLYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 173 - protein, 116 aa, Homo sapiens; identical to SEQ ID NO: 152

SEQ ID NO: 174 - protein, 116 aa, Homo sapiens; identical to SEQ ID NO: 156

SEQ ID NO: 175 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRAFDYWGQGTLVTVSS

SEQ ID NO: 176 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKSPRKFDYWGQGTLVTVSS

SEQ ID NO: 177 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKTPRKFDYWGRGTLVTVSS

SEQ ID NO: 178 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPREFDYWGQGTLVTVSS

SEQ ID NO: 179 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRRFDYWGQGTLVTVSS

SEQ ID NO: 180 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRFFDYWGQGTLVTVSS

SEQ ID NO: 181 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKTPIKFDYWGQGTLVTVSS

SEQ ID NO: 182 - protein, 116 aa, Homo sapiens
EVQLLESGGGLVQPGGSLRLSCAASGFTFKAYPIMWVRQAPGKGLEWVSEISPSGSHTYYADSVKGRFTISRDNSKNTLYLQMNSLRAEDTAVYYCAKDFSQFDYWGQGTLVTVSS

SEQ ID NO: 183 - protein, 116 aa, Homo sapiens
EVQLLESGGGLVQPGGSLRLSCAASGFTFKAYPILWVRQAPGKGLEWVSEISPSGSYTYYADSVKGRFTISRDNSKNTLYLQMNSLRAEDTAVYYCAKDPRKFDYWGQGTLVTVSS

SEQ ID NO: 184 - protein, 116 aa, Homo sapiens
EVQLLESGGGLVQPGGSLRLSCAASGFTFKAYPIMWVRQAPGKGLEWVSEISPSGKMKYYADSVKGRFTISRDNSKNTLYLQMNSLRAEDTAVYYCAKTPRKFDYWGRGTLVTVSS

SEQ ID NO: 185 - protein, 116 aa, Homo sapiens
EVQLLESGGGLVQPGGSLRLSCAASGFTFKAYPIMWVRQAPGKGLEWVSEISPTGSYTYYADSVKGRFTISRDNSKNTLYLQMNSLRAEDTAVYYCAKSPRKFDYWGQGTLVTVSS

SEQ ID NO: 186 - protein, 116 aa, Homo sapiens
EVQLLESGGGLVQPGGSLRLSCAASGFTFKAYPIMWVRQAPGKGLEWVSEISPSGSYTYYANSVKGRFTISRDNSKNTLYLQMNSLRAEDTAVYYCAKDPRAFDYWGQGTLVTVSS

SEQ ID NO: 187 - protein, 116 aa, Homo sapiens
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKTPRKFDYWGQGTLVTVSS

SEQ ID NO: 188 - protein, 116 aa, Homo sapiens
EVQLLESGGGLVQPGGSLRLSCAASGFTFKAYPIMWVRQAPGKGLEWVSEISPSGQMRYYADSVKGRFTISRDNSKNTLYLQMNSLRAEDTAVYYCAKDPREFDYWGQGTLVTVSS

SEQ ID NO: 189 - protein, 116 aa, Homo sapiens
EVQLLESGGGLVQPGGSLRLSCAASGFTFKAYPIMWVRQAPGKGLEWVSEISPSGRSRYYADSVKGRFTISRDNSKNTLYLQMNSLRAEDTAVYYCAKDPREFDYWGQGTLVTVSS

SEQ ID NO: 190 - protein, 116 aa, Homo sapiens
EVQLLESGGGLVQPGGSLRLSCAASGFTFKAYPIMWVRQAPGKGLEWVSEISPSGRYSYYADSVKGRFTISRDNSKNTLYLQMNSLRAEDTAVYYCAKDPRRFDYWGQGTLVTVSS

SEQ ID NO: 191 - protein, 116 aa, Homo sapiens
EVQLLESGGGLVQPGGSLRLSCAASGFTFKAYPIMWVRQAPGKGLEWVSEISPSGEARYYADSVKGRFTISRDNSKNTLYLQMNSLRAEDTAVYYCAKDPRAFDYWGQGTLVTVSS

SEQ ID NO: 192 - protein, 116 aa, Homo sapiens
EVQLLESGGGLVQPGGSLRLSCAASGFTFRAYPIMWVRQAPGKGLEWVSEISPSGEKRYYADSVKGRFTISRDNSKNTLYLQMNSLRAEDTAVYYCAKDPRAFDYWGQGTLVTVSS

SEQ ID NO: 193 - protein, 116 aa, Homo sapiens
EVQLLESGGGLVQPGGSLRLSCAASGFTFKAYPIMWVRQAPGKGLEWVSEISPSGEEEYYADSVKGRFTISRDNSKNTLYLQMNSLRAEDTAVYYCAKDPRAFDYWGQGTLVTVSS

SEQ ID NO: 194 - protein, 116 aa, Homo sapiens
EVQLLESGGGLVQPGGSLRLSCAASGFTFKAYPIMWVRQAPGKGLEWVSEISPSGKKTYYADSVQGRFTISRDNSKNTLYLQMNSLRAEDTAVYYCAKDPRAFDYWGQGTLVTVSS

SEQ ID NO: 195 - protein, 116 aa, Homo sapiens
EVQLLESGGGLVQPGGSLRLSCAASGFTFKAYPIMWVRQAPGKGLEWVSEISPSGKKKYYADSVKGRFTISRDNSKNTLYLQMNSLRAEDTAVYYCAKDPRAFDYWGQGTLVTVSS

SEQ ID NO: 196 - protein, 116 aa, Homo sapiens
EVQLLESGGGLVQPGGSLRLSCAASGFTFKAYPIMWVRQAPGKGLEWVSEISPSGGYTYYADSVKGRFTISRDNSKNTLYLQMNSLRAEDTAVYYCAKDPRKFDYWGQGTLVTVSS

SEQ ID NO: 197 - protein, 116 aa, Homo sapiens
EVQLLESGGGLVQPGGSLRLSCAASGFTFKAYPIMWVRQAPGKGLEWVSEISPSGSYTYYADEVKGRFTISRDNSKNTLYLQMNSLRAEDTAVYYCAKDPRKFDYWGQGTLVTVSS

SEQ ID NO: 198 - protein, 116 aa, Homo sapiens
EVQLLESGGGLVQPGGSLRLSCAASGFTFKAYPIMWVRQAPGKGLEWVSEISPSGHNKYYADSVKGRFTISRDNSKNTLYLQMNSLRAEDTAVYYCAKDPRRFDYWGQGTLVTVSS

SEQ ID NO: 199 - DNA, 326 bp, Homo sapiens
gacatccagatgacccagtctccatcctccctgtctgcatctgtaggagaccgtgtcaccatcacttgccgggcaagtcagtatattggtgcggagttatcctggtaccagcagaaaccagggaaagcccctaagctcctgatctattggatttccgagttgcaaagtggggtcccatcacgtttcagtggcagtggatctgggacagatttcactctcaccatcagcagtctgcaacctgaagattttgctacgtactactgtcaacagtctagtaatactccttatacgttcggccaagggaccaaggtggagatcaaacgggc

SEQ ID NO: 200 - DNA, 326 bp, Homo sapiens
gacatccagatgacccagtctccatcctccctgtctgcatctgtaggagaccgtgtcaccatcacttgccgggcaagtcagtatattggtattaatttaatttggtaccagcagaaaccagggaaagcccctaagctcctgatctattgggcttccgttttgcaaagtggggtcccatcacgtttcagtggcagtggatctgggacagatttcactctcaccatcagcagtctgcaacctgaagattttgctacgtactactgtcaacagtcggtgtatgatcctcctacgtacggccaagggaccaaggtggaaatcaaacgggc

SEQ ID NO: 201 - DNA, 326 bp, Homo sapiens
gacatccagatgacccagtctccatcctccctgtctgcatctgtaggagaccgtgtcaccatcacttgccgggcaagtcaggatattttttggttgttatcttggtaccagcagaaaccagggaaagcccctacgctcctgatctattctacttccattttgcaaagtggggtcccatcacgtttcagtggcagtggatctgggacagatttcactctcaccatcagcagtctgcaacctgaagattttgctacgtactactgtcaacagtattatcttgatcctcctacgttcagccaagggaccaaggtggaaatcaaacgggc

SEQ ID NO: 202 - DNA, 326 bp, Homo sapiens
gacatccagatgacccagtctccatcctccctgtctgcatctgtaggagaccgtgtcaccatcacttgccgggcaagtcagtttattggtgttaatttaaattggtaccagcagaaaccagggaaagcccctaggctcctgatctatttgtcgtccattctgcaaagtggggtcccatcacgtttcagtggcagtggatctgggacagatttcactctcaccatcagcagtctgcaacctgaagattttgctacgtactactgtcaacagacttatgatattcctactacgttcggccaagggaccaaggtggaaatcaaacgggc

SEQ ID NO: 203 - DNA, 326 bp, Homo sapiens
gacatccagatgacccagtctccatcctccctgtctgcatctgtaggagaccgtgtcaccatcacttgccgggcaagtcagaacattggtattaatttgcagtggtatcagcagaaaccaaggaaagcccctaagctcctgatctattatgcttccattttgcaaagtggggtcccatcacgtttcagtggcagtggatctgggacagatttcactctcaccatcagcagtctgcaacctgaagattttgctacgtactactgtcaacaggattatgatactccttttacgttcggccaagggaccaaggtggaaatcaaacgggc

SEQ ID NO: 204 - DNA, 326 bp, Homo sapiens
gacatccagatgacccagtttccatcctccctgtctgcatctgtaggagaccgtgtcaccatcacttgccgggcgagtcagcatattgagaggtggttaaattggtatcagcagaaaccagggaaagcccctaagctcctgatctatcgttcgtcctatttgcaaagtggggtcccatcacgtttcagtggcagtggatctgggacagatttcactctcaccatcagcagtctgcaacctgaagattttgctacgtactactgtcaacaggatgctattcttcctcatacgttcggccaagggaccaaggtggaaatcaaacgggc

SEQ ID NO: 205 - DNA, 326 bp, Homo sapiens
gacatccagatgacccagtctccatcctccctgtctgcatctgtaggagaccgtgtcaccatcacttgccgggcaagtcaggggattggggtgaatttacagtggtaccagcagaaaccagggaaagcccctaagctcctgatctattttagttccgtgttgcaaagtggggtcccatcacgtttcagtggcagtggatctgggacagatttcactctcaccatcagcagtctgcaacctgaagattttgctacgtactactgtcaacaggattttgattttcctcagacgttcggccaagggaccaaggtggaaatcaaacgggc

SEQ ID NO: 206 - DNA, 326 bp, Homo sapiens
gacatccagatgacccagtctccatcctccctgtctgcatctgtaggagaccgtgtcaccatcacttgccgggcaagtcagaatattggtattaatttgcagtggtatcagcagaaaccaaggaaagcccctaagctcctgatctattatgcttccattttgcaaagtggggtcccatcacgtttcagtggcagtggatctgggacagatttcactctcaccatcagcagtctgcaacctgaagattttgctacgtactactgtcaacaggagtatgattatcctaatacgttcggccaagggaccaaggtggaaatcaaacgggc

SEQ ID NO: 207 - DNA, 326 bp, Homo sapiens
gacatccagatgacccagtctccatcctccctgtctgcatctgtaggagaccgtgtcaccatcacttgccgggcaagtagtagtattgggtcggggttagagtggtaccagcagaaaccagggaaagcccctaagctcctgatcgttgggtggtccgggttgcaaagtggggtcccatcacgtttcagtggcagtggatctgggacagatttcactctcaccatcagcagtctgcaacctgaagattttgctacgtactactgtcggcagtgtgtgggtttgccttgtacgttcggccaagggaccaaggtggaaatcaaacgggc

SEQ ID NO: 208 - DNA, 326 bp, Homo sapiens
gacatccagatgacccagtctccatcctccctgtctgcatctgtaggagaccgtgtcaccatcacttgccgggcaagtcagtggattggggtggagttaagttggtatcagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacagttggcgttacctccttttacgttcggccaagggaccaaggtggaaatcaaacgggc

SEQ ID NO: 209 - DNA, 326 bp, Homo sapiens
gacatccagatgacccagtctccatcctccctgtctgcatctgtaggagaccgtgtcaccatcacttgccgggcaagtcatgatattggggtgagtttagattggtaccagcagaaaccagggaaagcccctaagctcctgatcgtgtgggcgtccgtgttgcaaagtggggtcccatcacgtttcagtggcagtggatctgggacagatttcactctcaccatcagcagtctgcaacctgaagattttgctacgtactactgtctgcaggtgggtgctgggcctatgacgttcggccaagggaccaaggtggaaatcaaacgggc

SEQ ID NO: 210 - DNA, 326 bp, Homo sapiens
gacatccagatgacccagtctccatcctccctgtctgcatctgtaggagacggtgtcaccatcacttgccgggcaagtcagtatattggtattgatttagcgtggtaccagcggaaaccagggaaagcccctaggctcctgatctataaggcttccgctttgcaaagtggggtcccatcacgtttcagtggcagtggatctgggacagatttcactctcaccatcagcagtctgcaacctgaagattttgctacgtactactgtcaacagtatgcggattatcctgctacgttcggccaagggaccaaggtggaaatcaaacgggc

SEQ ID NO: 211 - DNA, 326 bp, Homo sapiens
gacatccagatgacccagtctccatcctccctgtctgcatctgtaggagaccgtgtcaccatcacttgccgggcaagtcaggagattgagcattatttatcttggtaccagcagaaaccagggaaagcccctaagctcctgatctatctttcgtcccgtttgcaaagtggggtcccatcacgtttcagtggcagtggatctgggacagatttcactctcaccatcagcagtctgcaacccgaagattttgctacgtactactgtcaacagaatgtgcagctgcctattacgttcggccaagggaccaaggtggaaatcaaacgggc

SEQ ID NO: 212 - DNA, 326 bp, Homo sapiens
gacatccagatgacccagtctccatcctccctgtctgcatctgtaggagaccgtgtcaccatcacttgccgggcaagtcaggagattggtgttagtttatcgtggtaccagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacaggatcataattggcctatgacgttcggccaagggaccaaggtggaaatcaaacgggc

SEQ ID NO: 213 - DNA, 326 bp, Homo sapiens
gacatccagatgacccagtctccatcctccctgtctgcatctgtaggagaccgtgtcaccatcacttgccgggcaagtgctcttattatgggggatttagattggtaccagcagaaaccagggaaagcccctaagctcctgatcgcgggtgtgtcctttttgcaaagtggggtcccatcacgtttcagtggcagtggatctgggacagatttcactctcaccatcagcagtctgcaacctgaagattttgctacgtactactgtgctcagtctaggtcgtggccttatacgttcggccaagggaccaaggtggaaatcaaacgggc

SEQ ID NO: 214 - DNA, 326 bp, Homo sapiens
gacatccagatgacccagtctccatcctccctgtctgcatctgtaggagaccgtgtcaccatcacttgccgggcaagtaagatgattgatgagaatttagcttggtaccagcagaaaccagggaaagcccctaagctcctgatccttcggagttccgggttgcaaagtggggtcccatcacgtttcagtggcagtggatctgggacagatttcactctcaccatcagcagtctgcaacctgaagattttgctacgtactactgtcatcagggtcattctgctcctggtacgttcggccaagggaccaaggtggaaatcaaacgggc

SEQ ID NO: 215 - DNA, 326 bp, Homo sapiens
gacatccagatgacccagtctccatcctccctgtctgcatctgtaggagaccgtgtcaccatcacttgccgggcaagtcggtatattggggtgtctttagattggtaccagcagaaaccagggaaagcccctaagctcctgatcatgtggggttccgcgttgcaaagtggggtcccatcacgtttcagtggcagtggatctgggacagatttcactctcaccatcagcagtctgcaacctgaagattttgctacgtactactgtttgcagtctgcggcgccgcctgcgacgttcggccaagggaccaaggtggaaatcaaacgggc

SEQ ID NO: 216 - DNA, 326 bp, Homo sapiens
gacatccagatgacccagtctccatcctccctgtctgcatctgtaggagaccgtgtcaccatcacttgccgggcaagtcaggagattggtgtgagtttaagttggtaccagcagaaaccagggaaagcccctaagctcctgatctattgggcgtccgcgttgcaaagtggggtcccatcacgtttcagtggcagtggatctgggacagatttcactctcaccatcagcagtctgcaacctgaagattttgctacgtactactgtcaacagtcttatttgcctcctgatacgttcggccaagggaccaaggtggaaatcaaacgggc

SEQ ID NO: 217 - DNA, 326 bp, Homo sapiens
gacatccagatgacccagtctccatcctccctgtctgcatctgtaggagaccgtgtcaccatcacttgccgggcaagtcaggagattgctagtgatttactttggtatcagcagaaaccagggaaagcccctaagctcctgatctataatgggtcctcgttgcaaagtggggtcccatcacgtttcagtggcagtggatctgggacagatttcactctcaccatcagcagactgcaacctgaagattttgctacgtactactgtcaatggttgtggagtgagcctttgacgttcggccaagggaccaaggtggaaatcaaacgggc

SEQ ID NO: 218 - DNA, 326 bp, Homo sapiens
gacatccagatgacccagtctccatcctccctgtctgcatctgtaggagaccgtgtcaccatcacttgccgggcaagtcagcatattggtgatgcgttatggtggtatcagcagaaaccagggaaagcccctaagctcctgatctattggacttccaatttgcaaagtggggtcccatcacgtttcagtggcagtggatctgggacagatttcactctcaccatcagcagtctgcaacctgaagattttgctacgtactactgtcaacagactattcgtcggccttatacgttcggccaagggaccaaggtgaaaatcaaacgggc

SEQ ID NO: 219 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 220 - DNA, 326 bp, Homo sapiens
gacatccagatgacccagtctccatcctccctgtctgcatctgtaggagaccgtgtcaccatcacttgccgggcaagtcagaatattggtattaatttgcagtggtatcagcagaaaccaaggaaagcccctaagctcctgatctattatgcttccattttgcaaagtggggtcccatcacgtttcagtggcagtggatctgggacagatttcactctcaccatcagcagtctgcaacctgaagattttgctacgtactactgtcaacagtcttatgatttgcctaagacgttcggccaagggaccaaggtggaaatcaaacgggc

SEQ ID NO: 221 - DNA, 326 bp, Homo sapiens
gacatccagatgatccagtctccatcctccctgtctgcatctgtaggagaccgtgtcaccatcacttgccgggcaagtcaggagattggtgttagtttatcgtggtaccagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacagtggtatggttggcctgatacgttcggccaagggaccaaggtaggaatcaaacgggc

SEQ ID NO: 222 - DNA, 326 bp, Homo sapiens
gacatccagatgacccagtctccatcttccctgtctgcatctgtaggagaccgtgtcaccatcacttgccgggcaagtcagcatattgggattgagttaaattggtatcagcagaaaccagggaaagcccctaagctcctgatctattgggcttccgttttgcaaagtggggtcccatcacgtttcagtggcagtggatctgggacagatttcactctcaccatcagcagtctgcaacctgaagatttcgctacgtactactgtcaacagagtgtttatgttcctactacgttcggccaagggaccaaggtggaaatcaaacgggc

SEQ ID NO: 223 - DNA, 326 bp, Homo sapiens
gacatccagatgacccagtctccatcctccctgtctgcatctgtaggagaccgtgtcaccatcacttgccgggcaagtcagtggattggtgcggagttagtgtggtatcagcagaaaccagggaaagcccctaagctcctgatctattggagttccgttttgcaaagtggggtcccatcacgtttcagtggcagtggatctgggacagatttcactctcaccatcagcagtctgcaacctgaagattttgctacgtactactgtcagcaggctgctcatagtcctcctacgttcggccaagggaccaaggtggaaatcaaacgggc

SEQ ID NO: 224 - DNA, 326 bp, Homo sapiens
gacatccagatgacccagtctccatcctccctgtctgcatctgtaggagaccgtgtcaccatcacttgccgggcaagtcaggatattagtcgtagtttagcttggtaccagcggaaaccagggaaagcccctaggcttctgatctatatgtcttccactttgcaaagtggggtcccatcacgtttcagtggcagtggatctgggacagatttcactctcaccatcagcagtctgcaacctgaagattttgctacgtactactgtcaacagtatgattcttatccttcgacgttcggccaagggaccaaggtggaaatcaaacgggc

SEQ ID NO: 225 - DNA, 326 bp, Homo sapiens
gacatccagatgacccagtctccatcctccctgtctgcatctgtaggagaccgtgtcaccatcacttgccgggcaagtaggatgattgggggtatgttactttggtaccagcagaaaccagggaaagcccctaagctcctgatcacgtatgggtccgtgttgcaaagtggggtcccatcacgtttcagtggcagtggatctgggacagatttcactctcaccatcagcagtctgcaacctgaagattttgctacgtactactgtgctcaggagttttggtggcctcatacgttcggccaagggaccaaggtggaaatcaaacgggc

SEQ ID NO: 226 - DNA, 326 bp, Homo sapiens
gacatccagatgacccagtctccatcctccctgtctgcatctgtaggagaccgtgtcaccatcacttgccgggcaagtcggcctattggggataggttaacgtggtaccagcagaaaccagggaaagcccctaagctcctgatctcgtgggtttccgttttgcaaagtggggtcccatcacgtttcagtggcagtggatctgggacagatttcactctcaccatcagcagtctgcaacctgaagattttgctacgtactactgtcgtcagcttgggggtgggccttttacgttcggccaagggaccaaggtggaaatcaaacgggc

SEQ ID NO: 227 - DNA, 326 bp, Homo sapiens
gacatccagatgacccagtctccatcctccctgtctgcatctgtaggagaccgtgtcaccatcacttgccgggcaagtcagtatattggggtgtcgttaagttggtaccagcagaaaccagggaaagcccctaagctcctgatctattttgcttccgcgttgcaaagtggggtcccatcacgtttcagtggcagtggatctgggacagatttcactctcaccatcagcagtctgcaacctgaagattttgctacgtactactgtcaacaggatcgtgattggcctgcgacgttcggccaagggaccaaggtggaaatcaaacgggc

SEQ ID NO: 228 - DNA, 326 bp, Homo sapiens
gacatccagatgacccagtctccatcctccctgtctgcatctgtaggagaccgtgtcaccatcacttgccgggcaagtggtgctattggggatcgtttaaagtggtaccagcagaaaccagggaaagcccctaagctcctgatctcttgggcgtccgttttgcaaagtggggtcccatcacgtttcagtggcagtggatctgggacagatttcactctcaccatcagcagtctgcaacctgaagattttgctacgtactactgtgtgcaggggccgggggtgcctctgacgttcggccaagggaccaaggtggaaatcaaacgggc

SEQ ID NO: 229 - DNA, 326 bp, Homo sapiens
gacatccagatgactcagtctccatcctccctgtccgcatctgtaggagaccgtgtcaccatcacttgccgggcaagtcagccgattgctcgttggttagcttggtaccagcagaaaccagggaaagcccctaagctcctgatctatggttcttccgttttgcaaagtggggtcccatcacgtttcagtggcagtggatctgggacagatttcactctcaccatcagcagtctgcaacctgaagattttgctacgtactactgtcaacaggatttgaggttgcctccgacgttcggccaagggaccaaggtggaaatcaaacgggc

SEQ ID NO: 230 - DNA, 326 bp, Homo sapiens
gacatccagatgacccagtctccatcctccctgtctgcatctgtaggagaccgtgtcaccatcacttgccgggcaagtcagaatattggtgtgtcgttatcgtggtaccagcagaaaccagggaaagcccctaggctcctgatctattatgggtccaatttgctaagtggggtcccatcacgtttcagtggcagtggatctgggacagatttcactctcaccatcagcagtctgcaacctgaagattttgctacgtactactgtcaacaggagttttcgtggcctgtgacgttcggccaagggaccaaggtggaaatcaaacgggc

SEQ ID NO: 231 - DNA, 326 bp, Homo sapiens
gacatccagatgacccagtctccatcctccctgtctgcatctgtaggagaccgtgtcaccatcacttgccgggcaagtcttccgattgatgatggtttaggttggtaccagcagaaaccagggaaagcccctaagctcctgatctgtggggtgtccggtttgcaaagtggggtcccattacgtttcagtggcagtggatctgggacagatttcactctcaccatcagcagtctgcaacctgaagattttgctacgtactactgtggtcaggggcaggttcagcctagtacgttcggccaagggaccaaggtggaaatcaaacgggc

SEQ ID NO: 232 - DNA, 326 bp, Homo sapiens
gacatccagatgacccagtctccatcctccctgtctgcatctgtaggagaccgtgtcaccatcacttgccgggcaagtcagaatattggtattaatttgcagtggtatcagcagaaaccaaggaaagcccctaagctcctgatctattatgcttccattttgcaaagtggggtcccatcacgtttcagtggcagtggatctgggacagatttcactctcaccatcagcagtctgcaacctgaagattttgctacgtactactgtcaacagtcgtatgatgcgcctactacgttcggccaagggaccaaggtggaaatcaaacgggc

SEQ ID NO: 233 - DNA, 326 bp, Homo sapiens
gacatccagatgacccagtctccatcctccctgtctgcatctgtaggagaccgtgtcaccatcacttgccgggcaagtcagtctattggggttaatttaatgtggtaccagcagaaaccagggaaagcccctaagctcctgatctattttgcttccattttgcaaagtggggtcccatcacgtttcagtggcagtggatctgggacagatttcactctcaccatcagcagtctgcaacctgaagactttgctacgtactactgtcaacagaattatgatattcctaagacgttcggccaagggaccaaggtggaaatcaaacgggc

SEQ ID NO: 234 - DNA, 326 bp, Homo sapiens
gacatccagatgacccagtctccatcctccctgtctgcatctgtaggagaccgtgtcaccatcacttgccgggcaagtcagtggattggtatttctttatcttggtatcagcagaaaccagggaaagcccctaagctcctaatctattatgggtccgtgttgcaaagtggggtcccatcccgtttcagtggcagtggatctgggacagatttcactctcaccatcagcagtctgcaacctgaagattttgctacgtactactgtcaacagtctcatgatcttcctgtgacgtttggccaagggaccaaggtggaaatcaaacgggc

SEQ ID NO: 235 - DNA, 326 bp, Homo sapiens
gacatccagatgacccagtctccatcctccctgtctgcatctgtaggagaccgtgtcaccatcacttgccgggcaagtcagtggattggtgttagtttatcttggtatcagcagaaaccagggaaagcccctaagctcctgatctattatgcgtccattttgcaaagtggggtcccatcacgtttcagtggcagtggatctgggacagatttcactctcaccatcagcagtctgcaacctgaagattttgctacgtactactgtcaacaggagatgtcgtatcctcctacgttcggccaagggaccaaggtggaaatcaaacgggc

SEQ ID NO: 236 - DNA, 326 bp, Homo sapiens
gacatccagatgacccagtctccatcctccctgtctgcatctgtaggagaccgtgtcaccatcacttgccgggcaagccaggatattggtgttagtttagagtggtatcagcagaaaccagggaaagcccctaagctcctgatctattggagttccgctttgcaaagtggggtcccatcacgtttcagtggcagtggatctgggacagatttcactctcaccatcagcagtctgcaacctgaagatttggctacgtactactgtcaacaggggcatacgtatcctagtacgttcggccaagggaccaaggtggaaatcaaacgggc

SEQ ID NO: 237 - DNA, 326 bp, Homo sapiens
gacatccagatgacccagtctccatcctccctgtctgcatctgtaggagaccgtgtcaccatcacttgccgggcaagtcagtatattggtgtttatttaagttggtatcagcagaaaccagggaaagcccctaagctcctgatctattgggcgtcccttttgcaaagtggggtcccatcacgtttcagtggcagtggatctgggacagatttcactctcaccatcagcagtctgcaacctgaagattttgctacgtactactgtcaacagactgttagggatcctattacgttcggccaagggaccaaggtggaaatcaaacgggc

SEQ ID NO: 238 - DNA, 326 bp, Homo sapiens
gacatccagatgacccagtctccatccttcctgtctgcatctgtaggagaccgtgtcaccatcacttgccgggcaagtcagtctatttatactatgttaaattggtatcagcagaaaccagggaaagcccctaagctcctgatctatcgtgcttcctatttgcaaagtggggtcccatcacgtttcagtggcagtggatctgggacagatttcactctcaccatcagcagtctgctacctgaagattctgctacgtactactgtcaacaggatttttcgtatcctagtacgttcggccaagggaccaaggtggaaatcaaacgggc

SEQ ID NO: 239 - DNA, 326 bp, Homo sapiens
gacatccagatgacccagtctccatcctccctgtctgcatctgtaggagaccgtgtcaccatcacttgccgggcaagtcagtatattggggcgaatttaagttggtatcagcagaaaccagggaaagcccctaagctcctgatctattatatttccgttttgcaaagtggggtcccatcacgtttcagtggcagtggatctgggacagatttcactctcaccatcagcagtctgcaacctgaagattttgctacgtactactgtcaacaggagctttatactcctcatacgttcggccaagggaccaaggtggaaatcaaacgggc

SEQ ID NO: 240 - DNA, 326 bp, Homo sapiens
gacatccagttgacccagtctccatcctccctgtctgcatctgtaggagaccgtgtcaccatcacttgccgggcaagtcagtatattggtgtgactttaatgtggtatcagcagaaaccagggaaagcccctaagctcctgatctattatgcttcccagttgcaaagtggggtcccatcacgtttcagtggcagtggatctgggacagatttcactctcaccatcagcagtctgcaacctgaagattttgctacgtactactgtcaacaggaggttagttatccttatacgttcggccaagggaccaaggtggaaatcaaacgggc

SEQ ID NO: 241 - DNA, 326 bp, Homo sapiens
gacatccagatgacccagtctccatcctctctgtctgcatctgtaggagaccgtgtcaccatcacttgccgggcaagtcagcatattggtgtgagtttaacttggtaccagcagaaaccagggaaagcccctaagctcctgatctattttgcttccattttgcaaagtggggtcccatcacgtttcagtggcagtggatctgggacagatttcactctcaccatcagcagtctgcaacctgaagattatgctacgtactactgtcaacaggatatgtcttatcctcctacgttcggccaagggaccaaggtgggaatcaaacgggc

SEQ ID NO: 242 - DNA, 326 bp, Homo sapiens
gacatccagatgacccagtctccatcctccctgtctgcatctgtaggagaccgtgtcaccatcacttgccgggcaagtcaggatattggtatcagtttagagtggtatcagcagaaaccagggaaagcccctaagctcctgatctattttgcttcccagttgcaaagtggggtcccatcacgtttcagtggcagtggatctgggacagatttcactctcaccatcagcagtctgcaacctgaagattttgctacgtactactgtcaacaggtgtatgattttcctaatacgttcggccaagggaccaaggtggaaatcaaacgggc

SEQ ID NO: 243 - DNA, 326 bp, Homo sapiens
gacatccagatgacccagtctccatcctccctgtctgcatctgtaggagaccgtgtcaccatcacttgccgggcaagtcagcatattggtgtgagtttaaattggtatcagcagaaaccagggaaagtccctaagctcctgatctattgggcttccattttgcaaagtggggtcccatcacgtttcagtggcagtggatctgggacagatttcactctcaccatcagcagtctgcaacctgaagattttgctacgtactactgtcaacaggagcatactattccttctacgttcggccaagggaccaaggtggaaatcaaacgggc

SEQ ID NO: 244 - DNA, 326 bp, Homo sapiens
gacatccagatgacccagtctccatcctccctgtctgcatctgtaggagaccgtgtcaccatcacttgccgggcaagtcagcatattggggtttcgttagattggtaccagcagaaaccagggaaagcccctaagctcctgatctattatggttccgagttgcaaagtggggtcccatcacgtttcagtggcagtggatctgggacagatttcactctcaccatcagcagtctgcaacctgaagattttgctacgtactactgtcaacagtatgttactcatcctacgacgttcggccaagggaccaaggtggaaatcaaacgggc

SEQ ID NO: 245 - DNA, 326 bp, Homo sapiens
gacatccagatgacccagtctccatcctccctgtctgcatctgtaggagaccgtgtcaccatcacttgccgggcaagtcagaggattggtatgatgttagattggtatcagcagaaaccagggaaagcccctaagctcctgatctatgggggttccaagttgcaaagtggggtcccatcacgtttcagtggcagtggatctgggacagatttcactctcaccatcagcagtctgcaacctgaagattttgctacgtactactgtcaacaccggggttggtatcctctgacgttcggccaagggaccaaggtggaaatcaaacgggc

SEQ ID NO: 246 - DNA, 326 bp, Homo sapiens
gacatccagatgacccagtctccatcctccctgtctgcatctgtaggagaccgtgtcaccatcacttgccgggcaagtcagcctattggtgatcgtttaagttggtaccagcagaaaccagggaaagcccctaagctcctgatctatttttcttccgttttgcaaagtggggtcccatcacgtttcagtggcagtggatctgggacagatttcactctcaccatcagcagtctgcaacctgaagattttgctacgtactactgtcaacagcatggtttgcggcctgatacgttcggccaagggaccaaggtggaaatcaaacgggc

SEQ ID NO: 247 - DNA, 326 bp, Homo sapiens
gacatccagatgacccagtctccatcctccctgtctgcatcagtaggagaccgtgtcaccatcacttgccgggcaagtcagtggattggtagtagtttaatgtggtatcagcagaaaccagggaaagcccctaagctcctgatctattgggcttccgagttgcaaagtggggtcccatcgcgtttcagtggcagtggatctgggacagatttcactctcaccatcagcagtctgcaacctgaagattttgctacgtactactgtcaacaggagtatagttatcctagtacgttcggccaagggaccaaagtggaaatcaaacgggc

SEQ ID NO: 248 - DNA, 326 bp, Homo sapiens
gacatccagatgacccagtctccatcctccctgtctgcatctgtaggagaccgtgtcaccatcacttgccgggcaagtcaggatattggtgtttcgttagcttggtaccagcagaaaccggggaaagcccctaagctcctgatctattttggttccgtgtcgctaagtggggtcccatcacgtttcagtggcagtggatctgggacagatttcactctcaccatcagcagtctgcaacctgaagattttgctacgtactactgtcaacagtctcatcttcctcctactacgttcggccaagggaccaaggtggaaatcaaacgggc

SEQ ID NO: 249 - DNA, 326 bp, Homo sapiens
gacatccagatgacccagtctccatcctccctgtctgcatctgtaggagaccgtgtcaccatcacttgccgggcaagtcagtggattggggtggagttaaattggtatcagcagaaaccagggaaagcccctaagctcctgatctattggacgtccattttgcaaagtggggtcccatcacgtttcagtggcagtggatctgggacagatttcactctcaccatcagcagtctgcaacctgaagattttgctacgtactactgtcaacaggttattaatagtccctatacgttcggccaagggaccaaggtggaaatcaaacgggc

SEQ ID NO: 250 - DNA, 326 bp, Homo sapiens
gacatccagatgacccagtctccatcctccctgtctgcatctgtaggagaccgtgtcaccatcacttgccgggcaagtcaggatattggtaagtggttagagtggtaccagcagaaaccagggaaagcccctaagctcctgatcggggcgacgtcctggttgcaaagtggggtcccatcacgtttcagtggcagtggatctgggacagatttcactctcaccatcagcagtctgcaacctgaagattttgctacgtactactgtgttcagcaggggaggcgtcctgggacgttcggccaagggaccaaggtggaaatcaaacgggc

SEQ ID NO: 251 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacggtcggatctgtctcctctgacgttcggccaagggaccaaggtggaaatcaaacgggc

SEQ ID NO: 252 - DNA, 326 bp, Homo sapiens
gacatccagatgacccagtctccatcctccctgtctgcatctgtaggagaccgtgtcaccatcacttgccgggcaagtcagaatatttatatgaatttagagtggtaccagcagaaaccagggaaagcccctaagctcctgatcgtttttggttcctggttgcaaagtggggtcccatcacgtttcagtggcagtggatctgggacagatttcactctcaccatcagcagtctgcaacctgaagattttgctacgtactactgtcggcagactgaggcgccgccttctacgttcggccaagggaccaaggtggaaatcaaacgggc

SEQ ID NO: 253 - DNA, 326 bp, Homo sapiens
gacatccggatgacccagtctccatcctccctgtctgcatctgtaggagaccgtgtcaccatcacttgccgggcaagtcagcatattgggtcgtcgttatcttggtaccagcagaaaccagggaaagcccctaagctcctgatctattatgcctccgttttgcaaagtggggtcccatcacgtttcagtggcagtggatctgggacagatttcactctcaccatcagcagtctgcaacctgaagattttgctacgtactactgtcaacaggagtattcttggcctcctacgctcggccaagggaccaaggtggaaatcaaacgggc

SEQ ID NO: 254 - DNA, 326 bp, Homo sapiens
gacatccagatgacccagtctccatcctccctgtctgcatctgtaggagaccgtgtcaccatcacttgccgggcaagtcaggatattcggacgctgttacggtggtaccagcagaaaccagggaaagcccctaagctcctgatctattggtcttccgagttgcaaagtggggtcccatcacgtttcagtggcagtggatctgggacagatttcactctcaccatcagcagtctgcaacctgaagattttgctacgtactactgtcaacagacgtttcatgcgcctaatacgttcggccaagggaccaaggtggaaatcaaacgggc

SEQ ID NO: 255 - DNA, 326 bp, Homo sapiens
gacatccggatgacccagtctccatcctccctgtctgcatctgtaggagaccgtgtcaccatcacttgccgggcaagtcagtatattgggaagtatttaagttggtaccagcagaaaccagggaaagcccctaagctcctgatctatttgtcgtccacgttgcaaagtggggtcccatcacgtttcagtggcagtggatctgggacagatttcactctcaccatcagcagtctgcaacctgaagattttgctacgtactactgtcaacagaatgatcgtttacctcttacgctcggccaagggaccaaggtggaaatcaaacgggc

SEQ ID NO: 256 - DNA, 326 bp, Homo sapiens
gacatccagatgacccagtctccatcctccctgtctgcatctgtaggagaccgtgtcaccatcacttgccaggcaagtcagctgattgggaatatgttatcttggtaccagcagaaaccagggaaagcccctacgctcctgatctatattggttcctctttgcaaagtggggtcccatcacgtttcagtggcagtggatctgggacagatttcactctcaccatcagcagtctgcaacctgaagattttgctacgtactactgtcaacagacgtattttgatcctcctacgttcggccaagggaccaaggtggaaatcaaacgggc

SEQ ID NO: 257 - DNA, 326 bp, Homo sapiens
gacatccagatgacccagtctccatcctccctgtctgcatctgtaggagaccgtgtcaccatcacttgccgggcaagtcagtatattggtattaatttaaggtggtaccagcagaaaccagggaaagcccctaagctcctgatctattatagttccactttgctaagtggggtcccatcacgtttcagtggcagtggatctgggacagatttcactctcaccatcagcagtctgcaacctgaagattttgctacgtactactgtcaacagtcttatgattctcctgttacgttcggccaagggaccaaggtggaaatcaaacgggc

SEQ ID NO: 258 - DNA, 326 bp, Homo sapiens
gacatccagatgacccagtctccatcctccctgtctgcatctgtaggagaccgtgtcaccatcacttgccgggcaagttgggctattggtgatcgtttagagtggtaccagcagaaaccagggaaagcccctaagctcctgatcgcgtgggggtccgtgttgcaaagtggggtcccatcacgtttcagtggcagtggatctgggacagatttcactctcaccatcagcagtctgcaacctgaagattttgctacgtactactgttctcagctgggttcgcggcctcgtacgttcggccaagggaccaaggtggaaatcaaacgggc

SEQ ID NO: 259 - DNA, 326 bp, Homo sapiens
gacatccagatgacccagtctccatcctccctgtctgcatctgtaggagaccgtgtcaccatcacttgccgggcaagtcagtcgattgataattggttagcgtggtaccagcagaaaccaggggaagcccctaagctcctgatctatggtacgtcccggttgcaaagtggggtcccatcgcgtttcagtggcagtggatctgggacagattttactctcaccatcagcagtctgcaacctgaagattttgctacgtactactgtcaacagtataatttttttccttctacgttcggccaagggaccaaggtggaaatcaaacgggc

SEQ ID NO: 260 - DNA, 326 bp, Homo sapiens
gacatccagatgacccagtctccatcctccctgtctgcatctgtaggagaccgtgtcaccatcacttgccgggcaagtacttttattggtaatgtgttaaattggtaccagcagaaaccagggaaagcccctaagctcctgatctcttatgtgtccatgttgcaaagtggggtcccatcacgtttcagtggcagtggatctgggacagatttcactctcaccatcagcagtctgcaacctgaagattttgctacgtactactgttgtcagtcgtatgatgtgccttttacgttcggccaagggacccaggtggaaatcaaacgggc

SEQ ID NO: 261 - DNA, 326 bp, Homo sapiens
gacatccagatgacccagtctccatcctccctgtctgcatctgtaggagaccgtgtcaccatcacttgccgggcaagtcagtggattggggttagtttagtttggtatcagcagaaaccagggaaagcccctaagctcctgatctattgggcttccgttttgcaaagtggagtcccatcacgtttcagtggcagtggatctgggacagatttcactctcaccatcagcagtctgcaacctgaggattttgctacgtactactgtcaacagacgcatgcagggcctcatacgttcggccaagggaccaaggtggaaatcaaacgggc

SEQ ID NO: 262 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacagtcttctgtggatcctcttacgttcggccaagggaccaaggtggaaatcaaacgggc

SEQ ID NO: 263 - DNA, 326 bp, Homo sapiens
gacatccagatgacccagtctccatcctccctgtctgcatctgtaggagaccgtgtcaccatcacttgccgggcaagtcaggatattggtgtgtcgttaaggtggtatcagcagaaaccagggaaagcccctaagctcctgatctattgggcttccgagttgcaaagtggggtcccatcacgtttcagtggcagtggatctgggacagatttcactctcaccatcagcagtctgcaacctgaagattttgctacgtactactgtcaacagctttatgattatcctccgacgttcggccaagggaccaaggtggaaatcaaacgggc

SEQ ID NO: 264 - DNA, 326 bp, Homo sapiens
gacatccagatgacccagtctccatcctccctgtctgcatctgtaggagaccgtgtcaccatcacttgccgggcaagtcgttttattgcttctggtttagattggtaccagcagaaaccagggaaagcccctaagctcctgatctcgcggttttccgggttgcaaagtggggtcccatcacgtttcagtggcagtggatctgggacagatttcactctcaccatcagcagtctgcaacctgaagattttgctacgtactactgtaagcaggggtttggggctcctgcgacgttcggccaagggaccaaggtggaaatcaaacgggc

SEQ ID NO: 265 - DNA, 329 bp, Homo sapiens
gacatccagatgacccagtctccatcctccctgtctgcatctgtaggagaccgtgtcaccatcacttgccgggcaagtcagtatatttctacggagttagagtggtatcagcagaaaccagggaaagcccctaagctcctgatctattcgagttccatgttgcaaagtggggtcccatcacgtttcagtggcagtggatctgggacagatttcactctcaccatcagcagtctgcaacctgaagattttgctacgtactactgtcaacagagtgcttcggcgcttcctctgacgttcggccaagggaccaaggtggaaatcaaacgggc

SEQ ID NO: 266 - DNA, 326 bp, Homo sapiens
gacatccagatgacccagtctccatcctccctgtctgcatctgtaggagaccgtgtcaccatcacttgccgggcaagtcagtggattggtgcgtctttacagtggtaccagcagaaaccagggcaagcccctaagctcctgatctattatatgtccgtgttgcaaagtggggtcccatcacgtttcagtggcagtggatctgggacagatttcactctcaccatcagcagtctgcaacctgaagattttgctacgtactactgtcaacagacggctttgactcctgctacgttcggccaagggaccaaggtggaaatcaaacgggc

SEQ ID NO: 267 - DNA, 326 bp, Homo sapiens
gacatccagatgacccagtctccatcctccctgtctgcatctgtaggagaccgtgtcaccatcacttgccgggcaagtcaggttattggggattatttatcttggtaccagcagaaaccagggaaagcccctaagctcctgatctattttcgttccgttttgcaaagtggggtcccatcacgtttcagtggcagtggatctgggacagatttcactctcaccatcagcagtctgcaacctgaagattttgctacgtactactgtcaacagaattggaatttgcctgttacgttcggccaagggaccaaggtggaaatcaaacgggc

SEQ ID NO: 268 - DNA, 326 bp, Homo sapiens
gacatccagatgacccagtctccatcctccctgtctgcatctgtaggagaccgtgtcaccatcacttgccgggcaagtcagtatattggtgtgaatttatcgtggtaccagcagaaaccagggaaagcccctaagctcctgatctattatgtttccgttttgcaaagtggggtcccatcacgtttcagtggcagtggatctgggacagatttcactctcaccatcagcagtctgcaacctgaagattttgctacgtactactgtcaacagacttatgatattccttctacgttcggccaagggaccaaggtggaaattaaacgggc

SEQ ID NO: 269 - DNA, 326 bp, Homo sapiens
gacatccagatgacccagtctccatccaccctgtctgcatctgtaggagaccgtgtcaccatcacttgccgggcaagtcagtggattggtgtgtctttatcgtggtaccagcagaaaccagggaaagcccctaagctcctgatctattatacgtcctatttgcaaagtggggtcccatcacgtttcagtggcagtggatctgggacagatttcactctcaccatcagcagtctgcaacctgaagattttgctacatactactgtcaacaggagacgacgtggccttatacgttcggccaagggaccaaggtggaaatcaaacgggc

SEQ ID NO: 270 - DNA, 326 bp, Homo sapiens
gacatccagatgacccagtctccatcctccttgtctgcatctgtaggagaccgtgtcaccatcacttgccgggcaagtcagtatattggggcggagttaaattggtaccagcagaaaccagggaaagcccctaagctcctgatctattggacttccgtgttgcaaagtggggtcccatcacgtttcagtggcagtggatctgggacagatttcactctcaccatcagcagtctgcaacctgaagattttgctacgtactactgtcaacaggcgattctggcgcctcttacgttcggccaagggaccaaggtggaaatcaaacgggc

SEQ ID NO: 271 - DNA, 326 bp, Homo sapiens
gacatccagatgacccagtctccatcctccctgtctgcatctgtaggagaccgtgtcaccatcacttgccgggcaagtcagtggattggtgttagtttaaattggtatcagcagaaaccagggaaagcccctaagctcctgatctactttgcgtccgtgttgcagagtggggtcccatcacgtttcagtggcagtggatctgggacagatttcactctcaccatcagcagtctgcaacctgaagattttgctacgtactactgtcaacagaatgcgttttatcctgatacgttcggccaagggaccaaggtggaaatcaaacgggc

SEQ ID NO: 272 - DNA, 326 bp, Homo sapiens
gacatccagatgacccagtctccatcctccctgtctgcatctgtaggagaccgtgtcaccatcacttgccgggcaagtcagtggattggtgcggagttaaattggtatcagcagaaaccagggaaagcccctaagctcctgatctattggatgtccgtgttgcaaagtggggtcccatcacgtttcagtggcagtggatctgggacagatttcactctcaccatcagcagtctgcaacctgaagattttgctacgtactactgtcaacagacttctttttttcctattacgttcggccaagggaccaaggtggaaatcaaacgggc

SEQ ID NO: 273 - DNA, 326 bp, Homo sapiens
gacatccagatgacccagtctccatcctccctgtctgcctctgtaggagaccgtgtcgccatcacttgccgggcaagtcaggatattcggacgcttttagcttggtaccagcagaaaccagggaaagcccctatgctcctgatctattgggcttccgagttgcaaagtggggtcccatcacgtttcagtggcagtggatctgggacagatttcactctcaccatcagcagtctgcaacctgaagattttgctacgtactactgtcaacagtctctttcttggccttcgacgttcggccaagggaccaaggtggaaatcaaacgggc

SEQ ID NO: 274 - DNA, 325 bp, Homo sapiens
acatccagatgacccagtctccatcctccctgtctgcatctgtaggagaccgtgtcaccatcacttgccgggcaagtcagtatattggggtgagtttagattggtaccagcagaaaccagggaaagcccctaagctcctgatctattatagttccatgttgcaaagtggggtcccatcacgtttcagtggcagtggatctgggacagatttcactctcaccatcagcagtctgcaacctgaagattttgctacgtactactgtcaacagtattatactgttcctgatacgttcggccaagggaccaaggtggaaatcaaacgggc

SEQ ID NO: 275 - DNA, 326 bp, Homo sapiens
gacatccagatgacccagtctccatcctccctgtctgcatctgtaggagaccgtgtcaccatcacttgccgggcaagttggccgattggtgatcgtttaaattggtaccagcagaaaccagggaaagcccctaagctcctgatcgcttgggtttccgttttgcaaagtggggtcccatcacgtttcagtggcagtggatctgggacagatttcactctcaccatcagcagtctgcaacctgaagattttgctacgtactactgtgggcagttggggggtgggcctcggacgttcggccaagggaccaaggtggaaatcaaacgggc

SEQ ID NO: 276 - DNA, 326 bp, Homo sapiens
gacatccagatgacccagtctccatcctccctgtctgcatctgtaggagaccgtgtctccatcacttgccgggcaagtcagtttattgggtgggagttagcttggtaccagcagaaaccagggaaagcccctatgctcctgatctatccgtattccacgttgcaaagtggggtcccatcacgtttcagtggcagtggatctgggacagatttcactctcaccatcagcagtctgcaacctgaagattttgctacgtactactgtcaacagctggctggttttccttatacgttcggccaagggaccaaggtggaaatcaaacgggc

SEQ ID NO: 277 - DNA, 326 bp, Homo sapiens
gacatccagatgacccagtctccatcctccctgtctgcatctgtaggagaccgtgtcaccatcacttgccgggcaagtcagcctattggtgatcgtttatcttggtaccagcagaaaccagggaaagcccctaagctcctgatctattttgtgtcccagttgcaaagtggggtcccatcacgtttcagtggcagtggatctgggacagatttcactctcaccatcagcagtctgcaacctgaagattttgctacgtactactgtcaacagagtcatcctaatcctaagacgtttggccaagggaccaaggtggaaatcaaacgggc

SEQ ID NO: 278 - DNA, 326 bp, Homo sapiens
gacatccagatgacccagtctccatcctccctgtctgcatctgtaggagacggtgtcaccatcacttgccgggcaagtcagtggattggggtggagttaagttggtatcagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacagttggcgttacctccttttacgttcggccaagggaccaaggtggaaatcaaacgggc

SEQ ID NO: 279 - DNA, 326 bp, Homo sapiens
gacatccagatgacccagtctccatcctccctgtctgcatctgtaggagaccgtgtcaccatcacttgccgggcaagtcaggagatcggggctagtttagagtggtaccagcagaaaccagggaaagcccctaagctcctgatctattgggcttccgtgttgcaaagtggggtcccatcacgtttcagtggcagtggatctgggacagatttcactctcaccatcagcagtctgcaacctgaagattttgctacgtactactgtcaacagatgcatcatactccttttacgttcggccaagggaccaaggtggaaatcaaacgggc

SEQ ID NO: 280 - DNA, 326 bp, Homo sapiens
gacatccagatgacccaatctccatcctccctgtccgcatctgtaggagaccgtgtcaccatcacttgccgggcaagtcagcatattgggcagtttttaagttggtaccagcagaagccagggaaagcccctaagctcctgatctatttggcttccaggttgcaaagtggggtcccatcacgtttcagtggtagtggatctgggacagatttcactctcaccatcagcagtctgcaacctgaagattttgctacgtactactgtcaacaggttgataggattcctgttacgttcggccaagggaccaaggtggaaatcaaacgggc

SEQ ID NO: 281 - DNA, 326 bp, Homo sapiens
gacatccagatgacccagtctccatcctccctgtctgcatctgtaggagaccgtgtcaccatcacttgccgggcaagtcaggggattgatcattttttatcgtggtaccagcagaaaccagggaaagcccctaagctcctgatctattttgcgtccacgttgcaaagtggggtcccatcacgtttcagtggcagtggatctgggacagatttcactctcaccatcagcagtctgcaacctgaagattttgctacgtactactgtcaacagaatgcgagtattcctattacgttcggccaagggaccaaggtggaaatcaaacgggc

SEQ ID NO: 282 - DNA, 326 bp, Homo sapiens
gacatccagatgacccagtctccatcctccctgtctgcatctgtaggagaccgtgtcaccatcacttgccgggcaagtcagaatattggtactaatttaaagtggtatcagcagaaaccagagaaagcccctaagctcctgatctattatgggtcccttttgcaaagtggggtcccatcacgtttcagtggcagtggatctgggacagatttcactctcaccatcagcagtctgcaacctgaagattttgctacgtactactgtcaacaggattatgattttccttatacgttcggccaagggaccaaggtggaaatcaaacgggc

SEQ ID NO: 283 - DNA, 326 bp, Homo sapiens
gacatccagatgacccagtctccatcttccctgtctgcatctgtaggagaccgtgtctccatcacttgccgggcaagtcagtggattgggggagagttaaattggtaccagcagaaaccagggaaagcccctaagctcctgatctattgggtttccacgttgcaaagtggggtcccatcacgtttcagtggcagtggatctgggacagatttcactctcaccatcagcagtctgcaaccagaagattttgctacgtactactgtcaacagattgcgcggtatcctgcgacgttcggccaagggaccaaggtggaaatcaaacgggc

SEQ ID NO: 284 - DNA, 326 bp, Homo sapiens
gacatccagatgacccagtctccatcctccctgtctgcatctgtaggagaccgtgtcaccatcacttgccgggcaagtcagaatattggtgtgaatttaatttggtaccagcagaaaccagggaaagcccctaggctcctgatctatttttcttcccttttgcaaagtggggtcccatcacgtttcagtggcagtggatctgggacagatttcactctcaccatcagcagtctgcaacctgaagattttgctacgtactactgtcaacaggattatgatgttcctcagacgttcggccaagggaccaaggtggaaatcaaacgggc

SEQ ID NO: 285 - DNA, 326 bp, Homo sapiens
gacatccagatgacccagtctccatcctccctgtctgcatctgtaggagaccgtgtcaccatcacttgccgggcaagtcagaatattgggagtgggttacattggtaccagcagaaaccagggaaagcccctaagctcctgatcgtttcttggtccggtttgcaaagtggggtcccatcacgtttcagtggcagtggatctgggacagatttcactctcaccatcagcagtctgcaacctgaagattttgctacgtactactgtggtcaggatgtgttgggtcctcctacgttcggccaagggaccaaggtggaaatcaaacgggc

SEQ ID NO: 286 - DNA, 326 bp, Homo sapiens
gacatccagatgacccagtctccatcctccctgtctgcatctgtaggagaccgtgtcaccatcacttgccgggcaagtcagtggattggggcgtcgttagcttggtaccagcagaaaccagggaaagcccctaagctcctgatctattttatgtccgagttgcaaagtggggtcccatcacgttttagtggcagtggatctgggacagatttcactctcaccatcagcagtctgcaacctgaagattttgctacgtactactgtcaacaggattatggttatcctactacgttcggccaagggaccaaggtggaaatcaaacgggc

SEQ ID NO: 287 - DNA, 326 bp, Homo sapiens
gacatccagatgacccagtctccatcctccctgtctgcatctgtaggagaccgtgtcaccatcacttgccgggcaagtcagtggattggtgttaatttattgtggtaccagcagaaaccagggaaagcccctaagctcctgatctattatggttccattttgcaaagtggggtcccatcacgtttcagtggcagtggatctgggacagatttcactctcaccatcagcagtctgcaacctgaagattttgctacgtactactgtcaacaggattatcatgggccttatacgttcggccaagggaccaaggtggaaatcaaacgggc

SEQ ID NO: 288 - DNA, 326 bp, Homo sapiens
gacatccagatgacccagtctccatcctccctgtctgcatctgtaggagaccgtgtcaccatcacttgccggacaagtcaggatattgggtctctgttatcgtggtaccagcagaaaccagggaaagcccctaagctcctgatctatatggtttccatgttgcaaagtggggtcccatcacgtttcagtggcagtggatctgggacagatttcactctcaccatcagcagtctgcaacctgaagattttgctacgtactactgtcaacataattcgtggtatcctattacgttcggccaagggaccaaggtggaaatcaaacgggc

SEQ ID NO: 289 - DNA, 325 bp, Homo sapiens
acatccagatgacccagtctccatcctccctgtctgcatctgtaggagaccgtgtcaccatcacttgccgggcaagtcagtttatttatactatgttaaattggtaccagcagaaaccagggaaagcccctaagctcctgatctataggacgtcctggttgcaaagtggggtcccatcacgtttcagtggcagtggatctgggacagatttcactctcaccatcagcagtctgcaacctgaagattttgctacgtactactgtcaacaggattatgcgtcgccttttacgttcggccaagggaccaaggtggaaatcaaacgggc

SEQ ID NO: 290 - DNA, 326 bp, Homo sapiens
gacatccagatgacccagtctccatcctccctgtctgcatctgttggagaccgtgtcaccatcacttgccgggcaagtcagtggattggtattttgttagattggtaccagcagaaaccaggggaagcccctaagctcctgatctattatgcttcctttttgcaaagcggggtcccatcacgtttcagtggcagtggatttgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 291 - DNA, 326 bp, Homo sapiens
gacatccagatgacccagtctccatcctccctgtctgcatctgtaggagaccgtgtcaccatcacttgccgggcaagtcagtggattggtattttgatagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattctgctacgtactactgtcaacaggctaatccggcgcctctgacgttcggccaagggaccaaggtggaaatcaaacgggc

SEQ ID NO: 292 - DNA, 326 bp, Homo sapiens
gacatccagttgacccagtctccatcctccctgtctgcatctgtaggagaccgagtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcaacagtctgcaacctgaagattttgctacgtactactgtcaacaggctaatccggcgcctctgacgttcggccaagggaccaaggtggaaatcaaacgggc

SEQ ID NO: 293 - DNA, 326 bp, Homo sapiens
gacatccagatgacccagtctccatccttcctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttaggccaagggaccaaggtggaaatcaaacgggc

SEQ ID NO: 294 - DNA, 326 bp, Homo sapiens
gacatccagatgacccagtctccatcctccctgtctgcatctgtaggagaccgtgtcaccatcacttgccgggcaagtcagtggattggtattttggtagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgaggttcggccaagggaccaaggtggaaatcaaacgggc

SEQ ID NO: 295 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaaatggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 296 - DNA, 326 bp, Homo sapiens
gacatccagatgatccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagtaatctgcaacctgaagattttgctacgtactactgtcaacaggctaatccggcgcctctgacgttcggccaagggaccaaggtggaaatcaaacgggc

SEQ ID NO: 297 - DNA, 326 bp, Homo sapiens
gacatccagatgacccagtctccatcctccctgtctgcatctgtgggagaccgtgtcaccatcacttgccgggcaagtcagtggattggtattttggtagattggtaccagcagaaaccaggggaagcccctaagctcctgatctattatgcttcctttttgcaaagtggggtcccatcacgtttcagcggcagtggatttgggacagatttcactctcaccatcagcagtctgcatcctgaagattttgctacgtactactgtcaacaggctaatccggcgcctctgacgttcggccaagggaccaaggtggaaatcaaacgggc

SEQ ID NO: 298 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcaatctgcaacctgaagattttgctacgtactactgtcaacaggctgatccggcgcctctgacgttcggccaagggaccaaggtggaaatcaaacgggc

SEQ ID NO: 299 - DNA, 326 bp, Homo sapiens
gacatccagatgacccagtctccatcttccctgtctgcatctgtaggagacagtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggacaaaggtggaaatcaaacgggc

SEQ ID NO: 300 - DNA, 326 bp, Homo sapiens
gacatccagatgacccagtatccatcctccctgtctgcatctgtaggagaccgtgtcaccatcacttgccgggcaagtcagtggattggtattttgttagattggtaccagcagaaaccaggggaagcccctaagctcctgatctattatgcctcctttttgcagagtggggtcccatcacgtttcagtggcagtggatctgggacagatttcactctcaccatcagcagtctgcaacctgaagattttggtacgtactactgtcaacaggctaatccggcgcctctgacgttcggccaagggaccaaggtggaaatcaaacgggc

SEQ ID NO: 301 - DNA, 326 bp, Homo sapiens
gacatccagatgacccagtctccatcctccctgtatgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 302 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaaccggctaatccggcacctctgacgttcggccaagggaccaaggtggaaatcaaacgggc

SEQ ID NO: 303 - DNA, 326 bp, Homo sapiens
gacatccagatgacccagtctccatcctccctgtctgcatctgtaggagaccgtgtcaccatcacttgccggtcaagtcggtggattggtattttgttagattggtaccagcagaaaccaggggaagcccctaagctcctgatctattatgcttcctttttgcaaagtggggtcccctcacgtttcagtggcagtgggtctgggacagatttcactctcaccatcagcagtctgcaacctgaagatgttgctacgtactactgtcaacaggctaatccggcgcctctgacgttcggccaagggaccaaggtggaaatcaaacgggc

SEQ ID NO: 304 - DNA, 326 bp, Homo sapiens
gacatccagatgacccagtctccatcctccctgtctgcatctgtaggagaccgtgtcaccatcacttgccgggcaagtcagtggatcggtattttggtagattggtaccagcagaaaccaggggaagcccctaagctcctgatctattatgcttcctttttgcaaagtggggtcccatcacgtttcagtggcagtggatcagggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 305 - DNA, 326 bp, Homo sapiens
gacatccagttgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactattgtcaacaggctaatccggcgcctctgacgttcggccaagggaccaaggtggaaatcaaacgggc

SEQ ID NO: 306 - DNA, 326 bp, Homo sapiens
gacatccagatgacccagtctccaacctccctgtctgcatctgtaggagaccgtgtcaccatcacttgccgggcaagtcagtggattggtaatttgttagattggtaccagcagaaaccaggggaagcccctaagctcctgatctattatgcttcctttttgcaaagtggggtcccatcacgtttcagtggcggtggatttgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 307 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctccgacgttcggccaagggaccaaggtggaaatcaaacgggc

SEQ ID NO: 308 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagaccctaagctcctgatctattatgcttcctttttgcaaagtggggtcccatcacgtttcagtggcagtggatctgggacagatttcactctcaccatcagcagtctgcaacctgaagattttgctacgtactactgccaacaggctaatccggcgcctctgacgttcggccaagggaccaaggtggaaatcaaacgggc

SEQ ID NO: 309 - DNA, 326 bp, Homo sapiens
gacatccagatgacccagtctccaacctccctgtctgcatctgtaggagaccgtgtcaccatcacctgcagggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 310 - DNA, 326 bp, Homo sapiens
gacatccagatgacccagtctccatcctccctgtctgcatctgtaggagaccgtgtcaccatcacttgccgggcaagtcagtggattggtattttggtagattggtaccagcagaaaccaggggaagcccctaagctcctgatctattatggttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 311 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcagacgggc

SEQ ID NO: 312 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatatgggacagatttcactctcaccatcagcagtttgcaacctgaagattttgctacgtactactgccaacaggctaatccggcgcctctgacgttcggccaagggaccaaggtggaaatcaaacgggc

SEQ ID NO: 313 - DNA, 326 bp, Homo sapiens
gacatccagatgacccagtctccatcctccctgtctgcatctgtaggagaccgtgtcaccatcacttgccgggcaagtcagtggattggtattttgttagattggtatcagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatgaaacgggc

SEQ ID NO: 314 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagaccctaagctcctgatctattatgcttcctttttgcaaagtggggtcccatcacgtttcagtggcagcggatctgggacagatttcactctcaccatcagcagtctgcaacctgaagattttgctacgtactactgtcaacaggctaatccggcgcctctgatgttcggccaagggaccaaggtggaaatcaaacgggc

SEQ ID NO: 315 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 316 - DNA, 326 bp, Homo sapiens
gacatccagatgacccagtctccatcctccctgtctgcatctgtaggagaccgtgtcaccatcacttgccgggcaagtcagtggattggtattttggtagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 317 - DNA, 326 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgaggttcggccaagggaccaaggtggaaatcaaacgggc

SEQ ID NO: 318 - DNA, 326 bp, Homo sapiens
gacatccagatgacccagtctccatcctccctgtctgcatctgtaggagaccgtgtcaccatcacttgccgggcaagtcagtggattggtattttgatagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 319 - DNA, 326 bp, Homo sapiens
gacatccagatgacccagtctccatcctccctgtctgcatctgtaggagaccgtgtcaccatcacttgccgggcaagtcagtggattggtaatttggtagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 320 - DNA, 326 bp, Homo sapiens
gacatccagatgacccagtctccatcctccctgtctgcatctgtaggagaccgtgtcaccatcacttgccgggcaagtcagtggattggtaatttggtagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctccgacgttcggccaagggaccaaggtggaaatcaaacgggc

SEQ ID NO: 321 - DNA, 326 bp, Homo sapiens
gacatccagatgacccagtctccatcctccctgtctgcatctgtaggagaccgtgtcaccatcacttgccgggcaagtcagtggattggtatcaacttagactggtaccagcagaaaccagggaaagcccctaagctcctgatctattatgcttcctttttgcaaagtggggtcccatcacgtttcagtggcagtggatctgggacagatttcactctcaccatcagcagtctgcaaccggaagattttgctacgtactactgtcaacaggctaatccggcgcctctgacgttcggccaagggaccaaggtggaaatcaaacgggc

SEQ ID NO: 322 - DNA, 326 bp, Homo sapiens
gacatccagatgacccagtctccatcctccctgtctgcatctgtaggagaccgtgtcaccatcacttgccgggcaagtcagaacattggcaacttgttagattggtaccagcagaaaccagggaaagcccctaagctcctgatctattatgcttcctttttgcaaagtggggtcccatcacgtttcagtggcagtggatttgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 323 - DNA, 326 bp, Homo sapiens
gacatccagatgacccagtctccatcctccctgtctgcatctgtaggagaccgtgtcaccatcacttgccgggcaagtcagtggattggtatcaacttagactggtaccagcagaaaccagggaaagcccctaggctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgggc

SEQ ID NO: 324 - DNA, 357 bp, Homo sapiens
gaggtgcagctgttggagtctgggggaggcttggtacagcctggggggtccctgcgtctctcctgtgctgcctccggattcacctttgctgagcagccgatgacttgggcccgccaggctccagggaagggtctagagtgggtctcaagtatttctagttttggtgatcttacatactacgcagactccgtgaagggccggttcaccatctcccgcgacaattccaagaacacgctgtatctgcaaatgaacagcctgcgtgccgaggacaccgcggtatattactgtgcgaaaggtgtgtatcggattagtcggtttgactactggggtcagggaaccctggtcaccgtctcgagc

SEQ ID NO: 325 - protein, 108 aa, Homo sapiens
DIQMTQSPSSLSASVGDRVTITCRASQYIGAELSWYQQKPGKAPKLLIYWISELQSGVPSRFSGSGSGTDFTLTISSLQPEDFATYYCQQSSNTPYTFGQGTKVEIKR

SEQ ID NO: 326 - protein, 108 aa, Homo sapiens
DIQMTQSPSSLSASVGDRVTITCRASQYIGINLIWYQQKPGKAPKLLIYWASVLQSGVPSRFSGSGSGTDFTLTISSLQPEDFATYYCQQSVYDPPTYGQGTKVEIKR

SEQ ID NO: 327 - protein, 108 aa, Homo sapiens
DIQMTQSPSSLSASVGDRVTITCRASQDIFWLLSWYQQKPGKAPTLLIYSTSILQSGVPSRFSGSGSGTDFTLTISSLQPEDFATYYCQQYYLDPPTFSQGTKVEIKR

SEQ ID NO: 328 - protein, 108 aa, Homo sapiens
DIQMTQSPSSLSASVGDRVTITCRASQFIGVNLNWYQQKPGKAPRLLIYLSSILQSGVPSRFSGSGSGTDFTLTISSLQPEDFATYYCQQTYDIPTTFGQGTKVEIKR

SEQ ID NO: 329 - protein, 108 aa, Homo sapiens
DIQMTQSPSSLSASVGDRVTITCRASQNIGINLQWYQQKPRKAPKLLIYYASILQSGVPSRFSGSGSGTDFTLTISSLQPEDFATYYCQQDYDTPFTFGQGTKVEIKR

SEQ ID NO: 330 - protein, 108 aa, Homo sapiens
DIQMTQFPSSLSASVGDRVTITCRASQHIERWLNWYQQKPGKAPKLLIYRSSYLQSGVPSRFSGSGSGTDFTLTISSLQPEDFATYYCQQDAILPHTFGQGTKVEIKR

SEQ ID NO: 331 - protein, 108 aa, Homo sapiens
DIQMTQSPSSLSASVGDRVTITCRASQGIGVNLQWYQQKPGKAPKLLIYFSSVLQSGVPSRFSGSGSGTDFTLTISSLQPEDFATYYCQQDFDFPQTFGQGTKVEIKR

SEQ ID NO: 332 - protein, 108 aa, Homo sapiens
DIQMTQSPSSLSASVGDRVTITCRASQNIGINLQWYQQKPRKAPKLLIYYASILQSGVPSRFSGSGSGTDFTLTISSLQPEDFATYYCQQEYDYPNTFGQGTKVEIKR

SEQ ID NO: 333 - protein, 108 aa, Homo sapiens
DIQMTQSPSSLSASVGDRVTITCRASSSIGSGLEWYQQKPGKAPKLLIVGWSGLQSGVPSRFSGSGSGTDFTLTISSLQPEDFATYYCRQCVGLPCTFGQGTKVEIKR

SEQ ID NO: 334 - protein, 108 aa, Homo sapiens
DIQMTQSPSSLSASVGDRVTITCRASQWIGVELSWYQQKPGKAPKLLIYWGSELQSGVPSRFSGSGSGTDFTLTISSLQPEDFATYYCQQLALPPFTFGQGTKVEIKR

SEQ ID NO: 335 - protein, 108 aa, Homo sapiens
DIQMTQSPSSLSASVGDRVTITCRASHDIGVSLDWYQQKPGKAPKLLIVWASVLQSGVPSRFSGSGSGTDFTLTISSLQPEDFATYYCLQVGAGPMTFGQGTKVEIKR

SEQ ID NO: 336 - protein, 108 aa, Homo sapiens
DIQMTQSPSSLSASVGDGVTITCRASQYIGIDLAWYQRKPGKAPRLLIYKASALQSGVPSRFSGSGSGTDFTLTISSLQPEDFATYYCQQYADYPATFGQGTKVEIKR

SEQ ID NO: 337 - protein, 108 aa, Homo sapiens
DIQMTQSPSSLSASVGDRVTITCRASQEIEHYLSWYQQKPGKAPKLLIYLSSRLQSGVPSRFSGSGSGTDFTLTISSLQPEDFATYYCQQNVQLPITFGQGTKVEIKR

SEQ ID NO: 338 - protein, 108 aa, Homo sapiens
DIQMTQSPSSLSASVGDRVTITCRASQEIGVSLSWYQQKPGKAPKLLIYWGSELQSGVPSRFSGSGSGTDFTLTISSLQPEDFATYYCQQDHNWPMTFGQGTKVEIKR

SEQ ID NO: 339 - protein, 108 aa, Homo sapiens
DIQMTQSPSSLSASVGDRVTITCRASALIMGDLDWYQQKPGKAPKLLIAGVSFLQSGVPSRFSGSGSGTDFTLTISSLQPEDFATYYCAQSRSWPYTFGQGTKVEIKR

SEQ ID NO: 340 - protein, 108 aa, Homo sapiens
DIQMTQSPSSLSASVGDRVTITCRASKMIDENLAWYQQKPGKAPKLLILRSSGLQSGVPSRFSGSGSGTDFTLTISSLQPEDFATYYCHQGHSAPGTFGQGTKVEIKR

SEQ ID NO: 341 - protein, 108 aa, Homo sapiens
DIQMTQSPSSLSASVGDRVTITCRASRYIGVSLDWYQQKPGKAPKLLIMWGSALQSGVPSRFSGSGSGTDFTLTISSLQPEDFATYYCLQSAAPPATFGQGTKVEIKR

SEQ ID NO: 342 - protein, 108 aa, Homo sapiens
DIQMTQSPSSLSASVGDRVTITCRASQEIGVSLSWYQQKPGKAPKLLIYWASALQSGVPSRFSGSGSGTDFTLTISSLQPEDFATYYCQQSYLPPDTFGQGTKVEIKR

SEQ ID NO: 343 - protein, 108 aa, Homo sapiens
DIQMTQSPSSLSASVGDRVTITCRASQEIASDLLWYQQKPGKAPKLLIYNGSSLQSGVPSRFSGSGSGTDFTLTISRLQPEDFATYYCQWLWSEPLTFGQGTKVEIKR

SEQ ID NO: 344 - protein, 108 aa, Homo sapiens
DIQMTQSPSSLSASVGDRVTITCRASQHIGDALWWYQQKPGKAPKLLIYWTSNLQSGVPSRFSGSGSGTDFTLTISSLQPEDFATYYCQQTIRRPYTFGQGTKVKIKR

SEQ ID NO: 345 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPAPLTFGQGTKVEIKR

SEQ ID NO: 346 - protein, 108 aa, Homo sapiens
DIQMTQSPSSLSASVGDRVTITCRASQNIGINLQWYQQKPRKAPKLLIYYASILQSGVPSRFSGSGSGTDFTLTISSLQPEDFATYYCQQSYDLPKTFGQGTKVEIKR

SEQ ID NO: 347 - protein, 108 aa, Homo sapiens
DIQMIQSPSSLSASVGDRVTITCRASQEIGVSLSWYQQKPGKAPKLLIYWGSELQSGVPSRFSGSGSGTDFTLTISSLQPEDFATYYCQQWYGWPDTFGQGTKVGIKR

SEQ ID NO: 348 - protein, 108 aa, Homo sapiens
DIQMTQSPSSLSASVGDRVTITCRASQHIGIELNWYQQKPGKAPKLLIYWASVLQSGVPSRFSGSGSGTDFTLTISSLQPEDFATYYCQQSVYVPTTFGQGTKVEIKR

SEQ ID NO: 349 - protein, 108 aa, Homo sapiens
DIQMTQSPSSLSASVGDRVTITCRASQWIGAELVWYQQKPGKAPKLLIYWSSVLQSGVPSRFSGSGSGTDFTLTISSLQPEDFATYYCQQAAHSPPTFGQGTKVEIKR

SEQ ID NO: 350 - protein, 108 aa, Homo sapiens
DIQMTQSPSSLSASVGDRVTITCRASQDISRSLAWYQRKPGKAPRLLIYMSSTLQSGVPSRFSGSGSGTDFTLTISSLQPEDFATYYCQQYDSYPSTFGQGTKVEIKR

SEQ ID NO: 351 - protein, 108 aa, Homo sapiens
DIQMTQSPSSLSASVGDRVTITCRASRMIGGMLLWYQQKPGKAPKLLITYGSVLQSGVPSRFSGSGSGTDFTLTISSLQPEDFATYYCAQEFWWPHTFGQGTKVEIKR

SEQ ID NO: 352 - protein, 108 aa, Homo sapiens
DIQMTQSPSSLSASVGDRVTITCRASRPIGDRLTWYQQKPGKAPKLLISWVSVLQSGVPSRFSGSGSGTDFTLTISSLQPEDFATYYCRQLGGGPFTFGQGTKVEIKR

SEQ ID NO: 353 - protein, 108 aa, Homo sapiens
DIQMTQSPSSLSASVGDRVTITCRASQYIGVSLSWYQQKPGKAPKLLIYFASALQSGVPSRFSGSGSGTDFTLTISSLQPEDFATYYCQQDRDWPATFGQGTKVEIKR

SEQ ID NO: 354 - protein, 108 aa, Homo sapiens
DIQMTQSPSSLSASVGDRVTITCRASGAIGDRLKWYQQKPGKAPKLLISWASVLQSGVPSRFSGSGSGTDFTLTISSLQPEDFATYYCVQGPGVPLTFGQGTKVEIKR

SEQ ID NO: 355 - protein, 108 aa, Homo sapiens
DIQMTQSPSSLSASVGDRVTITCRASQPIARWLAWYQQKPGKAPKLLIYGSSVLQSGVPSRFSGSGSGTDFTLTISSLQPEDFATYYCQQDLRLPPTFGQGTKVEIKR

SEQ ID NO: 356 - protein, 108 aa, Homo sapiens
DIQMTQSPSSLSASVGDRVTITCRASQNIGVSLSWYQQKPGKAPRLLIYYGSNLLSGVPSRFSGSGSGTDFTLTISSLQPEDFATYYCQQEFSWPVTFGQGTKVEIKR

SEQ ID NO: 357 - protein, 108 aa, Homo sapiens
DIQMTQSPSSLSASVGDRVTITCRASLPIDDGLGWYQQKPGKAPKLLICGVSGLQSGVPLRFSGSGSGTDFTLTISSLQPEDFATYYCGQGQVQPSTFGQGTKVEIKR

SEQ ID NO: 358 - protein, 108 aa, Homo sapiens
DIQMTQSPSSLSASVGDRVTITCRASQNIGINLQWYQQKPRKAPKLLIYYASILQSGVPSRFSGSGSGTDFTLTISSLQPEDFATYYCQQSYDAPTTFGQGTKVEIKR

SEQ ID NO: 359 - protein, 108 aa, Homo sapiens
DIQMTQSPSSLSASVGDRVTITCRASQSIGVNLMWYQQKPGKAPKLLIYFASILQSGVPSRFSGSGSGTDFTLTISSLQPEDFATYYCQQNYDIPKTFGQGTKVEIKR

SEQ ID NO: 360 - protein, 108 aa, Homo sapiens
DIQMTQSPSSLSASVGDRVTITCRASQWIGISLSWYQQKPGKAPKLLIYYGSVLQSGVPSRFSGSGSGTDFTLTISSLQPEDFATYYCQQSHDLPVTFGQGTKVEIKR

SEQ ID NO: 361 - protein, 108 aa, Homo sapiens
DIQMTQSPSSLSASVGDRVTITCRASQWIGVSLSWYQQKPGKAPKLLIYYASILQSGVPSRFSGSGSGTDFTLTISSLQPEDFATYYCQQEMSYPPTFGQGTKVEIKR

SEQ ID NO: 362 - protein, 108 aa, Homo sapiens
DIQMTQSPSSLSASVGDRVTITCRASQDIGVSLEWYQQKPGKAPKLLIYWSSALQSGVPSRFSGSGSGTDFTLTISSLQPEDLATYYCQQGHTYPSTFGQGTKVEIKR

SEQ ID NO: 363 - protein, 108 aa, Homo sapiens
DIQMTQSPSSLSASVGDRVTITCRASQYIGVYLSWYQQKPGKAPKLLIYWASLLQSGVPSRFSGSGSGTDFTLTISSLQPEDFATYYCQQTVRDPITFGQGTKVEIKR

SEQ ID NO: 364 - protein, 108 aa, Homo sapiens
DIQMTQSPSFLSASVGDRVTITCRASQSIYTMLNWYQQKPGKAPKLLIYRASYLQSGVPSRFSGSGSGTDFTLTISSLLPEDSATYYCQQDFSYPSTFGQGTKVEIKR

SEQ ID NO: 365 - protein, 108 aa, Homo sapiens
DIQMTQSPSSLSASVGDRVTITCRASQYIGANLSWYQQKPGKAPKLLIYYISVLQSGVPSRFSGSGSGTDFTLTISSLQPEDFATYYCQQELYTPHTFGQGTKVEIKR

SEQ ID NO: 366 - protein, 108 aa, Homo sapiens
DIQLTQSPSSLSASVGDRVTITCRASQYIGVTLMWYQQKPGKAPKLLIYYASQLQSGVPSRFSGSGSGTDFTLTISSLQPEDFATYYCQQEVSYPYTFGQGTKVEIKR

SEQ ID NO: 367 - protein, 108 aa, Homo sapiens
DIQMTQSPSSLSASVGDRVTITCRASQHIGVSLTWYQQKPGKAPKLLIYFASILQSGVPSRFSGSGSGTDFTLTISSLQPEDYATYYCQQDMSYPPTFGQGTKVGIKR

SEQ ID NO: 368 - protein, 108 aa, Homo sapiens
DIQMTQSPSSLSASVGDRVTITCRASQDIGISLEWYQQKPGKAPKLLIYFASQLQSGVPSRFSGSGSGTDFTLTISSLQPEDFATYYCQQVYDFPNTFGQGTKVEIKR

SEQ ID NO: 369 - protein, 108 aa, Homo sapiens
DIQMTQSPSSLSASVGDRVTITCRASQHIGVSLNWYQQKPGKVPKLLIYWASILQSGVPSRFSGSGSGTDFTLTISSLQPEDFATYYCQQEHTIPSTFGQGTKVEIKR

SEQ ID NO: 370 - protein, 108 aa, Homo sapiens
DIQMTQSPSSLSASVGDRVTITCRASQHIGVSLDWYQQKPGKAPKLLIYYGSELQSGVPSRFSGSGSGTDFTLTISSLQPEDFATYYCQQYVTHPTTFGQGTKVEIKR

SEQ ID NO: 371 - protein, 108 aa, Homo sapiens
DIQMTQSPSSLSASVGDRVTITCRASQRIGMMLDWYQQKPGKAPKLLIYGGSKLQSGVPSRFSGSGSGTDFTLTISSLQPEDFATYYCQHRGWYPLTFGQGTKVEIKR

SEQ ID NO: 372 - protein, 108 aa, Homo sapiens
DIQMTQSPSSLSASVGDRVTITCRASQPIGDRLSWYQQKPGKAPKLLIYFSSVLQSGVPSRFSGSGSGTDFTLTISSLQPEDFATYYCQQHGLRPDTFGQGTKVEIKR

SEQ ID NO: 373 - protein, 108 aa, Homo sapiens
DIQMTQSPSSLSASVGDRVTITCRASQWIGSSLMWYQQKPGKAPKLLIYWASELQSGVPSRFSGSGSGTDFTLTISSLQPEDFATYYCQQEYSYPSTFGQGTKVEIKR

SEQ ID NO: 374 - protein, 108 aa, Homo sapiens
DIQMTQSPSSLSASVGDRVTITCRASQDIGVSLAWYQQKPGKAPKLLIYFGSVSLSGVPSRFSGSGSGTDFTLTISSLQPEDFATYYCQQSHLPPTTFGQGTKVEIKR

SEQ ID NO: 375 - protein, 108 aa, Homo sapiens
DIQMTQSPSSLSASVGDRVTITCRASQWIGVELNWYQQKPGKAPKLLIYWTSILQSGVPSRFSGSGSGTDFTLTISSLQPEDFATYYCQQVINSPYTFGQGTKVEIKR

SEQ ID NO: 376 - protein, 108 aa, Homo sapiens
DIQMTQSPSSLSASVGDRVTITCRASQDIGKWLEWYQQKPGKAPKLLIGATSWLQSGVPSRFSGSGSGTDFTLTISSLQPEDFATYYCVQQGRRPGTFGQGTKVEIKR

SEQ ID NO: 377 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQRSDLSPLTFGQGTKVEIKR

SEQ ID NO: 378 - protein, 108 aa, Homo sapiens
DIQMTQSPSSLSASVGDRVTITCRASQNIYMNLEWYQQKPGKAPKLLIVFGSWLQSGVPSRFSGSGSGTDFTLTISSLQPEDFATYYCRQTEAPPSTFGQGTKVEIKR

SEQ ID NO: 379 - protein, 108 aa, Homo sapiens
DIRMTQSPSSLSASVGDRVTITCRASQHIGSSLSWYQQKPGKAPKLLIYYASVLQSGVPSRFSGSGSGTDFTLTISSLQPEDFATYYCQQEYSWPPTLGQGTKVEIKR

SEQ ID NO: 380 - protein, 108 aa, Homo sapiens
DIQMTQSPSSLSASVGDRVTITCRASQDIRTLLRWYQQKPGKAPKLLIYWSSELQSGVPSRFSGSGSGTDFTLTISSLQPEDFATYYCQQTFHAPNTFGQGTKVEIKR

SEQ ID NO: 381 - protein, 108 aa, Homo sapiens
DIRMTQSPSSLSASVGDRVTITCRASQYIGKYLSWYQQKPGKAPKLLIYLSSTLQSGVPSRFSGSGSGTDFTLTISSLQPEDFATYYCQQNDRLPLTLGQGTKVEIKR

SEQ ID NO: 382 - protein, 108 aa, Homo sapiens
DIQMTQSPSSLSASVGDRVTITCQASQLIGNMLSWYQQKPGKAPTLLIYIGSSLQSGVPSRFSGSGSGTDFTLTISSLQPEDFATYYCQQTYFDPPTFGQGTKVEIKR

SEQ ID NO: 383 - protein, 108 aa, Homo sapiens
DIQMTQSPSSLSASVGDRVTITCRASQYIGINLRWYQQKPGKAPKLLIYYSSTLLSGVPSRFSGSGSGTDFTLTISSLQPEDFATYYCQQSYDSPVTFGQGTKVEIKR

SEQ ID NO: 384 - protein, 108 aa, Homo sapiens
DIQMTQSPSSLSASVGDRVTITCRASWAIGDRLEWYQQKPGKAPKLLIAWGSVLQSGVPSRFSGSGSGTDFTLTISSLQPEDFATYYCSQLGSRPRTFGQGTKVEIKR

SEQ ID NO: 385 - protein, 108 aa, Homo sapiens
DIQMTQSPSSLSASVGDRVTITCRASQSIDNWLAWYQQKPGEAPKLLIYGTSRLQSGVPSRFSGSGSGTDFTLTISSLQPEDFATYYCQQYNFFPSTFGQGTKVEIKR

SEQ ID NO: 386 - protein, 108 aa, Homo sapiens
DIQMTQSPSSLSASVGDRVTITCRASTFIGNVLNWYQQKPGKAPKLLISYVSMLQSGVPSRFSGSGSGTDFTLTISSLQPEDFATYYCCQSYDVPFTFGQGTQVEIKR

SEQ ID NO: 387 - protein, 108 aa, Homo sapiens
DIQMTQSPSSLSASVGDRVTITCRASQWIGVSLVWYQQKPGKAPKLLIYWASVLQSGVPSRFSGSGSGTDFTLTISSLQPEDFATYYCQQTHAGPHTFGQGTKVEIKR

SEQ ID NO: 388 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQSSVDPLTFGQGTKVEIKR

SEQ ID NO: 389 - protein, 108 aa, Homo sapiens
DIQMTQSPSSLSASVGDRVTITCRASQDIGVSLRWYQQKPGKAPKLLIYWASELQSGVPSRFSGSGSGTDFTLTISSLQPEDFATYYCQQLYDYPPTFGQGTKVEIKR

SEQ ID NO: 390 - protein, 108 aa, Homo sapiens
DIQMTQSPSSLSASVGDRVTITCRASRFIASGLDWYQQKPGKAPKLLISRFSGLQSGVPSRFSGSGSGTDFTLTISSLQPEDFATYYCKQGFGAPATFGQGTKVEIKR

SEQ ID NO: 391 - protein, 109 aa, Homo sapiens
DIQMTQSPSSLSASVGDRVTITCRASQYISTELEWYQQKPGKAPKLLIYSSSMLQSGVPSRFSGSGSGTDFTLTISSLQPEDFATYYCQQSASALPLTFGQGTKVEIKR

SEQ ID NO: 392 - protein, 108 aa, Homo sapiens
DIQMTQSPSSLSASVGDRVTITCRASQWIGASLQWYQQKPGQAPKLLIYYMSVLQSGVPSRFSGSGSGTDFTLTISSLQPEDFATYYCQQTALTPATFGQGTKVEIKR

SEQ ID NO: 393 - protein, 108 aa, Homo sapiens
DIQMTQSPSSLSASVGDRVTITCRASQVIGDYLSWYQQKPGKAPKLLIYFRSVLQSGVPSRFSGSGSGTDFTLTISSLQPEDFATYYCQQNWNLPVTFGQGTKVEIKR

SEQ ID NO: 394 - protein, 108 aa, Homo sapiens
DIQMTQSPSSLSASVGDRVTITCRASQYIGVNLSWYQQKPGKAPKLLIYYVSVLQSGVPSRFSGSGSGTDFTLTISSLQPEDFATYYCQQTYDIPSTFGQGTKVEIKR

SEQ ID NO: 395 - protein, 108 aa, Homo sapiens
DIQMTQSPSTLSASVGDRVTITCRASQWIGVSLSWYQQKPGKAPKLLIYYTSYLQSGVPSRFSGSGSGTDFTLTISSLQPEDFATYYCQQETTWPYTFGQGTKVEIKR

SEQ ID NO: 396 - protein, 108 aa, Homo sapiens
DIQMTQSPSSLSASVGDRVTITCRASQYIGAELNWYQQKPGKAPKLLIYWTSVLQSGVPSRFSGSGSGTDFTLTISSLQPEDFATYYCQQAILAPLTFGQGTKVEIKR

SEQ ID NO: 397 - protein, 108 aa, Homo sapiens
DIQMTQSPSSLSASVGDRVTITCRASQWIGVSLNWYQQKPGKAPKLLIYFASVLQSGVPSRFSGSGSGTDFTLTISSLQPEDFATYYCQQNAFYPDTFGQGTKVEIKR

SEQ ID NO: 398 - protein, 108 aa, Homo sapiens
DIQMTQSPSSLSASVGDRVTITCRASQWIGAELNWYQQKPGKAPKLLIYWMSVLQSGVPSRFSGSGSGTDFTLTISSLQPEDFATYYCQQTSFFPITFGQGTKVEIKR

SEQ ID NO: 399 - protein, 108 aa, Homo sapiens
DIQMTQSPSSLSASVGDRVAITCRASQDIRTLLAWYQQKPGKAPMLLIYWASELQSGVPSRFSGSGSGTDFTLTISSLQPEDFATYYCQQSLSWPSTFGQGTKVEIKR

SEQ ID NO: 400 - protein, 108 aa, Homo sapiens
DIQMTQSPSSLSASVGDRVTITCRASQYIGVSLDWYQQKPGKAPKLLIYYSSMLQSGVPSRFSGSGSGTDFTLTISSLQPEDFATYYCQQYYTVPDTFGQGTKVEIKR

SEQ ID NO: 401 - protein, 108 aa, Homo sapiens
DIQMTQSPSSLSASVGDRVTITCRASWPIGDRLNWYQQKPGKAPKLLIAWVSVLQSGVPSRFSGSGSGTDFTLTISSLQPEDFATYYCGQLGGGPRTFGQGTKVEIKR

SEQ ID NO: 402 - protein, 108 aa, Homo sapiens
DIQMTQSPSSLSASVGDRVSITCRASQFIGWELAWYQQKPGKAPMLLIYPYSTLQSGVPSRFSGSGSGTDFTLTISSLQPEDFATYYCQQLAGFPYTFGQGTKVEIKR

SEQ ID NO: 403 - protein, 108 aa, Homo sapiens
DIQMTQSPSSLSASVGDRVTITCRASQPIGDRLSWYQQKPGKAPKLLIYFVSQLQSGVPSRFSGSGSGTDFTLTISSLQPEDFATYYCQQSHPNPKTFGQGTKVEIKR

SEQ ID NO: 404 - protein, 108 aa, Homo sapiens
DIQMTQSPSSLSASVGDGVTITCRASQWIGVELSWYQQKPGKAPKLLIYWGSELQSGVPSRFSGSGSGTDFTLTISSLQPEDFATYYCQQLALPPFTFGQGTKVEIKR

SEQ ID NO: 405 - protein, 108 aa, Homo sapiens
DIQMTQSPSSLSASVGDRVTITCRASQEIGASLEWYQQKPGKAPKLLIYWASVLQSGVPSRFSGSGSGTDFTLTISSLQPEDFATYYCQQMHHTPFTFGQGTKVEIKR

SEQ ID NO: 406 - protein, 108 aa, Homo sapiens
DIQMTQSPSSLSASVGDRVTITCRASQHIGQFLSWYQQKPGKAPKLLIYLASRLQSGVPSRFSGSGSGTDFTLTISSLQPEDFATYYCQQVDRIPVTFGQGTKVEIKR

SEQ ID NO: 407 - protein, 108 aa, Homo sapiens
DIQMTQSPSSLSASVGDRVTITCRASQGIDHFLSWYQQKPGKAPKLLIYFASTLQSGVPSRFSGSGSGTDFTLTISSLQPEDFATYYCQQNASIPITFGQGTKVEIKR

SEQ ID NO: 408 - protein, 108 aa, Homo sapiens
DIQMTQSPSSLSASVGDRVTITCRASQNIGTNLKWYQQKPEKAPKLLIYYGSLLQSGVPSRFSGSGSGTDFTLTISSLQPEDFATYYCQQDYDFPYTFGQGTKVEIKR

SEQ ID NO: 409 - protein, 108 aa, Homo sapiens
DIQMTQSPSSLSASVGDRVSITCRASQWIGGELNWYQQKPGKAPKLLIYWVSTLQSGVPSRFSGSGSGTDFTLTISSLQPEDFATYYCQQIARYPATFGQGTKVEIKR

SEQ ID NO: 410 - protein, 108 aa, Homo sapiens
DIQMTQSPSSLSASVGDRVTITCRASQNIGVNLIWYQQKPGKAPRLLIYFSSLLQSGVPSRFSGSGSGTDFTLTISSLQPEDFATYYCQQDYDVPQTFGQGTKVEIKR

SEQ ID NO: 411 - protein, 108 aa, Homo sapiens
DIQMTQSPSSLSASVGDRVTITCRASQNIGSGLHWYQQKPGKAPKLLIVSWSGLQSGVPSRFSGSGSGTDFTLTISSLQPEDFATYYCGQDVLGPPTFGQGTKVEIKR

SEQ ID NO: 412 - protein, 108 aa, Homo sapiens
DIQMTQSPSSLSASVGDRVTITCRASQWIGASLAWYQQKPGKAPKLLIYFMSELQSGVPSRFSGSGSGTDFTLTISSLQPEDFATYYCQQDYGYPTTFGQGTKVEIKR

SEQ ID NO: 413 - protein, 108 aa, Homo sapiens
DIQMTQSPSSLSASVGDRVTITCRASQWIGVNLLWYQQKPGKAPKLLIYYGSILQSGVPSRFSGSGSGTDFTLTISSLQPEDFATYYCQQDYHGPYTFGQGTKVEIKR

SEQ ID NO: 414 - protein, 108 aa, Homo sapiens
DIQMTQSPSSLSASVGDRVTITCRTSQDIGSLLSWYQQKPGKAPKLLIYMVSMLQSGVPSRFSGSGSGTDFTLTISSLQPEDFATYYCQHNSWYPITFGQGTKVEIKR

SEQ ID NO: 415 - protein, 108 aa, Homo sapiens
DIQMTQSPSSLSASVGDRVTITCRASQFIYTMLNWYQQKPGKAPKLLIYRTSWLQSGVPSRFSGSGSGTDFTLTISSLQPEDFATYYCQQDYASPFTFGQGTKVEIKR

SEQ ID NO: 416 - protein, 108 aa, Homo sapiens
DIQMTQSPSSLSASVGDRVTITCRASQWIGILLDWYQQKPGEAPKLLIYYASFLQSGVPSRFSGSGFGTDFTLTISSLQPEDFATYYCQQANPAPLTFGQGTKVEIKR

SEQ ID NO: 417 - protein, 108 aa, Homo sapiens
DIQMTQSPSSLSASVGDRVTITCRASQWIGILIDWYQQKPGEAPKLLIYYASFLQSGVPSRFSGSGSGTDFTLTISSLQPEDSATYYCQQANPAPLTFGQGTKVEIKR

SEQ ID NO: 418 - protein, 108 aa, Homo sapiens
DIQLTQSPSSLSASVGDRVTITCRASQWIGILLDWYQQKPGEAPKLLIYYASFLQSGVPSRFSGSGSGTDFTLTINSLQPEDFATYYCQQANPAPLTFGQGTKVEIKR

SEQ ID NO: 419 - protein, 108 aa, Homo sapiens
DIQMTQSPSFLSASVGDRVTITCRASQWIGILLDWYQQKPGEAPKLLIYYASFLQSGVPSRFSGSGSGTDFTLTISSLQPEDFATYYCQQANPAPLTLGQGTKVEIKR

SEQ ID NO: 420 - protein, 108 aa, Homo sapiens
DIQMTQSPSSLSASVGDRVTITCRASQWIGILVDWYQQKPGEAPKLLIYYASFLQSGVPSRFSGSGSGTDFTLTISSLQPEDFATYYCQQANPAPLRFGQGTKVEIKR

SEQ ID NO: 421 - protein, 108 aa, Homo sapiens
DIQMTQSPSSLSASVGDRVTITCRASQWIGILLDWYQQKPGEAPKLLIYYASFLQNGVPSRFSGSGSGTDFTLTISSLQPEDFATYYCQQANPAPLTFGQGTKVEIKR

SEQ ID NO: 422 - protein, 108 aa, Homo sapiens
DIQMIQSPSSLSASVGDRVTITCRASQWIGILLDWYQQKPGEAPKLLIYYASFLQSGVPSRFSGSGSGTDFTLTISNLQPEDFATYYCQQANPAPLTFGQGTKVEIKR

SEQ ID NO: 423 - protein, 108 aa, Homo sapiens
DIQMTQSPSSLSASVGDRVTITCRASQWIGILVDWYQQKPGEAPKLLIYYASFLQSGVPSRFSGSGFGTDFTLTISSLHPEDFATYYCQQANPAPLTFGQGTKVEIKR

SEQ ID NO: 424 - protein, 108 aa, Homo sapiens
DIQMTQSPSSLSASVGDRVTITCRASQWIGILLDWYQQKPGEAPKLLIYYASFLQSGVPSRFSGSGSGTDFTLTISNLQPEDFATYYCQQADPAPLTFGQGTKVEIKR

SEQ ID NO: 425 - protein, 108 aa, Homo sapiens
DIQMTQSPSSLSASVGDSVTITCRASQWIGILLDWYQQKPGEAPKLLIYYASFLQSGVPSRFSGSGSGTDFTLTISSLQPEDFATYYCQQANPAPLTFGQGTKVEIKR

SEQ ID NO: 426 - protein, 108 aa, Homo sapiens
DIQMTQYPSSLSASVGDRVTITCRASQWIGILLDWYQQKPGEAPKLLIYYASFLQSGVPSRFSGSGSGTDFTLTISSLQPEDFGTYYCQQANPAPLTFGQGTKVEIKR

SEQ ID NO: 427 - protein, 108 aa, Homo sapiens
DIQMTQSPSSLYASVGDRVTITCRASQWIGILLDWYQQKPGEAPKLLIYYASFLQSGVPSRFSGSGSGTDFTLTISSLQPEDFATYYCQQANPAPLTFGQGTKVEIKR

SEQ ID NO: 428 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQPANPAPLTFGQGTKVEIKR

SEQ ID NO: 429 - protein, 108 aa, Homo sapiens
DIQMTQSPSSLSASVGDRVTITCRSSRWIGILLDWYQQKPGEAPKLLIYYASFLQSGVPSRFSGSGSGTDFTLTISSLQPEDVATYYCQQANPAPLTFGQGTKVEIKR

SEQ ID NO: 430 - protein, 108 aa, Homo sapiens
DIQMTQSPSSLSASVGDRVTITCRASQWIGILVDWYQQKPGEAPKLLIYYASFLQSGVPSRFSGSGSGTDFTLTISSLQPEDFATYYCQQANPAPLTFGQGTKVEIKR

SEQ ID NO: 431 - protein, 108 aa, Homo sapiens
DIQLTQSPSSLSASVGDRVTITCRASQWIGILLDWYQQKPGEAPKLLIYYASFLQSGVPSRFSGSGSGTDFTLTISSLQPEDFATYYCQQANPAPLTFGQGTKVEIKR

SEQ ID NO: 432 - protein, 108 aa, Homo sapiens
DIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKR

SEQ ID NO: 433 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPAPPTFGQGTKVEIKR

SEQ ID NO: 434 - protein, 108 aa, Homo sapiens
DIQMTQSPSSLSASVGDRVTITCRASQWIGILLDWYQQKPGEDPKLLIYYASFLQSGVPSRFSGSGSGTDFTLTISSLQPEDFATYYCQQANPAPLTFGQGTKVEIKR

SEQ ID NO: 435 - protein, 108 aa, Homo sapiens
DIQMTQSPTSLSASVGDRVTITCRASQWIGILLDWYQQKPGEAPKLLIYYASFLQSGVPSRFSGSGSGTDFTLTISSLQPEDFATYYCQQANPAPLTFGQGTKVEIKR

SEQ ID NO: 436 - protein, 108 aa, Homo sapiens
DIQMTQSPSSLSASVGDRVTITCRASQWIGILVDWYQQKPGEAPKLLIYYGSFLQSGVPSRFSGSGSGTDFTLTISSLQPEDFATYYCQQANPAPLTFGQGTKVEIKR

SEQ ID NO: 437 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPAPLTFGQGTKVEIRR

SEQ ID NO: 438 - protein, 108 aa, Homo sapiens
DIQMTQSPSSLSASVGDRVTITCRASQWIGILLDWYQQKPGEAPKLLIYYASFLQSGVPSRFSGSGYGTDFTLTISSLQPEDFATYYCQQANPAPLTFGQGTKVEIKR

SEQ ID NO: 439 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPAPLTFGQGTKVEMKR

SEQ ID NO: 440 - protein, 108 aa, Homo sapiens
DIQMTQSPSSLSASVGDRVTITCRASQWIGILLDWYQQKPGEDPKLLIYYASFLQSGVPSRFSGSGSGTDFTLTISSLQPEDFATYYCQQANPAPLMFGQGTKVEIKR

SEQ ID NO: 441 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPAPLTFGQGTKVEIKR

SEQ ID NO: 442 - protein, 108 aa, Homo sapiens
DIQMTQSPSSLSASVGDRVTITCRASQWIGILVDWYQQKPGKAPKLLIYYASFLQSGVPSRFSGSGSGTDFTLTISSLQPEDFATYYCQQANPAPLTFGQGTKVEIKR

SEQ ID NO: 443 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPAPLRFGQGTKVEIKR

SEQ ID NO: 444 - protein, 108 aa, Homo sapiens
DIQMTQSPSSLSASVGDRVTITCRASQWIGILIDWYQQKPGKAPKLLIYYASFLQSGVPSRFSGSGSGTDFTLTISSLQPEDFATYYCQQANPAPLTFGQGTKVEIKR

SEQ ID NO: 445 - protein, 108 aa, Homo sapiens
DIQMTQSPSSLSASVGDRVTITCRASQWIGNLVDWYQQKPGKAPKLLIYYASFLQSGVPSRFSGSGSGTDFTLTISSLQPEDFATYYCQQANPAPLTFGQGTKVEIKR

SEQ ID NO: 446 - protein, 108 aa, Homo sapiens
DIQMTQSPSSLSASVGDRVTITCRASQWIGNLVDWYQQKPGKAPKLLIYYASFLQSGVPSRFSGSGSGTDFTLTISSLQPEDFATYYCQQANPAPPTFGQGTKVEIKR

SEQ ID NO: 447 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCQQANPAPLTFGQGTKVEIKR

SEQ ID NO: 448 - protein, 108 aa, Homo sapiens
DIQMTQSPSSLSASVGDRVTITCRASQNIGNLLDWYQQKPGKAPKLLIYYASFLQSGVPSRFSGSGFGTDFTLTISSLQPEDFATYYCQQANPAPLTFGQGTKVEIKR

SEQ ID NO: 449 - protein, 108 aa, Homo sapiens
DIQMTQSPSSLSASVGDRVTITCRASQWIGINLDWYQQKPGKAPRLLIYYASFLQSGVPSRFSGSGSGTDFTLTISSLQPEDFATYYCQQANPAPLTFGQGTKVEIKR

SEQ ID NO: 450 - protein, 119 aa, Homo sapiens
EVQLLESGGGLVQPGGSLRLSCAASGFTFAEQPMTWARQAPGKGLEWVSSISSFGDLTYYADSVKGRFTISRDNSKNTLYLQMNSLRAEDTAVYYCAKGVYRISRFDYWGQGTLVTVSS

SEQ ID NO: 451 - protein, 127 aa, Unknown
  Camelid
EVQLVESGGGLVQAGGSLRLSCAASGRTFSNYVMGWFRQAPGKERDFVVGIGRSGGDNTYYADSVKGRFTISWDNAKNTMYLQMNSLKPEDTAVYYCAASTYSRDTIFTKWANYNYWGQGTQVTVSS

SEQ ID NO: 452 - protein, 127 aa, Unknown
  Camelid
QVQLQESGGGLVKAGGSLRLSCAASGRTFSSYVMGWFRQAPGKEREFVGAIHWSGGRTYYADSVKGRFTISSDNAKNTLYLQMNSLKPEDTAVYYCAASRIIYSYVNYVNPGEYDYWGQGTQVTVSS

SEQ ID NO: 453 - protein, 122 aa, Unknown
  Camelid
EVQLVESGGGLVQPGGSLRLSCAASGFTFSSHYMSWFRQAPGKEREFVAAITSSSRTYYTESVKGRFTISRDNAKNTVYLQMNSLKSEDTAVYYCAADRTFYGSTWSKYDYRGQGTQVTVSS

SEQ ID NO: 454 - protein, 129 aa, Unknown
  Camelid
QVQLQESGGGLVQAGGSLRLSCAASGRTFSKYAMGWFRQAPGKEREFVSAISWSDGSTYYADSVKGRFTISRDNAKNTVYLQVNSLKPEDTAVYYCAATYLVDVWAVHVPIRPYEYDYWGQGTQVTVSS

SEQ ID NO: 455 - protein, 125 aa, Unknown
  Camelid
QVQLQDSGGGLVQAGDSLRLSCAASGRSFGGYAMGWFRQAPGKEREFVAAISWSGGSTYYADSLKGRFTISRDNAKNTVYLQMNSLKPEDTALYYCAAGLRPSPNYNHERSYDYWGQGTQVTVSS

SEQ ID NO: 456 - protein, 129 aa, Unknown
  Camelid
QVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSS

SEQ ID NO: 457 - protein, 122 aa, Unknown
  Camelid
QVQLQESGGRLVQTGGSLRLSCAASGGTFGTYALGWFRQAPGKEREFVAAISRFGSTYYADSVKGRFTISRDNANNTVYLEMNSLKPEDTAVYYCAAREGVALGLRNDANYWGQGTQVTVSS

SEQ ID NO: 458 - protein, 121 aa, Unknown
  Camelid
QVQLQDSGGGLVQAGGSLRLSCAASGGTFSSYAMGWFRQAPGKEREFVAAIGLNTYYADSVKGRFTISRDNAKNTVYLQMNSLKPEDTAVYYCAARTSGVVGGTPKRYDYWGQGTQVTVSS

SEQ ID NO: 459 - protein, 122 aa, Unknown
  Camelid
QVQLQESGGGSVQAGGSLKLSCAASGRGFSRYAMGWFRQAPGQDREFVATISWTNSTDYADSVKGRFAISRDNAKNTAYLQMNSLKPEDTAVYYCAADKWASSTRSIDYDYWGQGIQVTVSS

SEQ ID NO: 460 - protein, 123 aa, Unknown
  Camelid
QVQLQESGGGLVQAGGSLRLSCAASGRTFSNYAMGWFRQAPGKEREFVAAINWGGGNTYYADSVKGRFTISRDNAKNTVYLQMNSLKPEDTAVYYCAASEWGGSDYDHDYDYWGQGTQVTVSS

SEQ ID NO: 461 - protein, 123 aa, Unknown
  Camelid
EVQLVESGGGLVQAGGSLRLSCAASGRSFSSYAMAWFRQAPGKEREFVAAISWGGGSTYYAVSVKGRFTISRDNAKNTVYLQMNSLKPEDTARYYCAADETFHSSAYGEYEYWGQGTQVTVSS

SEQ ID NO: 462 - protein, 133 aa, Unknown
  Camelid
EVQLVESGGGLVQAGGSLRLSCTASGRTFSSYAMGWFRQTPGKEREFVAAITSSGGSTYYADSVKGRFTISRDNAKSTMYLQMDSLMLDDTSVYYCAADSSRPQYSDSALRRILSLSNSYPYWGQGTQVTVSS

SEQ ID NO: 463 - protein, 120 aa, Unknown
  Camelid
EVXLVESGGGLVXPGGSLRLSCVASGFTFADYAMSWVRQAPGKGLQWVSSISYNGDTTYYAESMKDRFTISRDNAKNTLYLQMNSLKSEDTAVYYCASSGSYYPGHFESWGQGTQVTVSS

SEQ ID NO: 464 - protein, 121 aa, Unknown
  Camelid
QVQLQESGGGLVQAGGSLRLSCAASGRTFSGYAMGWFRQAPGEEREFVAAISWRGTSTYYGDSAKGRFTISRDNAKNTVYLQMNSLKPEDTAVYYCAAGSHSDYAPDYDYWGQGTQVTVSS

SEQ ID NO: 465 - protein, 123 aa, Unknown
  Camelid
QVQLQESGGGLVQAGGSLRLSCAASGRTFSSYAIGWFRQAPGKEREFVAAISWGGSNTYYADSVKGRFTISRDNAKNTVYLQMNSLKPEDTAVYYCAAGEVSNSDYAYEYDYWGQGTQVTVSS

SEQ ID NO: 466 - protein, 122 aa, Unknown
  Camelid
QVQLQESGGGLVQTGGSLRLSCAASGRYIMGWFRQAPGKEREFVAGISRSGASTAYADSVKDRFTISRDSALNTVYLQMNSLKAEDTAVYFCAAALAIRLGIPRGETEYEYWGQGTQVTVSS

SEQ ID NO: 467 - protein, 121 aa, Unknown
  Camelid
QVKLEESGGGLVQAGGSLRLSCSASGLTFSNYAMAWFRQAPGKEREFVATISQRGGMRHYLDSVKDRFTISRDNAKNTVYLQMNSLKPDDTAVYYCAADLMYGVDRRYDYWGRGTQVTVSS

SEQ ID NO: 468 - protein, 127 aa, Unknown
  Camelid
QVKLEESGGGLVQAGDSLRLSCAASGRSFSSITMGWFRQAPGKERQFVSAINSNGNRYYADSVKGRFTISRDNAKNTVYLQMNSLKPEDTAVYYCAAVQAYSSSSDYYSQEGAYDYWGQGTQVTVSS

SEQ ID NO: 469 - protein, 126 aa, Unknown
  Camelid
EVQLVESGGGLVQAGGSLRLSCAVSGRTFSSMGWFRQAPGKEREFVATINLSGDRTDYADSVKGRFTISRDNPKNTVYLQMDSLEPEDSAVYYCAGTSLYPSNLRYYTLPGTYADWGQGTQVTVSS

SEQ ID NO: 470 - protein, 126 aa, Unknown
  Camelid
QVKLEESGGGLVQAGGSLRLSCAASGSIFSINAMGWYRQAPGKQRELVARITGTGTGITGAVSTNYADSVKGRFTISRDNARNTVYLQMNSLKPEDTAVYYCAADRSRTIVVPDYWGQGTQVTVSS

SEQ ID NO: 471 - protein, 126 aa, Unknown
  Camelid
QVQLQDSGGGLVQAGGSLRLSCAASRFSSAQYAIGWFRQAPGKEREGVSYITFSGGPTGYADSVKGRFTVSRDNAKNTVYLQMNSLKPEDTAVYYCAARPYTRPGSMWVSSLYDNWGQGTQVTVSS

SEQ ID NO: 472 - protein, 123 aa, Unknown
  Camelid
QVQLQESGGRLVQAGGSLRLSCAASEHTFRGYAIGWFRQAPGKEREFVSSITYDGTLTNYADSVTGRFTISRDNAKNTVYLQMNSLKPEDTAVYVCAAGYSYRYTTLNQYDSWGQGTQVTVSS

SEQ ID NO: 473 - protein, 108 aa, Homo sapiens
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 474 - protein, 108 aa, Homo sapiens
DIQMTQSPSSLSASVGDRVTITCRASQSIFRHLKWYQQKPGKAPKLLIYAASRLQSGVPSRFSGSGSGTDFTLTISSLQPEDFATYYCQQVALYPKTFGQGTKVEIKR

SEQ ID NO: 475 - protein, 108 aa, Homo sapiens
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 476 - protein, 108 aa, Homo sapiens
DIQTTQSPSSLSASVGDRVTITCRASQYIGRYLRWYQQKPGKAPKLLIYDSSVLQSGVPSRFSGSGSGTDFTLTISSLQPEDFATYYCQQRYRMPYTFGQGTRVEIKR

SEQ ID NO: 477 - protein, 108 aa, Homo sapiens
DIQMTQSPSSLSASVGDRVTITCRASQYIGRYLRWYQQKPGKAPKLLIYDSSVLQSGVPSRFSGSGSGTDFTLTISSLQPEDFATYYCQQRYMQPFTFGQGTKVEIKR

SEQ ID NO: 478 - protein, 108 aa, Homo sapiens
DIQMTQSPSSLSASVGDRVTITCRASQWIGRYLRWYQQKPGKAPKLLIYNGSQLQSGVPSRFSGSGSGTDFTLTISSLQPEDFATYYCQQRYLQPYTFGQGTKVEIKR

SEQ ID NO: 479 - protein, 108 aa, Homo sapiens
DIQMTQSPSSLSASVGDRVTITCRASQYISRQLRWYQQKPGKAPRLLIYGASVLQSGIPSRFSGSGSGTDFTLTISSLQPEDFATYYCQQRYITPYTFGQGTKVEVKR

SEQ ID NO: 480 - protein, 108 aa, Homo sapiens
DIQMTQSPSSLSASVGDRVTITCRASQYIGRYLRWYQQKPGKAPKLLIYDSSVLQSGVPSRFSGSGSGTDFTLTISSLQPEDFATYYCQQRYSSPYTFGQGTKVEIKR

SEQ ID NO: 481 - protein, 108 aa, Homo sapiens
DIQMTQSPSSLSASVGDRVTITCRASQWIHRQLKWYQQKPGKAPKLLIYYASILQSGVPSRFSGSGSGTDFTLTISSLQPEDFATYYCQQTFSKPSTFGQGTKVEIKR

SEQ ID NO: 482 - protein, 108 aa, Homo sapiens
DIQMTQSPSSLSASVGDRVTITCRASQKIATYLNWYQQKPGKAPKLLIYRSSSLQSAVPSRFSGSGSGTVFTLTISSLQPEDFATYYCQQTYAVPPTFGQGTKVEIKR

SEQ ID NO: 483 - protein, 108 aa, Homo sapiens
DIQMTQSPSSLSASVGDRVTITCRASQWIDTGLAWYQQKPGKAPRLLIYNVSRLQSGVPSRFSGSGSGTDFTLTISSLQPEDFATYYCQQYWGSPTTFGQGTKVEIKR

SEQ ID NO: 484 - protein, 108 aa, Homo sapiens
DIQMTQSPSSLSASVGDRVTITCRASQEIYSWLAWYQQRPGKAPKLLIYNASHLQSGVPSRFSGSGSGTDFTLTISSLQPEDFATYYCQQVIGDPVTFGQGTKVEIKR

SEQ ID NO: 485 - protein, 108 aa, Homo sapiens
DIQMTQSPSSLSASVGDRVTITCRASQSISSYLNWYQQKPGKAPTLLIYRLSVLQSGVPSRFSGSGSGTDFTLTISSLQPEDFATYYCQQTYNVPPTFGQGTKVEIKR

SEQ ID NO: 486 - protein, 108 aa, Homo sapiens
DIQMTQSPSSLSASVGDRVTITCRASQSISSYLNWYQQKPGKAPKLLIYRNSFLQSGVPSRFSGSGSGTDFTLTISSLQPEDFATYYCQQTYTVPPTFGQGTKVEIKQ

SEQ ID NO: 487 - protein, 108 aa, Homo sapiens
DIQMTQSPSSLSASVGDRVTITCRASQSISSYLNWYQQKPGKAPKLLIYRNSQLQSGVPSRFSGSGSGTDFTLTISSLQPEDFATYYCQQTFAVPPTFGQGTKVEIKR

SEQ ID NO: 488 - protein, 35 aa, Homo sapiens
  Consensus
EVQLLESGGGLVQPGGSLRLSCAASGFTFXXYNMS

SEQ ID NO: 489 - protein, 123 aa, Homo sapiens
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 490 - protein, 118 aa, Homo sapiens
EVQLLESGGGLVQPGGSLRLSCAASGFTFYDYNMSWVRQAPGKGLEWVSTITHTGGVTYYADSVKGRFTISRDNSKNTLYLQMNSLRAEDTAVYYCAKQNPSYQFDYWGQGTLVTVSS

SEQ ID NO: 491 - protein, 118 aa, Homo sapiens
EVQLLESGGGLVQPGGSLRLSCAASGFTFHRYSMSWVRQAPGKGLEWVSTILPGGDVTYYADSVKGRFTISRDNSKNTLYLQMNSLRAEDTAVYYCAKQTPDYMFDYWGQGTLVTVSS

SEQ ID NO: 492 - protein, 117 aa, Homo sapiens
EVQLLESGGGLVQPGGSLRLSCAASGFTFWKYNMAWVRQAPGKGLEWVSTILGEGNNTYYADSVKGRFTISRDNSKNTLYLQMNSLRAEDTAVYYCAKTMDYKFDYWGQGTLVTVSS

SEQ ID NO: 493 - protein, 118 aa, Homo sapiens
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 494 - protein, 120 aa, Homo sapiens
EVQLLESGGGLVQPGGSLRLSCAASGFTFDLYDMSWVRQAPGKGLEWVSSIVNSGVRTYYADSVKGRFTISRDNSKNTLYLQMNSLRAEDTAVYYCAKLNQSYHWDFDYWGQGTLVTVSS

SEQ ID NO: 495 - protein, 118 aa, Homo sapiens
EVQLLESGGGLVQPGGSLRLSCAASGFTFSDYRMSWVRQAPGKGLEWVSTIISNGKFTYYADSVKGRFTISRDNSKNTLYLQMNSLRAEDTAVYYCAKQDWMYMFDYWGQGTLVTVSS

SEQ ID NO: 496 - protein, 108 aa, Homo sapiens
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 497 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQKYLPPYTFGQGTKVEIKR

SEQ ID NO: 498 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQRYRVPYTFGQGTKVEIKR

SEQ ID NO: 499 - protein, 108 aa, Homo sapiens
DIQMTQSPSSLSASVGDRVTITCRASQSIGRRLKWYQQKPGAAPRLLIYRTSWLQSGVPSRFSGSGSGTDFTLTISSLQPEDFATYYCQQTSQWPHTFGQGTKVEIKR

SEQ ID NO: 500 - protein, 108 aa, Homo sapiens
DIQMTQSPSSLSASVGDRVTITCRASQKIYKNLRWYQQKPGKAPKLLIYNSSILQSGVPSRFSGSGSGTDFTLTISSLQPEDFATYYCQQRYLSPYTFGQGTKVEIKR

SEQ ID NO: 501 - protein, 108 aa, Homo sapiens
DIQMTQSPSSLSASVGDRVTITCRASQKIYNNLRWYQQKPGKAPKLLIYNTSILQSGVPSRFSGSGSGTDFTLTISSLQPEDFATYYCQQRWRAPYTFGQGTKVEIKR

SEQ ID NO: 502 - protein, 108 aa, Homo sapiens
DIQMTQSPSSLSASVGDRVTITCRASQWIYKSLGWYQQKPGKAPKLLIYQSSLLQSGVPSRFSGSGSGTDFTLTISSLQPEDFATYYCQQYHQMPRTFGQGTKVEIKR

SEQ ID NO: 503 - protein, 108 aa, Homo sapiens
DIQMTQSPSSLSASVGDRVTITCRASQWIYRHLRWYQQKPGKAPKLLIYDASRLQSGVPTRFSGSGSGTDFTLTISSLQPEDFATYYCQQTHNPPKTFGQGTKVEIKR

SEQ ID NO: 504 - protein, 116 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKGGKDFDYWGQGTLVTVSS

SEQ ID NO: 505 - protein, 117 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKGNLEPFDYWGQGTLVTVSS

SEQ ID NO: 506 - protein, 117 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKKLSNGFDYWGQGTLVTVSS

SEQ ID NO: 507 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKVVKDNTFDYWGQGTLVTVSS

SEQ ID NO: 508 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKNTGGKQFDYWGQGTLVTVSS

SEQ ID NO: 509 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKKTGPSSFDYWGQGTLVTVSS

SEQ ID NO: 510 - protein, 120 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKRTENRGVSFDYWGQGTLVTVSS

SEQ ID NO: 511 - protein, 122 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKSDVLKTGLDGFDYWGQGTLVTVSS

SEQ ID NO: 512 - protein, 120 aa, Homo sapiens
EVQLLESGGGLVQPGGSLRLSCAASGFTFMAYQMAWVRQAPGKGLEWVSTIHQTGFSTYYADSVKGRFTISRDNSKNTLYLQMNSLRAEDTAVYYCAKVRSMRPYKFDYWGQGTLVTVSS

SEQ ID NO: 513 - protein, 120 aa, Homo sapiens
EVQLLESGGGLVQPGGSLRLSCAASGFTFKDYDMTWVRQAPGKGLEWVSMISSSGLWTYYADSVKGRFTISRDNSKNTLYLQMNSLRAEDTAVYYCAKGFRLFPRTFDYWGQGTLVTVSS

SEQ ID NO: 514 - protein, 121 aa, Homo sapiens
EVQLLESGGGLVQPGGSLRLSCAASGFTFHDYVMGWARQAPGKGLEWVSLIKPNGSPTYYADSVKGRFTISRDNSKNTLYLQMNSLRAEDTAVYYCAKGRGRFNVLQFDYWGQGTLVTVSS

SEQ ID NO: 515 - protein, 118 aa, Homo sapiens
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYMGDRFDYWGQGTLVTVSS

SEQ ID NO: 516 - protein, 116 aa, Homo sapiens
EVQLLESGGGLVQPGGSLRLSCAASGFTFMWDKMGWVRQAPGKGLEWVSFIGREGYGTYYADSVKGRFTISRDNSKNTLYLQMNSLRAEDTAVYYCAKSVASFDYWGQGTLVTVSS

SEQ ID NO: 517 - protein, 117 aa, Homo sapiens
EVQLLESGGGLVQPGGSLRLSCAASGFTFWAYPMSWVRQAPGKGLEWVSSISSWGTGTYYADSVKGRFTISRDNSKNTLYLQMNSLRAEDTAVYYCAKGGQGSFDYWGQGTLVTVSS

SEQ ID NO: 518 - protein, 115 aa, Unknown
  Camelid
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 519 - protein, 115 aa, Unknown
  Camelid
QVQLQESGGGLVQPGNSLRLSCAASGFTFRNFGMSWVRQAPGKEPEWVSSISGSGSNTIYADSVKDRFTISRDNAKSTLYLQMNSLKPEDTAVYYCTIGGSLSRSSQGTQVTVSS

SEQ ID NO: 520 - protein, 114 aa, Unknown
  Camelid
QVQLQESGGGLVQPGGSLRLTCTASGFTFSSFGMSWVRQAPGKGLEWVSAISSDSGTKNYADSVKGRFTISRDNAKKMLFLQMNSLRPEDTAVYYCVIGRGSPSSQGTQVTVSS

SEQ ID NO: 521 - protein, 114 aa, Unknown
  Camelid
QVQLQESGGGLVQPGGSLRLTCTASGFTFRSFGMSWVRQAPGKGLEWVSAISADGSDKRYADSVKGRFTISRDNGKKMLTLDMNSLKPEDTAVYYCVIGRGSPASQGTQVTVSS

SEQ ID NO: 522 - protein, 128 aa, Unknown
  Camelid
AVQLVESGGGLVQAGDSLRLSCVVSGTTFSSAAMGWFRQAPGKEREFVGAIKWSGTSTYYTDSVKGRFTISRDNVKNTVYLQMNNLKPEDTGVYTCAADRDRYRDRMGPMTTTDFRFWGQGTQVTVSS

SEQ ID NO: 523 - protein, 124 aa, Unknown
  Camelid
QVKLEESGGGLVQTGGSLRLSCAASGRTFSSFAMGWFRQAPGREREFVASIGSSGITTNYADSVKGRFTISRDNAKNTVYLQMNSLKPEDTGLCYCAVNRYGIPYRSGTQYQNWGQGTQVTVSS

SEQ ID NO: 524 - protein, 120 aa, Unknown
  Camelid
EVQLEESGGGLVQPGGSLRLSCAASGLTFNDYAMGWYRQAPGKERDMVATISIGGRTYYADSVKGRFTISRDNAKNTVYLQMNSLKPEDTAIYYCVAHRQTVVRGPYLLWGQGTQVTVSS

SEQ ID NO: 525 - protein, 123 aa, Unknown
  Camelid
QVQLVESGGKLVQAGGSLRLSCAASGRTFSNYAMGWFRQAPGKEREFVAGSGRSNSYNYYSDSVKGRFTISRDNAKNTVYLQMNSLKPEDTAVYYCAASTNLWPRDRNLYAYWGQGTQVTVSS

SEQ ID NO: 526 - protein, 125 aa, Unknown
  Camelid
EVQLVESGGGLVQAGDSLRLSCAASGRSLGIYRMGWFRQVPGKEREFVAAISWSGGTTRYLDSVKGRFTISRDSTKNAVYLQMNSLKPEDTAVYYCAVDSSGRLYWTLSTSYDYWGQGTQVTVSS

SEQ ID NO: 527 - protein, 125 aa, Unknown
  Camelid
QVQLVEFGGGLVQAGDSLRLSCAASGRSLGIYKMAWFRQVPGKEREFVAAISWSGGTTRYIDSVKGRFTLSRDNTKNMVYLQMNSLKPDDTAVYYCAVDSSGRLYWTLSTSYDYWGQGTQVTVSS

SEQ ID NO: 528 - protein, 124 aa, Unknown
  Camelid
EVQLVESGGGLVQAGGSLSLSCAASGRTFSPYTMGWFRQAPGKEREFLAGVTWSGSSTFYGDSVKGRFTASRDSAKNTVTLEMNSLNPEDTAVYYCAAAYGGGLYRDPRSYDYWGRGTQVTVSS

SEQ ID NO: 529 - protein, 131 aa, Unknown
  Camelid
AVQLVESGGGLVQAGGSLRLSCAASGFTLDAWPIAWFRQAPGKEREGVSCIRDGTTYYADSVKGRFTISSDNANNTVYLQTNSLKPEDTAVYYCAAPSGPATGSSHTFGIYWNLRDDYDNWGQGTQVTVSS

SEQ ID NO: 530 - protein, 126 aa, Unknown
  Camelid
EVQLVESGGGLVQAGGSLRLSCAASGFTFDHYTIGWFRQVPGKEREGVSCISSSDGSTYYADSVKGRFTISSDNAKNTVYLQMNTLEPDDTAVYYCAAGGLLLRVEELQASDYDYWGQGIQVTVSS

SEQ ID NO: 531 - protein, 128 aa, Unknown
  Camelid
AVQLVDSGGGLVQPGGSLRLSCTASGFTLDYYAIGWFRQAPGKEREGVACISNSDGSTYYGDSVKGRFTISRDNAKTTVYLQMNSLKPEDTAVYYCATADRHYSASHHPFADFAFNSWGQGTQVTVSS

SEQ ID NO: 532 - protein, 120 aa, Unknown
  Camelid
EVQLVESGGGLVQAGGSLRLSCAAYGLTFWRAAMAWFRRAPGKERELVVARNWGDGSTRYADSVKGRFTISRDNAKNTVYLQMNSLKPEDTAVYYCAAVRTYGSATYDIWGQGTQVTVSS

SEQ ID NO: 533 - protein, 123 aa, Unknown
  Camelid
EVQLVESGGGLVQDGGSLRLSCIFSGRTFANYAMGWFRQAPGKEREFVAAINRNGGTTNYADALKGRFTISRDNTKNTAFLQMNSLKPDDTAVYYCAAREWPFSTIPSGWRYWGQGTQVTVSS

SEQ ID NO: 534 - protein, 125 aa, Unknown
  Camelid
DVQLVESGGGWVQPGGSLRLSCAASGPTASSHAIGWFRQAPGKEREFVVGINRGGVTRDYADSVKGRFAVSRDNVKNTVYLQMNRLKPEDSAIYICAARPEYSFTAMSKGDMDYWGKGTLVTVSS

SEQ ID NO: 535 - DNA, 324 bp, Homo sapiens
gacatccagatgacccagtctccatcctccctgtctgcatctgtaggagaccgtgtcaccatcacttgccgggcaagtcagtggattggtcctgagttaagttggtaccagcagaaaccagggaaagcccctaagctcctgatctatcatggttccattttgcaaagtggggtcccatcacgtttcagtggcagtggatctgggacagatttcactctcaccatcagcagtctgcaacctgaagattttgctacgtactactgtcaacagtatatgacgtatcctccgacgttcggccaagggaccaaggtggaaatcaaacgg

SEQ ID NO: 536 - DNA, 324 bp, Homo sapiens
gacatccagatgacccagtctccatcctccctgtctgcatccgtaggagaccgtgtcaccatcacttgccgggcaagtcagtttattgggaaggagttacgttggtatcagcagaaaccagggaaagcccctaagctcctgatctatcatcagtccttgttgcaaagtggggtcccatcacgtttcagtggcagtggatctgggacagatttcattctcaccatcagcagtctgcaacctgaagattttgctacgtactactgtcaacagcatatgtataggccttttacgttcggccaagggaccaaggtggaaatcaaacga

SEQ ID NO: 537 - DNA, 348 bp, Homo sapiens
gaggtgcagctgttggagtctgggggaggcttggtacagcctggggggtccctgcgtctctcctgtgcagcctccggattcacctttaaggcttatccgataatgtgggtccgccaggctccagggaagggtctagagtgggtctcagagatttcgccttcgggttctgagacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 538 - DNA, 348 bp, Homo sapiens
gaggtgcagctgttggagtctgggggaggcttggtacagcctggggggtccctgcgtctctcctgcgcagcctccggattcacctttaaggcttatccgataatgtgggtccgccaggctccagggaagggtctagagtgggtctcagagatttcgccttcgggttctcggacatactacgcagactctgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 539 - protein, 116 aa, Homo sapiens
EVQLLESGGGLVQPGGSLRLSCAASGFTFKAYPIMWVRQAPGKGLEWVSEISPSGSETYYADSVKGRFTISRDNSKNTLYLQMNSLRAEDTAVYYCAKDPRKFDYWGQGTLVTVSS

SEQ ID NO: 540 - protein, 116 aa, Homo sapiens
EVQLLESGGGLVQPGGSLRLSCAASGFTFKAYPIMWVRQAPGKGLEWVSEISPSGSRTYYADSVKGRFTISRDNSKNTLYLQMNSLRAEDTAVYYCAKDPRKFDYWGQGTLVTVSS

SEQ ID NO: 541 - protein, 108 aa, Homo sapiens
DIQMTQSPSSLSASVGDRVTITCRASQWIGILLDWYQQKPGKAPRLLIYYASFLREGVPSRFSGSGSGTDFTLTISSLQPEDFATYYCQQANPAPLTFGQGTKVEIKR

SEQ ID NO: 542 - protein, 108 aa, Homo sapiens
DIQMTQSPSSLSASVGDRVTITCRASQWIGILLDWYQQKPGKAPKLLIYYASFLAEGVPSRFSGSGSGTDFTLTISSLQPEDFATYYCQQANPAPLTFGQGTKVEIKR

SEQ ID NO: 543 - protein, 108 aa, Homo sapiens
DIQMTQSPSSLSASVGDRVTITCRASQWIGILLDWYQQKPGKAPKLLIYYASFLNKGVPSRFSGSGSGTDFTLTISSLQPEDFATYYCQQANPAPLTFGQGTKVEIKR

SEQ ID NO: 544 - protein, 108 aa, Homo sapiens
DIQMTQSPSSLSASVGDRVTITCRASQWIGILLDWYQQKPGKAPKLLIYYASFLRPGVPSRFSGSGSGTDFTLTISSLQPEDFATYYCQQANPAPLTFGQGTKVEIKR

SEQ ID NO: 545 - protein, 108 aa, Homo sapiens
DIQMTQSPSSLSASVGDRVTITCRASQWIGILLDWYQQKPGKAPKLLIYYASFLPEGVPSRFSGSGSGTDFTLTISSLQPEDFATYYCQQANPAPLTFGQGTKVEIKR

SEQ ID NO: 546 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPVPTTFGQGTKVEIKR

SEQ ID NO: 547 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPHPTTFGQGTKVEIKR

SEQ ID NO: 548 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPVPPTFGQGTKVEIKR

SEQ ID NO: 549 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPHPPTFGQGTKVEIKR

SEQ ID NO: 550 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPTPPTFGQGTKVEIKR

SEQ ID NO: 551 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPLPPTFGQGTKVEIKR

SEQ ID NO: 552 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPIPPTFGQGTKVEIKR

SEQ ID NO: 553 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQPANPTPPTFGQGTKVEIKR

SEQ ID NO: 554 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPVPVTFGQGTKVEIKR

SEQ ID NO: 555 - protein, 108 aa, Homo sapiens
DIQMTQSPSSLSASVGDRVTITCRASQWIGILLDWYQQKPGKAPKLLIYYASFLNNGVPSRFSGSGSGTDFTLTISNLQPEDFATYYCQQANPAPLTFGQGTKVEIKR

SEQ ID NO: 556 - protein, 108 aa, Homo sapiens
DIQMTQSPSSLSASVGDRVTITCRASQWIGILLDWYQQKPGKAPKLLIYYASFLAPGVPSRFSGSGSGTDFTLTISSLQPEDFATYYCQQANPAPLTFGQGTKVEIKR

SEQ ID NO: 557 - protein, 108 aa, Homo sapiens
DIQMTQSPSSLSASVGDRVTITCRASQWIGILLDWYQQKPGKAPKLLIYYASFLNSGVPSRFSGSGSGTDFTLTISSLQPEDFATYYCQQANPAPLTFGQGTKVEIKR

SEQ ID NO: 558 - protein, 108 aa, Homo sapiens
DIQMTQSPSSLSASVGDRVTITCRASQWIGILLDWYQQKPGKAPELLIYYASFLKQGVPSRFSGSGSGTDFTLTISSLQPEDFATYYCQQANPAPLTFGQGTKVEIKR

SEQ ID NO: 559 - protein, 108 aa, Homo sapiens
DIQMTQSPSSLSASVGDRVTITCRASQWIGILLDWYQQKPGKAPKLLIYYASFLNNGVPSRFSGSGSGTDFTLTISSLQPEDFATYYCQQANPAPLTFGQGTKVEIKR

SEQ ID NO: 560 - protein, 108 aa, Homo sapiens
DIQMTQSPSSLSASVGDRVTITCRASQWIGILLDWYQQKPGKAPKLLIYYASFLSPGVPSRFSGSGSGTDFTLTISSLQPEDFATYYCQQANPAPLTFGQGTKVEIKR

SEQ ID NO: 561 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPTPPTFGQGTKVEIKR

SEQ ID NO: 562 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQPANPTPPTFGQGTKVEIKR

SEQ ID NO: 563 - protein, 108 aa, Homo sapiens
DIQMTQSPSSLSASVGDRVTITCRASQWIGILLDWYQQKPGEAPKLLIYYASFLQSGVPSRFSGSGSGTDFTLTISGLQPEDFATYYCQQANPYPTTFGQGTKVEIKR

SEQ ID NO: 564 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPYPTTFGQGTKVEIKR

SEQ ID NO: 565 - protein, 108 aa, Homo sapiens; identical to SEQ ID NO: 556

SEQ ID NO: 566 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQPANPHPPTFGQGTKVEIKR

SEQ ID NO: 567 - protein, 108 aa, Homo sapiens
DIQMTQSPSSLSASVGDRVTITCRASQWIGILVDWYQQKPGKAPKLLIYYASFLPEGVPSRFSGSGSGTDFTLTISSLQPEDFATYYCQQANPAPLTFGQGTKVEIKR

SEQ ID NO: 568 - protein, 108 aa, Homo sapiens
DIQMTQSPSSLSASVGDRVTITCRASQWIGILVDWYQQKPGKAPKLLIYYASFLQSGVPSRFSGSGSGTDFTLTISSLQPEDFATYYCQQANPVPPTFGQGTKVEIKR

SEQ ID NO: 569 - protein, 108 aa, Homo sapiens
DIQMTQSPSSLSASVGDRVTITCRASQWIGILIDWYQQKPGKAPKLLIYYASFLPEGVPSRFSGSGSGTDFTLTISSLQPEDFATYYCQQANPAPLTFGQGTKVEIKR

SEQ ID NO: 570 - protein, 108 aa, Homo sapiens
DIQMTQSPSSLSASVGDRVTITCRASQWIGILIDWYQQKPGKAPKLLIYYASFLQSGVPSRFSGSGSGTDFTLTISSLQPEDFATYYCQQANPVPPTFGQGTKVEIKR

SEQ ID NO: 571 - protein, 108 aa, Homo sapiens
DIQMTQSPSSLSASVGDRVTITCRASQWIGILLDWYQQKPGKAPKLLIYYASFLPEGVPSRFSGSGSGTDFTLTISSLQPEDFATYYCQQANPVPPTFGQGTKVEIKR

SEQ ID NO: 572 - protein, 108 aa, Homo sapiens
DIQMTQSPSSLSASVGDRVTITCRASQWIGINLDWYQQKPGKAPKLLIYYASFLPEGVPSRFSGSGSGTDFTLTISSLQPEDFATYYCQQANPAPLTFGQGTKVEIKR

SEQ ID NO: 573 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCQQANPVPPTFGQGTKVEIKR

SEQ ID NO: 574 - protein, 108 aa, Homo sapiens
DIQMTQSPSSLSASVGDRVTITCRASQWIGILVDWYQQKPGKAPKLLIYYASFLPEGVPSRFSGSGSGTDFTLTISSLQPEDFATYYCQQANPVPPTFGQGTKVEIKR

SEQ ID NO: 575 - protein, 108 aa, Homo sapiens
DIQMTQSPSSLSASVGDRVTITCRASQWIGILIDWYQQKPGKAPKLLIYYASFLPEGVPSRFSGSGSGTDFTLTISSLQPEDFATYYCQQANPVPPTFGQGTKVEIKR

SEQ ID NO: 576 - protein, 108 aa, Homo sapiens
DIQMTQSPSSLSASVGDRVTITCRASQWIGINLDWYQQKPGKAPKLLIYYASFLPEGVPSRFSGSGSGTDFTLTISSLQPEDFATYYCQQANPVPPTFGQGTKVEIKR

SEQ ID NO: 577 - protein, 108 aa, Homo sapiens; identical to SEQ ID NO: 573

SEQ ID NO: 578 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCLQANPVPPTFGQGTKVEIKR

SEQ ID NO: 579 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCGQANPVPPTFGQGTKVEIKR

SEQ ID NO: 580 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCQQANPVPPGFGQGTKVEIKR

SEQ ID NO: 581 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCQQANPVPVTFGQGTKVEIKR

SEQ ID NO: 582 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCQQANPVPTTFGQGTKVEIKR

SEQ ID NO: 583 - protein, 108 aa, Homo sapiens
DIQMTQSPSSLSASVGDRVTITCRASQWIGINLDWYQQKPGKAPKLLIYYASFLQSGVPSRFSGSGSGTDFTLTISSLQPEDFATYYCQQANPYPTTFGQGTKVEIKR

SEQ ID NO: 584 - protein, 108 aa, Homo sapiens
DIQMTQSPSSLSASVGDRVTITCRASQWIGNLLDWYQQKPGKAPKLLIYYASFLQSGVPSRFSGSGSGTDFTLTISSLQPEDFATYYCQQANPAPLTFGQGTKVEIKR

SEQ ID NO: 585 - protein, 108 aa, Homo sapiens
DIQMTQSPSSLSASVGDRVTITCRASQWIGNLLDWYQQKPGKAPKLLIYYASFLQSGVPSRFSGSGFGTDFTLTISSLQPEDFATYYCQQANPAPLTFGQGTKVEIKR

SEQ ID NO: 586 - protein, 108 aa, Homo sapiens
DIQMTQSPSSLSASVGDRVTITCRASQWIGNLLDWYQQKPGKAPKLLIYYASFLQSGVPSRFSGSGYGTDFTLTISSLQPEDFATYYCQQANPAPLTFGQGTKVEIKR

SEQ ID NO: 587 - protein, 108 aa, Homo sapiens
DIQMTQSPSSLSASVGDRVTITCRASQWIGNLLDWYQQKPGKAPKLLIYYASFLQSGVPSRFSGSGWGTDFTLTISSLQPEDFATYYCQQANPAPLTFGQGTKVEIKR

SEQ ID NO: 588 - protein, 108 aa, Homo sapiens
DIQMTQSPSSLSASVGDRVTITCRASQWIGNLLDWYQQKPGKAPKLLIYYASFLQSGVPSRFSGSGRGTDFTLTISSLQPEDFATYYCQQANPAPLTFGQGTKVEIKR

SEQ ID NO: 589 - protein, 108 aa, Homo sapiens
DIQMTQSPSSLSASVGDRVTITCRASQWIGNLLDWYQQKPGKAPKLLIYYASFLQSGVPSRFSGSGEGTDFTLTISSLQPEDFATYYCQQANPAPLTFGQGTKVEIKR

SEQ ID NO: 590 - protein, 108 aa, Homo sapiens; identical to SEQ ID NO: 447

SEQ ID NO: 591 - protein, 108 aa, Homo sapiens
DIQMTQSPSSLSASVGDRVTITCRASQWIGINLDWYQQKPGKAPKLLIYYASFLQSGVPSRFSGSGFGTDFTLTISSLQPEDFATYYCQQANPAPLTFGQGTKVEIKR

SEQ ID NO: 592 - protein, 108 aa, Homo sapiens
DIQMTQSPSSLSASVGDRVTITCRASQWIGINLDWYQQKPGKAPKLLIYYASFLQSGVPSRFSGSGYGTDFTLTISSLQPEDFATYYCQQANPAPLTFGQGTKVEIKR

SEQ ID NO: 593 - protein, 108 aa, Homo sapiens
DIQMTQSPSSLSASVGDRVTITCRASQWIGINLDWYQQKPGKAPKLLIYYASFLQSGVPSRFSGSGWGTDFTLTISSLQPEDFATYYCQQANPAPLTFGQGTKVEIKR

SEQ ID NO: 594 - protein, 108 aa, Homo sapiens
DIQMTQSPSSLSASVGDRVTITCRASQWIGINLDWYQQKPGKAPKLLIYYASFLQSGVPSRFSGSGRGTDFTLTISSLQPEDFATYYCQQANPAPLTFGQGTKVEIKR

SEQ ID NO: 595 - protein, 108 aa, Homo sapiens
DIQMTQSPSSLSASVGDRVTITCRASQWIGINLDWYQQKPGKAPKLLIYYASFLQSGVPSRFSGSGEGTDFTLTISSLQPEDFATYYCQQANPAPLTFGQGTKVEIKR

SEQ ID NO: 596 - protein, 108 aa, Homo sapiens
DIQMTQSPSSLSASVGDRVTITCRASQYIGNLLDWYQQKPGKAPKLLIYYASFLQSGVPSRFSGSGYGTDFTLTISSLQPEDFATYYCQQANPAPLTFGQGTKVEIKR

SEQ ID NO: 597 - protein, 108 aa, Homo sapiens
DIQMTQSPSSLSASVGDRVTITCRASQHIGNLLDWYQQKPGKAPKLLIYYASFLQSGVPSRFSGSGYGTDFTLTISSLQPEDFATYYCQQANPAPLTFGQGTKVEIKR

SEQ ID NO: 598 - protein, 108 aa, Homo sapiens
DIQMTQSPSSLSASVGDRVTITCRASQQIGNLLDWYQQKPGKAPKLLIYYASFLQSGVPSRFSGSGYGTDFTLTISSLQPEDFATYYCQQANPAPLTFGQGTKVEIKR

SEQ ID NO: 599 - protein, 108 aa, Homo sapiens
DIQMTQSPSSLSASVGDRVTITCRASQNIGNLLDWYQQKPGKAPKLLIYYASFLQSGVPSRFSGSGYGTDFTLTISSLQPEDFATYYCQQANPAPLTFGQGTKVEIKR

SEQ ID NO: 600 - protein, 108 aa, Homo sapiens
DIQMTQSPSSLSASVGDRVTITCRASQYIGINLDWYQQKPGKAPKLLIYYASFLQSGVPSRFSGSGYGTDFTLTISSLQPEDFATYYCQQANPAPLTFGQGTKVEIKR

SEQ ID NO: 601 - protein, 108 aa, Homo sapiens
DIQMTQSPSSLSASVGDRVTITCRASQHIGINLDWYQQKPGKAPKLLIYYASFLQSGVPSRFSGSGYGTDFTLTISSLQPEDFATYYCQQANPAPLTFGQGTKVEIKR

SEQ ID NO: 602 - protein, 108 aa, Homo sapiens
DIQMTQSPSSLSASVGDRVTITCRASQQIGINLDWYQQKPGKAPKLLIYYASFLQSGVPSRFSGSGYGTDFTLTISSLQPEDFATYYCQQANPAPLTFGQGTKVEIKR

SEQ ID NO: 603 - protein, 108 aa, Homo sapiens
DIQMTQSPSSLSASVGDRVTITCRASQNIGINLDWYQQKPGKAPKLLIYYASFLQSGVPSRFSGSGYGTDFTLTISSLQPEDFATYYCQQANPAPLTFGQGTKVEIKR

SEQ ID NO: 604 - protein, 108 aa, Homo sapiens
DIQMTQSPSSLSASVGDRVTITCRASQWIGNNLDWYQQKPGKAPKLLIYYASFLQSGVPSRFSGSGYGTDFTLTISSLQPEDFATYYCQQANPAPLTFGQGTKVEIKR

SEQ ID NO: 605 - protein, 108 aa, Homo sapiens
DIQMTQSPSSLSASVGDRVTITCRASQWIGTLLDWYQQKPGKAPKLLIYYASFLQSGVPSRFSGSGYGTDFTLTISSLQPEDFATYYCQQANPAPLTFGQGTKVEIKR

SEQ ID NO: 606 - protein, 108 aa, Homo sapiens
DIQMTQSPSSLSASVGDRVTITCRASQWIGIQLDWYQQKPGKAPKLLIYYASFLQSGVPSRFSGSGYGTDFTLTISSLQPEDFATYYCQQANPAPLTFGQGTKVEIKR

SEQ ID NO: 607 - protein, 108 aa, Homo sapiens
DIQMTQSPSSLSASVGDRVTITCRASQWIGITLDWYQQKPGKAPKLLIYYASFLQSGVPSRFSGSGYGTDFTLTISSLQPEDFATYYCQQANPAPLTFGQGTKVEIKR

SEQ ID NO: 608 - protein, 108 aa, Homo sapiens
DIQMTQSPSSLSASVGDRVTITCRASQWIGNLLDWYQQKPGKAPKLLIYYASFLQSGVPSRFSGSGYGTDFTLTISSLQPEDFATYYCQQANPVPPTFGQGTKVEIKR

SEQ ID NO: 609 - protein, 108 aa, Homo sapiens
DIQMTQSPSSLSASVGDRVTITCRASQYIGNLLDWYQQKPGKAPKLLIYYASFLQSGVPSRFSGSGYGTDFTLTISSLQPEDFATYYCQQANPVPPTFGQGTKVEIKR

SEQ ID NO: 610 - protein, 108 aa, Homo sapiens
DIQMTQSPSSLSASVGDRVTITCRASQHIGNLLDWYQQKPGKAPKLLIYYASFLQSGVPSRFSGSGYGTDFTLTISSLQPEDFATYYCQQANPVPPTFGQGTKVEIKR

SEQ ID NO: 611 - protein, 108 aa, Homo sapiens
DIQMTQSPSSLSASVGDRVTITCRASQWIGINLDWYQQKPGKAPKLLIYYASFLQSGVPSRFSGSGYGTDFTLTISSLQPEDFATYYCQQANPVPPTFGQGTKVEIKR

SEQ ID NO: 612 - protein, 108 aa, Homo sapiens
DIQMTQSPSSLSASVGDRVTITCRASQYIGINLDWYQQKPGKAPKLLIYYASFLQSGVPSRFSGSGYGTDFTLTISSLQPEDFATYYCQQANPVPPTFGQGTKVEIKR

SEQ ID NO: 613 - protein, 108 aa, Homo sapiens
DIQMTQSPSSLSASVGDRVTITCRASQWIGNNLDWYQQKPGKAPKLLIYYASFLQSGVPSRFSGSGYGTDFTLTISSLQPEDFATYYCQQANPVPPTFGQGTKVEIKR

SEQ ID NO: 614 - protein, 108 aa, Homo sapiens
DIQMTQSPSSLSASVGDRVTITCRASQWIGNLLDWYQQKPGKAPKLLIYYASFLQSGVPSRFSGSGYGTDFTLTISSLQPEDFATYYCQQANPTPPTFGQGTKVEIKR

SEQ ID NO: 615 - protein, 108 aa, Homo sapiens
DIQMTQSPSSLSASVGDRVTITCRASQYIGNLLDWYQQKPGKAPKLLIYYASFLQSGVPSRFSGSGYGTDFTLTISSLQPEDFATYYCQQANPTPPTFGQGTKVEIKR

SEQ ID NO: 616 - protein, 108 aa, Homo sapiens
DIQMTQSPSSLSASVGDRVTITCRASQHIGNLLDWYQQKPGKAPKLLIYYASFLQSGVPSRFSGSGYGTDFTLTISSLQPEDFATYYCQQANPTPPTFGQGTKVEIKR

SEQ ID NO: 617 - protein, 108 aa, Homo sapiens
DIQMTQSPSSLSASVGDRVTITCRASQWIGINLDWYQQKPGKAPKLLIYYASFLQSGVPSRFSGSGYGTDFTLTISSLQPEDFATYYCQQANPTPPTFGQGTKVEIKR

SEQ ID NO: 618 - protein, 108 aa, Homo sapiens
DIQMTQSPSSLSASVGDRVTITCRASQYIGINLDWYQQKPGKAPKLLIYYASFLQSGVPSRFSGSGYGTDFTLTISSLQPEDFATYYCQQANPTPPTFGQGTKVEIKR

SEQ ID NO: 619 - protein, 108 aa, Homo sapiens
DIQMTQSPSSLSASVGDRVTITCRASQHIGINLDWYQQKPGKAPKLLIYYASFLQSGVPSRFSGSGYGTDFTLTISSLQPEDFATYYCQQANPTPPTFGQGTKVEIKR

SEQ ID NO: 620 - protein, 108 aa, Homo sapiens
DIQMTQSPSSLSASVGDRVTITCRASQWIGNNLDWYQQKPGKAPKLLIYYASFLQSGVPSRFSGSGYGTDFTLTISSLQPEDFATYYCQQANPTPPTFGQGTKVEIKR

SEQ ID NO: 621 - protein, 108 aa, Homo sapiens
DIQMTQSPSSLSASVGDRVTITCRASQWIGNLLDWYQQKPGKAPKLLIYYASFLQSGVPSRFSGSGFGTDFTLTISSLQPEDFATYYCQQANPVPPTFGQGTKVEIKR

SEQ ID NO: 622 - protein, 108 aa, Homo sapiens
DIQMTQSPSSLSASVGDRVTITCRASQWIGNLLDWYQQKPGKAPKLLIYYASFLPEGVPSRFSGSGFGTDFTLTISSLQPEDFATYYCQQANPVPPTFGQGTKVEIKR

SEQ ID NO: 623 - DNA, 324 bp, Homo sapiens
gacatccagatgacccagtctccatcctccctgtctgcatctgtaggagaccgtgtcaccatcacttgccgggctagtcagtggattggtattttgttagattggtaccagcagaaaccagggaaagcccctaggctcctgatctattatgcttccttcttgagggagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 624 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttggcggagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 625 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccccaccctcccacgttcggccaagggaccaaggtggaaatcaaacgg

SEQ ID NO: 626 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatcccacccctcccacgttcggccaagggaccaaggtggaaatcaaacgg

SEQ ID NO: 627 - DNA, 324 bp, Homo sapiens
gacatccagatgacccagtctccatcctccctgtctgcatctgtaggagaccgtgtcaccatcacttgccgggcaagtcagtggattggtattttgttagattggtatcagcagaaaccagggaaagcccctaagctcctgatctattatgcttccttcttgaacaagggggtcccatcacgtttcagtggcagtggatctgggacagatttcactctcaccatcagcagtctgcaacctgaagattttgctacgtactactgtcaacaggctaatccggcgcctctgacgttcggccaaggaaccaaggtggaaatcaaacgg

SEQ ID NO: 628 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttgaggcccggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 629 - DNA, 324 bp, Homo sapiens
gacatccagatgacccagtctccatcctccctgtctgcatctgtaggagaccgtgtcaccatcacttgccgggcaagtcagtggattggtattttgttagattggtatcagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 630 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccgcaccctaccacgttcggccaagggaccaaggtggaaatcaaacgg

SEQ ID NO: 631 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagatttcgctacgtactactgtcaacaggctaatcccgtccctaccacgttcggccaagggaccaaggtggaaatcaaacgg

SEQ ID NO: 632 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 633 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccgctgcctcccacgttcggccaagggaccaaggtggaaatcaaacgg

SEQ ID NO: 634 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatcccatccctcccacgttcggccaagggaccaaggtggaaatcaaacgg

SEQ ID NO: 635 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaaccggctaatcccacccctcccacgttcggccaagggaccaaggtggaaatcaaacgg

SEQ ID NO: 636 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatcccgtccctgtcacgttcggccaagggaccaaggtggaaatcaaacgg

SEQ ID NO: 637 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttgaacaacggggtcccatcacgtttcagtggcagtggatctgggacagatttcactctcaccatcagcaatctgcaacctgaagattttgctacgtactactgtcaacaggctaatccggcgcctctgacgttcggccaagggaccaaggtggaaatcaaacgg

SEQ ID NO: 638 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttggcgcccggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 639 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttgaactccggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 640 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctgagctcctgatctattatgcttccttcttgaagcagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 641 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttgaacaacggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 642 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttccttcttgtcgcccggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 643 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatcccacccctcccacgttcggccaagggaccaaggtggaaatcaaacgg

SEQ ID NO: 644 - DNA, 324 bp, Homo sapiens
gacatccagatgacccagtctccatcctccctgtctgcatctgtaggagaccgtgtcaccatcacttgccgggcaagtcagtggattggtattttgttagattggtatcagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaaccggctaatcccacccctcccacgttcggccaagggaccaaggtggaaatcaaacgg

SEQ ID NO: 645 - DNA, 324 bp, Homo sapiens
gacatccagatgacccagtctccatcctccctgtctgcatctgtaggagaccgtgtcaccatcacttgccgggcaagtcagtggattggtattttggtagattggtaccagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 646 - DNA, 324 bp, Homo sapiens
gacatccagatgacccagtctccatcctccctgtctgcatctgtaggagaccgtgtcaccatcacttgccgggcaagtcagtggattggtattttgatagattggtaccagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 647 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcggtctgcaacctgaagattttgctacgtactactgtcaacaggctaatccctaccctaccacgttcggccaagggaccaaggtggaaatcaaacgg

SEQ ID NO: 648 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccaggggaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccctaccctaccacgttcggccaagggaccaaggtggaaatcaaacgg

SEQ ID NO: 649 - DNA, 324 bp, Homo sapiens
gacatccagatgactcagtctccatcctccctgtctgcatctgtaggagaccgtgtcaccatcacttgccgggcaagtcagtggattggtattttgttagattggtatcagcagaaaccagggaaagcccctaagctcctgatctattatgcttccttcttggcgcccggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 650 - DNA, 324 bp, Homo sapiens
gacatccagatgacccagtctccatcctccctgtctgcatctgtaggagaccgtgtcaccatcacttgccgggcaagtcagtggattggtattttgttagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaaccggctaatccccaccctcccacgttcggccaagggaccaaggtggaaatcaaacgg

SEQ ID NO: 651 - DNA, 324 bp, Homo sapiens
gacatccagatgacccagtctccatcctccctgtctgcatctgtaggagaccgtgtcaccatcacttgccgggcaagtcagtggattggtattttgatagattggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 652 - DNA, 324 bp, Homo sapiens
gacatccagatgacccagtctccatcctccctgtctgcatctgtaggagaccgtgtcaccatcacttgccgggcaagtcagtggattggtattttgttagattggtatcagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 653 - DNA, 324 bp, Homo sapiens
gacatccagatgacccagtctccatcctccctgtctgcatctgtaggagaccgtgtcaccatcacttgccgggcaagtcagtggattggtatcaacttagactggtaccagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggcgcctctgacgttcggccaagggaccaaggtggaaatcaaacgg

SEQ ID NO: 654 - DNA, 324 bp, Homo sapiens
gacatccagatgacccagtctccatcctccctgtctgcatctgtaggagaccgtgtcaccatcacttgccgggcaagtcagtggattggtatcaacttagactggtaccagcagaaaccagggaaagcccctaagctcctgatctattatgcttcctttttgcaaagtggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 655 - DNA, 324 bp, Homo sapiens
gacatccagatgacccagtctccatcctccctgtctgcatctgtaggagaccgtgtcaccatcacttgccgggcaagtcagtggattggtattttggtagattggtaccagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 656 - DNA, 324 bp, Homo sapiens
gacatccagatgacccagtctccatcctccctgtctgcatctgtaggagaccgtgtcaccatcacttgccgggcaagtcagtggattggtattttgatagattggtaccagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 657 - DNA, 324 bp, Homo sapiens
gacatccagatgacccagtctccatcctccctgtctgcatctgtaggagaccgtgtcaccatcacttgccgggcaagtcagtggattggtatcaacttagactggtaccagcagaaaccagggaaagcccctaagctcctgatctattatgcttccttcttgccggagggggtcccatcacgtttcagtggcagtggatctgggacagatttcactctcaccatcagcagtctgcaacctgaagattttgctacgtactactgtcaacaggctaatccggtccctcccacgttcggccaagggaccaaggtggaaatcaaacgg

SEQ ID NO: 658 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 659 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgcctgcaggccaaccctgtgcccccgaccttcggccagggtaccaaggtggagatcaagcgt

SEQ ID NO: 660 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgcgggcaggccaaccctgtgcccccgaccttcggccagggtaccaaggtggagatcaagcgt

SEQ ID NO: 661 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaaccctgtgccccctggcttcggccagggtaccaaggtggagatcaagcgt

SEQ ID NO: 662 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaaccctgtgcccaccaccttcggccagggtaccaaggtggagatcaagcgt

SEQ ID NO: 663 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaacccttaccccaccaccttcggccagggtaccaaggtggagatcaagcgt

SEQ ID NO: 664 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaaccctgtgcccgtgaccttcggccagggtaccaaggtggagatcaagcgt

SEQ ID NO: 665 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 666 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggcttcggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 667 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 668 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggctggggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 669 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggccggggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 670 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggcgagggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 671 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctccggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 672 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggcttcggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 673 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 674 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctggggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 675 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggccggggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 676 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggcgagggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 677 - DNA, 324 bp, Homo sapiens
gatatccagatgacccagagcccttcaagcttgagcgccagcgtgggcgacagagtgaccatcacctgccgggccagccagtacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 678 - DNA, 324 bp, Homo sapiens
gatatccagatgacccagagcccttcaagcttgagcgccagcgtgggcgacagagtgaccatcacctgccgggccagccagcacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 679 - DNA, 324 bp, Homo sapiens
gatatccagatgacccagagcccttcaagcttgagcgccagcgtgggcgacagagtgaccatcacctgccgggccagccagcagatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 680 - DNA, 324 bp, Homo sapiens
gatatccagatgacccagagcccttcaagcttgagcgccagcgtgggcgacagagtgaccatcacctgccgggccagccagaacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 681 - DNA, 324 bp, Homo sapiens
gatatccagatgacccagagcccttcaagcttgagcgccagcgtgggcgacagagtgaccatcacctgccgggccagccagtacatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 682 - DNA, 324 bp, Homo sapiens
gatatccagatgacccagagcccttcaagcttgagcgccagcgtgggcgacagagtgaccatcacctgccgggccagccagcacatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 683 - DNA, 324 bp, Homo sapiens
gatatccagatgacccagagcccttcaagcttgagcgccagcgtgggcgacagagtgaccatcacctgccgggccagccagcagatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 684 - DNA, 324 bp, Homo sapiens
gatatccagatgacccagagcccttcaagcttgagcgccagcgtgggcgacagagtgaccatcacctgccgggccagccagaacatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 685 - DNA, 324 bp, Homo sapiens
gatatccagatgacccagagcccttcaagcttgagcgccagcgtgggcgacagagtgaccatcacctgccgggccagccagtggatcggcaacaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 686 - DNA, 324 bp, Homo sapiens
gatatccagatgacccagagcccttcaagcttgagcgccagcgtgggcgacagagtgaccatcacctgccgggccagccagtggatcggcaccctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 687 - DNA, 324 bp, Homo sapiens
gatatccagatgacccagagcccttcaagcttgagcgccagcgtgggcgacagagtgaccatcacctgccgggccagccagtggatcggcattcagctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 688 - DNA, 324 bp, Homo sapiens
gatatccagatgacccagagcccttcaagcttgagcgccagcgtgggcgacagagtgaccatcacctgccgggccagccagtggatcggcattaccctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccagggtaccaaggtggagatcaagcgt

SEQ ID NO: 689 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 690 - DNA, 324 bp, Homo sapiens
gatatccagatgacccagagcccttcaagcttgagcgccagcgtgggcgacagagtgaccatcacctgccgggccagccagtacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 691 - DNA, 324 bp, Homo sapiens
gatatccagatgacccagagcccttcaagcttgagcgccagcgtgggcgacagagtgaccatcacctgccgggccagccagcacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 692 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 693 - DNA, 324 bp, Homo sapiens
gatatccagatgacccagagcccttcaagcttgagcgccagcgtgggcgacagagtgaccatcacctgccgggccagccagtacatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 694 - DNA, 324 bp, Homo sapiens
gatatccagatgacccagagcccttcaagcttgagcgccagcgtgggcgacagagtgaccatcacctgccgggccagccagtggatcggcaacaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgtgccccctaccttcggccagggtaccaaggtggagatcaagcgt

SEQ ID NO: 695 - DNA, 324 bp, Homo sapiens
gatatccagatgacccagagcccttcaagcttgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 696 - DNA, 324 bp, Homo sapiens
gatatccagatgacccagagcccttcaagcttgagcgccagcgtgggcgacagagtgaccatcacctgccgggccagccagtacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 697 - DNA, 324 bp, Homo sapiens
gatatccagatgacccagagcccttcaagcttgagcgccagcgtgggcgacagagtgaccatcacctgccgggccagccagcacatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 698 - DNA, 324 bp, Homo sapiens
gatatccagatgacccagagcccttcaagcttgagcgccagcgtgggcgacagagtgaccatcacctgccgggccagccagtggatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 699 - DNA, 324 bp, Homo sapiens
gatatccagatgacccagagcccttcaagcttgagcgccagcgtgggcgacagagtgaccatcacctgccgggccagccagtacatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 700 - DNA, 324 bp, Homo sapiens
gatatccagatgacccagagcccttcaagcttgagcgccagcgtgggcgacagagtgaccatcacctgccgggccagccagcacatcggcattaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 701 - DNA, 324 bp, Homo sapiens
gatatccagatgacccagagcccttcaagcttgagcgccagcgtgggcgacagagtgaccatcacctgccgggccagccagtggatcggcaacaacctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctaccccccctaccttcggccagggtaccaaggtggagatcaagcgt

SEQ ID NO: 702 - DNA, 324 bp, Homo sapiens
gatattcaaatgacccagagcccttcaagcttgagcgccagcgtgggcgacagagtgaccatcacctgccgggccagccagtggattggcaacctgctggactggtatcagcagaagcccggcaaggcccccaagctgctgatctactacgccagcttcctgcagagcggcgtgcccagccggtttagcggcagcggcttcggcaccgacttcaccctgaccatcagcagcctgcagcccgaggacttcgccacctactactgccagcaggccaaccctgtgccccctaccttcggccaggggaccaaggtggaaatcaaacgg

SEQ ID NO: 703 - DNA, 324 bp, Homo sapiens
gacatccagatgacccagtctccttcaagcttgagcgccagcgtgggcgacagagtgaccatcacctgccgggccagccagtggattggcaacctgctggactggtatcagcagaagcccggcaaggcccccaagctgctgatctactacgccagcttcctgccggagggcgtgcccagccggtttagcggcagcggcttcggcaccgacttcaccctgaccatcagcagcctgcagcccgaggacttcgccacctactactgccagcaggccaaccctgtgccccctaccttcggccaggggaccaaggtggaaatcaaacgg

SEQ ID NO: 704 - protein, 116 aa, Homo sapiens
EVQLLESGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 705 - DNA, 348 bp, Homo sapiens
gaggtgcagctgttggagtctgggggaggcttggtacagcctggggggtccctgcgtctctcctgtgcagcctccggattcacctttaaggcttatccgatgatgtgggtccgccaggctccagggaagggtctagagtgggtctcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagtttgactactggggtcagggaaccctggtcaccgtctcgagc

SEQ ID NO: 706 - protein, 12 aa, Homo sapiens
KVEIKRTVAAPS

SEQ ID NO: 707 - protein, 9 aa, Artificial Sequence
  Linker
LVTVSSAST

SEQ ID NO: 708 - protein, 15 aa, Artificial Sequence
  Linker
LVTVSSGGGGSGGGS

SEQ ID NO: 709 - protein, 16 aa, Homo sapiens
GQGTKVEIKRTVAAPS

SEQ ID NO: 710 - protein, 16 aa, Artificial Sequence
  Linker
GQGTNVEINRTVAAPS

SEQ ID NO: 711 - protein, 16 aa, Artificial Sequence
  Linker
GQGTNVEINQTVAAPS

SEQ ID NO: 712 - protein, 16 aa, Artificial Sequence
  Linker
GQGTNVEIQRTVAAPS

SEQ ID NO: 713 - protein, 16 aa, Artificial Sequence
  Linker
GQGTLVTVSSTVAAPS

SEQ ID NO: 714 - protein, 6 aa, Artificial Sequence
  Linker
TVAAPS

SEQ ID NO: 715 - protein, 454 aa, Artificial Sequence
  Dual specific fusion
DIQMTQSPSSLPASVGDRVTITCRASQWIGPELSWYQQKPGKAPKLLIYHTSILQSGVPSRFSGSGSGTDFTLTISSLQPEDFATYYCQQYMFQPRTFGQGTKVEIKRTVAAPSDIQMTQSPSSLSASVGDRVTITCRASQWIGINLDWYQQKPGKAPKLLIYYASFLQSGVPSRFSGSGSGTDFTLTISSLQPEDFATYYCQQANPAPLTFGQGTKVEIKRTVAAPSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 716 - protein, 454 aa, Artificial Sequence
  Dual specific fusion
DIQMTQSPSSLPASVGDRVTITCRASQWIGINLDWYQQKPGKAPKLLIYYASFLQSGVPSRFSGSGSGTDFTLTISSLQPEDFATYYCQQANPAPLTFGQGTKVEIKRTVAAPSDIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIKRTVAAPSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 717 - protein, 463 aa, Artificial Sequence [sequence omitted]

SEQ ID NO: 718 - protein, 463 aa, Artificial Sequence
  Dual specific fusion
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRKFDYWGQGTLVTVSSASTKGPSDIQMTQSPSSLSASVGDRVTITCRASQWIGINLDWYQQKPGKAPKLLIYYASFLQSGVPSRFSGSGSGTDFTLTISSLQPEDFATYYCQQANPAPLTFGQGTKVEIKRTVAAPSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 719 - protein, 222 aa, Artificial Sequence
  Dual specific fusion
DIQMTQSPSSLPASVGDRVTITCRASQWIGPELSWYQQKPGKAPKLLIYHTSILQSGVPSRFSGSGSGTDFTLTISSLQPEDFATYYCQQYMFQPRTFGQGTKVEIKRTVAAPSDIQMTQSPSSLSASVGDRVTITCRASQWIGINLDWYQQKPGKAPKLLIYYASFLQSGVPSRFSGSGSGTDFTLTISSLQPEDFATYYCQQANPAPLTFGQGTKVEIKR

SEQ ID NO: 720 - protein, 222 aa, Artificial Sequence
  Dual specific fusion
DIQMTQSPSSLPASVGDRVTITCRASQWIGINLDWYQQKPGKAPKLLIYYASFLQSGVPSRFSGSGSGTDFTLTISSLQPEDFATYYCQQANPAPLTFGQGTKVEIKRTVAAPSDIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIKR

SEQ ID NO: 721 - protein, 230 aa, Artificial Sequence
  Dual specific fusion
DIQMTQSPSSLSASVGDRVTITCRASQWIGINLDWYQQKPGKAPKLLIYYASFLQSGVPSRFSGSGSGTDFTLTISSLQPEDFATYYCQQANPAPLTFGQGTKVEIKRTVAAPSEVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 722 - protein, 231 aa, Artificial Sequence
  Dual specific fusion
EVQLLESGGGLVQPGGSLRLSCAASGFTFKAYPIMWVRQAPGKGLEWVSEISPSGSYTYYADSVKGRFTISRDNSKNTLYLQMNSLRAEDTAVYYCAKDPRKFDYWGQGTLVTVSSASTKGPSDIQMTQSPSSLSASVGDRVTITCRASQWIGINLDWYQQKPGKAPKLLIYYASFLQSGVPSRFSGSGSGTDFTLTISSLQPEDFATYYCQQANPAPLTFGQGTKVEIKR

SEQ ID NO: 723 - protein, 7 aa, Artificial Sequence
  Linker
ASTKGPS

SEQ ID NO: 724 - protein, 13 aa, Artificial Sequence
  Linker
LVTVSSASTKGPS

SEQ ID NO: 725 - protein, 108 aa, Homo sapiens
DIQMTQSPSSLSASVGDRVTITCRASQWIGILVDWYQQKPGKAPKLLIYYASFLQSGVPSRFSGSGFGTDFTLTISSLHPEDFATYYCQQANPAPLTFGQGTKVEIKR

SEQ ID NO: 726 - protein, 108 aa, Homo sapiens
DIQMTQSPTSLSASVGDRVTITCRASQWIGNLLDWYQQKPGKAPKLLIYYASFLQSGVPSRFSGGGFGTDFTLTISSLQPEDFATYYCQQANPAPLTFGQGTKVEIKR

SEQ ID NO: 727 - DNA, 324 bp, Homo sapiens
gacatccagatgacccagtctccatcctccctgtctgcatctgtgggagaccgtgtcaccatcacttgccgggcaagtcagtggattggtattttggtagattggtaccagcagaaaccagggaaagcccctaagctcctgatctattatgcttcctttttgcaaagtggggtcccatcacgtttcagcggcagtggatttgggacagatttcactctcaccatcagcagtctgcatcctgaagattttgctacgtactactgtcaacaggctaatccggcgcctctgacgttcggccaagggaccaaggtggaaatcaaacgt

SEQ ID NO: 728 - DNA, 324 bp, Homo sapiens
gacatccagatgacccagtctccaacctccttgtctgcatctgtaggagaccgtgtcaccatcacttgccgggcaagtcagtggattggtaatttgttagattggtaccagcagaaaccagggaaagcccctaagctcctgatctattatgcttcctttttgcaaagtggggtcccatcacgtttcagtggcggtggatttgggacagatttcactctcaccatcagcagcctgcaacctgaagattttgctacgtactactgtcaacaggctaatccggcgcctctgacgttcggccaagggaccaaggtggaaatcaaacgt